(19) United States
(12) Patent Application Publication
(10) Pub. No.: US 2024/0366582 A1
(43) Pub. Date: Nov. 7, 2024

(54) TREATMENT OF RAYNAUD'S DISEASE

(71) Applicant: Aisa Pharma, Inc., Boston, MA (US)

(72) Inventor: Andrew Sternlicht, Boston, MA (US)

(21) Appl. No.: 18/611,280

(22) Filed: Mar. 20, 2024

Related U.S. Application Data
(63) Continuation of application No. 17/535,106, filed on Nov. 24, 2021, now abandoned.
(60) Provisional application No. 63/118,368, filed on Nov. 25, 2020.

(51) Int. Cl. A61K 31/445; A61K 31/4985; A61K 45/06; A61P 7/00

(52) U.S. Cl. CPC A61K 31/445; A61K 31/4985; A61K 45/06; A61P 7/00

(57) ABSTRACT

Disclosed herein are methods of treating Raynaud's syndrome (e.g., secondary Raynaud's syndrome) in a subject in need thereof, using a dual N-type and L-type calcium channel blocker selective for the N-type calcium channel and/or a phosphodiesterase type 5 inhibitor.

### CROSS-REFERENCE TO RELATED APPLICATION

This application is a continuation of U.S. application Ser. No. 17/535,106, filed on Nov. 24, 2021, which claims priority to U.S. Provisional Application No. 63/118,368, filed on Nov. 25, 2020, the contents of which are incorporated by reference herein in their entirety.

### BACKGROUND

Raynaud's disease (also referred to herein as “Raynaud's syndrome”, “primary Raynaud's disease”, or “Raynaud's phenomenon” is a medical condition that is characterized by episodic and periodic reduction of blood flow to, e.g., the extremities, causing pain, numbness, discoloration, burning sensation, and neuropathic pain. Discoloration of the skin whose blood supply is reduced can accompany these symptoms. Tissue ischemia from reduced blood flow, as well as reperfusion when vasoconstriction ceases, produces, painful burning sensations which can be experienced by the subject during the ischemic attack as well as when blood flow is reestablished. Symptoms of Raynaud's phenomenon can be experienced after, e.g., changes in temperature (cold or hot) in body tissues and/or the experience of strong emotions (e.g., stress) by the subject. In some severe cases, symptoms can progress to digital ulceration and/or gangrene. It is estimated that about 6% of the population is afflicted with Raynaud's syndrome See, e.g., https://www.raynauds.org/which is incorporated by reference herein in its entirety.

While Raynaud's disease occurs as an isolated and idiopathic condition, known as primary Raynaud's, secondary Raynaud's disease occurs as a manifestation and in conjunction with certain other disorders such as systemic sclerosis (also referred to herein as “scleroderma” or “SSc”), as well as lupus, rheumatoid arthritis, and a number of other diseases and conditions. Early diagnosis of SSc can be difficult to detect. Early symptoms can include painful episodes in the extremities with color changes of the fingers or toes, digital ulcerations, skin thickening or hardening, capillary changes (assessed using nailfold capillaroscopy), swelling of the hands or legs, and general pain.

Vascular injury and endothelial damage are detected at initial evaluation in a majority of SSc patients. Most SSc patients (>90%) have secondary Raynaud's disease, which is often a presenting symptom of SSc. Raynaud's is an abnormal imbalance between vasoconstriction and vasodilation in blood vessels in the extremities in response to cold or emotional stress that results in poor blood flow producing pain (e.g., neuropathic pain), numbness, tingling, discoloration of the affected regions of the body (e.g., color changes to white, blue, or red, which can signify ischemia, cyanosis, and reperfusion, respectively) in the digits or other areas. Tissue ischemia from reduced blood flow, as well as reperfusion when vasoconstriction ceases, produces, painful burning sensations which can be experienced by the subject during the ischemic attack as well as when blood flow is reestablished. Symptoms of Raynaud's phenomenon can be experienced after, e.g., changes in temperature (cold or hot) in body tissues and/or the experience of strong emotions (e.g., stress) by the subject. In some severe cases, symptoms can progress to digital ulceration and/or gangrene. Secondary Raynaud's phenomenon is typically more severe than primary Raynaud's disease and is part of the pathology associated with SSc or another underlying disorder, such as lupus, Sjogrens, rheumatoid arthritis, or as a result of surgery or chemotherapy.

No cure exists for patients suffering from secondary Raynaud's syndrome with SSc. The exact cause is unknown, however the cause is thought to result from a combination of factors, including autoimmune, genetic, and environmental triggers. A simple review of the federal database of clinical trials (https://clinicaltrials.gov/) reveals that in recent record, there have been 87 studies and 30 different therapeutic modalities comprising multiple drugs, topicals, devices and physical treatments targeted at improving symptoms of Raynaud's syndrome but few of these have yielded any measurable success. Available therapies only provide symptomatic treatment with limited efficacy and safety. Existing treatments for scleroderma include L-type calcium channel blockers, prostaglandins, phosphodiesterase type 5 (PDE-5) inhibitors, and botulinum toxin. However, the efficacy of most currently available drug treatments is modest to moderate, and their use is often limited by side effects or the intensity and invasiveness of the treatments and because they address only symptoms rather than the underlying disease processes.

The common dihydropyridine calcium channel blockers (CCBs) used to treat secondary Raynaud's phenomenon associated with SSc are primarily L-type calcium channel antagonists, and thus principally exhibit only a direct vasodilating effect on afferent smooth muscle arterioles by relaxing vascular smooth muscle cells and providing peripheral vasodilation. However, in addition to vasoconstriction, SSc involves overactivity of the sympathetic nervous system, as well as abnormalities of the endothelium of blood vessels that disrupt digital blood flow and normal blood vessel responses to cold temperature or stress, changes in the microvascular architecture, and reduced release of nitric oxide (NO), which promotes vasodilation in response to cold-induced vasoconstriction. These intrinsic disease contributors are not targeted by the available L-type calcium channel blockers currently used to treat the disease as first line therapy. Non-N-selective calcium channel blockers with primarily L-type blocker activity and minimal N-type activity, such as nifedipine and amlodipine, have primarily L-type calcium channel activity in vascular smooth muscle and a low level of N-type calcium channel activity, producing dilation of arterioles without similar dilation of venules and thus commonly produce peripheral edema symptoms and other side effects that limit the broad application and use of these CCB agents. Primarily L-type CCBs also increase reflex sympathetic activity, causing increases in heart rate and flushing and decreased renal blood flow, as a result of the body's attempt to maintain homeostasis.

Further, a large survey of patients with secondary Raynaud's syndrome found that only 21% of patients considered their treatment to be even modestly effective (Source: Rheumatology (Oxford) 2015; 54:1443-1447.) A published review of a large, randomized trials in over 290 patients determined the minimally important difference in Raynaud scores for patients with the disease, which was a 14-15 point improvement from baseline in their Raynaud Condition Score (approximately a 25% improvement from baseline scores) and set the bar for defining an effective treatment. (Source: Ann.Rheum.Dis 2010 March; 69 (3): 588-591). Only intravenous iloprost infusions, given daily for 6 continuous hours, on 5 consecutive days every month, has achieved any significant level of improvement, but this is costly, intrusive, and resource intensive. No oral daily medicine has achieved this level of effect.

Based on the above considerations, there is a pressing need for a safer, more efficacious, and less invasive and costly treatment of Raynaud's syndrome that, e.g., (1) addresses the underlying mechanisms of the disease rather than ameliorates the symptoms, (2) and has fewer side effects than currently used treatments.

### SUMMARY

Described herein are combinations of dual N-type and L-type calcium channel blockers selective for the N-type calcium channel and phosphodiesterase type 5 inhibitors for the treatment of Raynaud's disease in subjects, including subjects having scleroderma or other diseases and disorders that may be associated with secondary Raynaud's disease.

It is understood that, in contrast to L-type calcium channel blockade, dual N-type and L-type calcium channel blockade selective for the N-type calcium channel can decrease sympathetic activity, as well as dilate both arterioles and the venous system, resulting in less adverse events than patients treated with dual L and N-calcium channel antagonists with lower levels of N type calcium channel selectivity (such as, e.g., a lack of peripheral edema caused by lesser dilation of the venous system). It is therefore postulated that this may enable dosing of a dual N-type and L-type calcium channel blockers selective for the N-type calcium channel at higher levels than non-N selective calcium channel blockers, thus increasing potency at producing disease modifying effects. In addition, dual L-type and N-channel type calcium channel blockade selective for the N-type calcium channel is believed to stimulate the release of NO and endothelial nitric oxide synthase (eNOS) expression, which are critical for normal endothelial function. Further, N-type calcium channels are more widely located in the body than L-type channels, and have been identified in the nervous system, heart, kidney, venules, and the endothelium. In the spine, N-type channels are located pre-synaptically and regulate sympathetic nerve activity; these channels may involve suppression of both arteriole and venule constriction in the fingers and toes that occur in secondary Raynaud's syndrome in subjects having scleroderma.

A beneficial effect of L-type calcium channel inhibition is the dilation of the arteries in smooth muscle, causing an increase in arterial diameter, referred to as vasodilation. However, L-type calcium channel inhibition induces a homeostatic reflex mechanism in which norepinephrine is produced. The norepinephrine induces vasoconstriction, as well as elevating heart rate, and thus partially offsets the vasodilating effects of the L-type calcium channel inhibition. A useful complementary effect of N-type calcium channel inhibition is the decrease of norepinephrine release and sympathetic outflow presynaptically in the spinal cord at the level of the dorsal root ganglion, which can counteract the homeostasis mechanism triggered by blockade of the L-type calcium channel. It is believed that dual N-type and L-type calcium channel blockers selective for the N-type calcium channel are able to achieve an optimal balance of N-vs. L-type calcium channel inhibition to realize these effects.

Additional advantages include an increase in bone density in certain subjects (e.g., subjects afflicted with osteoporosis), beneficial renal effects, and improvement in vascular modeling which has been shown to reduce the progression of arterial disease with long term use. The beneficial renal effects are understood to be an effect of reduced renal constriction and improved blood flow in the kidney.

Cilnidipine is understood to exert an optimum balance of selective N-vs. L-type calcium channel inhibition (which can have a 5 fold to 50-fold to 100-fold selectivity for N-type calcium channel over L-type calcium channel), which can make it surprisingly effective at treating neuropathic pain and vasoconstriction, particularly when they occur concurrently, such as in Raynaud's syndrome. The potential role of cilnidipine and dual N-type and L-type calcium channel blockers selective for the N-type calcium channels in treating Raynaud's syndrome has not been recognized. Cilnidipine also has activity against the Nav 1.7 sodium channel, which may further contribute to its efficacy in the treatment of Raynaud's syndrome. See WO 2021/178903, which is incorporated by reference herein in its entirety.

Phosphodiesterase inhibitors have been explored for treatment in patients having, e.g., neuropathic pain and/or vasoconstriction; however, at the doses that have been explored in these studies, common side effects occur such as headache, flushing, and dyspepsia and occasionally hypotension when used concurrently with nitrates.

Based on these considerations, a dual N-type and L-type calcium channel blocker selective for the N-type calcium channel such as cilnidipine, and a phosphodiesterase type 5 inhibitor such as tadalafil can be surprisingly useful to treat Raynaud's disease (e.g., secondary Raynaud's disease (e.g., secondary Raynaud's disease in subjects having scleroderma)). It is believed that the selective inhibition of the N-type calcium channel by the dual N-type and L-type calcium channel blocker selective for the N-type calcium channel enables the use of a lower dose of the phosphodiesterase type 5 inhibitor that would otherwise be required, therefore improving tolerability in the subject. Furthermore, the combination of cilnidipine and tadalafil is particularly advantageous due, e.g., to the similar time it takes for each drug to achieve maximal plasma concentrations (2 hr_{Tmax} for cilnidipine and 2 hr_{Tmax} for tadalafil. This combination reaches maximal plasma concentrations faster than the commonly used non-N-selective calcium channel blockers amlodipine (10 hours) or extended-release nifedipine (3 hours), which is an additional benefit of using both cilnidipine and tadalafil for treating SSc patients that have secondary Raynaud's disease compared to other non-N-selective calcium channel blockers. Further, 15 both drugs can be dosed at similar intervals (e.g., once daily), providing further basis for their complementarity.

In one aspect, disclosed herein is a method of treating Raynaud's syndrome in a subject in need thereof, comprising administering to the subject a therapeutically effective amount of a dual N-type and L-type calcium channel blocker selective for the N-type calcium channel and a phosphodiesterase type 5 inhibitor.

In another aspect, disclosed herein is a method of reducing the frequency of one or more symptoms associated with Raynaud's syndrome in a subject, comprising administering to the subject a therapeutically effective amount of a dual N-type and L-type calcium channel blocker selective for the N-type calcium channel and a phosphodiesterase type 5 inhibitor.

In another aspect, disclosed herein is a method of reducing the severity of one or more symptoms associated with Raynaud's syndrome in a subject, comprising administering to the subject a therapeutically effective amount of a dual N-type and L-type calcium channel blocker selective for the N-type calcium channel and a phosphodiesterase type 5 inhibitor.

In another aspect, disclosed herein is a method of reducing pain or discomfort caused by a reduction of body temperature in a subject, comprising administering to the subject a therapeutically effective amount of a dual N-type and L-type calcium channel blocker selective for the N-type calcium channel and a phosphodiesterase type 5 inhibitor, wherein the reduction of body temperature in the subject is caused by an exposure of the subject to air having a temperature of less than 25° C.

In another aspect, disclosed herein is a method of reducing susceptibility of a subject to cold-induced pain or discomfort, comprising administering to the subject a therapeutically effective amount of a dual N-type and L-type calcium channel blocker selective for the N-type calcium channel and a phosphodiesterase type 5 inhibitor.

### Definitions

As used herein, the terms “about” and “approximately” are used interchangeably, and when used to refer to modify a numerical value, encompass a range of uncertainty of the numerical value of from 0% to 10% of the numerical value.

As used herein, the singular forms “a,” “an,” and “the” include plural referents unless the context clearly dictates otherwise.

The terms “treat,” “treating,” and “treatment,” in the context of treating a disease, disorder, or condition, are meant to include alleviating a disorder, disease, or condition, or one or more of the symptoms associated with the disorder, disease, or condition; or to slow the progression of a disease, disorder or condition or of one or more symptoms thereof.

As used herein, the terms “subject” “individual,” or “patient,” are used interchangeably, refers to any animal, including mammals such as mice, rats, other rodents, rabbits, dogs, cats, swine, cattle, sheep, horses, primates, and humans. In some embodiments, the patient is a human. In some embodiments, the subject has experienced and/or exhibited at least one symptom of the disease or disorder to be treated and/or prevented. In some embodiments, the disease or disorder is associated with dysregulation of blood flow and sympathetic nervous system overactivity. In some embodiments, the disease or disorder is characterized by neuropathic pain, vasoconstriction, dysesthetic pain, burning pain, body temperature changes of the subject, hyperesthesia, changes in skin or tissue color, edema, changes in skin turgor, ruble, pallor, cyanosis, vasospasm, or any combination thereof.

As used herein, the phrase “fixed dosage form” refers to the simultaneous administration of two or more therapeutic agents to a subject in the form of a single composition or dosage.

As used herein, the term “dual N-type and L-type calcium channel blocker selective for the N-type calcium channel” refers to an agent that inhibits both N- and L-type calcium channels, and inhibits the N-type calcium channel to a greater degree than the L-type calcium channel. The terms “dual N-type and L-type calcium channel blocker selective for the N-type calcium channel” and “dual N-type and L-type selective calcium blocker” are used interchangeably herein. In some embodiments, the dual N-type and L-type calcium channel blocker selective for the N-type calcium channel has at least a 5-fold selectivity for the N-type calcium channel over the L-type calcium channel. For example, the dual N-type and L-type calcium channel blocker selective for the N-type calcium channel exhibits at least a 10-fold, at least a 30-fold, at least a 50-fold, at least a 80-fold, at least a 100-fold, at least a 300-fold, at least a 500-fold, at least a 800-fold, at least a 900-fold, or at least a 1000-fold selectivity for the N-type calcium channel over the L-type calcium channel. For example, the dual N-type and L-type calcium channel blocker selective for the N-type calcium channel exhibits at least a 50-fold to 100-fold selectivity for the N-type calcium channel over the L-type calcium channel. Examples of dual N-type and L-type calcium channel blocker selective for the N-type calcium channel include, but are not limited to, cilnidipine, Z-160, ralfinamide, CNV2197944, or pharmaceutically acceptable salts thereof.

As used herein, the term “non-N-selective calcium channel blocker” refers to an agent that blocks one or more calcium channels, but either (1) does not block the N-type calcium channel, or (2) blocks the N-type calcium channel, but not selectively over the L-type calcium channel (e.g., selectively blocks the L-type calcium channel over the N-type calcium channel). Examples of non-N-selective calcium channel blockers include, but are not limited to, nifedipine, nicardipine, amlodipine, Z-944, nimodipine, verapamil, diltiazem, felodipine, isradipine, nisoldipine, nitrendipine, and pharmaceutical salts thereof.

As used herein, the term “vasoconstriction” refers to the reduction in diameter of a blood vessel (e.g., an artery, vein, or capillary) resulting in reduced blood flow to the tissue the vasoconstricted blood vessels circulate blood to and from.

As used herein, the term “reducing susceptibility of a subject to cold-induced pain or discomfort” refers to reducing the pathologic response of a subject to experience pain or discomfort when subjected to an environment that lowers the temperature of a body part of the subject. In some embodiments, reducing susceptibility of a subject to cold-induced pain or discomfort can include reducing the likelihood that a subject will experience pain or discomfort when subjected to an environment that lowers the temperature of a body part of the subject. In some embodiments, reducing susceptibility of a subject to cold-induced pain or discomfort can include reducing the magnitude or intensity of pain or discomfort that a subject feels when subjected to an environment that lowers the temperature of a body part of the subject.

As used herein, the term “body temperature” refers to the temperature range of the body in a healthy, awake subject under normal conditions of thermoregulation as measured in the mouth, the rectum, the armpit, or the car. For example, the temperature range in a healthy human subject under normal conditions of thermoregulation is 36.1° C. to 37.8° C.

The term “therapeutically effective amount,” as used herein, refers to a sufficient amount of a chemical entity being administered which will relieve to an extent one or more of the symptoms of the disease or condition being treated. The result includes reduction and/or alleviation of the signs, symptoms, or causes of a disease, or any other desired alteration of a biological system. For example, an “effective amount” for therapeutic uses is the amount of the composition comprising a compound as disclosed herein required to provide a clinically significant decrease in disease symptoms. An appropriate “effective” amount in any individual case is determined using any suitable technique, such as a dose escalation study. When a combination of two or more chemical entities is administered (e.g., a dual N-type and L-type calcium channel blocker selective for the N-type calcium channel, and a phosphodiesterase type 5 inhibitor), a therapeutically effective amount of each component of the combination is understood to be the therapeutically effective amount of each component when used in conjunction with the other component, which can be different (e.g., lower) than the therapeutically effective amount of each component when administered alone.

The term “pharmaceutically acceptable excipient” means a pharmaceutically-acceptable material, composition, or vehicle, such as a liquid or solid filler, diluent, carrier, solvent, or encapsulating material. In one embodiment, each component is “pharmaceutically acceptable” in the sense of being compatible with the other ingredients of a pharmaceutical formulation, and suitable for use in contact with the tissue or organ of humans and animals without excessive toxicity, irritation, allergic response, immunogenicity, or other problems or complications, commensurate with a reasonable benefit/risk ratio. See, e.g., Remington: The Science and Practice of Pharmacy, 21st ed.; Lippincott Williams & Wilkins: Philadelphia, PA, 2005; Handbook of Pharmaceutical Excipients, 6th ed.; Rowe et al., Eds.; The Pharmaceutical Press and the American Pharmaceutical Association: 2009; Handbook of Pharmaceutical Additives, 3rd ed.; Ash and Ash Eds.; Gower Publishing Company: 2007; Pharmaceutical Preformulation and Formulation, 2nd ed.; Gibson Ed.; CRC Press LLC: Boca Raton, FL, 2009.

The term “pharmaceutically acceptable salt” may refer to pharmaceutically acceptable addition salts prepared from pharmaceutically acceptable non-toxic acids including inorganic and organic acids. In certain instances, pharmaceutically acceptable salts are obtained by reacting a compound described herein, with acids such as hydrochloric acid, hydrobromic acid, sulfuric acid, nitric acid, phosphoric acid, methanesulfonic acid, ethanesulfonic acid, p-toluenesulfonic acid, salicylic acid and the like. The term “pharmaceutically acceptable salt” may also refer to pharmaceutically acceptable addition salts prepared by reacting a compound having an acidic group with a base to form a salt such as an ammonium salt, an alkali metal salt, such as a sodium or a potassium salt, an alkaline earth metal salt, such as a calcium or a magnesium salt, a salt of organic bases such as dicyclohexylamine, N-methyl-D-glucamine, tris(hydroxymethyl)methylamine, and salts with amino acids such as arginine, lysine, and the like, or by other methods previously determined. The pharmacologically acceptable salt s not specifically limited as far as it can be used in medicaments. Examples of a salt that the compounds described herein form with a base include the following: salts thereof with inorganic bases such as sodium, potassium, magnesium, calcium, and aluminum; salts thereof with organic bases such as methylamine, ethylamine and ethanolamine; salts thereof with basic amino acids such as lysine and ornithine; and ammonium salt. The salts may be acid addition salts, which are specifically exemplified by acid addition salts with the following: mineral acids such as hydrochloric acid, hydrobromic acid, hydroiodic acid, sulfuric acid, nitric acid, and phosphoric acid: organic acids such as formic acid, acetic acid, propionic acid, oxalic acid, malonic acid, succinic acid, fumaric acid, maleic acid, lactic acid, malic acid, tartaric acid, citric acid, methanesulfonic acid, and ethanesulfonic acid; acidic amino acids such as aspartic acid and glutamic acid.

The term “pharmaceutical composition” refers to a mixture of a compound described herein with other chemical components (referred to collectively herein as “excipients”), such as carriers, stabilizers, diluents, dispersing agents, suspending agents, and/or thickening agents. The pharmaceutical composition facilitates administration of the compound to an organism. Multiple techniques of administering a compound exist in the art including, but not limited to: transdermal, intranasally, sublingual, intraspinal, or ocular administration.

For purposes of clarification, when a parameter, score, state, condition, or statistic in a subject is increased, decreased, or improved after administration of the dual N-type and L-type selective calcium blocker and/or the phosphodiesterase type 5 inhibitor, the increase, decrease, or improvement is, for example, measured, assessed, or obtained in relation to the parameter, score, state, condition, or statistic measured, assessed, or obtained before administration of the dual N-type and L-type selective calcium blocker and/or the phosphodiesterase type 5 inhibitor, unless otherwise specified herein. The parameter, score, state, condition, or statistic can be a single measurement, score, or assessment, an average of a plurality of measurements, scores, or assessments, or a daily average of a plurality of measurements, scores, or assessments. Unless otherwise specified herein, measurements, scores, or assessments are typically taken within 1 month (e.g., within 3 weeks, 2 weeks, 10 days, 9 days, 8 days, 7 days, 6 days, 5 days, 4 days, 3 days, 2 days, 1 day, 18 hours, 12 hours, or 6 hours) of the administration of the dual N-type and L-type selective calcium blocker and/or the phosphodiesterase type 5 inhibitor.

The details of one or more embodiments of the invention are set forth in the description below. Other features and advantages of the invention will be apparent from the description and drawings, and from the claims.

### DESCRIPTION OF DRAWINGS

FIG. 1 shows a schematic of a study to assess the safety and efficacy of cilnidipine alone and in combination with tadalafil in subjects having secondary Raynaud's disease.

FIG. 2 shows a schedule of assessments in the double-blind parallel group.

FIG. 3A and FIG. 3B show a schedule of assessments in the double-blind 4-way crossover group.

### DETAILED DESCRIPTION

Herein is described the effect of specifically targeting the ‘N’ calcium channel (Cav 2.2) with a small molecule antagonist and further describes that the efficacy may be improved by addition of a modest dose of a phosphodiesterase-V inhibitor, below the dose most clinicians would begin as a starting dose for the treatment of symptoms of Raynaud's syndrome.

In one aspect, disclosed herein is a method of treating Raynaud's syndrome in a subject in need thereof, comprising administering to the subject a therapeutically effective amount of: a dual N-type and L-type calcium channel blocker selective for the N-type calcium channel (“dual N-type and L-type selective calcium blocker”), and/or a phosphodiesterase type 5 inhibitor. In some embodiments, a dual N-type and L-type selective calcium blocker and a phosphodiesterase type 5 inhibitor are administered to the subject. In some embodiments, a dual N-type and L-type selective calcium blocker is administered to the subject. In some embodiments, a phosphodiesterase type 5 inhibitor is administered to the subject.

In one aspect, disclosed herein is a method of treating Raynaud's syndrome in a subject in need thereof, comprising administering to the subject a therapeutically effective amount of a dual N-type and L-type calcium channel blocker selective for the N-type calcium channel and a phosphodiesterase type 5 inhibitor.

Examples of N-type calcium channels include, but are not limited to, the Cav2.2 Type, which has two subunits, Cav 2.2a and Cav2.2b, both of which have an alpha 1 subunit of 2.2 and are affected by N type current.

In some embodiments, the Raynaud's syndrome is secondary Raynaud's syndrome. In some embodiments, the subject has lupus (e.g., systemic lupus erythematosus (SLE)), scleroderma, scleroderma with interstitial lung disease, rheumatoid arthritis, atherosclerosis, cryoglobulinemia, polycythemia, dermatomyositis, polymyositis, Sjögren's syndrome, or any combination thereof. In some embodiments, the subject has scleroderma. In some embodiments, the subject has scleroderma and the Raynaud's syndrome is secondary Raynaud's syndrome.

The combination of the dual N-type and L-type selective calcium blocker and the phosphodiesterase type 5 inhibitor is understood to improve the underlying pathological processes associated with Raynaud's syndrome (e.g., secondary Raynaud's syndrome). For example, the treating comprises dilating arterioles, dilating venules, increasing production of nitrous oxide, reducing norepinephrine, improving endothelial function, inhibiting calcitonin gene-related neuropeptide (CGRP), reducing inflammation, or any combination thereof in the subject.

In some embodiments, before administering the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) to the subject, the subject is diagnosed with Raynaud's syndrome (e.g., secondary Raynaud's syndrome). In some embodiments, before administering the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) to the subject, the subject is diagnosed with Raynaud's syndrome (e.g., secondary Raynaud's syndrome) and scleroderma.

In some embodiments, the treating comprises reducing fibrosis in the subject. In some embodiments, reducing fibrosis comprises reducing formation of collagen and extracellular matrix proteins in the subject. In some embodiments, the collagen is formed by fibroblasts. In some embodiments, the fibrosis is renal fibrosis or myocardial fibrosis.

In some embodiments, the treating comprises improving vascular function in the subject. In some embodiments, improving vascular function comprises decreasing intima media thickness (IMT), decreasing arterial stiffness, reducing urinary albumin excretion (UAE), reducing plaque in the arteries, or any combination thereof.

In some embodiments, the subject also has interstitial lung disease. In some embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) the interstitial lung disease is treated. In some embodiments, the method further comprises administering an agent selected from the group consisting of: a calcineurin inhibitor, cyclophosphamide, nintedanib, methotrexate, mycophenolate, a glucocorticoid (e.g., prednisone, dexamethasone, and hydrocortisone), a non-steroidal anti-inflammatory drug (e.g., aspirin, ibuprofen, or naproxen), D-penicillamine, a diuretic, omeprazole, bosentan, epoprostenol, enalapril, Lisinopril, captopril, or any combination thereof. For example, the method further comprises administering nintedanib. In some embodiments, the method further comprises administering a calcineurin inhibitor, a non-steroidal anti-inflammatory drug, or both. In some embodiments, treating interstitial lung disease in the subject comprises increasing Forced Vital Capacity (FVC), increasing percent of predicted value, increasing diffusing capacity of the lung for carbon monoxide (DLCO), increasing total lung capacity (TLC) or any combination thereof. For further information, see Caron, M. et al; Eur. Resp. Review; 2018 27:170102 which is incorporated by reference herein in its entirety.

In some embodiments, the treating comprises improving lung function in the subject. In some embodiments, improving lung function in the subject comprises increasing blood oxygen saturation. In some embodiments, increasing blood oxygen saturation comprises increasing one measurement or an average of a plurality of measurements of blood oxygen saturation by at least about 1% (e.g., at least about 2%, at least about 3%, at least about 4%, at least about 5%, at least about 8%, at least about 10%, at least about 15%, at least about 20%, at least about 25%, at least about 30%, or at least about 40%) after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to one measurement or an average of a plurality of measurements of blood oxygen saturation taken before administration of the dual N-type and L-type selective calcium blocker and/or the phosphodiesterase type 5 inhibitor. In some embodiments, when a plurality of measurements is taken before and after administration, they are taken over the same period of time (e.g., the same number of days).

In some embodiments, the subject has digital ulcerations. In some embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)), the number and/or severity of the digital ulcerations is reduced. In some embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)), one or more digital ulcerations in the subject exhibits healing. In some embodiments, the subject exhibits an improvement in digital ulcer severity after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, the improvement comprises a reduction in a score provided by the visual analog scale (VAS). In some embodiments, one score or an average of a plurality of scores is reduced by at least about 1% (e.g., at least about 2%, at least about 3%, at least about 4%, at least about 5%, at least about 8%, at least about 10%, at least about 15%, at least about 20%, at least about 25%, at least about 30%, at least about 40%, at least about 50%, at least about 60%, at least about 70%, at least about 80%, at least about 90%, or at least about 95%) after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to one score or an average of a plurality of scores taken before administration of the dual N-type and L-type selective calcium blocker and/or the phosphodiesterase type 5 inhibitor. In some embodiments, when a plurality of scores are obtained before and after administration, they are taken over the same period of time (e.g., the same number of days). In some embodiments, after administration of the dual N-type and L-type selective calcium blocker and/or the phosphodiesterase type 5 inhibitor, at least one (e.g., at least two, at least three, at least 5, at least 10, or at least 20) digital ulcers fully heal. Further information on the measurement of digital ulcer severity using VAS is in the Examples. In some embodiments, the method further comprises administering vitamin C and/or vitamin E to the subject.

In some embodiments, the treating comprises improving cardiac function in the subject. In some embodiments, improving cardiac function in the subject comprises reducing the frequency and/or severity of cardiac arrhythmias; reducing sympathomimetic increases in papillary muscle-developed tension (PMDT); reducing myocardial interstitial norepinephrine level; decreasing aortic pressure; increased aortic, vertebral, and coronary blood flow; reducing myocardial oxygen consumption; reducing blood pressure; atrial remodeling; or any combination thereof.

In some embodiments, the treating comprises alleviating one or more symptoms associated with Raynaud's syndrome (e.g., secondary Raynaud's syndrome) in the subject. In this context, alleviating one or more symptoms associated with the disease or disorder can, for example, comprise reducing the severity, duration, and/or frequency of the symptoms when compared to (1) the severity, duration, and/or frequency of the one or more symptoms in the subject before start of the treatment (e.g., before administration of the one or more therapeutic agents, and wherein the severity, duration, and/or frequency of the one or more symptoms before administration of the one or more therapeutic agents can, for example, be evaluated by a single measurement or assessment, or an average of a plurality of measurements or assessments taken, e.g., over the course of a 2 week period, a 7 day period, a 6 day period, a 5 day period, a 4 day period, a 3 day period, a 2 day period, or a 1 day period (e.g., a 7 day period)), wherein, for example, the reduction in severity, duration, and/or frequency of the symptoms is measured about 1 hour after treatment (e.g., after about 2 hours, 4 hours, 6 hours, 8 hours, 16 hours, 1 day, 2 days, 3 days, 4 days, 5 days, 6 days, 1 week, 1.5 weeks, 2 weeks, 3 weeks, 4 weeks, 6 weeks, 2 months, 3 months, or 1 year of treatment); (2) the severity, duration, and/or frequency of the one or more symptoms experienced by a subject after the subject was administered a placebo; and/or (3) the severity, duration, and/or frequency of the one or more symptoms experienced by a subject after the subject was administered an alternative treatment such as a non-N selective calcium channel blocker alone, a combination of a non-N selective calcium channel blocker and a phosphodiesterase type 5 inhibitor, or a phosphodiesterase type 5 inhibitor alone. In some embodiments, the reduction in severity, duration, and/or frequency of the symptoms is greatest within 2 days (e.g., within 1.5 days, within 1 day, within 20 hours, within 16 hours, within 12 hours, within 8 hours, within 6 hours, within 4 hours, within 2 hours, or within 1 hour after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor))). In some embodiments, the reduction in severity, duration, and/or frequency of the symptoms is greatest within 8 hours after administration of the dual N-type and L-type selective calcium blocker and the phosphodiesterase type 5 inhibitor.

In some embodiments, the subject experiences less frequent, less severe, and/or shorter episodes of the one or more symptoms of Raynaud's syndrome than when administered a therapeutically effective amount of a non-N-selective calcium channel blocker alone, a phosphodiesterase type 5 inhibitor alone, or a combination of a non-N-selective calcium channel blocker and a phosphodiesterase type 5 inhibitor useful to treat the secondary Raynaud's syndrome. In some embodiments, the subject experiences less frequent, less severe, and/or shorter episodes of the one or more symptoms of Raynaud's syndrome than when administered a therapeutically effective amount of a phosphodiesterase type 5 inhibitor alone.

In some embodiments, the treating comprises reducing the frequency of one or more symptoms associated with Raynaud's syndrome (e.g., secondary Raynaud's syndrome) in the subject.

In some embodiments, the treating comprises reducing the duration of one or more symptoms associated with secondary Raynaud's syndrome in the subject.

In some embodiments, the treating comprises reducing the severity of one or more symptoms associated with secondary Raynaud's syndrome in the subject.

In another aspect, disclosed herein is a method of reducing the frequency of one or more symptoms associated with Raynaud's syndrome (e.g., secondary Raynaud's syndrome) in a subject, comprising administering to the subject a therapeutically effective amount of a dual N-type and L-type calcium channel blocker selective for the N-type calcium channel and a phosphodiesterase type 5 inhibitor. In some embodiments, a dual N-type and L-type selective calcium blocker and a phosphodiesterase type 5 inhibitor are administered to the subject. In some embodiments, a dual N-type and L-type selective calcium blocker is administered to the subject. In some embodiments, a phosphodiesterase type 5 inhibitor is administered to the subject. In some embodiments, reducing the frequency of one or more symptoms associated with Raynaud's syndrome comprises measuring a reduction in the average frequency (e.g., average daily frequency) of the one or more symptoms measured after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to the average frequency (e.g., average daily frequency) of the one or more symptoms measured before administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) taken over the same time period (e.g., 7 days). In some embodiments, the frequency of the one or more symptoms associated with Raynaud's syndrome is reduced by at least 5%, for example, at least 10%, at least 20%, at least 25%, at least 30%, at least 35%, at least 40%, at least 45%, at least 50%, at least 55%, at least 60%, at least 70%, at least 80%, at least 90%, or at least 95% (e.g., at least 25%; e.g., at least 40%; e.g., at least 45%). In some embodiments, the frequency of one or more symptoms associated with Raynaud's syndrome in the subject is reduced by at least 25%. In some embodiments, the subject experiences no symptoms after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)).

In another aspect, disclosed herein is a method of reducing the duration of one or more symptoms associated with Raynaud's syndrome (e.g., secondary Raynaud's syndrome) in a subject, comprising administering to the subject a therapeutically effective amount of a dual N-type and L-type calcium channel blocker selective for the N-type calcium channel and a phosphodiesterase type 5 inhibitor. In some embodiments, a dual N-type and L-type selective calcium blocker and a phosphodiesterase type 5 inhibitor are administered to the subject. In some embodiments, a dual N-type and L-type selective calcium blocker is administered to the subject. In some embodiments, a phosphodiesterase type 5 inhibitor is administered to the subject. In some embodiments, reducing the duration of one or more symptoms associated with Raynaud's syndrome comprises reducing the collective duration of the one or more symptoms of Raynaud's syndrome measured over a timespan divided by the number of occurrences of the one or more symptoms that occur during the timespan after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to the collective duration of the one or more symptoms of Raynaud's syndrome divided by the number of occurrences of the one or more symptoms over the same timespan before administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, the timespan is from about 1 day to about 1 month, for example, from about 1 day to about 3 weeks, from about 1 day to about 2 weeks, from about 1 day to about 10 days, from about 1 day to about 7 days, from about 4 days to about 10 days, from about 5 days to about 9 days, from about 6 days to about 8 days, or about 7 days. In some embodiments, the duration of the one or more symptoms is reduced by at least 5%, at least 10%, at least 15%, at least 20%, at least 25%, at least 30%, at least 35%, at least 40%, at least 45%, at least 50%, at least 60%, at least 70%, at least 80%, at least 90%, at least 95%, or at least 98%. In some embodiments, the duration of the one or more symptoms associated with Raynaud's syndrome in the subject is reduced by at least 20%.

In some embodiments, the treating comprises reducing the severity of one or more symptoms associated with secondary Raynaud's syndrome in the subject. In some embodiments, reducing the severity of the one or more symptoms associated with Raynaud's syndrome comprises measuring a reduction in the visual analog scale (VAS) 0-10 cm. In some embodiments, reducing the severity of the one or more symptoms associated with Raynaud's syndrome comprises measuring a reduction in a score provided by the visual analog scale. In some embodiments, the reduction in the score provided by the visual analog scale comprises a reduction in a single score or the average of a plurality of scores measured after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to a single score or the average of a plurality of scores measured before administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, when a plurality of scores is obtained before and after administration, they are taken over the same period of time (e.g., the same number of days). In some embodiments, the score is reduced by at least 5%, at least 10%, at least 15%, at least 20%, at least 25%, at least 30%, at least 35%, at least 40%, at least 45%, at least 50%, at least 60%, at least 70%, at least 80%, at least 90%, at least 95%, or at least 98%. In some embodiments, the score is reduced by at least 20%.

In some embodiments, the subject has lupus, scleroderma, scleroderma with interstitial lung disease, rheumatoid arthritis, atherosclerosis, cryoglobulinemia, polycythemia, dermatomyositis, polymyositis, Sjögren's syndrome, or any combination thereof. In some embodiments, the subject has scleroderma. In some embodiments, the Raynaud's syndrome is secondary Raynaud's syndrome. In some embodiments, the subject has scleroderma and the Raynaud's syndrome is secondary Raynaud's syndrome.

In some embodiments, the symptoms are selected from the group consisting of: pain, anemia, fatigue, change in coloration of the skin, cyanosis, reperfusion, deoxygenation of the blood, digital ulcerations, reduced temperature in one or more parts of the body, changes in the endothelium of a blood vessel, swelling, impaired vision, or any combination thereof. In some embodiments, the symptom is pain.

In another aspect, disclosed herein is a method of reducing pain or discomfort caused by a reduction of body temperature in a subject, comprising administering to the subject a therapeutically effective amount of a dual N-type and L-type calcium channel blocker selective for the N-type calcium channel and/or a phosphodiesterase type 5 inhibitor, wherein the reduction of body temperature in the subject is caused by an exposure of the subject to air having a temperature of less than 25° C. In some embodiments, a dual N-type and L-type selective calcium blocker and the phosphodiesterase type 5 inhibitor are administered to the subject. In some embodiments, a dual N-type and L-type selective calcium blocker is administered to the subject. In some embodiments, a phosphodiesterase type 5 inhibitor is administered to the subject.

In some embodiments, the subject has lupus, scleroderma, scleroderma with interstitial lung disease, rheumatoid arthritis, atherosclerosis, cryoglobulinemia, polycythemia, dermatomyositis, polymyositis, Sjögren's syndrome, or any combination thereof. In some embodiments, the subject has scleroderma. In some embodiments, the subject has scleroderma.

In some embodiments, the reduction of body temperature in the subject is caused by an exposure of the subject to air having a temperature of less than 20° C., for example, less than 20° C., less than 15° C., less than 10° C., less than 5° C., less than 0° C., less than −5° C., less than −10° C., or less than −5° C. In some embodiments, the reduction of body temperature in the subject is caused by an exposure of the subject to air having a temperature of less than 10° C. In some embodiments, the pain or discomfort that is reduced occurs during the exposure of the subject to air having a temperature of less than 25° C.

In some embodiments, the reduction of body temperature in the subject is followed by a restoration to the normal body temperature in the subject, and the pain or discomfort that is reduced occurs after restoration to the normal body temperature in the subject.

In some embodiments, the reduction of body temperature in the subject comprises reduction in the temperature of a region of the body of the subject. In some embodiments, the reduction of body temperature in the subject comprises reduction in the temperature of a finger of the subject. In some embodiments, the reduction of body temperature in the subject comprises reduction in the temperature of a hand of the subject. In some embodiments, the reduction of body temperature in the subject comprises reduction in the temperature of a foot of the subject.

In another aspect, disclosed herein is a method of reducing susceptibility of a subject to cold-induced pain or discomfort, comprising administering to the subject a therapeutically effective amount of a dual N-type and L-type calcium channel blocker selective for the N-type calcium channel and/or a phosphodiesterase type 5 inhibitor. In some embodiments, a dual N-type and L-type selective calcium blocker and a phosphodiesterase type 5 inhibitor are administered to the subject. In some embodiments, a dual N-type and L-type selective calcium blocker is administered to the subject. In some embodiments, a phosphodiesterase type 5 inhibitor is administered to the subject.

In some embodiments, the subject has scleroderma. In some embodiments, the scleroderma is limited scleroderma. In some embodiments, the scleroderma is diffuse scleroderma.

In some embodiments, the subject experiences a lesser degree of the pain or discomfort upon exposure to air having a temperature of less than 25° C. than as compared to a subject that is not administered the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) and is exposed to air having a temperature of less than 25° C. For example, the subject experiences a lesser degree of the pain or discomfort upon exposure to air having a temperature of less than 20° C. than as compared to a subject that is not administered the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) and is exposed to air having a temperature of less than 20° C. For example, the subject experiences a lesser degree of the pain or discomfort upon exposure to air having a temperature of less than 15° C. than as compared to a subject that is not administered the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) and is exposed to air having a temperature of less than 15° C. For example, the subject experiences a lesser degree of the pain or discomfort upon exposure to air having a temperature of less than 10° C. than as compared to a subject that is not administered the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) and is exposed to air having a temperature of less than 10° C. For example, the subject experiences a lesser degree of the pain or discomfort upon exposure to air having a temperature of less than 5° C. than as compared to a subject that is not administered the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) and is exposed to air having a temperature of less than 5° C. For example, the subject experiences a lesser degree of the pain or discomfort upon exposure to air having a temperature of less than 0° C. than as compared to a subject that is not administered the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) and is exposed to air having a temperature of less than 0° C. For example, the subject experiences a lesser degree of the pain or discomfort upon exposure to air having a temperature of less than −10° C. than as compared to a subject that is not administered the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) and is exposed to air having a temperature of less than −10° C.

In some embodiments, vasoconstriction in the subject is reduced after administering the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) to the subject. In certain embodiments, the vasoconstriction comprises vasoconstriction of a body part, and the temperature of the vasoconstricted body part is lower than the subject's body temperature.

In another aspect, disclosed herein is a method of reducing a sensation of burning pain, paresthesia, dysesthesia, hypoesthesia, or hyperesthesia (e.g., burning pain) in a subject having scleroderma, comprising administering a dual N-type and L-type calcium channel blocker selective for the N-type calcium channel and/or a phosphodiesterase type 5 inhibitor. A sensation of burning pain can be measured using one or more of the following: the Galer neuropathic pain scale, the ID pain Scale, NPQ, PainDETECT, LANS S, DN4 scales, and/or the Standardized Evaluation of Pain (StEP) tool. See, for example, Cruccu G, Truini A. Tools for assessing neuropathic pain. PLOS Med. 2009; 6 (4): e 1000045. doi: 10.1371/journal.pmed.1000045, which is incorporated by reference herein in its entirety. In some embodiments, a dual N-type and L-type selective calcium blocker and a phosphodiesterase type 5 inhibitor are administered to the subject. In some embodiments, a dual N-type and L-type selective calcium blocker is administered to the subject. In some embodiments, a phosphodiesterase type 5 inhibitor is administered to the subject.

In another aspect, disclosed herein is a method of reducing the number and/or severity of digital ulcerations in a subject having secondary Raynaud's disease, comprising administering to the subject a therapeutically effective amount of a dual N-type and L-type calcium channel blocker selective for the N-type calcium channel and a phosphodiesterase type 5 inhibitor. In some embodiments, a dual N-type and L-type selective calcium blocker and a phosphodiesterase type 5 inhibitor are administered to the subject. In some embodiments, a dual N-type and L-type selective calcium blocker is administered to the subject. In some embodiments, a phosphodiesterase type 5 inhibitor is administered to the subject.

In some embodiments, the method further comprises administering vitamin C and/or vitamin E to the subject.

In another aspect, disclosed herein is a method of treating endothelial dysfunction in a subject having secondary Raynaud's disease, comprising administering to the subject a therapeutically effective amount of a dual N-type and L-type calcium channel blocker selective for the N-type calcium channel and/or a phosphodiesterase type 5 inhibitor. In some embodiments, a dual N-type and L-type selective calcium blocker and a phosphodiesterase type 5 inhibitor are administered to the subject. In some embodiments, a dual N-type and L-type selective calcium blocker is administered to the subject. In some embodiments, a phosphodiesterase type 5 inhibitor is administered to the subject.

In some embodiments, the dual N-type and L-type calcium channel blocker selective for the N-type calcium channel exhibits at least a 2-fold selectivity (e.g., at least a 4-fold, 10-fold, 20-fold, 30-fold, 40-fold, 50-fold, 60-fold, 80-fold, 100-fold, 130-fold, 150-fold, or 200-fold selectivity) for the N-type calcium channel over an L-type calcium channel. In some embodiments, the dual N-type and L-type calcium channel blocker selective for the N-type calcium channel exhibits at least a 50-fold selectivity for the N-type calcium channel over an L-type calcium channel. In some embodiments, the dual N-type and L-type calcium channel blocker selective for the N-type calcium channel exhibits a 50-fold to 100-fold selectivity for the N-type calcium channel over an L-type calcium channel.

In some embodiments, the dual N-type and L-type selective calcium blocker is selected from the group consisting of: cilnidipine, Z-160, CNV2197944, or pharmaceutically acceptable salts thereof. In some embodiments, the dual N-type and L-type selective calcium blocker is cilnidipine or a pharmaceutically acceptable salt thereof.

In some embodiments, the phosphodiesterase type 5 inhibitor is selected from sildenafil, tadalafil, or pharmaceutically acceptable salts thereof. In some embodiments, the phosphodiesterase type 5 inhibitor is tadalafil or a pharmaceutically acceptable salt thereof. In some embodiments, the phosphodiesterase type 5 inhibitor is sildenafil or a pharmaceutically acceptable salt thereof.

In some embodiments, the amount of the dual N-type and L-type selective calcium blocker used in the method is less (e.g., at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, or at least 95% less) than the therapeutically effective amount of the dual N-type and L-type selective calcium blocker useful to treat secondary Raynaud's syndrome in a subject having scleroderma when administered alone.

In some embodiments, the amount of the phosphodiesterase type 5 inhibitor used in the method is less (e.g., at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, or at least 95% less) than the therapeutically effective amount of the phosphodiesterase type 5 inhibitor useful to treat secondary Raynaud's syndrome in a subject having scleroderma when administered in combination with a non-N selective calcium channel blocker.

In some embodiments, the amount of the phosphodiesterase type 5 inhibitor used in the method is less (e.g., at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, or at least 95% less) than the therapeutically effective amount of the phosphodiesterase type 5 inhibitor useful to treat secondary Raynaud's syndrome in a subject having scleroderma when administered alone.

In some embodiments, the subject experiences less side effects than a therapeutically effective amount of a non-N-selective calcium channel blocker useful to treat the secondary Raynaud's syndrome. In some embodiments, the side effects are selected from the group consisting of: constipation, nausea, headache, fatigue, rash, edema, pulmonary edema, drowsiness, dizziness, muscle weakness, muscle cramps, abnormal heartbeat, liver dysfunction, overgrowth of oral gums, flushing, low blood pressure, gastroesophageal reflux, bradycardia, tachycardia, QT interval prolongation, increased appetite, tenderness or bleeding of the gums, sexual dysfunction, abdominal pain, fainting, shortness of breath, altered taste, asthenia, muscle cramps, and itching.

In some embodiments, the dosage of the dual N-type and L-type selective calcium blocker is about 1 mg to about 50 mg (e.g., about 3 mg to about 35 mg, about 5 mg to about 25 mg, about 8 mg to about 28 mg, about 12 mg to about 28 mg, about 9 mg to about 21 mg, about 15 mg to about 25 mg, about 17 mg to about 23 mg, about 8 mg to about 12 mg, about 8 mg, about 10 mg, about 12 mg, about 18 mg, about 20 mg, or about 22 mg). In some embodiments, the dosage of the dual N-type and L-type selective calcium blocker is about 5 mg to about 25 mg. In some embodiments, the dosage of the dual N-type and L-type selective calcium blocker is about 9 mg to about 21 mg. In some embodiments, the dosage of the dual N-type and L-type selective calcium blocker is about 10 mg. In some embodiments, the dosage of the dual N-type and L-type selective calcium blocker is about 20 mg.

In some embodiments, the dosage of the phosphodiesterase type 5 inhibitor is about 1 mg to about 50 mg (e.g., about 2 mg to about 40 mg, about 8 mg to about 40 mg, about 2 mg to about 20 mg, about 2 mg to about 12 mg, about 3 mg to about 7 mg, about 4 mg to about 6 mg, about 3 mg, about 4 mg, about 5 mg, about 6 mg, or about 7 mg). In some embodiments, the dosage of the phosphodiesterase type 5 inhibitor is about 2 mg to about 8 mg. In some embodiments, the dosage of the phosphodiesterase type 5 inhibitor is about 5 mg.

In some embodiments of the methods disclosed herein, the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) are administered orally, parenterally, transdermally, intranasally, sublingually, neuraxially, or ocularly. In some embodiments, the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) are administered orally.

In some embodiments, the dual N-type and L-type selective calcium blocker and/or the phosphodiesterase type 5 inhibitor are administered separately, sequentially, or simultaneously. In some embodiments, the dual N-type and L-type selective calcium blocker and/or the phosphodiesterase type 5 inhibitor are administered separately. In some embodiments, the dual N-type and L-type selective calcium blocker and/or the phosphodiesterase type 5 inhibitor are administered sequentially. In some embodiments, the dual N-type and L-type selective calcium blocker and/or the phosphodiesterase type 5 inhibitor are administered simultaneously. In some embodiments, the dual N-type and L-type selective calcium blocker and/or the phosphodiesterase type 5 inhibitor are administered simultaneously as a fixed dosage form.

In some embodiments, each administration of the dual N-type and L-type selective calcium blocker and the phosphodiesterase type 5 inhibitor is less frequent than the frequency of administering either the dual N-type and L-type selective calcium blocker or the phosphodiesterase type 5 inhibitor alone useful to treat the Raynaud's syndrome (e.g., the secondary Raynaud's syndrome).

In some embodiments, each administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) is separated by at least about 1 hour (e.g., at least about 2 hours, at least about 4 hours, at least about 6 hours, at least about 8 hours, at least about 12 hours, at least about 15 hours, at least about 20 hours, at least about 24 hours, at least about 36 hours, at least about 48 hours, at least about 72 hours, at least about 4 days, at least about 5 days, at least about 3 days, at least about 5 days, at least about 1 week). In some embodiments, each administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) is separated by at least about 24 hours. In some embodiments, each administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) is separated by at least about 48 hours. In some embodiments, each administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) is separated by at least about 72 hours. In some embodiments, each administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) is separated by at least about 1 week.

In some embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)), the subject experiences gastrointestinal symptoms that are ameliorated by the consumption of food prior to administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, the subject consumes food up to about 6 hours before administering the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). For example, the subject consumes food up to about 5 hours, about 4 hours, about 3 hours, about 2 hours, about 1 hour, about 30 minutes, about 20 minutes, about 15 minutes, about 10 minutes, about 5 minutes, about 1 minute, about 30 seconds, or about 5 seconds before administering the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). For example, the subject consumes food concurrently with administering the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)).

In some embodiments, the subject experiences less frequent, less severe, and/or shorter episodes of adverse events than when administered a therapeutically effective amount of a non-N-selective calcium channel blocker alone, a phosphodiesterase type 5 inhibitor alone, or a combination of a non-N-selective calcium channel blocker and a phosphodiesterase type 5 inhibitor useful to treat the secondary Raynaud's syndrome. In some embodiments, the adverse events are one or more events selected from the group consisting of: tachycardia, headaches, flushing, increased heart rate, flushing, decreased renal blood flow, myalgia, pain (e.g., chest pain), heart palpitation, and/or pedal enema. In some embodiments, the subject experiences less frequent, less severe, and/or shorter episodes of tachycardia, headaches, flushing, increased heart rate, flushing, decreased renal blood flow, myalgia, pain (e.g., chest pain), heart palpitation, and/or pedal enema than when administered a therapeutically effective amount of a non-N-selective calcium channel blocker alone, a phosphodiesterase type 5 inhibitor alone, or a combination of a non-N-selective calcium channel blocker and a phosphodiesterase type 5 inhibitor useful to treat the secondary Raynaud's syndrome. In some embodiments, the subject experiences less frequent, less severe, and/or shorter episodes of pain (e.g., chest pain) than when administered a therapeutically effective amount of a non-N-selective calcium channel blocker alone, a phosphodiesterase type 5 inhibitor alone, or a combination of a non-N-selective calcium channel blocker and a phosphodiesterase type 5 inhibitor useful to treat the secondary Raynaud's syndrome.

In some embodiments, the non-N-selective calcium channel blocker is a dihydropyridine. In some other embodiments, the non-N-selective calcium channel blocker is not a dihydropyridine. In some embodiments, the non-N-selective calcium channel blocker is selected from the group consisting of: nifedipine, nicardipine, amlodipine, Z-944, nimodipine, verapamil, diltiazem, felodipine, isradipine, nisoldipine, and nitrendipine.

In some embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; or the dual N-type and L-type selective calcium blocker (e.g., the dual N-type and L-type selective calcium blocker and the phosphodiesterase type 5 inhibitor)) in the subject, sympathetic tone diminution, direct smooth muscle relaxation, or both occur in the subject.

In some embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; or the dual N-type and L-type selective calcium blocker (e.g., the dual N-type and L-type selective calcium blocker and the phosphodiesterase type 5 inhibitor)) in the subject, norepinephrine is reduced in the subject.

It is understood that dual N-type and L-type calcium channel blocker selective for the N-type calcium channels may decrease the blood pressure of subjects that are hypertensive. As such, it may be beneficial to administer an agent that increases blood pressure in combination with the dual N-type and L-type selective calcium blocker and/or the phosphodiesterase type 5 inhibitor (e.g., the dual N-type and L-type selective calcium blocker and the phosphodiesterase type 5 inhibitor; or the dual N-type and L-type selective calcium blocker (e.g., the dual N-type and L-type selective calcium blocker and the phosphodiesterase type 5 inhibitor)). In some embodiments of the methods disclosed herein, the method further comprises administering to the subject a therapeutically effective amount of an agent that increases blood pressure. In certain embodiments, the agent that increases blood pressure is selected from the group consisting of: midodrine, cortisone, prednisone, trimipramine, venlafaxine, anabolic steroids, antidepressants, anti-obesity drugs, CETP inhibitors, herbal preparations, immunosuppressants, mineralocorticoids, NSAIDS/coxibs, serotonergics, stimulants, sulfonylureas, and sympathomimetic amines. In certain embodiments, the blood pressure of the subject before and after administration of the dual N-type and L-type selective calcium blocker, the phosphodiesterase type 5 inhibitor, and the agent that increases blood pressure is substantially the same.

In some embodiments, the treating comprises reducing pulmonary hypertension in the subject.

In some embodiments, the subject is also diagnosed with hypertension; and wherein after administration of the dual N-type and L-type selective calcium blocker and/or the phosphodiesterase type 5 inhibitor (e.g., the dual N-type and L-type selective calcium blocker and the phosphodiesterase type 5 inhibitor; or the dual N-type and L-type selective calcium blocker (e.g., the dual N-type and L-type selective calcium blocker and the phosphodiesterase type 5 inhibitor)) to the subject, the blood pressure (e.g., systolic blood pressure) of the subject is reduced. In some embodiments, the subject was not diagnosed with hypertension; and wherein after administration of the dual N-type and L-type calcium channel blocker selective for the N-type calcium channel to the subject, the blood pressure (e.g., the systolic blood pressure) of the subject is not reduced. Without wishing to be bound by theory, it is believed that when the subject has hypertension, the dual N-type and L-type calcium channel blocker selective for the N-type calcium channel reduces the blood pressure of the subject; however, when the subject does not have hypertension (i.e., the subject is normotensive), the dual N-type and L-type calcium channel blocker selective for the N-type calcium channel does not reduce the blood pressure of the subject.

In some embodiments, the systolic blood pressure of the subject is reduced by greater than about 1 mm Hg (e.g., greater than about 2 mm Hg, greater than about 5 mm Hg, greater than about 10 mm Hg, greater than about 15 mm Hg, greater than about 20 mm Hg, greater than about 30 mm Hg, or greater than about 40 mm Hg).

In some embodiments, the therapeutically effective amount of the dual N-type and L-type selective calcium blocker and the phosphodiesterase type 5 inhibitor decreases the blood pressure of the subject to a lesser degree than a therapeutically effective amount of a combination of a non-N-selective calcium channel blocker and a phosphodiesterase type 5 inhibitor useful to treat the secondary Raynaud's syndrome. In some embodiments, the dual N-type and L-type selective calcium blocker and the phosphodiesterase type 5 inhibitor decreases the blood pressure of the subject at least 5% less than a therapeutically effective amount of a combination of a non-N-selective calcium channel blocker and a phosphodiesterase type 5 inhibitor. For example, the dual N-type and L-type selective calcium blocker and the phosphodiesterase type 5 inhibitor decreases the blood pressure of the subject at least 10% less, at least 15% less, at least 20% less, at least 25% less, at least 30% less, at least 35% less, at least 40% less, at least 45% less, at least 50% less, at least 55% less, at least 60% less, at least 65% less, at least 70% less, at least 75% less, at least 80% less, at least 85% less, at least 90% less, or at least 95% less, than a therapeutically effective amount of a combination of a non-N-selective calcium channel blocker and a phosphodiesterase type 5 inhibitor.

In certain embodiments, the subject has scleroderma with interstitial lung disease.

In some embodiments, the method further comprises administering an agent selected from the group consisting of: a calcineurin inhibitor, cyclophosphamide, nintedanib, methotrexate, mycophenolate, a glucocorticoid (e.g., prednisone, dexamethasone, and hydrocortisone), a non-steroidal anti-inflammatory drug (e.g., aspirin, ibuprofen, or naproxen), D-penicillamine, a diuretic, omeprazole, bosentan, epoprostenol, enalapril, Lisinopril, captopril, or any combination thereof. For example, the method further comprises administering nintedanib. In some embodiments, the method further comprises administering a calcineurin inhibitor, a non-steroidal anti-inflammatory drug, or both. In some embodiments, the calcineurin inhibitor is a cyclosporine. In some embodiments, the non-steroidal anti-inflammatory drug is aspirin.

In certain embodiments, the subject has lupus (e.g., systemic lupus erythematosus (SLE)). In some embodiments, the method further comprises administering an agent selected from the group consisting of: an antimalarial drug (e.g., hydroxychloroquine), a non-steroidal anti-inflammatory drug (e.g., aspirin, ibuprofen, or naproxen), belimumab, a corticosteroid (e.g., prednisone or prednisolone), an immunosuppressant (e.g., azathioprine, cyclophosphamide, methotrexate, and mycophenolate mofetil), or any combination thereof.

In certain embodiments, the subject has rheumatoid arthritis. In some embodiments, the method further comprises administering an agent selected from the group consisting of: disease-modifying anti-rheumatic drugs (e.g., methotrexate or sulfasalazine), a non-steroidal anti-inflammatory drug (e.g., aspirin, ibuprofen, or naproxen), a corticosteroid (e.g., prednisone or prednisolone), a biologic (e.g., anakinra or tocilizumab), or any combination thereof.

In certain embodiments, the subject has Sjogren's syndrome. In some embodiments, the method further comprises administering an agent selected from the group consisting of: plaquenil, an antimalarial drug (e.g., hydroxychloroquine), evoxac, cevimeline, infliximab, or any combination thereof.

In certain embodiments, the subject has idiopathic pulmonary fibrosis. In some embodiments, the method further comprises administering an agent selected from the group consisting of: nintedanib, pirfenidone, or any combination thereof.

In certain embodiments, the subject has atherosclerosis. In some of these embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; or the dual N-type and L-type selective calcium blocker (e.g., the dual N-type and L-type selective calcium blocker and the phosphodiesterase type 5 inhibitor)), the atherosclerosis is treated. In some embodiments, treating the atherosclerosis comprises reducing the thickness and/or mass of a plaque in an artery of the subject.

In some embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; or the dual N-type and L-type selective calcium blocker (e.g., the dual N-type and L-type selective calcium blocker and the phosphodiesterase type 5 inhibitor)), the bone density of the subject does not decrease. In some of these embodiments, the bone density of the subject increases. This may occur through a reduction in the number of osteoclasts in the subject and/or an increase in the ratio of alkaline phosphate to tartrate resistant acid phosphatase (TRAP).

In some embodiments, the method further comprises selecting a subject identified or diagnosed as having reduced bone density for the treatment. In some embodiments, the subject identified or diagnosed as having reduced bone density has osteoporosis. In some embodiments, the subject is female.

In some embodiments, the method further comprises selecting a subject identified or diagnosed as having reduced renal function for the treatment. In some embodiments, the renal function of the patient is not reduced after treatment.

In some embodiments, the treating comprises improving renal function in the subject. In some embodiments, improving renal function comprises reducing intrarenal arterial stiffness, improving blood flow to the kidneys, increasing expression levels of podocyte proteins, or any combination thereof.

In some embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; or the dual N-type and L-type selective calcium blocker (e.g., the dual N-type and L-type selective calcium blocker and the phosphodiesterase type 5 inhibitor)), a reduction in sympathetic tone diminution, direct smooth muscle relaxation, dysesthetic pain, burning pain, body temperature changes of the subject, hyperesthesia, changes in skin or tissue color, edema, changes in skin turgor, ruble, pallor, cyanosis, vasospasm, or any combination thereof is observed the subject.

In some embodiments, a reduction in the Raynaud's severity scale (RSS) is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, the reduction is a reduction in one measurement or the average of a plurality of measurements taken after administration of the dual N-type and L-type selective calcium blocker and/or the phosphodiesterase type 5 inhibitor relative to one measurement or the average of a plurality of measurements taken before administration of the dual N-type and L-type selective calcium blocker and/or the phosphodiesterase type 5 inhibitor. In some embodiments, when a plurality of measurements is taken before and after administration, they are taken over the same period of time (e.g., the same number of days). For example, a reduction of at least about 2% (e.g., at least about 4%, 5%, 8%, 10%, 20%, 30%, 40%, 50%, 70%, 90%, 95%) is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). Information on the Raynaud's severity scale can be found at Wigley F M, Wisc R A, Seibold J R et al. Intravenous iloprost infusion in patients with Raynaud phenomenon secondary to systemic sclerosis. A multicenter, placebo-controlled, double-blind study. Ann Intern Med 1994; 120:199-206, which is incorporated by reference herein in its entirety.

In some embodiments, an improvement (e.g., reduction) in the Raynaud's condition scale (RCS) is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, an improvement (e.g., reduction) in the Raynaud's condition scale (RCS) is a reduction in one score or the average (e.g., daily average) of a plurality of scores measured after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to one score or the average (e.g., daily average) of a plurality of scores measured before administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, when a plurality of scores is measured before and after administration, they are taken over the same period of time (e.g., the same number of days). In some embodiments, the period of time is 2 weeks, 7 days, 6 days, 5 days, 4 days, 3 days, 2 days, or 1 day (e.g., 7 day). In some embodiments, a daily average of a plurality of scores is the sum of the plurality of scores divided by the number of days during which the scores were obtained. In some embodiments, the reduction in the Raynaud's condition scale is at least about 2% (e.g., at least about 4%, 5%, 8%, 10%, 20%, 30%, 40%, 45%, 50%, 70%, 90%, 95%) after administration of the dual N-type and L-type selective calcium blocker and/or the phosphodiesterase type 5 inhibitor. In some embodiments, the reduction in the Raynaud's condition scale is at least about 25% after administration of the dual N-type and L-type selective calcium blocker and/or the phosphodiesterase type 5 inhibitor. In some embodiments, the reduction in the Raynaud's condition scale is at least about 45% after administration of the dual N-type and L-type selective calcium blocker and/or the phosphodiesterase type 5 inhibitor. Information on the Raynaud's severity scale can be found in the Examples and at Black C M, Halkier-Sorensen L, Belch J J et al. Oral iloprost in Raynaud's phenomenon secondary to systemic sclerosis: a multicentre, placebo-controlled, dose-comparison study. Br J Rheumatol 1998; 37:952-60, which is incorporated by reference herein in its entirety.

In some embodiments, a reduction in the average pain score (e.g., average weekly pain score) is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). For example, a reduction of at least about 2% (e.g., at least about 4%, 5%, 8%, 10%, 20%, 30%, 40%, 50%, 70%, 90%, 95%) is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, a reduction in the pain score is a reduction in one score or the average of a plurality of scores measured after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to one score the average of a plurality of scores measured before administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, when a plurality of scores are obtained before and after administration, they are taken over the same period of time (e.g., the same number of days). In some embodiments, the time period is 2 weeks, 7 days, 6 days, 5 days, 4 days, 3 days, 2 days, or 1 day (e.g., 7 day). Various scales used to measure pain include the Galer neuropathic pain scale, the Likert pain score, the ID pain Scale, NPQ, PainDETECT, LANS S, DN4 scales, and/or the Standardized Evaluation of Pain (StEP) tool. See, for example, the Examples or Cruccu G, Truini A. Tools for assessing neuropathic pain. PLOS Med. 2009; 6 (4): e1000045. doi: 10.1371/journal.pmed.1000045, which is incorporated by reference herein in its entirety.

In some embodiments, an increase in the temperature of a body part is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). For example, an increase of at least about 0.5% (e.g., at least about 1%, 2%, 3%, 4%, 5%, 8%, 10%, 12%, 15%, or 20%) in the temperature of the body part is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, the increase in the temperature of the body part is an increase in one measurement or the average of a plurality of measurements taken after administration of the dual N-type and L-type selective calcium blocker and/or the phosphodiesterase type 5 inhibitor relative to one measurement or the average of a plurality of measurements taken before administration. In some embodiments, when a plurality of measurements is taken before and after administration, they are taken over the same period of time (e.g., the same number of days). In some embodiments, the increase in temperature is measured by thermography. In some embodiments, the body part is a finger (e.g., an index finger, middle finger, or ring finger (e.g., an index finger)).

In some embodiments, an improvement in the SF-12 index of functional wellbeing is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, the improvement is an improvement in one score or the average of a plurality of scores taken after administration of the dual N-type and L-type selective calcium blocker and/or the phosphodiesterase type 5 inhibitor relative to one score or the average of a plurality of scores taken before administration. In some embodiments, when a plurality of scores are taken before and after administration, they are taken over the same period of time (e.g., the same number of days). In some embodiments, an improvement of at least about 2% (e.g., at least about 4%, 5%, 8%, 10%, 20%, 30%, 40%, 50%, 70%, 90%, 95%) is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). Information on the SF-12 index of functional wellbeing can be found at https://www.physio-pedia.com/12-Item_Short_Form_Survey_(SF-12), which is incorporated by reference herein in its entirety.

In some embodiments, an improvement in the Scleroderma Health Assessment Questionnaire (SHAQ®) is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, an improvement in the Scleroderma Health Assessment Questionnaire comprises an improvement in at least one (e.g., 1 to 8, 2 to 6, 2 to 4, 4 to 6, 6 to 8, 1, 2, 3, 4, 5, 6, 7, or 8) of 1) dressing and grooming, 2) arising, 3) eating, 4) walking, 5) hygiene, 6) reach, 7) grip, and 8) common daily activities in the subject. Information on the Scleroderma Health Assessment Questionnaire (SHAQ®) can be found in the Examples and at Steen V D, Medsger T A Jr. The value of the Health Assessment Questionnaire and special patient-generated scales to demonstrate change in systemic sclerosis patients over time. Arthritis Rheum. 1997 November; 40 (11): 1984-91 and Poole J L, Steen V D. The use of the Health Assessment Questionnaire (HAQ) to determine physical disability in systemic sclerosis. Arthritis Care Res. 1991 March; 4 (1): 27-31, each of which is incorporated by reference herein in its entirety.

In some embodiments, an improvement (i.e., an increase) in the Reactive Hyperemia Index (LnRHI) as measured by Endo PAT is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, the improvement is an improvement in one score or the average of a plurality of scores taken after administration of the dual N-type and L-type selective calcium blocker and/or the phosphodiesterase type 5 inhibitor relative to one score or the average of a plurality of scores taken before administration. In some embodiments, when a plurality of scores are taken before and after administration, they are taken over the same period of time (e.g., the same number of days). For example, an increase of at least about 2% (e.g., at least about 4%, 5%, 8%, 10%, 20%, 30%, 40%, 50%, 70%, 90%, 95%) is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, an improvement in the in the Reactive Hyperemia Index (LnRHI) is an improvement in the average of a plurality of values measured after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to the average of a plurality of values measured before administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) taken over the same time period. In some embodiments, the time period is 2 weeks, 7 days, 6 days, 5 days, 4 days, 3 days, 2 days, or 1 day (e.g., 7 days). In some embodiments, the subject exhibits a reactive hyperemia index of less than 0.51 before administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) and a reactive hyperemia index of at least 0.51 (e.g., 0.51 to 0.7; or at least 0.71) after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, the subject exhibits a reactive hyperemia index of 0.51 to 0.7 before administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) and a reactive hyperemia index of at least 0.71 after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, an improvement in endothelial function as measured by Endo PAT is measured in the subject. In some embodiments, the improvement in endothelial function as measured by Endo PAT is an improvement in one measurement or an average of plurality of measurements taken after administration of the dual N-type and L-type selective calcium blocker and/or the phosphodiesterase type 5 inhibitor relative to one measurement or an average of plurality of measurements taken before administration of the dual N-type and L-type selective calcium blocker and/or the phosphodiesterase type 5 inhibitor. For example, an improvement of at least about 2% (e.g., at least about 4%, 5%, 8%, 10%, 20%, 30%, 40%, 50%, 70%, 90%, 95%) is measured in the subject after administration of the dual N-type and L-type selective calcium blocker and/or the phosphodiesterase type 5 inhibitor. In some embodiments, nitric oxide levels in the endothelium are increased after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) as measured by Endo PAT in the subject. In some embodiments, the increase in nitric oxide levels in the endothelium as measured by Endo PAT is an increase in one measurement or an average of plurality of measurements taken after administration of the dual N-type and L-type selective calcium blocker and/or the phosphodiesterase type 5 inhibitor relative to one measurement or an average of plurality of measurements taken before administration of the dual N-type and L-type selective calcium blocker and/or the phosphodiesterase type 5 inhibitor. For example, an increase of at least about 2% (e.g., at least about 4%, 5%, 8%, 10%, 20%, 30%, 40%, 50%, 70%, 90%, 95%) is measured in the subject after administration of the dual N-type and L-type selective calcium blocker and/or the phosphodiesterase type 5 inhibitor Information on the Reactive Hyperemia Index can be found at https://study.com/academy/lesson/reactive-hyperemia-definition-test.html, which is incorporated by reference herein in its entirety. Information on Endo PAT can be found at https://www.aimil.com/products/endopat#:˜:text=The%20EndoPAT%E2%84%A2%20is%20the%20only%20FDA%20approved%20diagnostic,in%20peripheral%20arterioles%20in%20response%20to%20oxidative%20stress, which is incorporated by reference herein in its entirety.

In some embodiments, an improvement (i.e., a decrease) in the University of California Los Angeles Scleroderma Clinical Trials Consortium Gastrointestinal Tract 2.0 (UCLA SCTC GIT 2.0) questionnaire is observed in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, a decrease in the UCLA SCTC GIT 2.0 questionnaire is a decrease in one score or the average of a plurality of total scores measured after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to one score or the average of a plurality of total scores measured before administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). In some embodiments, when a plurality of scores is taken before and after administration, they are taken over the same period of time (e.g., the same number of days). In some embodiments, the time period is 2 weeks, 7 days, 6 days, 5 days, 4 days, 3 days, 2 days, or 1 day (e.g., 7 day). In some embodiments, a decrease of at least about 2% (e.g., at least about 4%, 5%, 8%, 10%, 20%, 30%, 40%, 50%, 70%, 90%, 95%) is measured in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). More information on the UCLA SCTC GIT 2.0 questionnaire, including how the total score is obtained, can be found in the Examples section.

In some embodiments, an improvement in at least one question (e.g., 2, 3, 4, 5, 6, 7, 8, 9, or 10 questions) in the Assessment of Systemic sclerosis-associated Raynaud's Phenomenon (ASRAP) is observed in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to before administration of the dual N-type and L-type selective calcium blocker and/or the phosphodiesterase type 5 inhibitor. In some embodiments, the improvement in a question is an improvement of at least one degree. An improvement of a degree is defined as an improvement from “very much/a lot” to “quite a bit”, “quite a bit” to “somewhat”, “somewhat” to “a little bit”, or “a little bit” to “not at all”. In some embodiments, the improvement in a questions is an improvement of two degrees, three degrees, or four degrees. More information on the Assessment of Systemic sclerosis-associated Raynaud's Phenomenon (ASRAP), can be found in the Examples section and Pauling et. al. American College of Rheumatology Convergence 2021, Abstract Number 401 (https://acrabstracts.org/abstract/item-reduction-for-the-assessment-of-systemic-sclerosis-associated-raynauds-phenomenon-asrap-questionnaire-using-data-from-the-international- multicentre-asrap-validation-study/), which is incorporated by reference herein in its entirety.

In some embodiments, the method comprises administering at least one additional therapeutic agent to the subject. The at least one additional therapeutic agent can be administered simultaneously, separately, sequentially, or in combination with the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)). Non-limiting examples of additional therapeutic agents include calcium channel blockers, sodium channel blockers (e.g., Nav 1.7 sodium channel blocker), agents that increase blood pressure, and therapeutic agents that relieve pain.

In certain embodiments, the at least one additional therapeutic agent is selected from the group consisting of: riociguat, amlodipine, nifedipine, nicardipine, conotoxins, cadmium, caroverine, gabapentin, levetiracetam, lamotrigine, NP078585, pregabalin, TROX-1, and ziconotide.

In certain embodiments, the at least one additional therapeutic agent is selected from the group consisting of: oxycodone, tramadol, Dilaudid, OxyContin, Cymbalta, Fentanyl Transdermal System, acetaminophen/oxycodone, Roxicodone, Ultram, hydromorphone, Percocet, MS Contin, Butrans, morphine, methadone, buprenorphine, duloxetine, fentanyl, Duragesic, Endocet, Roxanol, Kadian, Roxicet, ConZip, Methadose, Oxyfast, Dazidox, Fentora, Irenka, Methadone Diskets, Oramorph SR, Roxicodone Intensol, Xtampza ER, Actiq, Belbuca, ETH-Oxydose, Infumorph, naloxone/pentazocine, Oxaydo, Oxydose, OxyIR, ziconotide, Abstral, Astramorph PF, Buprenex, Dolophine, Duramorph, Duramorph PF, Embeda, Lazanda, MorphaBond ER, morphine/naltrexone, Prialt, RMS, Roxanol-T, Sublimaze, Subsys, Talwin Nx, Magnacet, Nalocet, Narvox, Perloxx, Primlev, Xolox, and Prolate.

In some embodiments, the agent that increases blood pressure is selected from the group consisting of: midodrine, cortisone, prednisone, trimipramine, venlafaxine, anabolic steroids, antidepressants, anti-obesity drugs, CETP inhibitors, herbal preparations, immunosuppressants, mineralocorticoids, NSAIDS/coxibs, serotonergics, stimulants, sulfonylureas, and sympathomimetic amines.

In some embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) endothelial dysfunction in the subject is improved.

In some embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) oxidative stress in the subject is decreased. In some embodiments, decreasing oxidative stress in the subject comprises decreasing oxidative stress in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to oxidative stress in the subject before administration of the dual N-type and L-type selective calcium blocker and/or the phosphodiesterase type 5 inhibitor.

In some embodiments, after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) blood flow in the subject is increased. In some embodiments, increasing blood flow in the subject comprises increasing blood flow in the subject after administration of the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) relative to blood flow in the subject before administration of the dual N-type and L-type selective calcium blocker and/or the phosphodiesterase type 5 inhibitor. In some embodiments, improving blood flow comprises improving arterial and venous blood flow.

In some embodiments, an antioxidant is not administered to the subject. In some embodiments, the anti-oxidant is selected from the group consisting of a hydralazine compound, a glutathione, vitamin C, cysteine, β-carotene, a ubiquinone, a ubiquinol-10, a tocopherol, coenzyme Q, or a mixture thereof.

In some embodiments, the occurrence of atrial fibrillation is decreased in the subject. It is understood that decreasing the occurrence of atrial fibrillation in the subject occurs by means of a decrease in autonomic dysfunction.

In another aspect, disclosed herein is a method of reducing atrial remodeling in a subject with atrial fibrillation and scleroderma, comprising administering a dual N-type and L-type calcium channel blocker selective for the N-type calcium channel and/or a phosphodiesterase type 5 inhibitor to the subject. In some embodiments, the dual N-type and L-type selective calcium blocker and the phosphodiesterase type 5 inhibitor are administered to the subject. In some embodiments, the dual N-type and L-type selective calcium blocker is administered to the subject. In some embodiments, the phosphodiesterase type 5 inhibitor is administered to the subject.

In some embodiments, the dual N-type and L-type selective calcium blocker is formulated to maintain the plasma level of the dual N-type and L-type selective calcium blocker in the subject at 10% or greater (e.g., 15% or greater, 20% or greater, 25% or greater, 30% or greater, 35% or greater, 40% or greater, 45% or greater, 50% or greater, 55% or greater, 60% or greater, 65% or greater, 70% or greater, 75% or greater, 80% or greater, 85% or greater, 90% or greater, or 95% or greater) of the peak dual N-type and L-type selective calcium blocker plasma level for at least 6 hours (e.g., at least 8 hours, at least 12 hours, at least 16 hours, at least 20 hours, at least 24 hours, at least 36 hours, or at least 48 hours) after administration of the dual N-type and L-type selective calcium blocker. It is understood that the peak dual N-type and L-type selective calcium blocker plasma level is the highest plasma concentration of the dual N-type and L-type selective calcium blocker observed in the subject after administration of the dual N-type and L-type selective calcium blocker.

### Pharmaceutical Compositions and Formulations

In another aspect, disclosed herein is a pharmaceutical composition comprising a dual N-type and L-type calcium channel blocker selective for the N-type calcium channel and a phosphodiesterase type 5 inhibitor.

In some embodiments, the pharmaceutical composition further comprises a pharmaceutically acceptable excipient.

In some embodiments, the composition is in the form of a tablet or capsule.

In some embodiments, the phosphodiesterase type 5 inhibitor is selected from sildenafil, tadalafil, or pharmaceutically acceptable salts thereof. In some embodiments, the phosphodiesterase type 5 inhibitor is tadalafil or a pharmaceutically acceptable salt thereof. In some embodiments, the phosphodiesterase type 5 inhibitor is sildenafil or a pharmaceutically acceptable salt thereof.

In some embodiments, the amount of the dual N-type and L-type calcium channel blocker selective for the N-type calcium channel in the composition is about 5 mg to about 25 mg.

In some embodiments, the amount of the dual N-type and L-type calcium channel blocker selective for the N-type calcium channel in the composition is about 9 mg to about 21 mg.

In some embodiments, the amount of the dual N-type and L-type calcium channel blocker selective for the N-type calcium channel in the composition is about 10 mg.

In some embodiments, the amount of the dual N-type and L-type calcium channel blocker selective for the N-type calcium channel in the composition is about 20 mg.

In some embodiments, the amount of the phosphodiesterase type 5 inhibitor in the composition is about 2 mg to about 8 mg.

In some embodiments, the amount of the phosphodiesterase type 5 inhibitor in the composition is about 5 mg.

In some embodiments, the dual N-type and L-type selective calcium blocker is cilnidipine in an amount of about 20 mg and the phosphodiesterase type 5 inhibitor is tadalafil in an amount of about 5 mg. In some embodiments, the dual N-type and L-type selective calcium blocker is cilnidipine in an amount of about 10 mg and the phosphodiesterase type 5 inhibitor is tadalafil in an amount of about 5 mg.

In some embodiments, the pharmaceutical composition further comprises an agent that increases blood pressure.

In some embodiments, the agent that increases blood pressure is selected from the group consisting of: midodrine, cortisone, prednisone, trimipramine, venlafaxine, anabolic steroids, antidepressants, anti-obesity drugs, CETP inhibitors, herbal preparations, immunosuppressants, mineralocorticoids, NSAIDS/coxibs, serotonergics, stimulants, sulfonylureas, and sympathomimetic amines.

In some embodiments, the pharmaceutical composition further comprises a non-steroidal anti-inflammatory drug (e.g., aspirin, ibuprofen, or naproxen).

In some embodiments, the pharmaceutical composition further comprises an agent selected from the group consisting of: a calcineurin inhibitor, cyclophosphamide, nintedanib, methotrexate, mycophenolate, a glucocorticoid (e.g., prednisone, dexamethasone, and hydrocortisone), a non-steroidal anti-inflammatory drug (e.g., aspirin, ibuprofen, or naproxen), D-penicillamine, a diuretic, omeprazole, bosentan, epoprostenol, enalapril, lisinopril, captopril, or any combination thereof. For example, the pharmaceutical composition further comprises administering nintedanib. In some embodiments, the pharmaceutical composition further comprises a calcineurin inhibitor, a non-steroidal anti-inflammatory drug, or both.

In some embodiments, the pharmaceutical composition further comprises an agent selected from the group consisting of: a calcineurin inhibitor, cyclophosphamide, nintedanib, methotrexate, mycophenolate, a glucocorticoid (e.g., prednisone, dexamethasone, and hydrocortisone), a non-steroidal anti-inflammatory drug (e.g., aspirin, ibuprofen, or naproxen), D-penicillamine, a diuretic, omeprazole, bosentan, epoprostenol, enalapril, Lisinopril, captopril, or any combination thereof. For example, the method further comprises administering nintedanib. In some embodiments, the pharmaceutical composition further comprises a calcineurin inhibitor, a non-steroidal anti-inflammatory drug, or both. In some embodiments, the calcineurin inhibitor is a cyclosporine. In some embodiments, the non-steroidal anti-inflammatory drug is aspirin.

In some embodiments, the pharmaceutical composition further comprises an agent selected from the group consisting of: an antimalarial drug (e.g., hydroxychloroquine), a non-steroidal anti-inflammatory drug (e.g., aspirin, ibuprofen, or naproxen), belimumab, a corticosteroid (e.g., prednisone or prednisolone), an immunosuppressant (e.g., azathioprine, cyclophosphamide, methotrexate, and mycophenolate mofetil), or any combination thereof.

In some embodiments, the pharmaceutical composition further comprises an agent selected from the group consisting of: disease-modifying anti-rheumatic drugs (e.g., methotrexate or sulfasalazine), a non-steroidal anti-inflammatory drug (e.g., aspirin, ibuprofen, or naproxen), a corticosteroid (e.g., prednisone or prednisolone), a biologic (e.g., anakinra or tocilizumab), or any combination thereof.

In some embodiments, the pharmaceutical composition further comprises an agent selected from the group consisting of: plaquenil, an antimalarial drug (e.g., hydroxychloroquine), evoxac, cevimeline, infliximab, or any combination thereof.

In some embodiments, the pharmaceutical composition further comprises an agent selected from the group consisting of: nintedanib, pirfenidone, or any combination thereof.

In some embodiments, the dual N-type and L-type calcium channel blocker selective for the N-type calcium channel is selected from the group consisting of: cilnidipine, Z-160, zicinotide, ralfinamide, CNV2197944, or pharmaceutically acceptable salts thereof. In some embodiments, the dual N-type and L-type calcium channel blocker selective for the N-type calcium channel is selected from the group consisting of: cilnidipine, Z-160, ralfinamide, CNV2197944, or pharmaceutically acceptable salts thereof. In some embodiments, the dual N-type and L-type calcium channel blocker selective for the N-type calcium channel is selected from the group consisting of: cilnidipine, Z-160, CNV2197944, or pharmaceutically acceptable salts thereof. In some embodiments, the dual N-type and L-type calcium channel blocker selective for the N-type calcium channel is selected from the group consisting of: cilnidipine, Z-160, or pharmaceutically acceptable salts thereof. In certain embodiments, the dual N-type and

L-type calcium channel blocker selective for the N-type calcium channel is a dihydropyridine N-type calcium channel blocker. In certain of these embodiments, the dual N-type and L-type calcium channel blocker selective for the N-type calcium channel is cilnidipine or a pharmaceutically acceptable salt thereof. In some embodiments, the dual N-type and L-type calcium channel blocker selective for the N-type calcium channel is CNV2197944.

In some embodiments, a combination of chemical entities (e.g., a dual N-type and L-type calcium channel blocker selective for the N-type calcium channel and a phosphodiesterase type 5 inhibitor) is administered as a pharmaceutical composition that includes the a dual N-type and L-type calcium channel blocker selective for the N-type calcium channel and a phosphodiesterase type 5 inhibitor, and one or more pharmaceutically acceptable excipients, and optionally one or more additional therapeutic agents as described herein.

In some embodiments, the dual N-type and L-type calcium channel blocker selective for the N-type calcium channel and the phosphodiesterase type 5 inhibitor can be administered in combination with one or more conventional pharmaceutical excipients. Pharmaceutically acceptable excipients include, but are not limited to, ion exchangers, alumina, aluminum stearate, lecithin, self-emulsifying drug delivery systems (SEDDS) such as d-α-tocopherol polyethylene glycol 1000 succinate, surfactants used in pharmaceutical dosage forms such as Tweens, poloxamers or other similar polymeric delivery matrices, serum proteins, such as human serum albumin, buffer substances such as phosphates, tris, glycine, sorbic acid, potassium sorbate, partial glyceride mixtures of saturated vegetable fatty acids, water, salts or electrolytes, such as protamine sulfate, disodium hydrogen phosphate, potassium hydrogen phosphate, sodium-chloride, zinc salts, colloidal silica, magnesium trisilicate, polyvinyl pyrrolidone, cellulose-based substances, polyethylene glycol, sodium carboxymethyl cellulose, polyacrylates, waxes, polyethylene-polyoxypropylene-block polymers, and wool fat. Cyclodextrins such as α-, β, and γ-cyclodextrin, or chemically modified derivatives such as hydroxyalkylcyclodextrins, including 2- and 3-hydroxypropyl-β-cyclodextrins, or other solubilized derivatives can also be used to enhance delivery of compounds described herein. Dosage forms or compositions containing a chemical entity as described herein in the range of 0.005% to 100% with the balance made up from non-toxic excipient may be prepared. The contemplated compositions may contain 0.001%-100% of a chemical entity provided herein, in one embodiment 0.1-95%, in another embodiment 75-85%, in a further embodiment 20-80%. Actual methods of preparing such dosage forms are known, or will be apparent, to those skilled in this art; for example, see Remington: The Science and Practice of Pharmacy, 22^{nd} Edition (Pharmaceutical Press, London, U K. 2012).

### Routes of Administration and Composition Components

In some embodiments, the chemical entities described herein or a pharmaceutical composition thereof can be administered to subject in need thereof by any accepted route of administration. Acceptable routes of administration include, but are not limited to, oral, parenteral, transdermal, intranasal, sublingual, neuraxial, or ocular (e.g., oral).

In some embodiments, the compounds described herein or a pharmaceutical composition thereof are suitable for local delivery to the digestive or GI tract by way of oral administration (e.g., solid or liquid dosage forms).

Solid dosage forms for oral administration include capsules, tablets, pills, powders, and granules. In such solid dosage forms, the chemical entity is mixed with one or more pharmaceutically acceptable excipients, such as sodium citrate or dicalcium phosphate and/or: a) fillers or extenders such as starches, lactose, sucrose, glucose, mannitol, and silicic acid, b) binders such as, for example, carboxymethylcellulose, alginates, gelatin, polyvinylpyrrolidinone, sucrose, and acacia, c) humectants such as glycerol, d) disintegrating agents such as agar-agar, calcium carbonate, potato or tapioca starch, alginic acid, certain silicates, and sodium carbonate, e) solution retarding agents such as paraffin, f) absorption accelerators such as quaternary ammonium compounds, g) wetting agents such as, for example, cetyl alcohol and glycerol monostearate, h) absorbents such as kaolin and bentonite clay, and i) lubricants such as talc, calcium stearate, magnesium stearate, solid polyethylene glycols, sodium lauryl sulfate, and mixtures thereof. In the case of capsules, tablets and pills, the dosage form may also comprise buffering agents. Solid compositions of a similar type may also be employed as fillers in soft and hard-filled gelatin capsules using such excipients as lactose or milk sugar as well as high molecular weight polyethylene glycols and the like.

In some embodiments, the compositions will take the form of a unit dosage form such as a pill or tablet and thus the composition may contain, along with a chemical entity provided herein, a diluent such as lactose, sucrose, dicalcium phosphate, or the like; a lubricant such as magnesium stearate or the like; and a binder such as starch, gum acacia, polyvinylpyrrolidine, gelatin, cellulose, cellulose derivatives or the like. In another solid dosage form, a powder, marume, solution or suspension (e.g., in propylene carbonate, vegetable oils, PEG's, poloxamer 124 or triglycerides) is encapsulated in a capsule (gelatin or cellulose base capsule). Unit dosage forms in which one or more chemical entities provided herein or additional active agents are physically separated are also contemplated; e.g., capsules with granules (or tablets in a capsule) of each drug; two-layer tablets; two-compartment gel caps, etc. Enteric coated or delayed release oral dosage forms are also contemplated.

Other physiologically acceptable compounds include wetting agents, emulsifying agents, dispersing agents or preservatives that are particularly useful for preventing the growth or action of microorganisms. Various preservatives are well known and include, for example, phenol and ascorbic acid.

In certain embodiments the excipients are sterile and generally free of undesirable matter. These compositions can be sterilized by conventional, well-known sterilization techniques. For various oral dosage form excipients such as tablets and capsules sterility is not required. The USP/NF standard is usually sufficient.

In certain embodiments, solid oral dosage forms can further include one or more components that chemically and/or structurally predispose the composition for delivery of the chemical entity to the stomach or the lower GI; e.g., the ascending colon and/or transverse colon and/or distal colon and/or small bowel. Exemplary formulation techniques are described in, e.g., Filipski, K. J., et al., Current Topics in Medicinal Chemistry, 2013, 13, 776-802, which is incorporated herein by reference in its entirety.

Examples include upper-GI targeting techniques, e.g., Accordion Pill (Intec Pharma), floating capsules, and materials capable of adhering to mucosal walls.

Other examples include lower-GI targeting techniques. For targeting various regions in the intestinal tract, several enteric/pH-responsive coatings and excipients are available. These materials are typically polymers that are designed to dissolve or erode at specific pH ranges, selected based upon the GI region of desired drug release. These materials also function to protect acid labile drugs from gastric fluid or limit exposure in cases where the active ingredient may be irritating to the upper GI (e.g., hydroxypropyl methylcellulose phthalate series, Coateric (polyvinyl acetate phthalate), cellulose acetate phthalate, hydroxypropyl methylcellulose acetate succinate, Eudragit series (methacrylic acid-methyl methacrylate copolymers), and Marcoat). Other techniques include dosage forms that respond to local flora in the GI tract, Pressure-controlled colon delivery capsule, and Pulsincap.

In any of the foregoing embodiments, pharmaceutical compositions described herein can include one or more one or more of the following: lipids, interbilayer crosslinked multilamellar vesicles, biodegradeable poly(D,L-lactic-co-glycolic acid) [PLGA]-based or poly anhydride-based nanoparticles or microparticles, and nanoporous particle-supported lipid bilayers.

### Regimens

The foregoing dosages can be administered on a daily basis (e.g., as a single dose or as two or more divided doses) or non-daily basis (e.g., every other day, every two days, every three days, once weekly, twice weeks, once every two weeks, once a month).

In some embodiments, each administration of the dual N-type and L-type calcium channel blocker selective for the N-type calcium channel and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor)) is separated by at least about 12 hours. For example, each administration of the dual N-type and L-type calcium channel blocker selective for the N-type calcium channel and/or the phosphodiesterase type 5 inhibitor is separated by at least about 24 hours, at least about 30 hours, at least about 48 hours, at least about 60 hours, at least about 72 hours, at least about 4 days, at least about 5 days, at least about 6 days, at least about 1 week, at least about 9 days, at least about 12 days, or at least about 2 weeks. For example, each administration of the dual N-type and L-type calcium channel blocker selective for the N-type calcium channel and/or the phosphodiesterase type 5 inhibitor is separated by about 24 hours.

In some embodiments, the period of administration of a compound described herein (e.g., the dual N-type and L-type selective calcium blocker and/or the phosphodiesterase type 5 inhibitor (e.g., the dual N-type and L-type selective calcium blocker and the phosphodiesterase type 5 inhibitor; the dual N-type and L-type selective calcium blocker; or the phosphodiesterase type 5 inhibitor (e.g., the dual N-type and L-type selective calcium blocker and the phosphodiesterase type 5 inhibitor))) is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In an embodiment, a therapeutic compound is administered to an individual for a period of time followed by a separate period of time. In another embodiment, a therapeutic compound is administered for a first period and a second period following the first period, with administration stopped during the second period, followed by a third period where administration of the therapeutic compound is started and then a fourth period following the third period where administration is stopped. In an aspect of this embodiment, the period of administration of a therapeutic compound followed by a period where administration is stopped is repeated for a determined or undetermined period of time. In a further embodiment, a period of administration is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more. In a further embodiment, a period of during which administration is stopped is for 1 day, 2 days, 3 days, 4 days, 5 days, 6 days, 7 days, 8 days, 9 days, 10 days, 11 days, 12 days, 13 days, 14 days, 3 weeks, 4 weeks, 5 weeks, 6 weeks, 7 weeks, 8 weeks, 9 weeks, 10 weeks, 11 weeks, 12 weeks, 4 months, 5 months, 6 months, 7 months, 8 months, 9 months, 10 months, 11 months, 12 months, or more.

### Exemplary Embodiments

Embodiment 1. A method of treating Raynaud's syndrome in a subject in need thereof, comprising administering to the subject a therapeutically effective amount of a dual N-type and L-type calcium channel blocker selective for the N-type calcium channel and a phosphodiesterase type 5 inhibitor.

Embodiment 2. The method of embodiment 1, wherein the subject has scleroderma and the Raynaud's syndrome is secondary Raynaud's syndrome.

Embodiment 3. The method of any one of embodiments 1-2, wherein the treating comprises dilating arterioles, dilating venules, increasing production of nitrous oxide, reducing norepinephrine, improving endothelial function, inhibiting calcitonin gene-related neuropeptide (CGRP), reducing inflammation, or any combination thereof in the subject.

Embodiment 4. The method of any one of embodiments 1-3, wherein the treating comprises reducing fibrosis in the subject.

Embodiment 5. The method of embodiment 4, wherein reducing fibrosis comprises reducing formation of collagen and extracellular matrix proteins in the subject.

Embodiment 6. The method of embodiment 5, wherein the collagen is formed by fibroblasts.

Embodiment 7. The method of any one of embodiments 4-6, wherein the fibrosis is renal fibrosis or myocardial fibrosis.

Embodiment 8. The method of any one of embodiments 1-7, wherein the treating comprises improving vascular function in the subject.

Embodiment 9. The method of embodiment 8, wherein improving vascular function comprises decreasing intima media thickness (IMT), decreasing arterial stiffness, reducing urinary albumin excretion (UAE), reducing plaque in the arteries, or any combination thereof.

Embodiment 10. The method of any one of embodiments 1-9, wherein the subject also has interstitial lung disease.

Embodiment 11. The method of embodiment 10, wherein after administration of the dual N-type and L-type selective calcium blocker and the phosphodiesterase type 5 inhibitor the interstitial lung disease is treated.

Embodiment 12. The method of any one of embodiments 1-11, wherein the treating comprises improving lung function in the subject.

Embodiment 13. The method of any one of embodiments 1-12, wherein the subject has digital ulcerations.

Embodiment 14. The method of embodiment 13, wherein after administration of the dual N-type and L-type selective calcium blocker and the phosphodiesterase type 5 inhibitor, the number and/or severity of the digital ulcerations is reduced.

Embodiment 15. The method of any one of embodiments 1-14, wherein the treating comprises improving cardiac function in the subject.

Embodiment 16. The method of embodiment 15, wherein improving cardiac function in the subject comprises reducing the frequency and/or severity of cardiac arrhythmias; reducing sympathomimetic increases in papillary muscle-developed tension (PMDT); reducing myocardial interstitial norepinephrine level; decreasing aortic pressure; increased aortic, vertebral, and coronary blood flow; reducing myocardial oxygen consumption; reducing blood pressure; or any combination thereof.

Embodiment 17. The method of any one of embodiments 1-16, wherein the treating comprises reducing pulmonary hypertension in the subject.

Embodiment 18. The method of any one of embodiments 1-17, wherein the treating comprises improving renal function in the subject.

Embodiment 19. The method of embodiment 18, wherein improving renal function comprises reducing intrarenal arterial stiffness, improving blood flow to the kidneys, increasing expression levels of podocyte proteins, or any combination thereof.

Embodiment 20. The method of any one of embodiments 1-19, wherein the treating comprises reducing the frequency of one or more symptoms associated with Raynaud's syndrome in the subject.

Embodiment 21. The method of any one of embodiments 1-20, wherein the treating comprises reducing the severity of one or more symptoms associated with Raynaud's syndrome in the subject.

Embodiment 22. The method of any one of embodiments 1-21, wherein the treating comprises reducing the duration of one or more symptoms associated with Raynaud's syndrome in the subject.

Embodiment 23. The method of embodiment 22, wherein the duration of the one or more symptoms associated with Raynaud's syndrome in the subject is reduced by at least 20%.

Embodiment 24. The method of any one of embodiments 1-23, wherein starting before administration of the dual N-type and L-type selective calcium blocker and the phosphodiesterase type 5 inhibitor, the subject was administered an alternative therapy useful to treat the Raynaud's disease at regular intervals; and after administration of the dual N-type and L-type selective calcium blocker and the phosphodiesterase type 5 inhibitor to the subject, the dosage and/or frequency of the alternative therapy required to treat the subject is reduced.

Embodiment 25. The method of embodiment 24, wherein the alternative therapy is a non-N-selective calcium channel blocker.

Embodiment 26. A method of reducing the frequency of one or more symptoms associated with Raynaud's syndrome in a subject, comprising administering to the subject a therapeutically effective amount of a dual N-type and L-type calcium channel blocker selective for the N-type calcium channel and a phosphodiesterase type 5 inhibitor.

Embodiment 27. The method of any one of embodiments 20-26, wherein the frequency of one or more symptoms associated with Raynaud's syndrome in the subject is reduced by at least 25%.

Embodiment 28. A method of reducing the severity of one or more symptoms associated with Raynaud's syndrome in a subject, comprising administering to the subject a therapeutically effective amount of a dual N-type and L-type calcium channel blocker selective for the N-type calcium channel and a phosphodiesterase type 5 inhibitor.

Embodiment 29. The method of any one of embodiments 21-25 and 28, wherein reducing the severity of the one or more symptoms associated with Raynaud's syndrome comprises measuring a reduction in a score provided by the visual analog scale.

Embodiment 30. The method of embodiment 29, wherein the score is reduced by at least 20%.

Embodiment 31. The method of any one of embodiments 26-30, wherein the subject has scleroderma.

Embodiment 32. The method of any one of embodiments 26-31, wherein the Raynaud's syndrome is secondary Raynaud's syndrome.

Embodiment 33. The method of any one of embodiments 20-32, wherein the symptoms are selected from the group consisting of: pain, anemia, fatigue, change in coloration of the skin, cyanosis, reperfusion, deoxygenation of the blood, digital ulcerations, reduced temperature in one or more parts of the body, changes in the endothelium of a blood vessel, swelling, impaired vision, or any combination thereof.

Embodiment 34. The method of embodiment 33, wherein the symptom is pain.

Embodiment 35. A method of reducing pain or discomfort caused by a reduction of body temperature in a subject, comprising administering to the subject a therapeutically effective amount of a dual N-type and L-type calcium channel blocker selective for the N-type calcium channel and a phosphodiesterase type 5 inhibitor, wherein the reduction of body temperature in the subject is caused by an exposure of the subject to air having a temperature of less than 25° C.

Embodiment 36. The method of embodiment 35, wherein the reduction of body temperature in the subject is caused by an exposure of the subject to air having a temperature of less than 10° C.

Embodiment 37. The method of any one of embodiments 35-36, wherein the reduction of body temperature in the subject comprises reduction in the temperature of a finger of the subject.

Embodiment 38. The method of any one of embodiments 35-37, wherein the reduction of body temperature in the subject comprises reduction in the temperature of a hand of the subject.

Embodiment 39. The method of any one of embodiments 35-38, wherein the reduction of body temperature in the subject comprises reduction in the temperature of a foot of the subject.

Embodiment 40. A method of reducing susceptibility of a subject to cold-induced pain or discomfort, comprising administering to the subject a therapeutically effective amount of a dual N-type and L-type calcium channel blocker selective for the N-type calcium channel and a phosphodiesterase type 5 inhibitor.

Embodiment 41. The method of any one of embodiments 35-40, wherein the subject has scleroderma.

Embodiment 42. The method of embodiment 41, wherein the scleroderma is limited scleroderma.

Embodiment 43. The method of embodiment 41, wherein the scleroderma is diffuse scleroderma.

Embodiment 44. The method of any one of embodiments 1-43, wherein vasoconstriction in the subject is reduced after administering the dual N-type and L-type selective calcium blocker and the phosphodiesterase type 5 inhibitor to the subject.

Embodiment 45. The method of any one of embodiments 1-44, wherein the dual N-type and L-type calcium channel blocker selective for the N-type calcium channel exhibits at least a 50-fold selectivity for the N-type calcium channel over an L-type calcium channel.

Embodiment 46. The method of any one of embodiments 1-45, wherein the dual N-type and L-type calcium channel blocker selective for the N-type calcium channel exhibits a 50-fold to 100-fold selectivity for the N-type calcium channel over an L-type calcium channel.

Embodiment 47. The method of any one of embodiments 1-46, wherein the dual N-type and L-type calcium channel blocker selective for the N-type calcium channel is selected from the group consisting of: cilnidipine, Z-160, CNV2197944, or pharmaceutically acceptable salts thereof.

Embodiment 48. The method of any one of embodiments 1-47, wherein the dual N-type and L-type calcium channel blocker selective for the N-type calcium channel is cilnidipine or a pharmaceutically acceptable salt thereof.

Embodiment 49. The method of any one of embodiments 1-48, wherein the phosphodiesterase type 5 inhibitor is selected from sildenafil, tadalafil, or pharmaceutically acceptable salts thereof.

Embodiment 50. The method of any one of embodiments 1-48, wherein the phosphodiesterase type 5 inhibitor is tadalafil or a pharmaceutically acceptable salt thereof.

Embodiment 51. The method of any one of embodiments 1-50, wherein the amount of the phosphodiesterase type 5 inhibitor used in the method is less than the therapeutically effective amount of the phosphodiesterase type 5 inhibitor useful to treat Raynaud's syndrome in a subject when administered in combination with a non-N selective calcium channel blocker.

Embodiment 52. The method of any one of embodiments 1-51, wherein the dosage of the dual N-type and L-type calcium channel blocker selective for the N-type calcium channel is about 5 mg to about 25 mg.

Embodiment 53. The method of any one of embodiments 1-51, wherein the dosage of the dual N-type and L-type calcium channel blocker selective for the N-type calcium channel is about 9 mg to about 21 mg.

Embodiment 54. The method of any one of embodiments 1-54, wherein the dosage of the dual N-type and L-type calcium channel blocker selective for the N-type calcium channel is about 10 mg.

Embodiment 55. The method of any one of embodiments 1-54, wherein the dosage of the dual N-type and L-type calcium channel blocker selective for the N-type calcium channel is about 20 mg.

Embodiment 56. The method of any one of embodiments 1-55, wherein the dosage of the phosphodiesterase type 5 inhibitor is about 8 mg to 40 mg.

Embodiment 57. The method of any one of embodiments 1-56, wherein the dosage of the phosphodiesterase type 5 inhibitor is about 2 mg to about 8 mg.

Embodiment 58. The method of any one of embodiments 1-57, wherein the dosage of the phosphodiesterase type 5 inhibitor is about 5 mg.

Embodiment 59. The method of any one of embodiments 1-58, wherein the dual N-type and L-type selective calcium blocker and the phosphodiesterase type 5 inhibitor are administered orally.

Embodiment 60. The method of any one of embodiments 1-59, wherein the dual N-type and L-type selective calcium blocker and the phosphodiesterase type 5 inhibitor are administered separately, sequentially, or simultaneously.

Embodiment 61. The method of embodiment 60, wherein the dual N-type and L-type selective calcium blocker and the phosphodiesterase type 5 inhibitor are administered simultaneously.

Embodiment 62. The method of embodiment 61, wherein the dual N-type and L-type selective calcium blocker and the phosphodiesterase type 5 inhibitor are administered simultaneously as a fixed dosage form.

Embodiment 63. The method of embodiment 62, wherein each administration of the dual N-type and L-type selective calcium blocker and the phosphodiesterase type 5 inhibitor is less frequent than the frequency of administering the dual N-type and L-type selective calcium blocker or the phosphodiesterase type 5 inhibitor alone useful to treat the Raynaud's syndrome.

Embodiment 64. The method of any one of embodiments 1-63, wherein each administration of the dual N-type and L-type selective calcium blocker and the phosphodiesterase type 5 inhibitor is separated by at least about 8 hours.

Embodiment 65. The method of any one of embodiments 1-64, wherein each administration of the dual N-type and L-type selective calcium blocker and the phosphodiesterase type 5 inhibitor is separated by at least about 24 hours.

Embodiment 66. The method of any one of embodiments 1-65, wherein each administration of the dual N-type and L-type selective calcium blocker and the phosphodiesterase type 5 inhibitor is separated by at least about 48 hours.

Embodiment 67. The method of any one of embodiments 1-66, wherein each administration of the dual N-type and L-type selective calcium blocker and the phosphodiesterase type 5 inhibitor is separated by at least about 72 hours.

Embodiment 68. The method of any one of embodiments 1-67, wherein the subject experiences less frequent, less severe, and/or shorter episodes of tachycardia, headaches, flushing, increased heart rate, flushing, decreased renal blood flow, myalgia, chest pain, heart palpitation, and/or pedal enema than when administered a therapeutically effective amount of a non-N-selective calcium channel blocker alone, a phosphodiesterase type 5 inhibitor alone, or a combination of a non-N-selective calcium channel blocker and a phosphodiesterase type 5 inhibitor useful to treat the secondary Raynaud's syndrome.

Embodiment 69. The method of any one of embodiments 25, 27, 29-30, 33, and 51-68, wherein the non-N-selective calcium channel blocker is selected from the group consisting of: nifedipine, nicardipine, amlodipine, Z-944, nimodipine, verapamil, diltiazem, felodipine, isradipine, nisoldipine, and nitrendipine.

Embodiment 70. The method of any one of embodiments 1-69, wherein after administration of the dual N-type and L-type selective calcium blocker and the phosphodiesterase type 5 inhibitor in the subject, sympathetic tone diminution, direct smooth muscle relaxation, or both occur in the subject.

Embodiment 71. The method of any one of embodiments 1-70, after administration of the dual N-type and L-type selective calcium blocker and the phosphodiesterase type 5 inhibitor in the subject, norepinephrine is reduced in the subject.

Embodiment 72. The method of any one of embodiments 1-71, wherein the subject is also diagnosed with hypertension; and wherein after administration of the dual N-type and L-type selective calcium blocker and the phosphodiesterase type 5 inhibitor to the subject, the systolic blood pressure of the subject is reduced.

Embodiment 73. The method of embodiment 72, wherein the systolic blood pressure of the subject is reduced by greater than 10 mm Hg.

Embodiment 74. The method of any one of embodiments 1-73, wherein after administration of the dual N-type and L-type selective calcium blocker and the phosphodiesterase type 5 inhibitor, the bone density of the subject does not decrease.

Embodiment 75. The method of any one of embodiments 1-74, further comprising selecting a subject identified or diagnosed as having reduced bone density for the treatment.

Embodiment 76. The method of embodiment 75, wherein the subject identified or diagnosed as having reduced bone density has osteoporosis.

Embodiment 77. The method of any one of embodiments 75-76, wherein the subject is female.

Embodiment 78. The method of any one of embodiments 1-77, further comprising selecting a subject identified or diagnosed as having reduced renal function for the treatment.

Embodiment 79. The method of embodiment 78, wherein the renal function of the patient is not reduced after treatment.

Embodiment 80. The method of any one of embodiments 78-79, wherein the renal function of the patient is improved after treatment.

Embodiment 81. The method of any one of embodiments 1-80, wherein a reduction in the Raynaud's condition scale is measured in the subject.

Embodiment 82. The method of embodiment 81, wherein the reduction in the Raynaud's condition scale is at least about 25%.

Embodiment 83. The method of any one of embodiments 1-82, wherein a reduction in the Raynaud's severity scale is measured in the subject.

Embodiment 84. The method of any one of embodiments 1-83, wherein a reduction in the average weekly pain score is measured in the subject.

Embodiment 85. The method of any one of embodiments 1-84, wherein an increase in the temperature of a body part as measured by thermography is observed in the subject.

Embodiment 86. The method of embodiment 85, wherein the body part is an index finger.

Embodiment 87. The method of any one of embodiments 1-86, wherein an improvement in the SF-12 index of functional wellbeing is measured in the subject.

Embodiment 88. The method of any one of embodiments 1-87, wherein an improvement in Scleroderma Health Assessment Questionnaire (SHAQ®) is measured in the subject.

Embodiment 89. The method of any one of embodiments 1-88, wherein a reduction in the Reactive Hyperemia Index as measured by Endo PAT is measured in the subject.

Embodiment 90. The method of any one of embodiments 1-89, wherein an improvement in endothelial function as measured by Endo PAT is measured in the subject.

Embodiment 91. The method of any one of embodiments 1-90, wherein nitric oxide levels in the endothelium are increased as measured by Endo PAT in the subject.

Embodiment 92. A pharmaceutical composition comprising a dual N-type and L-type calcium channel blocker selective for the N-type calcium channel and a phosphodiesterase type 5 inhibitor.

Embodiment 93. The composition of embodiment 92, comprising a pharmaceutically acceptable excipient.

Embodiment 94. The composition of any one of embodiments 92-93, wherein the composition is in the form of a tablet or capsule.

Embodiment 95. The composition of any one of embodiments 92-94, wherein the dual N-type and L-type calcium channel blocker selective for the N-type calcium channel is selected from the group consisting of: cilnidipine, Z-160, CNV2197944, or pharmaceutically acceptable salts thereof.

Embodiment 96. The composition of any one of embodiments 92-95, wherein the dual N-type and L-type calcium channel blocker selective for the N-type calcium channel is cilnidipine or a pharmaceutically acceptable salt thereof.

Embodiment 97. The composition of any one of embodiments 92-96, wherein the phosphodiesterase type 5 inhibitor is selected from sildenafil, tadalafil, or pharmaceutically acceptable salts thereof.

Embodiment 98. The composition of any one of embodiments 92-97, wherein the phosphodiesterase type 5 inhibitor is tadalafil or a pharmaceutically acceptable salt thereof.

Embodiment 99. The composition of any one of embodiments 92-98, wherein the amount of the dual N-type and L-type calcium channel blocker selective for the N-type calcium channel in the composition is about 5 mg to about 25 mg.

Embodiment 100. The composition of any one of embodiments 92-99, wherein the amount of the dual N-type and L-type calcium channel blocker selective for the N-type calcium channel in the composition is about 9 mg to about 21 mg.

Embodiment 101. The composition of any one of embodiments 92-100, wherein the amount of the dual N-type and L-type calcium channel blocker selective for the N-type calcium channel in the composition is about 10 mg.

Embodiment 102. The composition of any one of embodiments 92-100, wherein the amount of the dual N-type and L-type calcium channel blocker selective for the N-type calcium channel in the composition is about 20 mg.

Embodiment 103. The composition of any one of embodiments 92-102, wherein the amount of the phosphodiesterase type 5 inhibitor in the composition is about 2 mg to about 8 mg.

Embodiment 104. The composition of any one of embodiments 92-103, wherein the amount of the phosphodiesterase type 5 inhibitor in the composition is about 5 mg.

### EXAMPLES

### Example 1. Clinical Trial Protocol

TABLE 1
Abbreviations.

| Abbreviation | Term |
|---|---|
| ACR | American College of Rheumatology |
| AE | Adverse Event |
| ARB | Angiotensin II receptor blocker |
| ATC | Anatomical Therapeutic Class |
| AUC_{0-inf} | Area under the plasma concentration time curve from |
| | time zero to infinity |
| BMI | Body Mass Index |
| BP | Blood pressure |
| Ca | Calcium |
| CCB | Calcium channel blocker |
| cGMP | Cyclic guanosine monophosphate |
| CKD | Chronic kidney disease |
| CL/F | Apparent total clearance of the drug from plasma after |
| | oral administration |
| CRF | Case Report Form |
| CRP | C-reactive protein |
| CSR | Clinical Study Report |
| CTN | Clinical Trials Notification |
| CV | Coefficient of Variation |
| DDD | Distal-dorsal difference |
| DSMB | Data and Safety Monitoring Board |
| eCRF | Electronic case report form |
| EOS | End of study |
| ESR | Erythrocyte sedimentation rate |
| EULAR | European League Against Rheumatism |
| FIH | First in human |
| GCP | Good Clinical Practice |
| GEE | Generalized Estimating Equation |
| HREC | Human Research Ethics Committee |
| IB | Investigator's Brochure |
| ICF | Informed Consent Form |
| ICH | International Council for Harmonization |
| IP | Investigational product |
| ITT | Intent-to-treat |
| IVRS | Interactive voice response system |
| kel | Elimination rate constant |
| LS | Least Squares |
| MedDRA | Medical Dictionary for Regulatory Activities |
| NHMRC | National Health and Medical Research Council |
| NSAID | Nonsteroidal anti-inflammatory drug |
| PAH | Pulmonary arterial hypertension |
| PDE5 | Phosphodiesterase type 5 |
| PI | Principal Investigator |
| PK | Pharmacokinetic |
| PP | Per-Protocol |
| PT | Preferred term |
| RCS | Raynaud's Condition Score |
| RHI | Reactive Hyperemia Index |
| RP | Raynaud's Phenomenon |
| SAE | Serious Adverse Event |
| SAP | Statistical Analysis Plan |
| SBP | Systolic blood pressure |
| SD | Standard deviation |
| SE | Standard Error |
| SHAQ | Scleroderma Health Assessment Questionnaire |
| SMC | Safety Monitoring Committee |
| SoA | Schedule of Assessments |
| SOC | System organ class |
| SSc | Systemic Sclerosis |
| SSc-RP | Raynaud's Phenomenon secondary mostly to systemic |
| | sclerosis |
| t½ | Elimination half-life |
| TEAE | Treatment-emergent Adverse Event |
| TGA | Therapeutic Goods Administration |
| UCLA SCTC | University of California at Los Angeles Scleroderma |
| GIT 2.0 | Clinical Trials Consortium Gastrointestinal Tract 2.0 |
| VAS | Visual analog scale |
| VMA | Vanylmandelic acids |
| Vz/F | Apparent volume of distribution during terminal phase |
| | after non-intravenous administration |
| WHO | World Health Organization |
| WOCBP | Woman of childbearing potential |

A randomized, placebo-controlled phase 2a study was performed and is in progress to assess the safety and efficacy of cilnidipine (10 mg and 20 mg) alone and in combination with 5 mg tadalafil, in participants with diagnosis of secondary Raynaud's disease.

```
Objectives:
Primary Efficacy:
  To evaluate the effect of cilnidipine alone and in combination with tadalafil on the
frequency of weekly Raynaud's Phenomenon (RP) attacks compared with placebo in
participants with RP secondary mostly to systemic sclerosis (SSc-RP).
Secondary Efficacy:
  To evaluate the effect of cilnidipine alone and in combination with tadalafil on the
clinical features of SSc-RP and the severity and burden of SSc-RP symptoms.
Safety:
  To evaluate the safety of cilnidipine alone and in combination with tadalafil compared to
placebo in participants with SSc-RP.
Exploratory (Part A, Dose Selection)
  To assess the endothelial function of participants with SSc-RP and impact of treatment on
sympathetic activity.
Methodology:
A randomized, placebo-controlled Phase 2a study to test the efficacy and safety of cilnidipine
alone and in combination with tadalafil in participants who have frequent attacks of SSc-RP.
The study consists of two parts.
Part A - Double-blind, Placebo-controlled, Parallel-group, Dose Selection to assess the
safety and efficacy of two doses of cilnidipine (10 mg and 20 mg), alone and in combination
with tadalafil. Participants were randomized to one of six pre-specified treatment arms. The
data from Part A of the study was and will be reviewed by an unblinded Data Safety and
Monitoring Board (DSMB) to select the cilnidipine dose and confirm the sample size
estimates for the randomized double-blind phase (Part B). The first review will occur after
approximately 50% of participants have completed.
Part B - Double-blind, Placebo-controlled, 4-way crossover to assess the safety and
efficacy of cilnidipine (at the dose selected in Part A) alone and in combination with 5 mg
tadalafil. Participants will be randomized to one of four pre-specified treatment sequences in
a 4-way crossover design. Each participant will undergo four Dosing Periods in which they
will receive a different treatment each Dosing Period followed by a 4 (+ 1) day washout
period.
During the study (Part A and Part B) participants received and/or will receive treatments in a
blinded fashion. Each Dosing Period lasted and/or will last for 12 days (+ 2 days) in which
participants took and/or will take daily doses of assigned treatment in the morning. For each
day of dosing, participants took and/or will take one capsule and one tablet to blind the active
therapy being received.
The schematic for dosing is presented for Part A (Table S1) and Part B (Table S2) below.
Table S1: Part A, Double-blind, Parallel-group, Dose Selection
```

| Arm | N | Treatment |
|---|---|---|
| A | 6 | P |
| B | 6 | C10 |
| C | 6 | C20 |
| D | 6 | T05 |
| E | 6 | C10 + T05 |
| F | 6 | C20 + T05 |

Abbreviations:
C= cilnidipine,
P= placebo,
T= tadalafil,
N = number of participants,
C10 = C 10 mg,
C20 = C 20 mg;
T05 = T 5mg.
Table S2: Part B, Double-blind, Placebo-controlled, 4-way crossover

| Sequence | | Dosing | Dosing | Dosing | Dosing |
|---|---|---|---|---|---|
| Number | N | Period 1 | Period 2 | Period 3 | Period 4 |
| 1 | 10 | P | C | C + T05 | T05 |
| 2 | 10 | T05 | C+ T05 | P | C |
| 3 | 10 | C + T05 | T05 | C | P |
| 4 | 10 | C | P | T05 | C + T05 |

```
Abbreviations:
C= cilnidipine,
P= placebo,
T= tadalafil,
N = number of participants,
T05= T 5 mg.
Dose of C, either 10 mg or 20 mg will be identified following completion of Part A.
Number of Participants (Planned):
Overall, 76 participants have been and/or will be enrolled in this study: 36 in the parallel-
group dose selection phase (Part A) and 40 in the 4-way crossover phase (Part B). Dropouts
will not be replaced.
Diagnosis and Main Criteria for Inclusion:
Participants at least 18 years of age diagnosed with severe secondary Raynaud's disease
(Raynaud's Condition Score [RCS] > 40 and at least a 2-phase color change in fingers of
pallor, cyanosis, and/or reactive hypermedia in response to cold exposure or emotion) mostly
resulting from SSc and exhibiting regular and frequent RP attacks (averaging at least one
attack per day) during the Screening period.
Investigational Product, Dosage and Mode of Administration:
Cilnidipine
For Part A, Cilnidipine 10 mg and cilnidipine 20 mg for oral administration, has been and
will be provided to the site in cartons containing 16 tablets sealed in blister packs.
For Part B, the cilnidipine dose selected in Part A will be provided to the site in cartons
containing 16 tablets sealed in blister packs.
Tadalafil
Tadalafil 5mg, for oral administration has been and will be over encapsulated (and back filled
with inert capsule filler consisting of only maize starch and pre-gelatinized maize starch, so
that the internal tablet cannot be detected) and provided in bottles containing 16 capsules to
the site.
Medications have been and will be dispensed during the preceding in-clinic study visit for
self-administration by the participant once daily in the morning for a 12 (+ 2) day period. The
duration of the Dosing Period will be confirmed by the study staff when the in-clinic study
visit is scheduled.
Duration of Treatment:
Part A, Double-blind, Parallel-group, Dose Selection: Participants enrolled participated
and will participate for up to 29 (+ 5) days:
  Screening: up to 10 days (- 3 days)
  Treatment: 12 (+ 2) days
  Follow-up: 7 days (+ 3 days)
Part B, Double-blind, Placebo-controlled, 4-way crossover: Participants enrolled will
participate for up to 77 days (+ 14 days):
  Screening: up to 10 days (- 3 days)
  Treatment: 60 days (four Dosing Periods each lasting for 12 (+ 2) days, separated by a 4
  (+1) day washout period)
  Follow-up: 7 days (+ 3 days)
The total duration of the study is expected to be approximately 6 months.
Reference Therapy, Dosage, and Mode of Administration:
Placebo
Placebo tablets (matching cilnidipine) and placebo capsules (matching tadalafil), for oral
administration, were and will be provided to the site.
Criteria for Evaluation:
Primary Efficacy Endpoint:
  Percentage change from baseline in frequency of weekly RP attacks.
Secondary Efficacy Endpoints:
  Change from baseline in frequency of weekly RP attacks
  Change from Baseline in average duration of weekly RP attacks
  Change from Baseline in average severity of weekly RP attacks
  Change from Baseline in average daily RCS
  Change from baseline in highest (most severe) pain score recorded during weekly RP attacks
  Change from baseline in average pain score recorded during weekly RP attacks
  Change from baseline in net digital ulcer burden
  Change from baseline in distal-dorsal difference (DDD) of the affected index finger sites
  Change from baseline in participant quality of life measured using the Scleroderma Health Assessment
  Questionnaire (SHAQ)
  Change from baseline in Raynaud-visual analog scale (VAS)
  Change from baseline in physician assessment of disease
  Measurement of cilnidipine drug levels taken 2 to 6 hours following the last dose
  The time to reach maximum degree of efficacy (in days) compared to baseline
  The time to return to baseline symptom severity after termination of dosing
  Impact of daily ambient temperature on symptomatic RP attacks
  Use of rescue medications for breakthrough symptoms.
Exploratory Efficacy Endpoints (Part A):
  Change from baseline in endothelial function as measured by reactive hyperemia index (RHI) using
  Endo-PAT
  Impact of treatment on sympathetic activity as measured by plasma vanylmandelic acids (VMA)
Safety Endpoints:
  Incidence of adverse events (AEs) and serious adverse events (SAEs), including clinically significant
vital signs from the time of randomization until 7 days following the last protocol dose.
Serious adverse events will be monitored by a DSMB on an ongoing basis throughout the study.
Adverse events were and will be coded using the Medical Dictionary for Regulatory Activities (MedDRA)
and summarized by System Organ Class (SOC), Preferred Term (PT), and treatment group.
Statistical Methods:
Part A data will be analyzed in exploratory fashion to support cilnidipine dose selection and treatment effect
check for sample size confirmation for Part B. For Part B data, mixed effects model will be used for
analysis of the continuous efficacy endpoints in the crossover design. For nominal data, Chi-square tests
will be applied. Generalized Estimating Equations method will be used, as appropriate, for adjusting for
potential confounding factors. Safety endpoints will be summarized by treatment group. No multiple
comparison adjustment will be used to control alpha for the multiple comparisons.
Statistical Analyses:
Data from Part A and Part B will be analyzed separately. The primary and secondary efficacy analyses were
and will be based on the Intent-To-Treat (ITT) population. Analyses based on the PP population for Part B
will be considered secondary and confirmatory. All safety analyses will be performed on the safety
population. Subgroup analyses will also be performed.
Additional exploratory analyses may be performed and will be documented in the statistical analysis plan.
Any deviation from planned analyses described in this protocol will also be documented in the statistical
analysis plan.
Data will be summarized by treatment group for treatment effect comparison according to crossover design;
participants receiving each treatment will be pooled from all periods. Baseline will be defined as Screening
assessments for change from baseline analyses for all periods.
Sample Size Calculation:
The sample size was calculated based on the confidence bound in literature report (Rirash 2017).
Assuming a 2-sided.05 alpha for treatment (cilnidipine or combination therapy or tadalafil) versus placebo
and a common standard deviation (SD) of 0.5 for a decrease of 25% attacks (moderate effect size of 0.5)
and without controlling alpha for multiple efficacy comparisons, a sample size of ten participants in each
paired comparison group (cilnidipine or combination therapy or tadalafil) is needed for 80% power in a four
period crossover design, assuming a 20% dropout rate.
```

### INTRODUCTION

This protocol describes a study that has been and will be performced to evaluate the effect of cilnidipine alone and in combination with tadalifil on the frequency of weekly Raynaud's attacks in participants with SSc-RP.

### Investigational Plan

### Overall Study Design

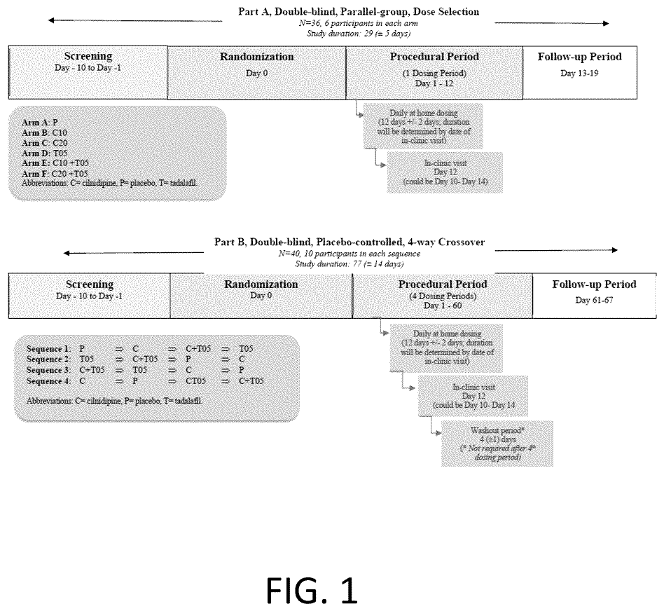

A schematic of the study design is provided in FIG. 1.

This was and is a randomized, placebo-controlled Phase 2a study to assess the safety and efficacy of cilnidipine alone and in combination with tadalafil, in participants who have frequent attacks of secondary RP mostly resulting from SSc. Oversight for the study will be provided by a DSMB.

Participants will underwent and/or will undergo a Screening period beginning up to 10 days prior to randomization. The initial screening and capacity was and will be conducted via phone at the start of the Screening period with eligibility finalized prior to randomization on Day 0. Participants were and will be required to provide informed consent in a 2-step process at Screening (upload of the E-Diary will be considered implied consent for the Screening period) and at Randomization (Day 0) before undertaking any study-specific procedures or assessments. Only participants who met/meet all of the inclusion and none of the exclusion criteria were and will be randomized.

The study consists of two parts.

Part A-Double-blind, Placebo-controlled, Parallel-group, Dose selection, will assess the safety and efficacy of two doses of cilnidipine (10 mg and 20 mg), alone and in combination with tadalafil. A total of 36 participants will be randomized to one of six pre-specified treatment arms. See Example 3 for data obtained in this phase of the study thus far, on 11 participants.

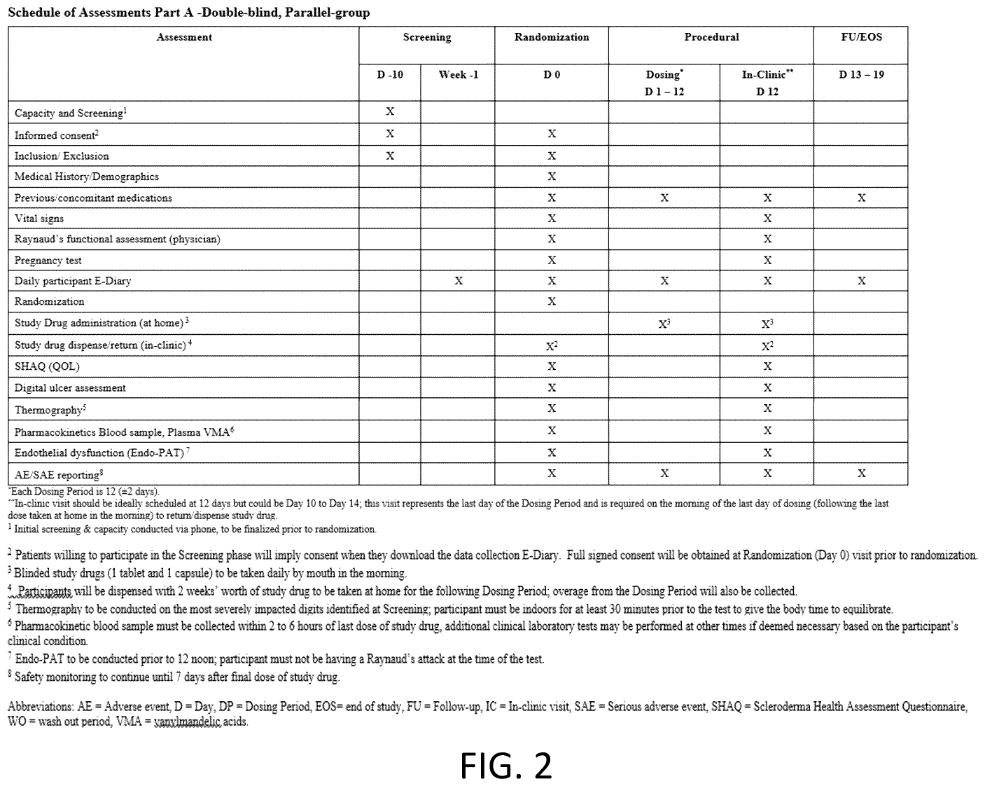

Please refer to FIG. 1 for treatment arms in Part A of the study. Dosing lasted or will last for 12 (±2) days in which participants self-administered or will self-administer daily doses of assigned treatment in the morning. Each participant took or will take one capsule and one tablet to blind the active therapy being received. Study visits and assessments occurred or will occur as delineated in the SoA presented in FIG. 2.

The data from Part A of the study will be reviewed by an unblinded DSMB prior to selecting the cilnidipine dose and confirming the sample size estimates for the randomized double-blind phase (Part B). The first review will occur after approximately 50% of participants have completed the study.

Part B—Double-blind, Placebo-controlled, 4-way Crossover will assess the safety and efficacy of cilnidipine (at the dose selected in Part A) alone and in combination with 5 mg tadalafil. A total of 40 participants (10 in each sequence) with a diagnosis of SSc-RP will be randomized into one of four pre-specified treatment sequences in a 4-way crossover design.

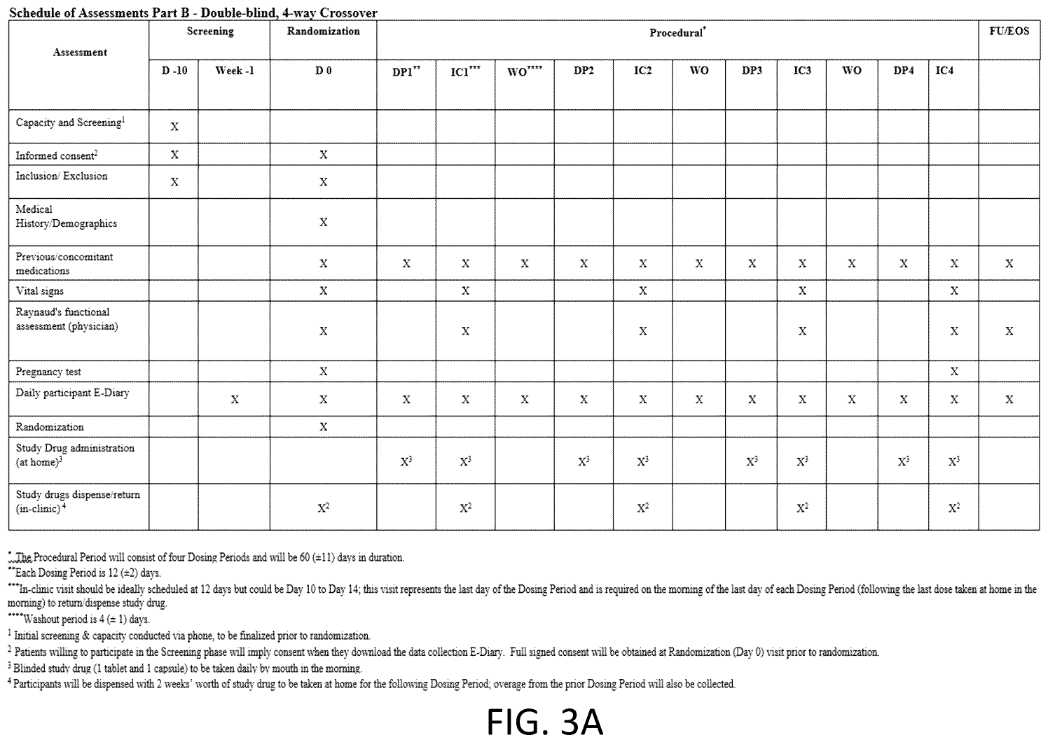

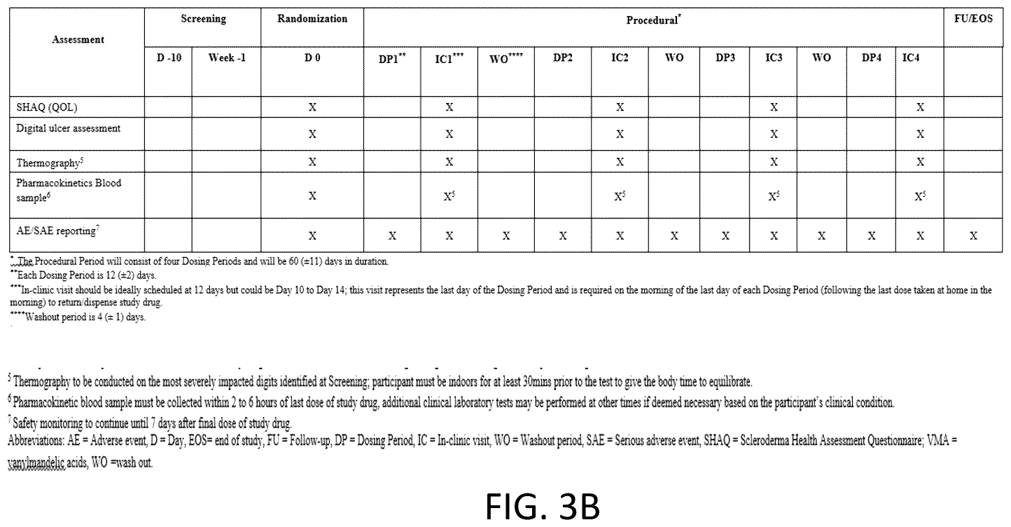

Please refer to FIG. 1 for treatment sequences in Part B of the study. Each participant will undergo four Dosing Periods in which they will receive a different treatment each Dosing Period followed by a 4 (±1) day washout period. Each Dosing Period will last for 12 (±2) days in which participants will self-administer daily doses of assigned treatment in the morning. At all Dosing Periods, each participant will take one capsule and one tablet to blind the active therapy being received. Study visits and assessments will occur as delineated in the SoA presented in FIG. 3A and/or FIG. 3B.

For both Part A and B of the study, participants were or are required to visit the clinic on last day of each Dosing Period (i.e., Day 10 to 14) to return/dispense study drug and conduct in person study assessments. Participants were or will be dispensed with 2 weeks' worth of study drug to be taken at home for the following Dosing Period; overage from the prior Dosing Period were or will also be collected.

Patients were or will be assessed for the occurrence of efficacy endpoints for each Dosing Period via the patient reported E-Diary and the in-clinic visit. Safety information was or will be collected from randomization until patient follow-up is complete (7 to 10) days after the last Dosing Period) and assessed for each Dosing Period.

### Dose Selection, Sample Size Confirmation and Safety Oversight

Safety oversight will be provided by a DSMB. The DSMB will conduct a review of the efficacy and safety data from Part A of the study. The first review occurred after data was available on the first 11 of the participants enrolled into the study. Subsequent reviews will occur as needed prior to commencement of Part B of the study to determine the optimal dose of cilnidipine to carry into Part 2 of the study and to assess the risk: benefit of cilnidipine (10 mg and 20 mg), alone and in combination with tadalafil.

Following review of the efficacy and safety data from Part A, the DSMB will make the following recommendations:
1. Select the dose of cilnidipine (10 mg or 20 mg) to be studied in Part B of the study
2. Confirm the sample size estimates for Part B of the study.

Serious adverse events have been and will be monitored by the DSMB on an ongoing basis throughout the study.

### Safety Criteria for Stopping or Unblinding Treatment

Administration of study drug may be paused, and emergency unblinding of treatment conducted following consultation between the Investigator, the Medical Monitor, and the Sponsor representative under the following circumstances:
Symptoms of possible allergic phenomena: rash, hives, urticaria, changes in breathing or wheezing.
Systolic BP (SBP)<90 mmHg
   Reduction in BP (relative to baseline values) considered to be significant in the opinion of the Investigator on an absolute basis or consistent with symptoms (dizziness, light-headedness) suggestive of being related to reduction in BP.
   Two separate SBP measurements of <90 mmHg, taken at rest (where baseline SBP exceeded 110 mmHg), even in the absence of symptoms, shall be considered sufficient reason to unblind the study for that patient.
Dizziness, when going from a recumbent to standing position and/or if accompanied by a reduction in BP.
   Dizziness in the absence of a reduction in BP should be considered on an individual participant basis, as to its relative degree of severity, as to whether the participant continues in the study.
Unexpected adverse event or reaction.

### Study Termination

The study will be completed as planned unless:
New information or other evaluation regarding the safety of the study medication indicates a change in the known risk/benefit profile for the compound, such that the risk/benefit is no longer acceptable for participants participating in the study. This may be determined by the Sponsor, the Investigator, the HREC or regulatory authorities.
The study is terminated by the Sponsor for administrative reasons.

The Sponsor, Investigator, and the HREC reserve the right to terminate or suspend the study at any time; however, this should be discussed between the relevant parties beforehand and the reason for such decision recorded. Should this occur, all data available will also be recorded in the eCRFs. If the Sponsor, the HREC, or regulatory authority elects to terminate or suspend the study or the participation of the investigational site, a study-specific procedure for early termination or suspension will be provided by the Sponsor. The procedure will be followed by the investigational site during termination or study suspension.

The Investigator should notify the relevant HREC in writing of the study's completion or early discontinuation.

### Participant Population

The study has been and will be conducted in participants at least 18 years of age diagnosed with severe secondary Raynaud's disease (RCS≥40 and at least a 2-phase color change in fingers of pallor, cyanosis, and/or reactive hypermedia in response to cold exposure or emotion) mostly resulting from SSc (defined by consensus criteria 2013 American College of Rheumatology [ACR]) and exhibiting a frequency of attacks (at least one per day) during the Screening period.

Women of childbearing potential were or will be included and have been or are subject to contraceptive requirements during the study from Screening until study completion, including the follow-up period, and for at least 30 days after the last dose of study drug (see Section 0). Women of childbearing potential must demonstrate negative pregnancy testing at Screening. This is in line with regulatory Guidance on Nonclinical Safety Studies for the Conduct of Human Clinical Trials and Marketing Authorization for Pharmaceuticals (US FDA Guidance document, January 2010).

### Number of Participants

Overall, 76 participants have or will be enrolled in this study: 36 in the parallel-group dose selection phase (Part A) and 40 in the 4-way crossover phase (Part B). Dropouts will not be replaced.

### Participant Inclusion Criteria

To be eligible for this study, each participant has met or has to meet all of the following inclusion criteria:
1. Male or female participants, aged 18 to 80 years (inclusive at the time of informed consent).
Severe secondary Raynaud's disease (defined as RCS at baseline of ≥40) based on ACR criteria mostly resulting from SSc.
Regular and frequent Raynaud's attacks (averaging at least one attack per day) during the Screening week (in participants with at least a 2-phase color change in fingers of pallor, cyanosis, and/or reactive hypermedia in response to cold exposure or emotion).
Willingness to complete the daily diary entry's during the Screening period.
Participants must be willing and able to provide written informed consent after the nature of the study has been explained and prior to the commencement of any study procedures.
Willingness to forego other routine therapies for Raynaud's during study participation. Ongoing treatment with CCBs for SSc-RP is permitted if it is not clinically feasible to stop therapy and the participant has been on a stable dose for the last 2 months. Rescue therapy (with acetaminophen, nonsteroidal anti-inflammatory drug [NSAIDs], other codeine analgesics, fluoxetine, angiotensin II receptor blockers (ARBs) such as losartan) or CCBs will be allowed to manage breakthrough symptoms of SSc-RP but must be documented).
Participants who are on a stable dose (no change in the last 2 months) of a CCB for hypertension or sildenafil for pulmonary hypertension may continue these agents at this stable dose as long as they meet other inclusion criteria during Screening.
Have agreed not to use (or initiate treatment with) other investigational therapies or unapproved therapies during the course of the study outside of protocol allowances for rescue medication (such medications include but are not limited to: nitroglycerin, topical creams, fenoldopam, nimodipine, amlodipine, fluoxetine, pregabalin, gabapentin, verapamil, sildenafil, tadalafil, vardenafil).
Women of childbearing potential (WOCBP) who have agreed to use an effective method of contraception during the study and for 30 days after the last study dose. Acceptable methods of birth control in this study include oral contraceptive, barrier control, or implanted devices. A woman must agree not to donate eggs (ova; oocytes) for purposes of reproduction for at least 30 days after last dose of study drug.
Post-menopausal females, aged over 45 years who have not had a period for at least 12 months, and are not using hormonal contraception, or females documented as permanently sterile (e.g., bilateral tubal ligation; hysterectomy, bilateral salpingectomy).
Negative urine pregnancy test on Randomization day (WOCBP only).
All sexually active men (due to potential risk of drug exposure through the ejaculate) who agree to a barrier method of birth control during the study and for 30 days after the last dose of the study drug. All men must agree not to donate sperm for at least 30 days after receiving last dose of study drug.
Participants must have clinical laboratory values within normal range as specified by the testing laboratory at their most recent pre-screening sample, unless deemed not clinically significant by the Investigator or delegate. Lab sample may be within the last 2 months as long as there have been no significant clinical changes since the last labs and no new medications (e.g. Diuretics) have been introduced that are known to be associated with changes in clinical chemistry or hematology values (e.g. potassium with diuretics or cyclosporine associated with aplastic anemia).

### Participant Exclusion Criteria

A participant who has met or meets any of the following exclusion criteria must be excluded from the study:
1. Primary Raynaud's disease.
History of Raynaud's attacks of sufficient severity as to require in-patient hospitalization (within the last 6 months).
The SBP of <95 mm Hg during Randomization visit (Day 0).
Pulmonary hypertension requiring specific therapy for this condition.
Participants with an allergy to dihydropyridine CCBs that results in clinical findings such as profound hypotension, hives, rash, urticaria, wheezing and changes in breathing (Common treatment limiting adverse events [AEs] that occur with CCBs nifedipine and amlodipine such as edema, headache, heart rate changes, tachycardia, fatigue, constipation, flushing, drowsiness, dizziness should not limit enrollment into this study).
History of other chronic pain condition that could confound recording of pain scores during the study period.
Any prior or ongoing medical conditions, medical history, physical findings, or laboratory abnormality that in the opinion of the Investigator(s), would contraindicate administration of the study medication, or interfere with the study evaluations, or interfere with the participants ability to comply with the study protocol.
Cognitive or language difficulties that would impair completion of the study assessments.
Use of any investigational product (IP) or investigational medical device or participation in investigational drug studies within 30 days prior to enrollment in the study.
Those receiving nitrates, alpha blockers, PDE inhibitors (outside of stable administration for PAH), prostacyclins or endothelin antagonists.
History of orthostatic hypertension, dizziness or fainting spells, acute coronary or cerebrovascular event within 3 months of study enrollment.
History of major thoracic, abdominal, or vascular surgery within 6 months of study enrollment; History of sympathectomy.
Severe cardiomyopathy, severe valvular heart disease, chronic kidney disease (CKD) stage 3 or greater, evidence of malignancy, end stage lung disease.
Pregnant or lactating women.
Women of childbearing potential (WOCBP) unable to comply with contraceptive requirements during the study period.
Males with partners who are WOCBP and are unable to comply with the contraceptive requirements during the study.
History of drug or excess alcohol use that in the opinion of the Investigator(s) would affect the participant's ability to reliably participate in the study. NHMRC guidelines for regular alcohol consumption in healthy adults are no more than 10 standard drinks per week and no more than four standard drinks on any one day.
Use of tobacco products of any type in the preceding one month and for the duration of the study.

### Screen Failures

Screen failures are defined as participants who consent to participate in the clinical study but are not subsequently randomized in the study. A minimal set of screen failure information is required to ensure transparent reporting of screen failure participants to meet the Consolidated Standards of Reporting Trials publishing requirements and to respond to queries from regulatory authorities. Minimal information includes screen failure details and eligibility criteria. Participants who withdraw from the study, for any reason, prior to randomization will be considered screen failures. Individuals who do not meet the criteria for participation in this study (screen failure) may be re-screened following a one month waiting period. Re-screened participants should be assigned a new participant number.

### Participant Replacement

All participants who were or are randomized were or will be followed and included in the primary ITT analysis. Dropouts were not and will not be replaced.

### Participant Withdrawal Criteria

Participants may withdraw their consent to participate in the study at any time. If a participant withdraws consent, the date and reason for consent withdrawal should be documented. Participants will be encouraged to remain in the clinic to complete all necessary assessments and until the Investigator deems that it is safe to be discharged.

Participant data will be included in the analysis up to the date of the withdrawal of consent.

Apart from withdrawal of consent, reasons for early termination of individual participants can include:
Protocol deviations or participant non-compliance (must be specified on the appropriate eCRF)
Serious or severe AEs
Administrative decision by the Investigator or the Sponsor
Death
Other (must be specified).

The primary reason for withdrawal will be identified and recorded on the appropriate eCRF, along with the date of withdrawal.

In accordance with applicable regulations, a participant has the right to withdraw from the study, at any time and for any reason, without prejudice to future medical care.

If a participant is withdrawn because of an AE, the Investigator must arrange for the participant to have appropriate follow-up care until the AE is resolved or has stabilized. Unresolved AEs will be followed until the last scheduled Follow-up/End of Study (EOS) visit or until the Investigator(s) determine that further follow-up is no longer indicated. In addition to AEs, other reasons for removal of participants from the study might include, but are not limited to, withdrawal of consent, administrative decision by the Investigator or the Sponsor, protocol deviation, or participant noncompliance.

If a participant asks or decides to withdraw from the study, all efforts will be made to complete and report the observations, especially those related to the listed primary and secondary objectives, as thoroughly as possible up to the date of withdrawal. Wherever possible, the tests and evaluations, including those listed for the EOS/follow-up visit, should be performed for all participants who discontinue prior to the completion of the study.

### Treatments

### Treatments Administered

### Investigational Product

### Cilnidipine

Cilnidipine is an orally administered dihydropyridine CCB that dilates blood vessels, increases blood flow, inhibits sympathetic nervous system activity, and improves endothelial structure and function. Please refer to IB for more information on composition of cilnidipine tablet (Profervia® Investigator's Brochure, 2020), which is incorporated by reference herein in its entirety.

Profervia® tablets are white film-coated tablets. Each tablet contains cilnidipine (10 or 20 mg) with microcrystalline cellulose, lactose, magnesium stearate, sodium starch glycollate, Opadry white, polyvinyl alcohol, titanium dioxide, macrogol, talc, and purified water.

Cilnidipine is commercially available and should be used only in accordance with this study protocol and IB.

Cilnidipine 10 mg and 20 mg oral tablets have been and will be provided to the site in cartons containing 16 tablets sealed in blister packs.

### Tadalafil

Tadalafil for oral administration belong to a class of medications called PDE5 inhibitors.

Tadalafil is commercially available and should be used only in accordance with this study protocol. Please refer to the pharmacy manual and product information sheet for more information on composition and storage information for tadalafil.

Tadalafil has been and will be over encapsulated (and back filled with inert capsule filler consisting only of maize starch and pre-gelatinized maize starch, so that the internal tablet cannot be detected) and provided in bottles containing 16 capsules to the site.

### Reference Products

Placebo tablets (matching cilnidipine) and placebo capsules (matching tadalafil) for oral administration have been and will be provided for this study.

### Dosage and Treatment Periods

At all Dosing Periods, each participant have taken or will take one capsule and one tablet to blind the active therapy being received. All medications for each Dosing Period (each Dosing Period will last for 12 days [+2 days]) have been or will be dispensed during the preceding in-clinic visit and then self-administered by the participant once daily, orally, in the morning.

If a participant accidentally misses a dose, they have been or should be advised to take the dose on the same day as soon as they realize. Only one tablet and capsule have been and should be taken each day. If more than one dose is lost, the participant should notify the study staff so that their in-clinic visit can be adjusted if needed.

Part A, Double-blind, Parallel-group, Dose Selection Study drug has been or will be self-administered daily for 12 (±2) days. Each participant has or will receive only one treatment. Please refer to FIG. 2 for more details.

Part B, Double-blind, Placebo-controlled, 4-way Crossover Study drug will be self-administered daily in four Dosing Periods separated by a four-day (±1) washout period. Each participant will receive a different treatment during each Dosing Period, with a total of four treatments received. Please refer to FIG. 3A and/or FIG. 3B for more details.

### Method of Assigning Participants to Treatment

### Randomization

A randomization list has been or will be prepared using a statistical software package by a Biostatistician.

Each participant has been or will be provided with a unique screening number post-documentation of informed consent. Once deemed eligible, the participant has been or will be assigned a sequential randomization number prior to first dosing. Participants who withdraw from the study or who fail eligibility, for any reason, prior to randomization will be considered screen failures.

### Concomitant Medications

All medications, including over the counter medications, vitamins, and herbal supplements, taken during the Screening period have been or will be reviewed by the Investigator to determine whether these medications render the participant as suitable for inclusion in the study.

Concomitant medications of interest have been or will be captured electronically from the start of the Screening period until study completion.

Treatment prior to enrollment with therapies for SSc-RP including but not limited to CCBs, nitroglycerin, topical creams fenoldopam, nimodipine, fluoxetine, pregabalin, gabapentin, sildenafil, tadalafil, vardenafil were or are permitted. In order to have been or be eligible, participants must be willing to forego these therapies for SSc-RP at the start of the Screening period and for the duration of the study. Participants who were or are on a stable dose (no change in dose in prior 2 months) of a CCB for hypertension or sildenafil for pulmonary hypertension may continue these agents at this stable dose as long as they meet other inclusion criteria during Screening. Similarly, participants who have been or are receiving CCBs to manage their symptoms of SSc-RP and are unwilling to stop treatment for the duration of the study would still be eligible if the participant's dose has been stable for the past 2 months. During the study participants were or will be able to decrease the dosage of their prior CCB for safety reasons only; no increase in dosage was or will be allowed during the study period.

The use of any other IP or investigational medical device within 30 days prior to Screening is prohibited.

Prior therapy or concomitant therapy (after study drug administration) with any medications, including both prescription and non-prescription drugs should be discussed with the Investigator and Sponsor's MM before study drug administration, except in the case of necessary treatment of AEs or where appropriate medical care necessitates that therapy should begin before the Investigator can consult with the Sponsor's MM.

### Rescue Medicine

Medications required as rescue therapy can be taken to manage breakthrough symptoms of SSc-RP but must be recorded in the participant E-Diary. First-line therapy may include acetaminophen, NSAIDs, or other codeine-based analgesics. These rescue medications may be taken for the duration of symptoms of a Raynaud's attack. For participants in whom first-line rescue therapy is not effective, additional rescue medication therapy may be started per Investigator discretion and could include fluoxetine, ARBs such as losartan or CCBs. Rescue therapy should continue as long as clinically needed during an acute attack, but then patients should return to the pre-rescue study medication regimen. All participants receiving rescue therapy should continue in the study undergoing subsequent Dosing Periods through study completion. If a participant requires regular rescue medicine during dosing through at least four sequential Dosing Periods, then the participant may drop out of the study at the discretion of the participant and the Investigator.

### Treatment Compliance

All doses have been or will be self-administered by participants remote from study sites (at home). For each Dosing Period, participants were or will be dispensed with two weeks' worth of study medication and will be asked to return the unused study medication on the last day of each Dosing Period at the time of in-clinic visit. The treatment compliance was or will be noted by the Investigator(s) during the in-clinic visit.

### Blinding

This study is double-blind. To maintain the blind, all study medication has been and will be provided to the site in a blinded fashion. Cilnidipine tablets and matching placebo was and will be supplied in cartons containing 16 tablets sealed in blister packs, identical in appearance. Tadalafil was and will be provided in an over encapsulated form. The capsules, tadalafil, and placebo were and will be identical in appearance and weight and will be supplied in bottles containing 16 capsules.

Each study drug was and will be labeled with a unique ID number. The interactive voice response system (IVRS) was or will have access to the treatment arm assignment for each individual ID number.

### In the Event of an Emergency: Unblinding a Code

It is recognized that, in the course of clinical practice, it may be necessary for the treating physician to have knowledge of the treatment assignment to ensure the safety of a study participant. This circumstance is extraordinary and will likely impact a minor fraction of the enrolled participants. Unblinding will be done via the IVRS. The treating physician is encouraged to contact the Sponsor MM in this circumstance. The Sponsor and DSMB will monitor all episodes of unblinding very carefully.

### Study Schedule

The SoAs for Part A and Part B of the study are provided in FIG. 2 and FIG. 3A and/or FIG. 3B.

Where possible, assessments have been and should be conducted in order of least invasive to most invasive.

This study consists of four periods:
Screening period (begins with initial participant contact through participant completion of the screening E-Diary)
Randomization period (from the time participant eligibility is confirmed and the participant randomized until immediately before the 1st dose of study drug)
Procedural period (from the first dose of study drug in the first Dosing Period until the last day of dosing)
Follow-up period (from the end of the procedural period through 7 days).

### Part A—Double-Blind, Placebo-Controlled, Parallel-Group, Dose Selection

In Part A, the procedural period has required and will require only one Dosing Period i.e. participants have or will receive only one treatment during the procedural period. Within the procedural period for Part A there have been and will be two sub-periods:
Daily at home dosing (first ten to fourteen days of the Dosing Period).
In-clinic visit (the last day of the Dosing Period that occurs on Day 12 [+2 days]).

### Part B—Double-Blind, Placebo-Controlled, 4-Way Crossover

In Part B, the procedural period will require four Dosing Periods i.e. participants will receive four different treatments in a 4-way crossover design. Within the procedural period for Part B there will be three sub-periods associated with each Dosing Period/treatment received:
Daily at home dosing (first ten to fourteen days of the Dosing Period).
In-clinic visit (the last day of the Dosing Period that occurs on Day 12 [+2 days])
Washout period (four days of no dosing that occurs between each Dosing Period).

### Screening (Day −10 to Day −1)

Prior to enrolling in the study, and before performance of any procedures, potential participants have been or will be contacted via phone to discuss the details of the study and assess their eligibility and willingness to comply with all study procedures and duration. If the participant seemed or seems eligible and was or is interested in participating in the Screening period, then they were or will be asked to upload and start using an E-Diary to record the daily clinical features and symptoms of their SSc-RP for the next 7 to 10 days. Upload of the E-Diary was and will be considered implied consent for the Screening period, the data from which was and will be used to confirm eligibility and future baseline analyses assuming the participant is randomized.

During Screening, the E-Diary collected or will collect the following data to confirm eligibility and serve as the baseline measure for efficacy endpoints should the participant be randomized:
Number of daily Raynaud's attacks
Duration of each attack
Symptoms of each attack, including numbness, pain, tingling, color changes
Severity of each attack (all symptoms of the attack to be considered including tingling, numbness, pain, color changes)
Location of participant during each attack (inside/outside)
Pain score of each attack-using 11-point Likert scale, a validated pain scale which can be used to record intensity of pain
Daily RCS—a validated outcome measure used to assess the level of difficulty experienced due to RP each day
Rescue medications taken to manage breakthrough symptoms of SSc-RP including acetaminophen, NSAIDs, other codeine-based medicines, fluoxetine, ARBs such as losartan, CCBs
Other concomitant medications of interest including allergy medications, stable medications for pre-existing hypertension or pulmonary hypertension, and prohibited medications for SSc-RP (including topical creams, fenoldopam, nimodipine, pregabalin, gabapentin, sildenafil, tadalafil, vardenafil).

### Randomization Day (Day 0)

Participants were or will be scheduled to visit the clinic for Randomization (Day 0) assessments between days 7 to 10 of the Screening period. Only participants who seemed or seem eligible based on E-Diary compliance and frequency of RP attacks were or will be requested to visit clinic for randomization. The participant was or will also be provided with an Informed Consent Form (ICF). Prior to being asked to sign the consent form, participants were or will be given time to review study information and ask any questions.

After the consent form is signed and the following assessments will be carried out:
Medical history/demographics
Previous/concomitant medications
Vital signs
Raynaud's functional assessment (by physician)
Pregnancy test
Review of daily participant E-dairy
Scleroderma Health Assessment Questionnaire (SHAQ) which includes Raynaud's VAS to be completed by the participant with review by physician
Digital ulcer assessment
Thermography
Endo-PAT (Part A only)
Blood sample for PK
Plasma VMA (Part A only)
Inclusion/Exclusion
Randomization
AE/SAE reporting
Dispensing study medication

Note: The data collected for assessments that were and are performed first time on Randomization (Day 0) visit (vital signs, digital ulcer assessment, and Endo-PAT) served and will serve as baseline measure for efficacy endpoints for those assessments.

Routine hospital tests including hematology, biochemistry, inflammatory markers (C-reactive protein [CRP] and erythrocyte sedimentation rate [ESR]), antibody status (serum anti-topoisomerase [anti-Scl 70]) and nailfold capillaroscopy should be conducted as clinically indicated per standard of care but are not required per protocol. If conducted, results will be collected in the eCRF and used to describe the severity of disease in the baseline demographic and disease data.

### Dosing Periods

### Daily at Home Dosing (12 Days (±2 Days))

During the Dosing Period, participants were or will be required to self-administer the assigned study medication once daily in the morning at home. Participants were or will also be required to complete their E-Diary daily to capture the clinical features and symptoms of their SSc-RP, and report concomitant medications including rescue therapy (if any).

### In-clinic Visit (Last Day of the Dosing Period)

Participants have been or will be required to visit the clinic following each Dosing Period—dosing Day 10 to Day 14. The day of the in-clinic visit is considered the last day of dosing in each Dosing Period.

After taking their last dose of study medication in the morning at home, the following assessments/procedures will take place during the in-clinic visit:

Following assessments/procedures have been and will take place on the day of the visit:
Vital signs
Blood sample for PK
Blood sample for Plasma VMA (Part A)
Daily participant E-dairy review for the most recent dosing sequence
Scleroderma Health Assessment Questionnaire (SHAQ) which includes
   Raynaud's VAS to be completed by the participant with review by physician. Physician has or will assess the below at the in-clinic visit, details of which will be recorded in the eCRF:
Concomitant medications
Rating severity of participant's Raynaud's disease
Rating severity of participant's ulcers
Rating overall health of participant for past week
Physician's assessment of digital ulcers-how many ulcers, how many considered new, for each ulcer-location and diameter of the ulcer
Thermography (for two most symptomatic areas in terms of Raynaud's)
Endothelial dysfunction (Endo-PAT, Part A only)
AE/SAE reporting
Dispensing/returning study medication

Note: On the last clinic visit at the end of last Dosing Period (Dosing Period 1 for Part A and Dosing Period 4 for Part B), pregnancy test will be performed for WOCBP.

### Washout Period

In Part B, each Dosing Period will be separated by a 4-day (±1 day) washout period. The washout period will commence the day after the in-clinic visit during which participants will not take any study medication. During the washout period, participants will be required to complete the daily participant E-Diary, reporting their symptoms of SSc-RP, and use of any concomitant medications. Once the 4-day washout is completed the participant will commence the daily at home dosing for the study medication dispensed at the pervious in-clinic visit. No washout period is required after the fourth and final in-clinic visit. After this visit participants will proceed directly to follow-up.

### Follow-Up

Participants have been or will be followed for 7 days following completion of the final Dosing Period.

Participants have or will be requested to complete the E-Diary for 7 days in Follow-up period. Participants have or will also be requested to report use of any concomitant medications and any AEs/SAEs during the Follow-up period.

This visit marks the end of participation in this study.

### Early Termination (if Applicable)

Participants who withdraw early from the study will be encouraged to return to the clinic for an EOS assessment.

The following procedures will be conducted:
Participants will be requested to complete the E-Diary for 7 days following termination
Participants will be requested to report use of concomitant medications of interest for 7 days following termination
Participants will be requested to report any AEs/SAEs for 7 days following termination.

This visit marks the end of participation for participants that withdraw early from the study.

### Pharmacokinetic Assessments

### Blood Sample Collection

One 4 mL blood sample has or will be obtained during each in-clinic visit within 2 to 6 hours of last dose of study drug in that Dosing Period as delineated in the SoA (FIG. 2 and FIG. 3A and/or FIG. 3B). The level of cilnidipine in blood has or will be measured following last dose of the Dosing Period. Sample handling details have been or will be provided in the PK manual. The actual collection time of each sample must be recorded in the source data documentation, on the collection tube and in the eCRF.

### Efficacy Assessments

### Efficacy Parameters

Study procedures should be completed as delineated in the SoAs ((FIG. 2 and FIG. 3A and/or FIG. 3B).

### E-Diary

The Sponsor-developed participant-informed E-Diary has been and will be used in this study to record data. Participants have been or will be required to keep and fill the E-Diary daily as delineated in the SoAs ((FIG. 2 and FIG. 3A and/or FIG. 3B).
The relevant metrics measured by this tool daily are:
Study medication
SSc-RP symptoms (Reporting an attack including duration of attack)
Severity of the attack considering all symptoms of the attack e.g. tingling, numbness, pain, color changes (VAS 0-10 cm scale)
Participant's location at the time of the attack (inside home/outside home)
Selecting symptoms experienced during the attack (tingling, numbness, pain, color changes, other)
Pain rating during the attack (11-point Likert scale)
The RCS based on how much difficulty participants had with Raynaud's today, how many attacks the participant had, and how long they lasted. Participants will also be asked to consider how much pain, numbness, or other symptoms the Raynaud's caused in fingers (including painful sores), and how much the Raynaud's alone affected the use of hands today (VAS 0-10 cm scale).
The relevant metrics measured by this tool at the in-clinic visit (once in a Dosing Period) are:
Digital ulcer severity (VAS 0-10 cm scale)—Applicable only if participant has digital ulcers.
Scleroderma Health Assessment Questionnaire which includes Raynaud's VAS measuring participant quality of life over the past
   Note: Digital ulcer severity (VAS 0-10 cm scale) and SHAQ scale can also be done on paper in-clinic, if necessary. Also, external temperature will be a feed into the E-Diary database based on participant's location.

### Physician Assessments

The Physician has and will assess the below at the in-clinic visit, details of which will be recorded in the eCRF. In Part A, thermography and Endo-PAT will also be reported.
Rating severity of participant's Raynaud's disease Rating severity of participant's ulcers
Rating overall health of participant for past week
Physician's assessment of digital ulcers-how many ulcers, how many considered new, for each ulcer: location and diameter of the ulcer
Recording temperature of the affected index finger sites at two symptomatic areas at baseline and every in-clinic visit using the Fluor thermographic camera
AE/SAE reporting.
Drug accountability, including dispensing and returning of the study medication will also be recorded at each visit.

### Scleroderma Health Assessment Questionnaire (SHAQ)

The standard, validated, patient reported outcome measures tool for SSc patients, the SHAQ, will be collected at the time points specified in the study schedules (FIG. 2 and FIG. 3A and/or FIG. 3B) To assess the participant's quality of life. The SHAQ includes a Raynaud's VAS that will also be reported separately.

### Thermography

Thermography assessments will be performed at the time points specified in the study schedules (FIG. 2 and FIG. 3A and/or FIG. 3B). Thermography will be conducted on the most severely impacted digits identified at Screening; the participant must be indoors for at least 30 minutes prior to the test to give the body time to equilibrate. Photos will be taken with the help of Fluor thermographic camera of these two areas at times specified in SoAs.

### Plasma Vanylmandelic Acids (VMA)

Plasma VMA is a metabolite of norepinephrine. One sample was and will be collected during each in-clinic visit in Part A of the study to assess if a difference in sympathetic activity with cilnidipine can be detected.

### Endothelial Dysfunction (Endo-PAT)

Assessments for endothelial dysfunction will be performed using Endo-PAT at timepoints specified in the Part A study schedule (FIG. 2); Endo-PAT assessment will not be performed in Part B of the study. The Endo-PAT is a diagnostic device used to assess endothelial vasodilator function in a rapid and non-invasive fashion.

The below points should be considered before assessment is started:
2. Prior to the study, the participant should fast for at least 4 hours, and should refrain from caffeine, vitamins or medications that might affect vascular tone for at least 8 hours. The participant must reconfirm their abstinence from tobacco and may use the restroom prior to the assessment.
The Endo-PAT assessment should be conducted in a quiet, dimly lit, temperature-controlled (25° C. for at least 30 minutes) exam room to reduce fluctuations in vascular tone.
Cell phones or paging devices should be silenced, and restrictive clothing that could interfere with blood flow to the arms should be removed. The participant should also remove watches, rings, or other jewelry on the hands or fingers.
Participant's fingers should be inspected for any deformities or injuries that could affect the study. The probes should not be placed on a finger that is cut or injured. Fingernails should not extend more than 5 mm or ⅕ of an inch beyond the tip of the finger tissue.
The index finger is recommended for the study; however, if this finger is unsuitable, a different digit (except the thumb) may be used, as long as the same finger is used on both hands.
The participant should be supine and comfortable for 15 minutes so as to attain a cardiovascular steady state.

### Safety Assessments

### Safety Parameters

Study procedures should be completed as delineated in the SoA (FIG. 2 and FIG. 3A and/or FIG. 3B). Any unscheduled procedures required for urgent evaluation of safety concerns must take precedence over all routine scheduled procedures

### Demographic/Medical History

Medical history (including alcohol and smoking status), date of birth, age (calculated), weight, sex, ethnicity, and race were and will be recorded at Randomization (Day 0) visit.

### Vital Signs

Vital signs (SBP, DBP, pulse rate, temperature) were measured and will be measured at the time points specified in the SoA (FIG. 2 and FIG. 3A and/or FIG. 3B) with participants resting for at least 5 minutes in a supine position. When the time of vital signs measurement coincides with a blood draw, the vital signs have been or will be taken before the scheduled blood draw where possible, ensuring the blood draw is within the window specified in the protocol.

Additional vital signs may be performed at other times if deemed necessary.

### Laboratory Assessments

Routine hospital laboratory tests including hematology, biochemistry, inflammatory markers (CRP and ESR), and antibody status (Scl-70) should be conducted as clinically indicated per standard of care but are not required per protocol. If conducted, results will be collected in the eCRF and used to describe the severity of disease in the baseline demographic and disease data.

Additional clinical laboratory tests may be performed at other times if deemed necessary, based on the participant's clinical condition.

### Pregnancy Testing

A urine pregnancy test will be performed at the Randomization (Day 0) visit and on the last clinic visit at the end of last Dosing Period for WOCBP only.

### Adverse and Serious Adverse Events

In this study, AEs and SAEs will be reported for all participants from the time of randomization until the completion of the Follow-up/EOS visit. Adverse events that are ongoing at the EOS visit will be marked as Not Recovered/Not resolved on the AE eCRF page.

The Investigator will do full AE review during in-clinic visit. All spontaneously volunteered and enquired for, as well as observed AEs, will be recorded in the participant's medical records and the eCRF.

### Complications of the Disease Under Study

Clinical features and symptoms of SSc-RP must be recorded as endpoints in the electronic data collection tools provided by the Sponsor, as well as in the source documents and should not be reported as AEs.

### Definition of Adverse Events

An AE is any event, side effect, or other untoward medical occurrence that occurs in conjunction with the use of a medicinal product in humans, whether or not considered to have a causal relationship to this treatment. An AE can, therefore, be any unfavorable and unintended sign (that could include a clinically significant abnormal laboratory finding), symptom, or disease temporally associated with the use of a medicinal product, whether or not considered related to the medicinal product.
Events meeting the definition of an AE include:
Exacerbation of a chronic or intermittent pre-existing condition including either an increase in frequency and/or intensity of the condition
New conditions detected or diagnosed after study drug administration that occur during the reporting periods, even though it may have been present prior to the start of the study
Signs, symptoms, or the clinical sequelae of a suspected interaction
Signs, symptoms, or the clinical sequelae of a suspected overdose of either study drug or concomitant medications (overdose per se will not be reported as an AE/SAE).
Events that do not meet the definition of an AE include:
Medical or surgical procedure (e.g., endoscopy, appendectomy); the condition that leads to the procedure should be reported as an AE if it meets the criteria of an AE
Situations where an untoward medical occurrence did not occur (e.g., social and/or convenience admission to a hospital)
Anticipated day-to-day fluctuations of pre-existing disease(s) or condition(s) present or detected at the start of the study that do not worsen.
If there is evidence of an AE through report or observation, the Investigator or designee will evaluate further and record the following information:
Time of onset and resolution
Severity
Seriousness
Causality/relation to study treatment
Action taken regarding study drug
Action taken regarding AE
Outcome.

### Severity of an Adverse Event

Severity of AEs will be graded by the Investigator as one of:
Mild (Grade 1): A type of AE that is usually transient and may require only minimal treatment or therapeutic intervention. The event does not generally interfere with usual activities of daily living.
Moderate (Grade 2): A type of AE that is usually alleviated with additional specific therapeutic intervention. The event interferes with usual activities of daily living, causing discomfort but poses no significant or permanent risk of harm to the research participant.
Severe (Grade 3): A type of AE that interrupts usual activities of daily living, or significantly affects clinical status, or may require intensive therapeutic intervention.
Life-threatening (Grade 4): A type of AE that places the participant at immediate risk of death.
Death (Grade 5): Events that result in death.

### Causal Relationship of an Adverse Event

The Investigator will assess the relationship between study drug and the occurrence of each AE. The Investigator's assessment of the relationship of each AE to study drug will be recorded in the source documents and the eCRF. Alternative causes, such as medical history, concomitant therapy, other risk factors, and the temporal relationship of the event to the study drug should be considered and investigated, if appropriate. The following definitions are general guidelines to help assign grade of attribution:
Not related: The event is clearly related to other factors such as the participant's environment or clinical state, therapeutic interventions or concomitant drugs administered to the participant. This is especially so when an event occurs prior to the commencement of treatment with the study drug.
Unlikely: The temporal association, participant history, and/or circumstances are such that the study drug is not likely to have had an association with the observed event. Other conditions, including concurrent illness, progression, or expression of the disease state, or reaction to a concomitant drug administered appear to explain the event.
Possible: The event follows a reasonable temporal sequence from the time of study drug administration or follows a known response to the study drug but could have been produced by other factors such as the participant's clinical state, other therapeutic interventions, or concomitant drugs administered to the participant.
Probable: The event follows a reasonable temporal sequence from the time of study drug administration and follows a known response to the study drug and cannot be reasonably explained by other factors such as the participant's clinical state, other therapeutic interventions, or concomitant drugs administered to the participant.
Definite: The event follows a reasonable temporal sequence from the time of study drug administration or control abates upon discontinuation or cannot be explained by known characteristics of the participant's clinical state.
   Action Taken with Investigational Products

Should the Investigator need to alter the administration of the study drug from the procedure described in the protocol due to the wellbeing and safety of the participant then the action taken will be recorded on the AE eCRF page, as one of the following options:
Dose Reduced
Drug Interrupted
Drug Withdrawn
Not Applicable
Other.

### Outcome

Outcome of an AE will be recorded on the AE eCRF as follows:
Recovered/Resolved
Recovering/Resolving
Recovered/Resolved with Sequelae
Not Recovered/Not Resolving
Fatal
Unknown.

### Definition of Serious Adverse Event

An SAE is an AE occurring during any study phase (i.e. baseline, treatment, washout, or follow-up), and at any dose of the study drug (active or placebo), that fulfills one or more of the following:
Results in death
It is immediately life-threatening
It requires in participant hospitalization or prolongation of existing hospitalization
It results in persistent or significant disability or incapacity
Results in a congenital abnormality or birth defect
It is an important medical event that may jeopardize the participant or may require medical intervention to prevent one of the outcomes listed above.

Important medical events that may not be one of the above may be considered an SAE by the Investigator when, based upon appropriate medical judgment, they are considered clinically significant and may jeopardize the participant, or may require medical or surgical intervention to prevent one of the outcomes listed above.

An AE is considered “life-threatening” if, in the opinion of either the Investigator or the Sponsor, its occurrence places the participant at immediate risk of death. It does not include an AE that, had it occurred in a more severe form, might have caused death.

### Statistics

Statistical methods will be further outlined in the statistical analysis plan (SAP, see Example 2) and approved by the Sponsor. Procedures outlined in the SAP will supersede protocol specified statistical methods in the event of divergence.

Part A and Part B data will be analyzed separately. Analysis of Part A data was and will be mainly exploratory to support cilnidipine dose selection and treatment effect check for sample size confirmation for Part B.

In general, descriptive statistics (e.g. arithmetic mean, SD, median, minimum and maximum) will be calculated for continuous safety data by treatment and protocol specified time point, while frequency summary (e.g. number of observed and percentage of each categories) will be applied for categorical safety data by treatment and protocol specified time point.

### Sample Size

The sample size was calculated based on the CCB data in confidence bound in literature report (Rirash 2017). Assuming a 2-sided 0.05 alpha for treatment (cilnidipine or combination therapy or tadalafil) versus placebo and without controlling alpha for the multiple efficacy comparisons, a sample size of eight participants in each paired comparison group (cilnidipine or combination therapy or tadalafil) is needed for 80% power in a 4×4 crossover design to detect a 25% difference at common SD of 0.5 (a moderate effect size) in percent change from baseline of Raynaud's attack per week. Assuming a 20% dropout rate for final efficacy analysis, ten participants in each group is planned.

After reviewing the results from Part A: Run-in phase, the sample size for Part B may be adjusted.

### Analysis Populations

Participant inclusion into each population has been or will be determined prior to the final analysis.

### Intent-To-Treat (ITT) Population

All participants who are entered into the study and complete screening, sign an informed consent for the study and randomized have been or will be included in the ITT population.

### Per Protocol Population

All participants who complete the study with all Dosing Periods (for Part B—at least 5 days of dosing within the last 7 days treatment for the first two periods, and 4 days of dosing within the last 7 days treatment for the second two periods) and meet all eligibility criteria, will be included in the PP Analysis Population.

### Pharmacokinetic Population

All participants who receive any amount of active study drug and have sufficiently evaluable concentration time profile to allow determination of at least one PK parameter will be included in the PK population. An evaluable PK profile will be determined at the discretion of the pharmacokineticist following examination of participants with dosing or protocol deviations that could potentially affect the PK profile. The PK population will be used for the summaries of all PK data.

### Safety Population

All randomized participants who received study drug have been or will be included in Safety population and will be classified according to the actual treatment received.

### Statistical Methods

### Participant Disposition

Participant disposition has been and will be analyzed using counts and percentages. The number and percentage of screened participants, enrolled participants, treated participants, participants discontinued from the study and study treatment, as well as the primary reason for discontinuation has been and will be analyzed and listed.

### Demographics, Medical History, and Baseline Characteristics

Demography and baseline characteristics data has been and will be analyzed using descriptive statistics. The following demographic variables will be summarized by dose level: race, gender, age, height and weight, concomitant diseases (Hypertension, peripheral vascular disease, diabetes, CKD and stage, osteoporosis, history and type of heart arrhythmia).

In addition, the following baseline characteristics of Raynauds Disease have been and will be analyzed will be summarized: age of onset, seasonality (months disease is worst), usual number of attacks/day, usual peak severity, baseline RCS assessment, how attacks are usually treated, how long attacks last in general, experience with other treatments both pharmacological and non-pharmacological.

Medical history terms are coded using the MedDRA® Version 22.0 or higher. Medical history has been and will be analyzed using descriptive statistics by MedDRA® SOC and PT.

### Prior and Concomitant Medication

Prior and concomitant medications were or will be coded using the most current version of the WHO drug dictionary available at the start of the study. Prior and concomitant medications will be listed by participant and summarized by treatment using anatomical therapeutic chemical (ATC) and preferred name.

### Treatment Compliance and Exposure

Treatment compliance and exposure has been and will be summarized and listed by treatment for all participants in the Safety population.

### Analysis of Primary Endpoint

Percent change from baseline evaluation for frequency of weekly RP attacks is and will be the primary efficacy endpoint. Data collected in the last 7 days of each Dosing Period was and will be used for this analysis. Screening assessments were and will be used as baseline for the analysis of all periods. No multiple comparison adjustment will be used to control alpha for the multiple comparisons. Mixed effects model will be used for analysis of the primary endpoint according to the crossover design. Other efficacy endpoints of continuous variables will be analyzed using similar methodology. For nominal data, Chi-square tests will be applied. Generalized Estimating Equations method will be used, as appropriate, for adjusting for potential confounding factors. In addition, the final analysis will assess whether in this study of severe Raynaud's disease participants, the minimally important difference, previously concluded of 14-15 points on the 100 point RCS scale (Khanna, 2010) has been achieved in the cilnidipine dose group. It also will record the percentage of participants achieving a PASS (34 point difference from baseline on a 0-100 VAS) (Khanna, 2010) in each treatment group.

### Analysis of Secondary Endpoints

The secondary endpoints of change from baseline evaluation will be compared among treatment groups using mixed effects model. Kaplan-Meier method will be used to evaluate time to event endpoints. To evaluate the impact of daily ambient temperature on symptomatic Raynaud's attack, logistic regression will be used for temperature versus the occurrence of Raynaud's attack (Yes/No). The effect of temperature on the severity score of Raynaud's attacks and difference of using rescue medication between treatment groups will be evaluated by Chi-square test. The impact of therapy in sympathetic activity will be assessed by mixed model for repeated measures.

### Safety Analyses

All safety assessments, including AEs, laboratory evaluations, vital signs, and other safety assessments will be analyzed using the Safety population.

### Adverse Event

Adverse events will be coded using the most current version of the MedDRA® Version 22.0 or higher. The analysis of AEs will be based on the concept of treatment emergent AEs. Treatment emergent AEs will be tabulated by treatment group and will include the number of participants for whom the event occurred, the severity, and relationship to study drug. Treatment emergent AEs (TEAEs) leading to discontinuation and SAEs with onset after the start of study drug will also be summarized.

All AEs and SAEs (including those with onset or worsening before the start of study drug) through the end of the study will be listed.

### Laboratory Evaluations

Baseline laboratory evaluations will be listed and summarized by treatment.

### Vital Signs

Vital signs (BP [systolic and diastolic], pulse rate, and oral temperature) will be listed and summarized by treatment and protocol specified collection time point. Observed and change from baseline will be summarized at each protocol specified collection time point.

### Other Safety Assessments

The following assessments will be listed by participant:
Pregnancy Test
Raynaud's function assessment by physician.

### Pharmacokinetics

Plasma concentrations and actual blood sampling times will be listed by treatment and protocol specified time point and summarized using descriptive statistics-number of measurements, arithmetic mean, SD, and % CV, geometric mean, minimum, median, and maximum—at each scheduled time point. Individual and mean plasma concentration-time profiles will also be presented graphically for each treatment.

Pharmacokinetic parameters will be computed from the individual plasma concentrations using a non-compartmental approach.

Value for elimination rate constant (kₑₗ), elimination half-life (t_{1/2}), Area under the plasma concentration-time curve from time zero to infinity (AUC_{0-inf}), apparent total clearance of the drug from plasma after oral administration (CL/F) or apparent volume of distribution during terminal phase after non-intravenous administration (Vz/F) will not be reported. Additional analyses will be performed as deemed necessary upon review of the data.

### REFERENCES

Khanna P P, Maranian P, Gregory J, Khanna D. The minimally important difference and patient acceptable symptom state for the Raynaud's condition score in patients with Raynaud's phenomenon in a large randomised controlled clinical trial. Ann Rheum Dis. 2010; 69 (3): 588-591. doi: 10.1136/ard.2009.107706
Rirash F, Tingey P C, Harding S E, et al. Calcium channel blockers for primary and secondary Raynaud's phenomenon. The Cochrane database of systematic reviews, 2017; 12 (12), CD000467.

### Example 2. Statistical Analysis Plan

### INTRODUCTION

The purpose of this statistical analysis plan (SAP) is to describe the procedures and the statistical methods that have been and will be used to analyze data and report results collected as described in Example 1.

### Study Objectives

This study is a placebo-controlled phase 2a study to test the efficacy and safety of cilnidipine alone and in combination with tadalafil in participants who have frequent attacks of secondary RP mostly resulting from SSc (e.g., SSc-RP). The study consists of two parts, Part A and Part B.

The primary purpose of Part A (a double-blind, placebo-controlled, parallel-group study testing 6 treatment combinations) was and is to generate efficacy and safety data that allows the DSMB to select the dose of cilnidipine (10 mg or 20 mg) to be studied in Part B and to confirm the sample size estimates for Part B of the study.

Part B will provide the primary evidence of efficacy and safety. Part B is a double-blind, placebo-controlled, 4-way crossover study, designed to assess the safety and efficacy of cilnidipine (at the dose selected in Part A) alone and in combination with 5 mg tadalafil. Participants will be randomized to one of four prespecified treatment sequences in a 4-way crossover design.

### Primary Efficacy Objective

The primary efficacy objective of the study was and is to evaluate the effect of cilnidipine alone and in combination with tadalafil on the frequency of weekly Raynaud's Phenomenon (RP) attacks compared with placebo in participants with Raynaud's Phenomenon secondary mostly to systemic sclerosis (SSc-RP).

### Secondary Efficacy Objective

The secondary efficacy objective of the study is to evaluate the effect of cilnidipine alone and in combination with tadalafil on the clinical, measured, and global features of SSc-RP and the severity and burden of these SSc-RP symptoms.

### Safety Objective

The safety objective of the study was and is to evaluate the safety of cilnidipine alone and in combination with tadalafil compared to placebo in participants with SSc-RP.

### Exploratory Objective (Part A, Dose Selection)

To assess the endothelial function of participants with SSc-RP and impact of treatment on sympathetic activity and vascular functioning (Part A).

### Study Endpoints

### Primary Efficacy Endpoint

The primary efficacy endpoint of this study is:
Percentage change from baseline in frequency of weekly RP attacks.

### Secondary Efficacy Endpoints

Secondary efficacy endpoints of the study include:
Change from baseline in frequency of weekly RP attacks
Change from baseline in average duration of weekly RP attacks
Change from baseline in average severity of weekly RP attacks
Change from baseline in average daily Raynaud's Condition Score (RCS)
Change from baseline in highest (most severe) pain score recorded during weekly RP attacks
Change from baseline in average pain score recorded during weekly RP attacks
Change from baseline in net digital ulcer burden
Change from baseline in Distal-dorsal difference (DDD) of the affected index finger sites
Change from baseline in participant quality of life measured using the Scleroderma Health Assessment Questionnaire (SHAQ)
Change from baseline in participant gastrointestinal symptoms (of sclerosis) as assessed with the UCLA SCTC GIT 2.0 questionnaire
Change from baseline in Raynaud-Visual analog scale (VAS)
Change from baseline in physician assessment of disease
Measurement of cilnidipine drug levels taken 2 to 6 hours following the last dose
The time to reach maximum degree of efficacy (in days) compared to baseline
The time to return to baseline symptom severity after termination of dosing
Impact of daily ambient temperature on symptomatic RP attacks
Use of rescue medications for breakthrough symptoms.

### Safety Endpoints

The safety endpoint of the study is:
Incidence of adverse events (AEs) and serious adverse events (SAEs), including clinically significant vital signs from the time of randomization until 7 days following the last protocol dose.

### Exploratory Endpoint (Part A Dose Selection).

Change from baseline in endothelial function as measured by Reactive Hyperemia Index (RHI) using Endothelial Dysfunction (Endo-PAT)

### Summary of the Study Design

### General Study Design and Plan

A schematic of the overall study design is provided in FIG. 1. Participants have or will undergo a screening period beginning up to 10 days prior to randomization. The initial screening and capacity was or will be conducted via phone at the start of the Screening period with eligibility finalized prior to randomization on Day 0. Participants were or will be required to provide informed consent in a 2-step process at Screening (by agreeing to complete the screening Diary) and at Randomization (Day 0) before undertaking any study-specific procedures or assessments. Only participants who met or will meet all of the inclusion and none of the exclusion criteria were or will be randomized.

### Part A—Double-Blind, Placebo-Controlled, Parallel-Group, Dose Selection

Assess the safety and efficacy of two doses of cilnidipine (10 mg and 20 mg), alone and in combination with tadalafil. A total of 36 participants were or will be randomized to one of six pre-specified parallel treatment arms.

Please refer to FIG. 1 for treatment arms in Part A of the study. Dosing lasted or will last for 12 (±2) days in which participants self-administered or will self-administer daily doses of assigned treatment in the morning. Each participant took or will take one capsule and one tablet to blind the active therapy being received.

The data from Part A of the study will be reviewed by a data and safety monitoring board (DSMB) including unblinded analysis results, to support selecting the cilnidipine dose and confirming the sample size estimates for the randomized, double-blind, crossover design, phase (Part B).

The first review by the DSMB will occur after 16 participants completed the study in Part A.

### Part B—Double-Blind, Placebo-Controlled, 4-Way Crossover

Assess the safety and efficacy of cilnidipine (at the dose selected in Part A) alone and in combination with 5 mg tadalafil. A total of 40 participants (10 in each sequence) with a diagnosis of SSc-RP will be randomized into one of four pre-specified treatment sequences in a 4-way crossover design. Part B is designed to provide the primary evidence for efficacy analyses.

FIG. 1 depicts the treatment sequences in Part B of the study. Each participant will undergo four dosing periods in which they will receive a different treatment each dosing period followed by a 4 (±1) day washout period. Each dosing period will last for 12 (±2) days in which participants will self-administer daily doses of assigned treatment in the morning. At all dosing periods, each participant will take one capsule and one tablet to blind the active therapy being received.

For both Part A and B of the study, participants were or are required to visit the clinic on last day of each dosing period (i.e., Day 10 to 14) to return/dispense study drug and conduct in person study assessments. Participants were or will be dispensed with 2 weeks' worth of study drug to be taken at home for the following dosing period; overage from the prior dosing period will also be collected.

Patients were and will be assessed for the occurrence of efficacy endpoints for each dosing period via the patient reported diary and the in-clinic visit. Safety information was and will be collected for each dosing period from randomization until patient follow-up is complete (7 to 10) days after the last dosing.

Patients completing Part A may also be enrolled in Part B, as long as they are eligible per the inclusion criteria of Example 1 is met.

### Randomization and Blinding

Each participant was or will be provided with a unique screening number post-documentation of informed consent. Once deemed eligible, the participant was or will be assigned a sequential randomization number prior to first dosing in each Part A and Part B as described below. Participants who withdraw from the study or who fail to meet inclusion criteria, for any reason, prior to randomization will be considered screen failures.

### Part A

A total of 36 participants were or will be randomized in a 1:1:1:1:1:1 ratio to receive one of six pre-specified, parallel treatment arms cilnidipine 10 mg, cilnidipine 20 mg, tadalafil 5 mg, cilnidipine 10 mg+tadalafil 5 mg, cilnidipine 20 mg+tadalafil 5 mg, or placebo.

### Part B

A total of 40 participants (10 in each sequence) will be randomized to 1 of 4 treatment sequences of 4 crossover periods, according to a 4×4 Williams square, as outlined in Table 1.

TABLE 1
Part B 4-way crossover

| | Dosing | Dosing | Dosing | Dosing |
|---|---|---|---|---|
| Sequence # | Period 1 | Period 2 | Period 3 | Period 4 |
| 1 | P | C | C + T05 | T05 |
| 2 | T05 | C + T05 | P | C |
| 3 | C + T05 | T05 | C | P |
| 4 | C | P | T05 | C + T05 |

Abbreviations:
C = cilnidipine,
P = placebo,
T = tadalafil,
N = number of participants,
T05 =T 5 mg.
Dose of C, either 10 mg or 20 mg will be identified following completion of Part A.

Once a randomization number was or is assigned, it cannot be reassigned to any other participant. All participants who are randomized will be followed and included in the primary ITT analysis. Dropouts will not be replaced.

All randomization codes were or will be generated by the designated unblinded independent statistician prior to the start of the study. Sealed code break envelopes will be provided prior to start of the study.

### Sample Size and Statistical Power Considerations

The sample size was calculated based on the CCB data in confidence bound in literature report (Rirash 2017 referenced in Example 1). Assuming a 2-sided 0.05 alpha for treatment (cilnidipine or combination therapy or tadalafil) versus placebo and without controlling alpha for the multiple efficacy comparisons, a sample size of eight participants in each paired comparison group (cilnidipine or combination therapy or tadalafil) is needed for 80% power in a 4×4 crossover design to detect a 25% difference at common SD of 0.5 (a moderate effect size) in percent change from baseline of Raynaud's attack per week. Assuming a 20% dropout rate for final efficacy analysis, ten participants in each group is planned for Part B.

After reviewing the results from Part A: Dose selection phase, the power assumptions will be reviewed and sample size for Part B may be adjusted.

### Statistical Considerations

### General Considerations

The statistical analysis will be conducted following the principles specified in the International Council for Harmonization (ICH) Topic E9 Statistical Principles for Clinical Trials. For more information, see https://www.ema.europa.cu/en/ich-e9-statistical-principles-clinical-trials, which is incorporated by reference herein in its entirety.

Unless otherwise noted, continuous variables will be summarized by number of subjects (n), mean, standard deviation (SD), first quartile (Q1), median, third quartile (Q3), minimum and maximum values. In addition, change from baseline values will be calculated at each time point and summarized descriptively. Categorical variables will be summarized by frequency count and the percentage of subjects in each category. Summaries will be generated for each treatment, where appropriate. Individual subject data will be presented in subject data listings.

The default significant level will be 5%; confidence intervals (CIs) will be 95% and all tests will be two-sided, unless otherwise specified in the description of the analyses. Min and max values will be rounded to the precision of the original value. Means, and medians will be rounded to one decimal place greater than the precision of the original value. SDs, SEs, and 95% CIs will be rounded to two decimal places greater than the precision of the original value. Percentages for summarizing categorical data will be rounded to one decimal place. P-values will be rounded to three decimal places. If a p-value is less than 0.001 it will be reported as “<0.001.” If a p-value is greater than 0.999 it will be reported as “>0.999.”

### Definitions of Analysis Populations

Participant inclusion into each analysis population will be determined prior to the final analysis.

### Intent-to-Treat (ITT) Population

All participants who enter or entered into the study and complete screening, sign or signed an informed consent for the study and randomized will be included in the ITT population. Treatment classification will be based on the randomized treatment for analysis.

### Per Protocol Population

All participants who complete the study with all Dosing Periods (for Part B—at least 5 days of dosing within the last 7 days treatment for the first two periods, and 4 days of doing within the last 7 days treatment for the second two periods), meet all eligibility criteria, and without any major/important protocol deviations, will be included in the per-protocol (PP) population. PP analysis population will be evaluated and finalized before database lock.

### Pharmacokinetic Population

All participants who receive any amount of active study drug and have sufficiently evaluable concentration time profile to allow determination of at least one pharmacokinetic (PK) parameter were or will be included in the PK population. An evaluable PK profile was or will be determined at the discretion of the pharmacokinetic specialist following examination of participants with dosing or protocol deviations that could potentially affect the PK profile. The PK population was or will be used for the summaries of all PK data.

### Safety Population

All randomized participants who received study drug were or will be included in Safety population and have been or will be classified according to the actual treatment received.

Data from Part A and Part B will be analyzed separately. The pooled analyses of Part A and Part B may be conducted as exploratory for participants with common treatment. The intent-to-treat (ITT) population will be used to summarize participant disposition. The primary and secondary efficacy analyses will be based on the ITT population. Analyses based on the PP population for Part B will be considered secondary and confirmatory. All safety analyses will be performed on the safety population.

### Definitions and Conventions for Data Handling

### Baseline Definition

In general, the non-missing measurements collected during the last 7 days prior to the date of randomization served and will serve as the data for calculation of baseline measurements for efficacy variables. The data collected for assessments that were or are performed first time on randomization (Day 0) visit (vital signs, digital ulcer assessment, and Endo-PAT) will serve as baseline measure for efficacy endpoints for those assessments. If there is no value on or prior to the date of randomization, then the baseline value will not be imputed, and will be set to missing.

### Study Day

Study day has been and will be calculated from the reference start date and was and will be used to show start/stop day of assessments and events.

Reference start date (Day 1) was or is defined as the date of first dose of study drug.
Study day=(date of event−reference start date+1) if the event date is on or later than reference start date
Study day=(date of event−reference start date) if the event date is earlier than reference start date
   In the situation where the event date is partial or missing, Study day, and any corresponding durations will appear partial or missing in the listings.

### Analysis Visit Windows

For all analyses for this study, the scheduled visit and/or time point from the case report form (CRF) (i.e., CRF visit) was or will be used as the analysis visit and/or time point.

Measurements collected from unscheduled visits will not be included in the by visit summary tables but will be included in the listings.

### Missing Data Handling Rules

Missing data as well as data from participants who drop out early was or will not be imputed.

### Examination of Subgroups

The following subgroup analysis will be performed for the primary efficacy analysis. Subgroup results need to be interpreted with caution if there are insufficient number of subjects in a subgroup.

### Part A/Part B:

Baseline RP attack frequency: >=13-<=20 attacks per week, >20-<=30 attacks per week, >30 attacks per week.
Average baseline RCS score: Baseline RCS<35, >=35-<=75, >75
Time of RP attacks since daily study medication: <8 hours, >=8-<=16 hours, >16-<=24 hours

### Part B (Additional Analyses):

Age group (<45, >=45 and <=64, >=65).
Gender.
Baseline Weight: <60 kg, >=60-<=100 kg, >100 kg.
Baseline scleroderma diagnosis: Y/N
Smoking status: active, former, non-smoker.
Duration of dosing: <10 days, >=10-<12 days, >=12-<=14 days
Stable use of concomitant CCBs: Y/N
Rescue medicines required: Y/N.
Behavioral modifications required: Y/N

### Primary, Secondary and Other Variables

### Primary Efficacy Variable

Percent change from baseline evaluation for frequency of weekly RP attacks were or will be used as the primary efficacy variable. The sponsor-developed participant-informed diary will be used to record data. Frequency of weekly RP attacks is defined as the total number of RP attacks divided by the number of days with available diary data within the last 7 days of each dosing period then multiplied by 7. Total number of RP attacks during the last 7 days of screening period divided by the number of days with available data then multiplied by 7 will be used as baseline for the analysis of all periods.

The variable will be calculated as follows:

Percentage change={(weekly RP attacks for the last 7 days of each dosing period−Baseline weekly RP attacks)/Baseline weekly RP attacks}*100%.

### Secondary Efficacy Variables

Change from Baseline in Frequency of Weekly RP Attacks

The absolute change in frequency of weekly RP attacks from baseline to the end of each Dosing Period was and will be a secondary outcome variable and calculated as follows:

Absolute change=weekly RP attacks for the last 7 days of each dosing period−Baseline weekly RP attacks

Change from Baseline in Average Duration of Weekly RP Attacks

The absolute change in average duration of weekly RP attacks from baseline to the end of each dosing period was and will be a secondary outcome variable. The sponsor-developed participant-informed diary was and will be used to record data. Average duration of weekly RP attacks is defined as the total duration of RP attacks divided by total number of RP attacks within the last 7 days of each dosing period. Total duration of RP attacks during the last 7 days of screening period divided by the total number of RP attacks will be used as baseline for the analysis of all periods. The variable will be calculated similar to frequency data as before.

Change from Baseline in Average Severity of Weekly RP Attacks

The absolute change in average severity of weekly RP attacks (VAS 0-10 cm scale) from baseline to the end of each Dosing Period was and will be a secondary outcome variable. The sponsor-developed participant-informed diary will be used to record data. Average severity of weekly RP attacks is defined as the total severity scores of RP attacks divided by total number of RP attacks within the last 7 days of each dosing period. Total severity scores of RP attacks during the last 7 days of screening period divided by the total number of RP attacks will be used as baseline for the analysis of all periods. The variable will be calculated similar to frequency data as before.

Change from Baseline in Average Daily RCS

The RCS is based on how much difficulty participants had with Raynaud's today, how many attacks the participant had, and how long they lasted. In addition participants were and will be asked to consider how much pain, numbness, or other symptoms the Raynaud's caused in fingers (including painful sores), and how much the Raynaud's along affected the use of hand today (VAS 0-10 cm scale).

The absolute change in average daily RCS from baseline to the end of each dosing period was and will be a secondary outcome variable. The sponsor-developed participant-informed diary has been and will be used to record data. The average daily RCS is defined as the total RCS divided by the number of days with available diary data of each dosing period. Total RCS during screening period divided by the number of days with available data will be used as baseline for the analysis of all periods. If there are multiple daily RCS scores, the latest daily RCS will be used. The variable will be calculated similar to frequency data as before.

Change from Baseline in Highest (Most Severe) Pain Score Recorded During Weekly RP Attacks

The absolute change in highest pain score (11-point Likert scale) of weekly RP attacks from baseline to the end of each dosing period will be a secondary outcome variable. The sponsor-developed participant-informed diary will be used to record data. The highest pain score collected during the last 7 days of each dosing period will be used for this analysis. The highest pain score among the last 7 days of screening assessments will be used as baseline for the analysis of all periods. The variable will be calculated similar to frequency data as before.

Change from Baseline in Average Pain Score Recorded During Weekly RP Attacks

The absolute change in average pain score (11-point Likert scale) of weekly RP attacks from baseline to the end of each Dosing Period will be a secondary outcome variable. The sponsor-developed participant-informed diary will be used to record data. The average pain score of weekly RP attacks is defined as the total pain scores divided by total number of RP attacks within the last 7 days of each dosing period. Average of the last 7 days of screening assessments will be used as baseline for the analysis of all periods. The variable will be calculated similar to frequency data as before.

Change from Baseline in Net Digital Ulcer Burden

If participant has digital ulcers, the absolute change in digital ulcer severity (VAS 0-10 cm scale) from baseline to the end of each dosing period will be a secondary outcome variable. The digital ulcer will be assessed by physician at screening and the in-clinic visit. Screening assessments will be used as baseline for the analysis of all periods. The variable will be calculated similar to frequency data as before.

Change from Baseline in Distal Dorsal Difference (DDD) of the Affected Index Finger Sites

Thermography assessments will be performed by physician at the in-clinic visit. Thermography will be conducted on the ring, middle and index digits of both hands; the participant must be indoors for at least 30 minutes prior to the test to give the body time to equilibrate. Photos will be taken with the help of Fluor thermographic camera of these two areas at the in-clinic visit.

The absolute change in DDD measured by thermography from baseline to the end of each dosing period will be a secondary outcome variable. Each participant will have 4 DDD scores at each visit: PIP nailbed Left, DIP-PIP Left, PIP nailbed Right and DIP-PIP Right. The variable will be calculated similar to frequency data as before and the analysis will be summarized by hand and location.

Change from Baseline in Participant Quality of Life Measured Using the SHAQ

The standard, validated, patient reported outcome measures tool for SSc patients, the Scleroderma Health Assessment Questionnaire (SHAQ), will be used to assess the participant qualify of life. Details are attached in the Appendix. The participant quality of life will be scored, and a disability index calculated for each questionnaire completed by the patient at Screening and each in-clinic visit. The absolute change in participant quality of life from baseline to the end of each dosing period will be calculated similar to frequency data as before.

Change from Baseline in Participant Gastrointestinal Symptoms (of Sclerosis) as Assessed with the UCLA SCTC GIT 2.0 Questionnaire

The standard, validated, patient reported outcome measures tool for SSc patients, the University of California at Los Angeles Scleroderma Clinical Trials Consortium Gastrointestinal Tract 2.0 (UCLA SCTC GIT 2.0), will be used to assess the participant gastrointestinal symptoms (of sclerosis). Details are attached in the Appendix. The participant gastrointestinal symptoms will be assessed at Screening and each in-clinic visit. Participant responses to the questionnaire will be scored and used to calculate a total score indicating the impact of gastrointestinal symptoms on quality of life. The constipation score is not included in the calculation of the total score and will be reported separately. The absolute change in participant gastrointestinal symptoms from baseline to the end of each dosing period will be calculated similar to frequency data as before.

Change from Baseline in Raynaud-Visual Analog Scale (VAS)

The SHAQ includes a Raynaud's VAS, which will be reported at screening and each in-clinic visit. The absolute change in Raynaud-VAS, assessed by the participants response to the SHAQ question ‘In the past 7 days, how much have your Raynaud's interfered with your daily activities?’ at baseline and the end of each dosing period will be calculated similar to frequency data as before.

Change from Baseline in Physician Assessment of Disease

The Physician will rate severity of participant's Raynaud's disease at Screening and in-clinic visit. The absolute change in physician assessment of disease from baseline to the end of each dosing period will be calculated similar to frequency data as before.

### Measurement of Cilnidipine Drug Levels Taken 2 to 6 Hours Following the Last Dose

One 4 mL blood sample will be obtained during each in-clinic visit within 2 to 6 hours of the last dose of study drug in that dosing period. The level of cilnidipine in blood will be measured following last dose of the dosing period. The actual sampling times will be used in the PK parameter calculations.

Concentrations are used as supplied by the analytical laboratory for PK analysis. The units of concentration and resulting PK parameters, with amount or concentration in the unit, will be presented as they are received from the analytical laboratory. If values below LLOQ is noted, half of the LLOQ will be imputed for summary analysis, but below LLOQ will be left as is in listings.

### The Time to Reach Maximum Degree of Efficacy (in Days) Compared to Baseline

The maximum degree of efficacy is defined as the least daily frequency of PR attacks. If there are more than one day with the same least frequency of PR attacks, the time to the first maximum degree of efficacy will be used for this analysis.

The Time to Return to Baseline Symptom Severity after Termination of Dosing

During each washout period and follow-up period, the time to return to the worst baseline severity of weekly PR attacks will be calculated as the first time of return to the worst baseline severity—the previous in-clinic visit date. A participant with no improvement after baseline or never returns to baseline symptom severity will be regarded as censored.

### Safety Variables

### Extent of Exposure

Extent of exposure in days for each Dosing Period was and will be derived from the following formula:

$$Extent of exposure ( days ) = ( date of first dose of each Dosing Period ) - ( date of last dose of each Dosing Period ) + 1$$

All doses were and will be self-administered by participants remote from study sites (at home). For each dosing period, participants were or will be dispensed with two weeks' worth of study medication and will be asked to return the unused study medication on the last day of each Dosing Period at the time of in-clinic visit. The treatment compliance was and will be noted by the Investigator(s) during the in-clinic visit.

### Adverse Events

AEs will be coded by System Organ Class (SOC) and Preferred Term (PT) using the MedDRAR Version 22.0 or higher. The verbatim term will be included in the AE listings.

Treatment-emergent AEs (TEAEs) are defined as AEs that occur or worsen after the dose of study drug. If the timing of the start of an AE could not be determined unambiguously from the start or end dates provided, it will be assumed to be a TEAE. An AE is considered related if the relationship to either of the study drug has been indicated as possibly or probably or definite related by the investigator. An AE leading to study withdrawal is defined as an AE that cause a subject early terminated from the study. AEs will be identified as emerging in the following parts:
TEAEs for Part A will be those that started during Part A (i.e., from the first dose in Part A to end of follow up of Part A).
TEAEs for Part B will be those that started during Part B (i.e., from the first dose in Part B to end of follow-up of Part B).

The TEAEs by treatment will be presented according to the last treatment received prior to the AE start date for crossover periods in Part B.

All AEs will be listed by participant but only TEAEs will be summarized.

### Clinical Laboratory Variables

Routine hospital laboratory tests including hematology, biochemistry, inflammatory markers (CRP and ESR), and antibody status (Scl-70) will be conducted as clinically indicated per standard of care but are not required per protocol.

### Vital Signs

Vital signs including Systolic Blood Pressure (SBP), Diastolic Blood Pressure (DBP), pulse rate, and temperature were and will be measured at Screening and each in-clinic visit.

Changes in vital signs variables between baseline and each subsequent scheduled assessment will be calculated. Absolute values will be compared to the relevant reference ranges and classified as LNH (low (below range), normal (within range or on limits) or high (above range)). All values (absolute and change) falling outside the reference ranges (see Table 2) will be flagged.

TABLE 2
Vital signs reference ranges

| | Standard | Lower | Upper |
|---|---|---|---|
| Parameter | Units | Limit | Limit |
| Diastolic Blood Pressure (DBP) | mmHg | 60 | 120 |
| Systolic Blood Pressure (SBP) | mmHg | 100 | 160 |
| Pulse Rate | Beats/min | 40 | 120 |
| Body Temperature | Celsius | 36.5 | 38 |
| Weight | kg | 40 | 200 |

Body mass index (BMI) will be calculated from the height and weight as follows:

$${BMI ( kg / {m}^{2} ) = weight ( kg ) / height ( m ) )}^{2}$$

### Other Safety Variables

A urine pregnancy test will be performed at the randomization (Day 0) visit and on the last clinic visit at the end of last dosing period for Woman of childbearing potential (WOCBP) only.

Raynaud's function assessment will be conducted by Physician at Randomization (Day 0) visit and each in-clinic visit.

### Exploratory Variables

### Endothelial Function

Assessments for endothelial dysfunction will be performed using Endo-PAT at randomization (Day 0) visit and each in-clinic visit for Part A only; Endo-PAT assessment will not be performed in Part B of the study. The Endo-PAT is a diagnostic device used to assess endothelial vasodilator function in a rapid and non-invasive fashion. Endothelial function will be measured using the reactive hyperemia index (LnRHI): (normal LnRHI is ≥0.7; Grey zone 0.51-0.7; Abnormal ≤0.51). The absolute change from baseline will be calculated similar to frequency data as before.

### Analysis Methods

### Subject Information

### Disposition of Subjects

All screened participants will be included in the summaries of participant disposition. Separate summaries will be provided for Part A and Part B. The summaries will include the number of screened participants, the number of randomized participants, the number and percentage of treated participants, participants discontinued from the study and study treatment, and the primary reason for discontinuation.

The number and percentage of participants in each of the ITT population, PP population, PK population, and Safety population will also be summarized.

Listings of participant disposition will be provided by participant.

### Protocol Deviations

Prior to database lock, all Protocol Deviations (PDs) will be identified and documented based on a blinded review of potential PDs. The potential PDs will be reviewed by study team and classified as major or minor. All PDs will be listed by participant.

Major PDs will be summarized by classification and treatment sequence.

### Demographics and Baseline Characteristics

Demography and baseline characteristics data will be summarized using descriptive statistics. The following demographic variables will be summarized by treatment sequence: race, gender, age (summarized both as a continuous variable and as a categorical variable, with categories <45 years, >=45 to <=64 years, and >=65 years), height and weight, concomitant diseases (hypertension, peripheral vascular disease, diabetes, CKD and stage, osteoporosis, history and type of heart arrhythmia). Separate summaries will be provided for Part A and Part B.

In addition, the following baseline characteristics of Raynaud's Disease will be summarized: age of onset, seasonality (months disease is worst), usual number of attacks/day, usual peak severity, baseline RCS assessment, how attacks are usually treated, how long attacks last in general, experience with other treatments both pharmacological and non-pharmacological.

Pregnancy test results will be listed but not summarized.

A listing of demographic and baseline characteristics will be provided by participant.

### Medical History

Medical history terms will be coded using the MedDRA® Version 22.0 or higher. Medical history will be summarized by MedDRA® SOC and PT. Medical history will be listed by participant.

### Prior and Concomitant Therapy

Medications will be coded using the most current version of the WHO drug dictionary available at the start of the study.

Those medications taken prior to first dose of randomized study drug will be denoted “Prior.” Those medications started at the same time or after the first dose of randomized study drug will be denoted “Concomitant.”

Medications will be presented according to whether they are “Prior” or “Concomitant,” as defined above. Note that a medication could be both prior and concomitant. If medication dates are incomplete and it is not clear whether the medication was concomitant, it will be assumed to be concomitant.

Prior and concomitant medications will be listed by participant and summarized by treatment using anatomical therapeutic chemical (ATC) and preferred name.

### Treatment Compliance and Exposure

Descriptive summary statistics will be provided for treatment exposure and compliance for each treatment sequence and all participants.

Mean, standard deviation, median, minimum, and maximum of amount of unused study medication returned will be provided. The cell frequencies and percentage of participants in each category (<50%, >=50% and <=75%, >75%) will be provided.

### Efficacy Analyses

### Primary Efficacy Analysis

### Primary Analysis

Percent change from baseline evaluation for frequency of weekly RP attacks was and will be the primary efficacy endpoint. Frequency of weekly RP attacks is defined as the total number of RP attacks divided by the number of days with available diary data within the last 7 days of each dosing period then multiplied by 7. Total number of RP attacks during the last 7 days of screening period divided by the number of days with available data then multiplied by 7 will be used as baseline for the analysis of all periods.

The absolute value, change and percent change from baseline for frequency of weekly RP attacks was or will be summarized via descriptive statistics by treatment.

Analysis will be performed in ITT population using a mixed model. The dependent variable is percent change from baseline in frequency of weekly RP attacks, and the independent variables include treatment, sequence, period, as fixed effects, and participant as a random effect. Kenward and Roger's method will be used to calculate the denominator degrees of freedom for the fixed effects (DDFM=KR). The lease square mean (95% CI) of percent change from baseline for each treatment and the least square difference between each treatment and placebo will be obtained from the LSMEANS statement.

Separate analyses were provided will be provided for Part A and Part B. The pooled analyses of Part A and Part B may be conducted as exploratory for participants with common treatment.

### Confirmatory Analysis

For the primary endpoint, a confirmatory analysis will be conducted in the same manner as the primary analysis in PP population.

### Subgroup Analysis

The following subgroups will be examined for the primary endpoint in the same manner as the primary analysis:

### Part A/Part B:

Baseline RP attack frequency: >=13-<=20 attacks per week, >20-<=30 attacks per week, >30 attacks per week
Average baseline RCS score: Baseline RCS<35, >=35-<=75, >75
Time of RP attacks since daily study medication: <8 hours, >=8-<=16 hours, >16-<=24 hours

### Part B (Additional Analyses):

Age group (<45, >=45 and <=64, >=65)
Gender
Baseline Weight: <60 kg, >=60-<=100 kg, >100 kg
Baseline scleroderma diagnosis: Y/N
Smoking status: active, former, non-smoker
Duration of dosing: <10 days, >=10-<12 days, >=12-<=14 days.
Stable use of concomitant CCBs: Y/N
Rescue medicines required: Y/N.
Behavioral modifications required: Y/N

### Secondary Efficacy Analyses

Change from Baseline Evaluation

The secondary endpoints of change from baseline evaluation including: frequency of weekly RP attacks, average duration of weekly RP attacks, average severity of weekly RP attacks, average daily RCS, highest pain score recorded during weekly RP attacks, average pain score recorded during weekly RP attacks, digital ulcer severity, Distal-dorsal difference (DDD) measured by thermography, quality of life measured by SHAQ, gastrointestinal symptoms assessed by UCLA SCTC GIT 2.0, Raynaud-VAS and physician assessment of disease, will be analyzed by treatment groups using mixed effect model in the same manner as the primary analysis.

### Time to Reach Maximum Degree of Efficacy

The mean time to reach maximum degree of efficacy will be summarized by treatment.

Time to reach maximum degree of efficacy (in days) will be evaluated using the Kaplan-Meier Survival Analysis approach. Descriptive summary, including time to event percent-tiles (25%, 50%, and 75%) and 95% confidence intervals and Kaplan-Meier mean (SE) will be estimated.

Time to Return to Baseline Symptom Severity after termination of dosing

The mean time to return to the worst baseline symptom severity will be summarized by treatment.

Time to return to the worst baseline symptom severity (in days) will be evaluated using the Kaplan-Meier Survival Analysis approach. If a participant doesn't return to baseline symptom severity during washout period or follow-up period, it will be regarded as censor for that Dosing Period. Descriptive summary, including number of participants (%) censored, time to event percent-tiles (25%, 50%, and 75%) and 95% confidence intervals and Kaplan-Meier mean (SE) will be estimated.

### Impact of Daily Ambient Temperature on Symptomatic RP Attacks

To examine the impact of daily ambient temperature on RP attacks, a Generalized Estimating Equation (GEE) with adjustment for the ambient temperature, treatment effect, period effect, and interaction between treatment and period will be conducted to investigate whether daily ambient temperature predicts the dichotomous dependent variable RP attacks (Yes/No). The outcome will be presented as odds ratio and 95% CI.

Mixed model will be adopted to test the effect of daily ambient temperature on the severity score of RP attacks.

### Use of Rescue Medications for Breakthrough Symptoms.

The difference of using rescue medication between treatment groups will be evaluated by Chi-square test.

### Exploratory Efficacy Analyses

Change from baseline in endothelial function as measured by LnRHI using Endo-PAT for Part A only will be analyzed by treatment groups using mixed effect model in the same manner as the primary analysis.

### Safety Analysis

All safety analyses will be performed on the Safety population. Safety data presented by treatment sequence will be summarized on an ‘as treated’ basis. Safety variables include treatment-emergent adverse events (TEAEs), laboratory evaluations, vital signs, and other safety assessments. Study Day 1 for all safety analyses is defined as the date of the first dose of study drug.

### Adverse Events

Adverse events will be coded using the most current version of the MedDRAR Version 22.0 or higher. Only those AEs that are treatment emergent will be included in summary tables. All AEs, treatment emergent or otherwise, will be presented in participant data listings. Separate summaries will be provided for Part A and Part B. as well as the pooled data. The pooled analyses of Part A and Part B may be conducted as exploratory for participants with common treatment.

An overview AE table, including number and percentage of participants with TEAEs, TEAEs by severity (mild, moderate, severe), TEAEs related to study drug, AEs leading to study withdrawal, AEs leading to study drug discontinuation, SAEs, SAEs related to study drug, and death will be provided.

In addition, number and percentage of subjects will be provided for the following summary tables:
TEAE by PT.
TEAE by SOC and PT
TEAE by SOC, PT, and relationship (related, not related)
TEAE by SOC, PT, and maximum severity (mild, moderate, and severe)

A participant having the same AE (as determined by the coded MedDRA preferred term) more than once will be counted only once in the number and percentage of participants calculation for that AE. Similarly, if a participant had more than one AE in a SOC, the participant will be counted only once in the number of subjects with an AE for that SOC. If a participant has multiple AEs with the same preferred term, the maximum severity (severe >moderate >mild) recorded for the events will be presented in the AEs by severity table; if severity is missing, these TEAEs will not be included in the severity table. Similarly, if a participant has multiple AEs with the same preferred term, the worst relationship (related worse than not related) for the event will be presented in the AEs by relationship table; if relationship is missing for an AT it is assumed to be related.

### Laboratory Evaluations

Baseline laboratory evaluations will be summarized by treatment sequence and listed by participant.

### Vital Signs

Descriptive statistics for vital signs parameters (diastolic and systolic blood pressure, pulse rate, oral temperature, weight (if collected) and changes from baseline will be presented by visit and treatment sequence.

All vital signs will be listed by participant.

### Other Safety Analyses

Urine pregnancy test and Raynaud's function assessment will be listed by participant.

### Pharmacokinetics

Plasma concentrations and actual blood sampling times will be listed by treatment and protocol specified time point and summarized using descriptive statistics—number of measurements, arithmetic mean, SD, and % CV, geometric mean, minimum, median, and maximum—at each scheduled time point. Individual and mean plasma concentration-time profiles will also be presented graphically for each treatment.

### Interim Analysis and Data Safety Monitoring Board

### Interim Analysis

No formal interim efficacy analyses are planned for this study.

### Data Safety Monitoring Board (DSMB)

Safety oversight was and will be provided by a DSMB, the details of which will be set out in a DSMB Charter. The DSMB plans on conducting a review of the efficacy and safety data from Part A of the study, when data is available on the first 16 to 25 patients that have completed the study. However an early review occurred after an initial 11 participants completed the study.

Following review of the efficacy and safety data from Part A, the DSMB will make the following recommendations:
1. Select the dose of cilnidipine (10 mg or 20 mg) to be studied in Part B of the study
2. Confirm the sample size estimates for Part B of the study.

Serious adverse events will be monitored by the DSMB on an ongoing basis throughout the study.

### Summary of Major Changes in the Planned Analyses Appendices

### Scleroderma Health Assessment Questionnaire

The SHAQ is used to calculate a Disability Index to assess the participant qualify of life. The eight categories assessed by the Disability Index are 1) dressing and grooming, 2) arising, 3) eating, 4) walking, 5) hygiene, 6) reach, 7) grip, and 8) common daily activities. For each of these categories, patients report the amount of difficulty they have in performing two or three specific activities.

Ratings such as SOME, MUCH, or USUAL are deliberately not defined for the patients; patients are instructed to respond idiomatically, using their own frame of reference. For example, if a patient asks what “SOME” means, an appropriate response would be “Whatever you think ‘SOME’ means to you”.

### Scoring Conventions for the Disability Index

There are four possible responses for the Disability Index questions:
Without any difficulty=0
With some difficulty=1
With much difficulty=2
Unable to do=3
   The highest score reported by the patient for any component question of the eight categories determines the score for that category.
   If a component question is left blank or the response is too ambiguous to assign a score, then the score for that category is determined by the remaining completed question(s).
   If all component questions are blank or if more than one answer is given, then follow up with the respondent is required.
   If the respondent's mark is between the response columns, then move it to the closest one. If it's directly between the two, move it to the higher one.

Each of the disability items on the SHAQ has a companion aids/devices variable that is used to record what type(s) of assistance, if any, the participant uses for his/her usual activities. These variables (see below) are coded as follows:
0=No assistance is needed.
1=A special device is used by the patient in his/her usual activities.
2=The patient usually needs help from another person.
3=The patient usually needs BOTH a special device AND help from another person.

### Computed Variables:

The scoring variables and scoring rules permit the computation of two disability indices, the Standard Disability Index and the Alternative Disability Index. For either of these, a disability index cannot be computed if the patient does not have scores for at least six (6) categories.

1) The Standard Disability Index. “What is the Disability Level of this Person?”

This question results in a new set of category scores that are computed by adjusting the score for each category, if necessary, based on the patient's use of an aid or device or assistance for that category. If either devices and/or help from another person are checked for a category, the score is set to “2”, unless the score is already “3” (i.e., scores of “0” or “1” are increased to “2”). For example, if the highest score for the dressing category is “1”, and the patient says they use a device for dressing, the computed category score would be “2”. The sum of the computed categories scores is then calculated and divided by the number of categories answered. This gives a score in the 0 to 3 range.

2) The Alternative Disability Index. “What is the Disability level of this patient when using aids and devices to compensate for disability?”

The aid and device variables are not used to calculate the alternative disability index; it is calculated by adding the scores for each of the categories and dividing by the number of categories answered. This gives a score in the 0 to 3 range.

### The UCLA SCTC GIT 2.0 Questionnaire

The UCLA SCTC GIT 2.0 Questionnaire contains 34 questions in 7 sections to ask about gastrointestinal symptoms and evaluate the Impact of life over the past 7 days. The 7 sections will obtain 7 scores: Reflux score (R), Distension/Bloating score (D/B), Faecal Soilage score(S), Diarrhoea score (D), Social functioning score (SF), Emotional wellbeing score (EWB) and Constipation score (C).

$$Total score = ( R + D / B + S + D + S F + EWB ) / 6.$$

Constipation score is not included in the calculation of total score.

### Partial Dates for Adverse Events and Prior/Concomitant Medication

Dates missing the day or both the day and month of the year will adhere to the following conventions in order to classify treatment-emergent AEs and to classify prior/concomitant medications:

### Adverse Events

The missing day of onset of an AE will be set to:
First day of the month that the event occurred, if the onset YYYY-MM is after the YYYY-MM of first study treatment
The day of the first study treatment, if the onset YYYY-MM is the same as YYYY-MM of the first study treatment
The date of informed consent, if the onset YYYY-MM is before the YYYY-MM of the first treatment.

The missing day of resolution of an AE will be set to:
The last day of the month of the occurrence. If the patient died in the same month, then set the imputed date as the death date.

If the onset date of an AE is missing both the day and month, the onset date will be set to:
January 1 of the year of onset, if the onset year is after the year of the first study treatment
The date of the first treatment, if the onset year is the same as the year of the first study treatment
The date of informed consent, if the onset year is before the year of the first treatment

If the resolution date of an AE or end date of an IP is missing both the day and month, the date will be set to:
December 31 of the year of occurrence. If the patient died in the same year, then set the imputed date as the death date.

### Prior/Concomitant Medication

The missing day of start date of a therapy will be set to the first day of the month that the event occurred.

The missing day of end date of a therapy will be set to the last day of the month of the occurrence.

If the start date of a therapy is missing both the day and month, the onset date will be set to January 1 of the year of onset.

If the end date of a therapy is missing both the day and month, the date will be set to December 31 of the year of occurrence.

If the start date of a therapy is null and the end date is not a complete date then the start date will be set to the earlier of the imputed partial end date and the date of the first study visit.

If the start date of a therapy is null and the end date is a complete date
and the end date is after the date of the first study visit then the start date will be set to the date of the first study visit.
otherwise the start date will be set to the end date of the therapy.

If the end date of a therapy is null and the start date is not a complete date then the end date will be set to the study end date.

If the end date of a therapy is null and the start date is a complete date
and the start date is prior to the study end date then the end date will be set to the study end date.
otherwise, the end date will be set to the start date of the therapy.

### Assessment of Systemic Sclerosis-Associated Raynaud's Phenomenon (ASRAP) Questionnaire

The Assessment of Systemic sclerosis-associated Raynaud's Phenomenon (ASRAP) questionnaire is a patient-reported outcome (PRO) instrument devised to assess the severity and impact of systemic sclerosis-associated Raynaud's Syndrome. See Pauling et. al. American College of Rheumatology Convergence 2021, Abstract Number 401 (https://acrabstracts.org/abstract/item-reduction-for-the-assessment-of-systemic-sclerosis-associated-raynauds-phenomenon-asrap-questionnaire-using-data-from-the-international-multicentre-asrap-validation-study/), which is incorporated by reference herein in its entirety.

### Example 3. Phase 2A Clinical Trial Data Collected

The following data was actually obtained in accordance with the protocol and plan delineated in Examples 1 and 2. The accompanying data involved 11 patients out of the planned 76 patient study. The study was done in patients with scleroderma (systemic sclerosis) who had relatively frequent Raynaud symptoms as they needed to average at least one attack per day during a screening period of up to two weeks. In this first phase of this double-blind placebo-controlled, prospective randomized study, patients were treated in parallel and after meeting study criteria were randomized to receive either placebo, cilnidipine 10 mg daily, cilnidipine 20 mg daily, tadalafil 5 mg daily, cilnidipine 10 mg plus 5 mg of tadalafil, or cilnidipine 20 mg plus 5 mg of tadalafil.

Tables A-1 to A-8 include data that relates to the frequency of symptomatic Raynaud's attacks.

TABLE A-1
Weekly Symptomatic RP Attacks Change and Percent Change from Baseline. ITT Population - Part A.
Frequency of weekly symptomatic RP attacks is defined as the total number of
symptomatic RP attacks divided by the number of days with available diary data within
the last 7 days of the dosing period (excluding the last dosing day) then multiplied by 7.
Baseline is calculated as total number of symptomatic RP attacks during the last 7 days of
screening period (excluding the first dosing day) divided by the number of days with
available data then multiplied by 7. Only symptomatic RP attacks, defined as RP attacks
with patient reported finger color changes associated with at least one symptom (pain,
numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 2 | 2 | 2 | 2 |
| Mean (SD) | 21.00 | 12.50 | 8.00 | 8.00 | 14.25 | 17.83 |
| | | (7.778) | (5.657) | (1.414) | (5.303) | (8.721) |
| Median | 21.00 | 12.50 | 8.00 | 8.00 | 14.25 | 17.83 |
| Q1, Q3 | 21.00, 21.00 | 7.00, 18.00 | 4.00, 12.00 | 7.00, 9.00 | 10.50, 18.00 | 11.67, 24.00 |
| Min, Max | 21.0, 21.0 | 7.0, 18.0 | 4.0, 12.0 | 7.0, 9.0 | 10.5, 18.0 | 11.7, 24.0 |
| Weekly | | | | | | |
| Symptomatic RP | | | | | | |
| Attacks | | | | | | |
| n | 1 | 2 | 2 | 2 | 2 | 2 |
| Mean (SD) | 14.00 | 8.00 | 12.50 | 4.50 | 11.50 | 8.83 |
| | | (4.243) | (7.778) | (0.707) | (2.121) | (5.893) |
| Median | 14.00 | 8.00 | 12.50 | 4.50 | 11.50 | 8.83 |
| Q1, Q3 | 14.00, 14.00 | 5.00, 11.00 | 7.00, 18.00 | 4.00, 5.00 | 10.00, 13.00 | 4.67, 13.00 |
| Min, Max | 14.0, 14.0 | 5.0, 11.0 | 7.0, 18.0 | 4.0, 5.0 | 10.0, 13.0 | 4.7, 13.0 |
| Change from | | | | | | |
| Baseline | | | | | | |
| n | 1 | 2 | 2 | 2 | 2 | 2 |
| Mean (SD) | −7.00 | −4.50 | 4.50 | −3.50 | −2.75 | −9.00 |
| | | (3.536) | (2.121) | (0.707) | (3.182) | (2.828) |
| Median | −7.00 | −4.50 | 4.50 | −3.50 | −2.75 | −9.00 |
| Q1, Q3 | −7.00, | −7.00, | 3.00, | −4.00, | −5.00, | −11.00, |
| | −7.00 | −2.00 | 6.00 | −3.00 | −0.50 | −7.00 |
| Min, Max | −7.0, −7.0 | −7.0, −2.0 | 3.0, 6.0 | −4.0, −3.0 | −5.0, −0.5 | −11.0, −7.0 |
| Percent Change | | | | | | |
| from Baseline | | | | | | |
| n | 1 | 2 | 2 | 2 | 2 | 2 |
| Mean (SD) | −33.33 | −33.73 | 62.50 | −43.65 | −16.27 | −52.92 |
| | | (7.296) | (17.678) | (1.122) | (16.275) | (10.017) |
| Median | −33.33 | −33.73 | 62.50 | −43.65 | −16.27 | −52.92 |
| Q1, Q3 | −33.33, | −38.89, | 50.00, | −44.44, | −27.78, | −60.00, |
| | −33.33 | −28.57 | 75.00 | −42.86 | −4.76 | −45.83 |
| Min, Max | −33.3, −33.3 | −38.9, −28.6 | 50.0, 75.0 | −44.4, −42.9 | −27.8, −4.8 | −60.0, −45.8 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg | 20 mg | |
| | Pooled | Cilnidipine + | Tadalafil + | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 11 |
| Mean (SD) | 15.33 | 11.13 | 12.33 | 13.38 | 12.92 |
| | (7.371) | (4.802) | (7.572) | (5.528) | (6.426) |
| Median | 18.00 | 9.75 | 9.00 | 14.25 | 11.67 |
| Q1, Q3 | 7.00, 21.00 | 8.00, 14.25 | 7.00, 21.00 | 8.75, 18.00 | 7.00, 18.00 |
| Min, Max | 7.0, 21.0 | 7.0, 18.0 | 7.0, 21.0 | 7.0, 18.0 | 4.0, 24.0 |
| Weekly | | | | | |
| Symptomatic RP | | | | | |
| Attacks | | | | | |
| n | 3 | 4 | 3 | 4 | 11 |
| Mean (SD) | 10.00 | 8.00 | 7.67 | 9.75 | 9.52 |
| | (4.583) | (4.243) | (5.508) | (3.403) | (4.689) |
| Median | 11.00 | 7.50 | 5.00 | 10.50 | 10.00 |
| Q1, Q3 | 5.00, 14.00 | 4.50, 11.50 | 4.00, 14.00 | 7.50, 12.00 | 5.00, 13.00 |
| Min, Max | 5.0, 14.0 | 4.0, 13.0 | 4.0, 14.0 | 5.0, 13.0 | 4.0, 18.0 |
| Change from | | | | | |
| Baseline | | | | | |
| n | 3 | 4 | 3 | 4 | 11 |
| Mean (SD) | −5.33 | −3.13 | −4.67 | −3.63 | −3.41 |
| | (2.887) | (1.931) | (2.082) | (2.926) | (4.893) |
| Median | −7.00 | −3.50 | −4.00 | −3.50 | −4.00 |
| Q1, Q3 | −7.00, −2.00 | −4.50, −1.75 | −7.00, −3.00 | −6.00, −1.25 | −7.00, −0.50 |
| Min, Max | −7.0, −2.0 | −5.0, −0.5 | −7.0, −3.0 | −7.0, −0.5 | −11.0, 6.0 |
| Percent Change | | | | | |
| from Baseline | | | | | |
| n | 3 | 4 | 3 | 4 | 11 |
| Mean (SD) | −33.60 | −29.96 | −40.21 | −25.00 | −18.32 |
| | (5.164) | (18.401) | (6.009) | (14.410) | (42.640) |
| Median | −33.33 | −35.32 | −42.86 | −28.17 | −33.33 |
| Q1, Q3 | −38.89, | −43.65, | −44.44, | −33.73, | −44.44, |
| | −28.57 | −16.27 | −33.33 | −16.27 | −4.76 |
| Min, Max | −38.9, −28.6 | −44.4, −4.8 | −44.4, −33.3 | −38.9, −4.8 | −60.0, 75.0 |

TABLE A-2
Weekly Symptomatic RP Attacks Change and Percent Change from Baseline
(Excluding Outliers). ITT Population - Part A.
Frequency of weekly symptomatic RP attacks is defined as the total number of
symptomatic RP attacks divided by the number of days with available diary data within
the last 7 days of the dosing period (excluding the last dosing day) then multiplied by 7.
Baseline is calculated as total number of symptomatic RP attacks during the last 7 days of
screening period (excluding the first dosing day) divided by the number of days with
available data then multiplied by 7. Only symptomatic RP attacks, defined as RP attacks
with patient reported finger color changes associated with at least one symptom (pain,
numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 1 | 2 | 2 | 2 |
| Mean (SD) | 21.00 | 12.50 | 12.00 | 8.00 | 14.25 | 17.83 |
| | | (7.778) | | (1.414) | (5.303) | (8.721) |
| Median | 21.00 | 12.50 | 12.00 | 8.00 | 14.25 | 17.83 |
| Q1, Q3 | 21.00, | 7.00, 18.00 | 12.00, | 7.00, 9.00 | 10.50, | 11.67, |
| | 21.00 | | 12.00 | | 18.00 | 24.00 |
| Min, Max | 21.0, 21.0 | 7.0, 18.0 | 12.0, 12.0 | 7.0, 9.0 | 10.5, 18.0 | 11.7, 24.0 |
| Weekly | | | | | | |
| Symptomatic | | | | | | |
| RP Attacks | | | | | | |
| n | 1 | 2 | 1 | 2 | 2 | 2 |
| Mean (SD) | 14.00 | 8.00 | 18.00 | 4.50 | 11.50 | 8.83 |
| | | (4.243) | | (0.707) | (2.121) | (5.893) |
| Median | 14.00 | 8.00 | 18.00 | 4.50 | 11.50 | 8.83 |
| Q1, Q3 | 14.00, 14.00 | 5.00, 11.00 | 18.00, 18.00 | 4.00, 5.00 | 10.00, 13.00 | 4.67, 13.00 |
| Min, Max | 14.0, 14.0 | 5.0, 11.0 | 18.0, 18.0 | 4.0, 5.0 | 10.0, 13.0 | 4.7, 13.0 |
| Change from | | | | | | |
| Baseline | | | | | | |
| n | 1 | 2 | 1 | 2 | 2 | 2 |
| Mean (SD) | −7.00 | −4.50 | 6.00 | −3.50 | −2.75 | −9.00 |
| | | (3.536) | | (0.707) | (3.182) | (2.828) |
| Median | −7.00 | −4.50 | 6.00 | −3.50 | −2.75 | −9.00 |
| Q1, Q3 | −7.00, | −7.00, | 6.00, | −4.00, | −5.00, | −11.00, |
| | −7.00 | −2.00, | 6.00 | −3.00 | −0.50 | −7.00 |
| Min, Max | −7.0, −7.0 | −7.0, −2.0 | 6.0, 6.0 | −4.0, −3.0 | −5.0, −0.5 | −11.0, −7.0 |
| Percent Change | | | | | | |
| from Baseline | | | | | | |
| n | 1 | 2 | 1 | 2 | 2 | 2 |
| Mean (SD) | −33.33 | −33.73 | 50.00 | −43.65 | −16.27 | −52.92 |
| | | (7.296) | | (1.122) | (16.275) | (10.017) |
| Median | −33.33 | −33.73 | 50.00 | −43.65 | −16.27 | −52.92 |
| Q1, Q3 | −33.33, | −38.89, | 50.00, | −44.44, | −27.78, | −60.00, |
| | −33.33 | −28.57 | 50.00 | −42.86 | −4.76 | −45.83 |
| Min, Max | −33.3, −33.3 | −38.9, −28.6 | 50.0, 50.0 | −44.4, −42.9 | −27.8, −4.8 | −60.0, −45.8 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 10 |
| Mean (SD) | 15.33 | 11.13 | 12.33 | 13.38 | 13.82 |
| | (7.371) | (4.802) | (7.572) | (5.528) | (6.013) |
| Median | 18.00 | 9.75 | 9.00 | 14.25 | 11.83 |
| Q1, Q3 | 7.00, 21.00 | 8.00, 14.25 | 7.00, 21.00 | 8.75, 18.00 | 9.00, 18.00 |
| Min, Max | 7.0, 21.0 | 7.0, 18.0 | 7.0, 21.0 | 7.0, 18.0 | 7.0, 24.0 |
| Weekly | | | | | |
| Symptomatic | | | | | |
| RP Attacks | | | | | |
| n | 3 | 4 | 3 | 4 | 10 |
| Mean (SD) | 10.00 | 8.00 | 7.67 | 9.75 | 9.77 |
| | (4.583) | (4.243) | (5.508) | (3.403) | (4.864) |
| Median | 11.00 | 7.50 | 5.00 | 10.50 | 10.50 |
| Q1, Q3 | 5.00, 14.00 | 4.50, 11.50 | 4.00, 14.00 | 7.50, 12.00 | 5.00, 13.00 |
| Min, Max | 5.0, 14.0 | 4.0, 13.0 | 4.0, 14.0 | 5.0, 13.0 | 4.0, 18.0 |
| Change from | | | | | |
| Baseline | | | | | |
| n | 3 | 4 | 3 | 4 | 10 |
| Mean (SD) | −5.33 | −3.13 | −4.67 | −3.63 | −4.05 |
| | (2.887) | (1.931) | (2.082) | (2.926) | (4.645) |
| Median | −7.00 | −3.50 | −4.00 | −3.50 | −4.50 |
| Q1, Q3 | −7.00, −2.00 | −4.50, −1.75 | −7.00, −3.00 | −6.00, −1.25 | −7.00, −2.00 |
| Min, Max | −7.0, −2.0 | −5.0, −0.5 | −7.0, −3.0 | −7.0, −0.5 | −11.0, 6.0 |
| Percent Change | | | | | |
| from Baseline | | | | | |
| n | 3 | 4 | 3 | 4 | 10 |
| Mean (SD) | −33.60 | −29.96 | −40.21 | −25.00 | −27.65 |
| | (5.164) | (18.401) | (6.009) | (14.410) | (30.918) |
| Median | −33.33 | −35.32 | −42.86 | −28.17 | −36.11 |
| Q1, Q3 | −38.89, | −43.65, | −44.44, | −33.73, | −44.44, |
| | −28.57 | −16.27 | −33.33 | −16.27 | −27.78 |
| Min, Max | −38.9, −28.6 | −44.4, −4.8 | −44.4, −33.3 | −38.9, −4.8 | −60.0, 50.0 |

TABLE A-3
Weekly Symptomatic RP Attacks Change and Percent Change from Baseline
by Baseline RCS. ITT Population - Part A.
Frequency of weekly symptomatic RP attacks is defined as the total number of
symptomatic RP attacks divided by the number of days with available diary data within
the last 7 days of the dosing period (excluding the last dosing day) then multiplied by 7.
Baseline is calculated as total number of symptomatic RP attacks during the last 7 days of
screening period (excluding the first dosing day) divided by the number of days with
available data then multiplied by 7. Only symptomatic RP attacks, defined as RP attacks
with patient reported finger color changes associated with at least one symptom (pain,
numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean (SD) | 21.00 | 12.50 | | 9.00 | 18.00 | 17.83 |
| | | (7.778) | | | | (8.721) |
| Median | 21.00 | 12.50 | | 9.00 | 18.00 | 17.83 |
| Q1, Q3 | 21.00, | 7.00, 18.00 | | 9.00, 9.00 | 18.00, | 11.67, |
| | 21.00 | | | | 18.00 | 24.00 |
| Min, Max | 21.0, 21.0 | 7.0, 18.0 | | 9.0, 9.0 | 18.0, 18.0 | 11.7, 24.0 |
| Weekly | | | | | | |
| Symptomatic RP | | | | | | |
| Attacks | | | | | | |
| n | 1 | 2 | | 1 | 1 | 2 |
| Mean (SD) | 14.00 | 8.00 | | 5.00 | 13.00 | 8.83 |
| | | (4.243) | | | | (5.893) |
| Median | 14.00 | 8.00 | | 5.00 | 13.00 | 8.83 |
| Q1, Q3 | 14.00, | 5.00, 11.00 | | 5.00, 5.00 | 13.00, | 4.67, 13.00 |
| | 14.00 | | | | 13.00 | |
| Min, Max | 14.0, 14.0 | 5.0, 11.0 | | 5.0, 5.0 | 13.0, 13.0 | 4.7, 13.0 |
| Change from | | | | | | |
| Baseline | | | | | | |
| n | 1 | 2 | | 1 | 1 | 2 |
| Mean (SD) | −7.00 | −4.50 | | −4.00 | −5.00 | −9.00 |
| | | (3.536) | | | | (2.828) |
| Median | −7.00 | −4.50 | | −4.00 | −5.00 | −9.00 |
| Q1, Q3 | −7.00, −7.00 | −7.00, −2.00 | | −4.00, −4.00 | −5.00, −5.00 | −11.00, |
| | | | | | | −7.00 |
| Min, Max | −7.0, −7.0 | −7.0, −2.0 | | −4.0, −4.0 | −5.0, −5.0 | −11.0, −7.0 |
| Percent Change | | | | | | |
| from Baseline | | | | | | |
| n | 1 | 2 | | 1 | 1 | 2 |
| Mean (SD) | −33.33 | −33.73 | | −44.44 | −27.78 | −52.92 |
| | | (7.296) | | | | (10.017) |
| Median | −33.33 | −33.73 | | −44.44 | −27.78 | −52.92 |
| Q1, Q3 | −33.33, | −38.89, | | −44.44, | −27.78, | −60.00, |
| | −33.33 | −28.57 | | −44.44 | −27.78 | −45.83 |
| Min, Max | −33.3, −33.3 | −38.9, −28.6 | | −44.4, −44.4 | −27.8, −27.8 | −60.0, −45.8 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean (SD) | 15.33 | 13.50 | 15.00 | 14.33 | 15.52 |
| | (7.371) | (6.364) | (8.485) | (6.351) | (6.380) |
| Median | 18.00 | 13.50 | 15.00 | 18.00 | 18.00 |
| Q1, Q3 | 7.00, 21.00 | 9.00, 18.00 | 9.00, 21.00 | 7.00, 18.00 | 9.00, 21.00 |
| Min, Max | 7.0, 21.0 | 9.0, 18.0 | 9.0, 21.0 | 7.0, 18.0 | 7.0, 24.0 |
| Weekly | | | | | |
| Symptomatic RP | | | | | |
| Attacks | | | | | |
| n | 3 | 2 | 2 | 3 | 7 |
| Mean (SD) | 10.00 | 9.00 | 9.50 | 9.67 | 9.38 |
| | (4.583) | (5.657) | (6.364) | (4.163) | (4.297) |
| Median | 11.00 | 9.00 | 9.50 | 11.00 | 11.00 |
| Q1, Q3 | 5.00, 14.00 | 5.00, 13.00 | 5.00, 14.00 | 5.00, 13.00 | 5.00, 13.00 |
| Min, Max | 5.0, 14.0 | 5.0, 13.0 | 5.0, 14.0 | 5.0, 13.0 | 4.7, 14.0 |
| Change from | | | | | |
| Baseline | | | | | |
| n | 3 | 2 | 2 | 3 | 7 |
| Mean (SD) | −5.33 | −4.50 | −5.50 | −4.67 | −6.14 |
| | (2.887) | (0.707) | (2.121) | (2.517) | (2.854) |
| Median | −7.00 | −4.50 | −5.50 | −5.00 | −7.00 |
| Q1, Q3 | −7.00, −2.00 | −5.00, −4.00 | −7.00, −4.00 | −7.00, −2.00 | −7.00, −4.00 |
| Min, Max | −7.0, −2.0 | −5.0, −4.0 | −7.0, −4.0 | −7.0, −2.0 | −11.0, −2.0 |
| Percent Change | | | | | |
| from Baseline | | | | | |
| n | 3 | 2 | 2 | 3 | 7 |
| Mean (SD) | −33.60 | −36.11 | −38.89 | −31.75 | −39.84 |
| | (5.164) | (11.785) | (7.857) | (6.199) | (11.396) |
| Median | −33.33 | −36.11 | −38.89 | −28.57 | −38.89 |
| Q1, Q3 | −38.89, | −44.44, | −44.44, | −38.89, | −45.83, |
| | −28.57 | −27.78 | −33.33 | −27.78 | −28.57 |
| Min, Max | −38.9, −28.6 | −44.4, −27.8 | −44.4, −33.3 | −38.9, −27.8 | −60.0, −27.8 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 2 | 1 | 1 | |
| Mean (SD) | | | 8.00 | 7.00 | 10.50 | |
| | | | (5.657) | | | |
| Median | | | 8.00 | 7.00 | 10.50 | |
| Q1, Q3 | | | 4.00, 12.00 | 7.00, 7.00 | 10.50, | |
| | | | | | 10.50 | |
| Min, Max | | | 4.0, 12.0 | 7.0, 7.0 | 10.5, 10.5 | |
| Weekly | | | | | | |
| Symptomatic | | | | | | |
| RP Attacks | | | | | | |
| n | | | 2 | 1 | 1 | |
| Mean (SD) | | | 12.50 | 4.00 | 10.00 | |
| | | | (7.778) | | | |
| Median | | | 12.50 | 4.00 | 10.00 | |
| Q1, Q3 | | | 7.00, 18.00 | 4.00, 4.00 | 10.00, | |
| | | | | | 10.00 | |
| Min, Max | | | 7.0, 18.0 | 4.0, 4.0 | 10.0, 10.0 | |
| Change from | | | | | | |
| Baseline | | | | | | |
| n | | | 2 | 1 | 1 | |
| Mean (SD) | | | 4.50 | −3.00 | −0.50 | |
| | | | (2.121) | | | |
| Median | | | 4.50 | −3.00 | −0.50 | |
| Q1, Q3 | | | 3.00, 6.00 | −3.00, −3.00 | −0.50, −0.50 | |
| Min, Max | | | 3.0, 6.0 | −3.0, −3.0 | −0.5, −0.5 | |
| Percent | | | | | | |
| Change from | | | | | | |
| Baseline | | | | | | |
| n | | | 2 | 1 | 1 | |
| Mean (SD) | | | 62.50 | −42.86 | −4.76 | |
| | | | (17.678) | | | |
| Median | | | 62.50 | −42.86 | −4.76 | |
| Q1, Q3 | | | 50.00, | −42.86, | −4.76, −4.76 | |
| | | | 75.00 | −42.86 | | |
| Min, Max | | | 50.0, 75.0 | −42.9, −42.9 | −4.8, −4.8 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 4 |
| Mean (SD) | | 8.75 | 7.00 | 10.50 | 8.38 |
| | | (2.475) | | | (3.591) |
| Median | | 8.75 | 7.00 | 10.50 | 8.75 |
| Q1, Q3 | | 7.00, 10.50 | 7.00, 7.00 | 10.50, | 5.50, 11.25 |
| | | | | 10.50 | |
| Min, Max | | 7.0, 10.5 | 7.0, 7.0 | 10.5, 10.5 | 4.0, 12.0 |
| Weekly | | | | | |
| Symptomatic RP | | | | | |
| Attacks | | | | | |
| n | | 2 | 1 | 1 | 4 |
| Mean (SD) | | 7.00 | 4.00 | 10.00 | 9.75 |
| | | (4.243) | | | (6.021) |
| Median | | 7.00 | 4.00 | 10.00 | 8.50 |
| Q1, Q3 | | 4.00, 10.00 | 4.00, 4.00 | 10.00, | 5.50, 14.00 |
| | | | | 10.00 | |
| Min, Max | | 4.0, 10.0 | 4.0, 4.0 | 10.0, 10.0 | 4.0, 18.0 |
| Change from | | | | | |
| Baseline | | | | | |
| n | | 2 | 1 | 1 | 4 |
| Mean (SD) | | −1.75 | −3.00 | −0.50 | 1.38 |
| | | (1.768) | | | (3.945) |
| Median | | −1.75 | −3.00 | −0.50 | 1.25 |
| Q1, Q3 | | −3.00, −0.50 | −3.00, −3.00 | −0.50, −0.50 | −1.75, 4.50 |
| Min, Max | | −3.0, −0.5 | −3.0, −3.0 | −0.5, −0.5 | −3.0, 6.0 |
| Percent Change | | | | | |
| from Baseline | | | | | |
| n | | 2 | 1 | 1 | 4 |
| Mean (SD) | | −23.81 | −42.86 | −4.76 | 19.35 |
| | | (26.937) | | | (53.190) |
| Median | | −23.81 | −42.86 | −4.76 | 22.62 |
| Q1, Q3 | | −42.86, | −42.86, | −4.76, −4.76 | −23.81, |
| | | −4.76 | −42.86 | | 62.50 |
| Min, Max | | −42.9, −4.8 | −42.9, −42.9 | −4.8, −4.8 | −42.9, 75.0 |

TABLE A-4
Weekly Symptomatic RP Attacks Change and Percent Change from Baseline
(Excluding Outliers) by Baseline RCS. ITT Population - Part A.
Frequency of weekly symptomatic RP attacks is defined as the total number of
symptomatic RP attacks divided by the number of days with available diary data within
the last 7 days of the dosing period (excluding the last dosing day) then multiplied by 7.
Baseline is calculated as total number of symptomatic RP attacks during the last 7 days of
screening period (excluding the first dosing day) divided by the number of days with
available data then multiplied by 7. Only symptomatic RP attacks, defined as RP attacks
with patient reported finger color changes associated with at least one symptom (pain,
numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean (SD) | 21.00 | 12.50 | | 9.00 | 18.00 | 17.83 |
| | | (7.778) | | | | (8.721) |
| Median | 21.00 | 12.50 | | 9.00 | 18.00 | 17.83 |
| Q1, Q3 | 21.00, | 7.00, 18.00 | | 9.00, 9.00 | 18.00, | 11.67, |
| | 21.00 | | | | 18.00 | 24.00 |
| Min, Max | 21.0, 21.0 | 7.0, 18.0 | | 9.0, 9.0 | 18.0, 18.0 | 11.7, 24.0 |
| Weekly | | | | | | |
| Symptomatic | | | | | | |
| RP Attacks | | | | | | |
| n | 1 | 2 | | 1 | 1 | 2 |
| Mean (SD) | 14.00 | 8.00 | | 5.00 | 13.00 | 8.83 |
| | | (4.243) | | | | (5.893) |
| Median | 14.00 | 8.00 | | 5.00 | 13.00 | 8.83 |
| Q1, Q3 | 14.00, | 5.00, 11.00 | | 5.00, 5.00 | 13.00, | 4.67, 13.00 |
| | 14.00 | | | | 13.00 | |
| Min, Max | 14.0, 14.0 | 5.0, 11.0 | | 5.0, 5.0 | 13.0, 13.0 | 4.7, 13.0 |
| Change from | | | | | | |
| Baseline | | | | | | |
| n | 1 | 2 | | 1 | 1 | 2 |
| Mean (SD) | −7.00 | −4.50 | | −4.00 | −5.00 | −9.00 |
| | | (3.536) | | | | (2.828) |
| Median | −7.00 | −4.50 | | −4.00 | −5.00 | −9.00 |
| Q1, Q3 | −7.00, −7.00 | −7.00, −2.00 | | −4.00, −4.00 | −5.00, −5.00 | −11.00, |
| | | | | | | −7.00 |
| Min, Max | −7.0, −7.0 | −7.0, −2.0 | | −4.0, −4.0 | −5.0, −5.0 | −11.0, −7.0 |
| Percent | | | | | | |
| Change from | | | | | | |
| Baseline | | | | | | |
| n | 1 | 2 | | 1 | 1 | 2 |
| Mean (SD) | −33.33 | −33.73 | | −44.44 | −27.78 | −52.92 |
| | | (7.296) | | | | (10.017) |
| Median | −33.33 | −33.73 | | −44.44 | −27.78 | −52.92 |
| Q1, Q3 | −33.33, | −38.89, | | −44.44, | −27.78, | −60.00, |
| | −33.33 | −28.57 | | −44.44 | −27.78 | −45.83 |
| Min, Max | −33.3, −33.3 | −38.9, −28.6 | | −44.4, −44.4 | −27.8, −27.8 | −60.0, −45.8 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean (SD) | 15.33 | 13.50 | 15.00 | 14.33 | 15.52 |
| | (7.371) | (6.364) | (8.485) | (6.351) | (6.380) |
| Median | 18.00 | 13.50 | 15.00 | 18.00 | 18.00 |
| Q1, Q3 | 7.00, 21.00 | 9.00, 18.00 | 9.00, 21.00 | 7.00, 18.00 | 9.00, 21.00 |
| Min, Max | 7.0, 21.0 | 9.0, 18.0 | 9.0, 21.0 | 7.0, 18.0 | 7.0, 24.0 |
| Weekly | | | | | |
| Symptomatic RP | | | | | |
| Attacks | | | | | |
| n | 3 | 2 | 2 | 3 | 7 |
| Mean (SD) | 10.00 | 9.00 | 9.50 | 9.67 | 9.38 |
| | (4.583) | (5.657) | (6.364) | (4.163) | (4.297) |
| Median | 11.00 | 9.00 | 9.50 | 11.00 | 11.00 |
| Q1, Q3 | 5.00, 14.00 | 5.00, 13.00 | 5.00, 14.00 | 5.00, 13.00 | 5.00, 13.00 |
| Min, Max | 5.0, 14.0 | 5.0, 13.0 | 5.0, 14.0 | 5.0, 13.0 | 4.7, 14.0 |
| Change from | | | | | |
| Baseline | | | | | |
| n | 3 | 2 | 2 | 3 | 7 |
| Mean (SD) | −5.33 | −4.50 | −5.50 | −4.67 | −6.14 |
| | (2.887) | (0.707) | (2.121) | (2.517) | (2.854) |
| Median | −7.00 | −4.50 | −5.50 | −5.00 | −7.00 |
| Q1, Q3 | −7.00, −2.00 | −5.00, −4.00 | −7.00, −4.00 | −7.00, −2.00 | −7.00, −4.00 |
| Min, Max | −7.0, −2.0 | −5.0, −4.0 | −7.0, −4.0 | −7.0, −2.0 | −11.0, −2.0 |
| Percent Change | | | | | |
| from Baseline | | | | | |
| n | 3 | 2 | 2 | 3 | 7 |
| Mean (SD) | −33.60 | −36.11 | −38.89 | −31.75 | −39.84 |
| | (5.164) | (11.785) | (7.857) | (6.199) | (11.396) |
| Median | −33.33 | −36.11 | −38.89 | −28.57 | −38.89 |
| Q1, Q3 | −38.89, | −44.44, | −44.44, | −38.89, | −45.83, |
| | −28.57 | −27.78 | −33.33 | −27.78 | −28.57 |
| Min, Max | −38.9, −28.6 | −44.4, −27.8 | −44.4, −33.3 | −38.9, −27.8 | −60.0, −27.8 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 1 | 1 | 1 | |
| Mean (SD) | | | 12.00 | 7.00 | 10.50 | |
| Median | | | 12.00 | 7.00 | 10.50 | |
| Q1, Q3 | | | 12.00, | 7.00, 7.00 | 10.50, | |
| | | | 12.00 | | 10.50 | |
| Min, Max | | | 12.0, 12.0 | 7.0, 7.0 | 10.5, 10.5 | |
| Weekly | | | | | | |
| Symptomatic RP | | | | | | |
| Attacks | | | | | | |
| n | | | 1 | 1 | 1 | |
| Mean (SD) | | | 18.00 | 4.00 | 10.00 | |
| Median | | | 18.00 | 4.00 | 10.00 | |
| Q1, Q3 | | | 18.00, | 4.00, 4.00 | 10.00, | |
| | | | 18.00 | | 10.00 | |
| Min, Max | | | 18.0, 18.0 | 4.0, 4.0 | 10.0, 10.0 | |
| Change from | | | | | | |
| Baseline | | | | | | |
| n | | | 1 | 1 | 1 | |
| Mean (SD) | | | 6.00 | −3.00 | −0.50 | |
| Median | | | 6.00 | −3.00 | −0.50 | |
| Q1, Q3 | | | 6.00, 6.00 | −3.00, −3.00 | −0.50, −0.50 | |
| Min, Max | | | 6.0, 6.0 | −3.0, −3.0 | −0.5, −0.5 | |
| Percent Change | | | | | | |
| from Baseline | | | | | | |
| n | | | 1 | 1 | 1 | |
| Mean (SD) | | | 50.00 | −42.86 | −4.76 | |
| Median | | | 50.00 | −42.86 | −4.76 | |
| Q1, Q3 | | | 50.00, | −42.86, | −4.76, −4.76 | |
| | | | 50.00 | −42.86 | | |
| Min, Max | | | 50.0, 50.0 | −42.9, −42.9 | −4.8, −4.8 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 3 |
| Mean (SD) | | 8.75 | 7.00 | 10.50 | 9.83 |
| | | (2.475) | | | (2.566) |
| Median | | 8.75 | 7.00 | 10.50 | 10.50 |
| Q1, Q3 | | 7.00, 10.50 | 7.00, 7.00 | 10.50, | 7.00, 12.00 |
| | | | | 10.50 | |
| Min, Max | | 7.0, 10.5 | 7.0, 7.0 | 10.5, 10.5 | 7.0, 12.0 |
| Weekly | | | | | |
| Symptomatic RP | | | | | |
| Attacks | | | | | |
| n | | 2 | 1 | 1 | 3 |
| Mean (SD) | | 7.00 | 4.00 | 10.00 | 10.67 |
| | | (4.243) | | | (7.024) |
| Median | | 7.00 | 4.00 | 10.00 | 10.00 |
| Q1, Q3 | | 4.00, 10.00 | 4.00, 4.00 | 10.00, | 4.00, 18.00 |
| | | | | 10.00 | |
| Min, Max | | 4.0, 10.0 | 4.0, 4.0 | 10.0, 10.0 | 4.0, 18.0 |
| Change from | | | | | |
| Baseline | | | | | |
| n | | 2 | 1 | 1 | 3 |
| Mean (SD) | | −1.75 | −3.00 | −0.50 | 0.83 |
| | | (1.768) | | | (4.646) |
| Median | | −1.75 | −3.00 | −0.50 | −0.50 |
| Q1, Q3 | | −3.00, −0.50 | −3.00, −3.00 | −0.50, −0.50 | −3.00, 6.00 |
| Min, Max | | −3.0, −0.5 | −3.0, −3.0 | −0.5, −0.5 | −3.0, 6.0 |
| Percent Change | | | | | |
| from Baseline | | | | | |
| n | | 2 | 1 | 1 | 3 |
| Mean (SD) | | −23.81 | −42.86 | −4.76 | 0.79 |
| | | (26.937) | | | (46.677) |
| Median | | −23.81 | −42.86 | −4.76 | −4.76 |
| Q1, Q3 | | −42.86, | −42.86, | −4.76, −4.76 | −42.86, |
| | | −4.76 | −42.86 | | 50.00 |
| Min, Max | | −42.9, −4.8 | −42.9, −42.9 | −4.8, −4.8 | −42.9, 50.0 |

TABLE A-5
Weekly RP Attacks (All Attacks) Change and Percent Change from Baseline.
ITT Population - Part A.
Frequency of weekly RP attacks is defined as the total number of RP attacks divided by
the number of days with available diary data within the last 7 days of the dosing period
(excluding the last dosing day) then multiplied by 7. Baseline is calculated as total
number of RP attacks during the last 7 days of screening period (excluding the first
dosing day) divided by the number of days with available data then multiplied by 7.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 2 | 2 | 2 | 2 |
| Mean | 32.00 | 14.67 | 10.00 | 10.00 | 15.92 | 17.83 |
| (SD) | | (7.542) | (4.243) | (1.414) | (4.360) | (8.721) |
| Median | 32.00 | 14.67 | 10.00 | 10.00 | 15.92 | 17.83 |
| Q1, Q3 | 32.00, | 9.33, 20.00 | 7.00, 13.00 | 9.00, 11.00 | 12.83, | 11.67, |
| | 32.00 | | | | 19.00 | 24.00 |
| Min, Max | 32.0, 32.0 | 9.3, 20.0 | 7.0, 13.0 | 9.0, 11.0 | 12.8, 19.0 | 11.7, 24.0 |
| Weekly | | | | | | |
| RP | | | | | | |
| Attacks | | | | | | |
| n | 1 | 2 | 2 | 2 | 2 | 2 |
| Mean | 23.00 | 8.00 | 13.00 | 5.50 | 13.00 | 9.83 |
| (SD) | | (4.243) | (8.485) | (0.707) | (2.828) | (7.307) |
| Median | 23.00 | 8.00 | 13.00 | 5.50 | 13.00 | 9.83 |
| Q1, Q3 | 23.00, | 5.00, 11.00 | 7.00, 19.00 | 5.00, 6.00 | 11.00, | 4.67, 15.00 |
| | 23.00 | | | | 15.00 | |
| Min, Max | 23.0, 23.0 | 5.0, 11.0 | 7.0, 19.0 | 5.0, 6.0 | 11.0, 15.0 | 4.7, 15.0 |
| Change | | | | | | |
| from | | | | | | |
| Baseline | | | | | | |
| n | 1 | 2 | 2 | 2 | 2 | 2 |
| Mean | −9.00 | −6.67 | 3.00 | −4.50 | −2.92 | −8.00 |
| (SD) | | (3.300) | (4.243) | (0.707) | (1.532) | (1.414) |
| Median | −9.00 | −6.67 | 3.00 | −4.50 | −2.92 | −8.00 |
| Q1, Q3 | −9.00, −9.00 | −9.00, −4.33 | 0.00, 6.00 | −5.00, −4.00 | −4.00, −1.83 | −9.00, −7.00 |
| Min, Max | −9.0, −9.0 | −9.0, −4.3 | 0.0, 6.0 | −5.0, −4.0 | −4.0, −1.8 | −9.0, −7.0 |
| Percent | | | | | | |
| Change | | | | | | |
| from | | | | | | |
| Baseline | | | | | | |
| n | 1 | 2 | 2 | 2 | 2 | 2 |
| Mean | −28.13 | −45.71 | 23.08 | −44.95 | −17.67 | −48.75 |
| (SD) | | (1.010) | (32.636) | (0.714) | (4.785) | (15.910) |
| Median | −28.13 | −45.71 | 23.08 | −44.95 | −17.67 | −48.75 |
| Q1, Q3 | −28.13, | −46.43, | 0.00, 46.15 | −45.45, | −21.05, | −60.00, |
| | −28.13 | −45.00 | | −44.44 | −14.29 | −37.50 |
| Min, Max | −28.1, −28.1 | −46.4, −45.0 | 0.0, 46.2 | −45.5, −44.4 | −21.1, −14.3 | −60.0, −37.5 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 11 |
| Mean | 20.44 | 12.96 | 17.33 | 15.29 | 15.35 |
| (SD) | (11.340) | (4.321) | (12.741) | (5.082) | (7.600) |
| Median | 20.00 | 11.92 | 11.00 | 15.92 | 12.83 |
| Q1, Q3 | 9.33, 32.00 | 10.00, | 9.00, 32.00 | 11.08, | 9.33, 20.00 |
| | | 15.92 | | 19.50 | |
| Min, Max | 9.3, 32.0 | 9.0, 19.0 | 9.0, 32.0 | 9.3, 20.0 | 7.0, 32.0 |
| Weekly | | | | | |
| RP | | | | | |
| Attacks | | | | | |
| n | 3 | 4 | 3 | 4 | 11 |
| Mean | 13.00 | 9.25 | 11.33 | 10.50 | 11.06 |
| (SD) | (9.165) | (4.646) | (10.116) | (4.123) | (6.270) |
| Median | 11.00 | 8.50 | 6.00 | 11.00 | 11.00 |
| Q1, Q3 | 5.00, 23.00 | 5.50, 13.00 | 5.00, 23.00 | 8.00, 13.00 | 5.00, 15.00 |
| Min, Max | 5.0, 23.0 | 5.0, 15.0 | 5.0, 23.0 | 5.0, 15.0 | 4.7, 23.0 |
| Change | | | | | |
| from | | | | | |
| Baseline | | | | | |
| n | 3 | 4 | 3 | 4 | 11 |
| Mean | −7.44 | −3.71 | −6.00 | −4.79 | −4.29 |
| (SD) | (2.694) | (1.336) | (2.646) | (3.017) | (4.527) |
| Median | −9.00 | −4.00 | −5.00 | −4.17 | −4.33 |
| Q1, Q3 | −9.00, −4.33 | −4.50, −2.92 | −9.00, −4.00 | −6.67, −2.92 | −9.00, −1.83 |
| Min, Max | −9.0, −4.3 | −5.0, −1.8 | −9.0, −4.0 | −9.0, −1.8 | −9.0, 6.0 |
| Percent | | | | | |
| Change | | | | | |
| from | | | | | |
| Baseline | | | | | |
| n | 3 | 4 | 3 | 4 | 11 |
| Mean | −39.85 | −31.31 | −39.34 | −31.69 | −26.92 |
| (SD) | (10.180) | (15.996) | (9.727) | (16.436) | (29.706) |
| Median | −45.00 | −32.75 | −44.44 | −33.03 | −37.50 |
| Q1, Q3 | −46.43, | −44.95, | −45.45, | −45.71, | −45.45, |
| | −28.13 | −17.67 | −28.13 | −17.67 | −14.29 |
| Min, Max | −46.4, −28.1 | −45.5, −14.3 | −45.5, −28.1 | −46.4, −14.3 | −60.0, 46.2 |

TABLE A-6
Weekly RP Attacks (All Attacks) Change and Percent Change from Baseline
(Excluding Outliers). ITT Population - Part A.
Frequency of weekly RP attacks is defined as the total number of RP attacks divided by
number of days with available diary data within the last 7 days of the dosing period
the (excluding the last dosing day) then multiplied by 7. Baseline is calculated as total
number of RP attacks during the last 7 days of screening period (excluding the first
dosing day) divided by the number of days with available data then multiplied by 7.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 1 | 2 | 2 | 2 |
| Mean | 32.00 | 14.67 | 13.00 | 10.00 | 15.92 | 17.83 |
| (SD) | | (7.542) | | (1.414) | (4.360) | (8.721) |
| Median | 32.00 | 14.67 | 13.00 | 10.00 | 15.92 | 17.83 |
| Q1, Q3 | 32.00, 32.00 | 9.33, 20.00 | 13.00, 13.00 | 9.00, 11.00 | 12.83, 19.00 | 11.67, 24.00 |
| Min, Max | 32.0, 32.0 | 9.3, 20.0 | 13.0, 13.0 | 9.0, 11.0 | 12.8, 19.0 | 11.7, 24.0 |
| Weekly | | | | | | |
| RP | | | | | | |
| Attacks | | | | | | |
| n | 1 | 2 | 1 | 2 | 2 | 2 |
| Mean | 23.00 | 8.00 | 19.00 | 5.50 | 13.00 | 9.83 |
| (SD) | | (4.243) | | (0.707) | (2.828) | (7.307) |
| Median | 23.00 | 8.00 | 19.00 | 5.50 | 13.00 | 9.83 |
| Q1, Q3 | 23.00, 23.00 | 5.00, 11.00 | 19.00, 19.00 | 5.00, 6.00 | 11.00, 15.00 | 4.67, 15.00 |
| Min, Max | 23.0, 23.0 | 5.0, 11.0 | 19.0, 19.0 | 5.0, 6.0 | 11.0, 15.0 | 4.7, 15.0 |
| Change | | | | | | |
| from | | | | | | |
| Baseline | | | | | | |
| n | 1 | 2 | 1 | 2 | 2 | 2 |
| Mean | −9.00 | −6.67 | 6.00 | −4.50 | −2.92 | −8.00 |
| (SD) | | (3.300) | | (0.707) | (1.532) | (1.414) |
| Median | −9.00 | −6.67 | 6.00 | −4.50 | −2.92 | −8.00 |
| Q1, Q3 | −9.00, −9.00 | −9.00, −4.33 | 6.00, 6.00 | −5.00, −4.00 | −4.00, −1.83 | −9.00, −7.00 |
| Min, Max | −9.0, −9.0 | −9.0, −4.3 | 6.0, 6.0 | −5.0, −4.0 | −4.0, −1.8 | −9.0, −7.0 |
| Percent | | | | | | |
| Change | | | | | | |
| from | | | | | | |
| Baseline | | | | | | |
| n | 1 | 2 | 1 | 2 | 2 | 2 |
| Mean | −28.13 | −45.71 | 46.15 | −44.95 | −17.67 | −48.75 |
| (SD) | | (1.010) | | (0.714) | (4.785) | (15.910) |
| Median | −28.13 | −45.71 | 46.15 | −44.95 | −17.67 | −48.75 |
| Q1, Q3 | −28.13, | −46.43, | 46.15, | −45.45, | −21.05, | −60.00, |
| | −28.13 | −45.00 | 46.15 | −44.44 | −14.29 | −37.50 |
| Min, Max | −28.1, −28.1 | −46.4, −45.0 | 46.2, 46.2 | −45.5, −44.4 | −21.1, −14.3 | −60.0, −37.5 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 10 |
| Mean | 20.44 | 12.96 | 17.33 | 15.29 | 16.18 |
| (SD) | (11.340) | (4.321) | (12.741) | (5.082) | (7.460) |
| Median | 20.00 | 11.92 | 11.00 | 15.92 | 12.92 |
| Q1, Q3 | 9.33, 32.00 | 10.00, | 9.00, 32.00 | 11.08, | 11.00, |
| | | 15.92 | | 19.50 | 20.00 |
| Min, Max | 9.3, 32.0 | 9.0, 19.0 | 9.0, 32.0 | 9.3, 20.0 | 9.0, 32.0 |
| Weekly | | | | | |
| RP | | | | | |
| Attacks | | | | | |
| n | 3 | 4 | 3 | 4 | 10 |
| Mean | 13.00 | 9.25 | 11.33 | 10.50 | 11.47 |
| (SD) | (9.165) | (4.646) | (10.116) | (4.123) | (6.454) |
| Median | 11.00 | 8.50 | 6.00 | 11.00 | 11.00 |
| Q1, Q3 | 5.00, 23.00 | 5.50, 13.00 | 5.00, 23.00 | 8.00, 13.00 | 5.00, 15.00 |
| Min, Max | 5.0, 23.0 | 5.0, 15.0 | 5.0, 23.0 | 5.0, 15.0 | 4.7, 23.0 |
| Change | | | | | |
| from | | | | | |
| Baseline | | | | | |
| n | 3 | 4 | 3 | 4 | 10 |
| Mean | −7.44 | −3.71 | −6.00 | −4.79 | −4.72 |
| (SD) | (2.694) | (1.336) | (2.646) | (3.017) | (4.530) |
| Median | −9.00 | −4.00 | −5.00 | −4.17 | −4.67 |
| Q1, Q3 | −9.00, −4.33 | −4.50, −2.92 | −9.00, −4.00 | −6.67, −2.92 | −9.00, −4.00 |
| Min, Max | −9.0, −4.3 | −5.0, −1.8 | −9.0, −4.0 | −9.0, −1.8 | −9.0, 6.0 |
| Percent | | | | | |
| Change | | | | | |
| from | | | | | |
| Baseline | | | | | |
| n | 3 | 4 | 3 | 4 | 10 |
| Mean | −39.85 | −31.31 | −39.34 | −31.69 | −29.61 |
| (SD) | (10.180) | (15.996) | (9.727) | (16.436) | (29.865) |
| Median | −45.00 | −32.75 | −44.44 | −33.03 | −40.97 |
| Q1, Q3 | −46.43, −28.13 | −44.95, −17.67 | −45.45, −28.13 | −45.71, | −45.45, |
| | | | | −17.67 | −21.05 |
| Min, Max | −46.4, −28.1 | −45.5, −14.3 | −45.5, −28.1 | −46.4, −14.3 | −60.0, 46.2 |

TABLE A-7
Weekly RP Attacks (All Attacks) Change and Percent Change from Baseline
by Baseline RCS. ITT Population - Part A.
Frequency of weekly RP attacks is defined as the total number of RP attacks divided by
number of days with available diary data within the last 7 days of the dosing period
(excluding the last dosing day) then multiplied by 7. Baseline is calculated as total
number of RP attacks during the last 7 days of screening period (excluding the first
dosing day) divided by the number of days with available data then multiplied by 7.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 32.00 | 14.67 | | 9.00 | 19.00 | 17.83 |
| (SD) | | (7.542) | | | | (8.721) |
| Median | 32.00 | 14.67 | | 9.00 | 19.00 | 17.83 |
| Q1, Q3 | 32.00, | 9.33, 20.00 | | 9.00, 9.00 | 19.00, | 11.67, |
| | 32.00 | | | | 19.00 | 24.00 |
| Min, Max | 32.0, 32.0 | 9.3, 20.0 | | 9.0, 9.0 | 19.0, 19.0 | 11.7, 24.0 |
| Weekly RP | | | | | | |
| Attacks | | | | | | |
| n | 1 | 2 | | 1 | 1 | 2 |
| Mean | 23.00 | 8.00 | | 5.00 | 15.00 | 9.83 |
| (SD) | | (4.243) | | | | (7.307) |
| Median | 23.00 | 8.00 | | 5.00 | 15.00 | 9.83 |
| Q1, Q3 | 23.00, | 5.00,11.00 | | 5.00,5.00 | 15.00, | 4.67,15.00 |
| | 23.00 | | | | 15.00 | |
| Min, Max | 23.0,23.0 | 5.0,11.0 | | 5.0,5.0 | 15.0,15.0 | 4.7,15.0 |
| Change | | | | | | |
| from | | | | | | |
| Baseline | | | | | | |
| n | 1 | 2 | | 1 | 1 | 2 |
| Mean | −9.00 | −6.67 | | −4.00 | −4.00 | −8.00 |
| (SD) | | (3.300) | | | | (1.414) |
| Median | −9.00 | −6.67 | | −4.00 | −4.00 | −8.00 |
| Q1, Q3 | −9.00, −9.00 | −9.00, −4.33 | | −4.00, −4.00 | −4.00, −4.00 | −9.00, −7.00 |
| Min, Max | −9.0, −9.0 | −9.0, −4.3 | | −4.0, −4.0 | −4.0, −4.0 | −9.0, −7.0 |
| Percent | | | | | | |
| Change | | | | | | |
| from | | | | | | |
| Baseline | | | | | | |
| n | 1 | 2 | | 1 | 1 | 2 |
| Mean | −28.13 | −45.71 | | −44.44 | −21.05 | −48.75 |
| (SD) | | (1.010) | | | | (15.910) |
| Median | −28.13 | −45.71 | | −44.44 | −21.05 | −48.75 |
| Q1, Q3 | −28.13, | −46.43, | | −44.44, | −21.05, | −60.00, |
| | −28.13 | −45.00 | | −44.44 | −21.05 | −37.50 |
| Min, Max | −28.1, −28.1 | −46.4, −45.0 | | −44.4, −44.4 | −21.1, −21.1 | −60.0, −37.5 |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine + | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | 3 | 2 | 2 | 3 | 7 | |
| Mean | 20.44 | 14.00 | 20.50 | 16.11 | 17.86 | |
| (SD) | (11.340) | (7.071) | (16.263) | (5.891) | (8.496) | |
| Median | 20.00 | 14.00 | 20.50 | 19.00 | 19.00 | |
| Q1, Q3 | 9.33, 32.00 | 9.00, 19.00 | 9.00, 32.00 | 9.33, 20.00 | 9.33, 24.00 | |
| Min, Max | 9.3, 32.0 | 9.0, 19.0 | 9.0, 32.0 | 9.3, 20.0 | 9.0, 32.0 | |
| Weekly RP | | | | | | |
| Attacks | | | | | | |
| n | 3 | 2 | 2 | 3 | 7 | |
| Mean | 13.00 | 10.00 | 14.00 | 10.33 | 11.24 | |
| (SD) | (9.165) | (7.071) | (12.728) | (5.033) | (6.925) | |
| Median | 11.00 | 10.00 | 14.00 | 11.00 | 11.00 | |
| Q1, Q3 | 5.00, 23.00 | 5.00, 15.00 | 5.00, 23.00 | 5.00, 15.00 | 5.00, 15.00 | |
| Min, Max | 5.0, 23.0 | 5.0, 15.0 | 5.0, 23.0 | 5.0, 15.0 | 4.7, 23.0 | |
| Change | | | | | | |
| from | | | | | | |
| Baseline | | | | | | |
| n | 3 | 2 | 2 | 3 | 7 | |
| Mean | −7.44 | −4.00 | −6.50 | −5.78 | −6.62 | |
| (SD) | (2.694) | (0.000) | (3.536) | (2.795) | (2.453) | |
| Median | −9.00 | −4.00 | −6.50 | −4.33 | −7.00 | |
| Q1, Q3 | −9.00, −4.33 | −4.00, −4.00 | −9.00, −4.00 | −9.00, −4.00 | −9.00, −4.00 | |
| Min, Max | −9.0, −4.3 | −4.0, −4.0 | −9.0, −4.0 | −9.0, −4.0 | −9.0, −4.0 | |
| Percent | | | | | | |
| Change | | | | | | |
| from | | | | | | |
| Baseline | | | | | | |
| n | 3 | 2 | 2 | 3 | 7 | |
| Mean | −39.85 | −32.75 | −36.28 | −37.49 | −40.36 | |
| (SD) | (10.180) | (16.541) | (11.540) | (14.256) | (12.854) | |
| Median | −45.00 | −32.75 | −36.28 | −45.00 | −44.44 | |
| Q1, Q3 | −46.43, | −44.44, | −44.44, | −46.43, | −46.43, | |
| | −28.13 | −21.05 | −28.13 | −21.05 | −28.13 | |
| Min, Max | −46.4, −28.1 | −44.4, −21.1 | −44.4, −28.1 | −46.4, −21.1 | −60.0, −21.1 | |
| | | | | Cilnidipine | Cilnidipine | |
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 2 | 1 | 1 | |
| Mean | | | 10.00 | 11.00 | 12.83 | |
| (SD) | | | (4.243) | | | |
| Median | | | 10.00 | 11.00 | 12.83 | |
| Q1, Q3 | | | 7.00, 13.00 | 11.00, | 12.83, | |
| | | | | 11.00 | 12.83 | |
| Min, Max | | | 7.0, 13.0 | 11.0, 11.0 | 12.8, 12.8 | |
| Weekly RP | | | | | | |
| Attacks | | | | | | |
| n | | | 2 | 1 | 1 | |
| Mean | | | 13.00 | 6.00 | 11.00 | |
| (SD) | | | (8.485) | | | |
| Median | | | 13.00 | 6.00 | 11.00 | |
| Q1, Q3 | | | 7.00, 19.00 | 6.00, 6.00 | 11.00, 11.00 | |
| Min, Max | | | 7.0, 19.0 | 6.0, 6.0 | 11.0, 11.0 | |
| Change | | | | | | |
| from | | | | | | |
| Baseline | | | | | | |
| n | | | 2 | 1 | 1 | |
| Mean | | | 3.00 | −5.00 | −1.83 | |
| (SD) | | | (4.243) | | | |
| Median | | | 3.00 | −5.00 | −1.83 | |
| Q1, Q3 | | | 0.00, 6.00 | −5.00, −5.00 | −1.83, −1.83 | |
| Min, Max | | | 0.0, 6.0 | −5.0, −5.0 | −1.8, −1.8 | |
| Percent | | | | | | |
| Change | | | | | | |
| from | | | | | | |
| Baseline | | | | | | |
| n | | | 2 | 1 | 1 | |
| Mean | | | 23.08 | −45.45 | −14.29 | |
| (SD) | | | (32.636) | | | |
| Median | | | 23.08 | −45.45 | −14.29 | |
| Q1, Q3 | | | 0.00, 46.15 | −45.45, | −14.29, | |
| | | | | −45.45 | −14.29 | |
| Min, Max | | | 0.0, 46.2 | −45.5, −45.5 | −14.3, −14.3 | |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine + | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | | 2 | 1 | 1 | 4 | |
| Mean | | 11.92 | 11.00 | 12.83 | 10.96 | |
| (SD) | | (1.296) | | | (2.790) | |
| Median | | 11.92 | 11.00 | 12.83 | 11.92 | |
| Q1, Q3 | | 11.00, | 11.00, | 12.83, | 9.00, | |
| | | 12.83 | 11.00 | 12.83 | 12.92 | |
| Min, Max | | 11.0, 12.8 | 11.0, 11.0 | 12.8, 12.8 | 7.0, 13.0 | |
| Weekly RP | | | | | | |
| Attacks | | | | | | |
| n | | 2 | 1 | 1 | 4 | |
| Mean | | 8.50 | 6.00 | 11.00 | 10.75 | |
| (SD) | | (3.536) | | | (5.909) | |
| Median | | 8.50 | 6.00 | 11.00 | 9.00 | |
| Q1, Q3 | | 6.00, | 6.00, | 11.00, | 6.50, | |
| | | 11.00 | 6.00 | 11.00 | 15.00 | |
| Min, Max | | 6.0, 11.0 | 6.0, 6.0 | 11.0, 11.0 | 6.0, 19.0 | |
| Change | | | | | | |
| from | | | | | | |
| Baseline | | | | | | |
| n | | 2 | 1 | 1 | 4 | |
| Mean | | −3.42 | −5.00 | −1.83 | −0.21 | |
| (SD) | | (2.239) | | | (4.626) | |
| Median | | −3.42 | −5.00 | −1.83 | −0.92 | |
| Q1, Q3 | | −5.00, −1.83 | −5.00, −5.00 | −1.83, −1.83 | −3.42, 3.00 | |
| Min, Max | | −5.0, −1.8 | −5.0, −5.0 | −1.8, −1.8 | −5.0, 6.0 | |
| Percent | | | | | | |
| Change | | | | | | |
| from | | | | | | |
| Baseline | | | | | | |
| n | | 2 | 1 | 1 | 4 | |
| Mean | | −29.87 | −45.45 | −14.29 | −3.40 | |
| (SD) | | (22.040) | | | (38.097) | |
| Median | | −29.87 | −45.45 | −14.29 | −7.14 | |
| Q1, Q3 | | −45.45, | −45.45, | −14.29, | −29.87, | |
| | | −14.29 | −45.45 | −14.29 | 23.08 | |
| Min, Max | | −45.5, −14.3 | −45.5, −45.5 | −14.3, −14.3 | −45.5, 46.2 | |

TABLE A-8
Weekly RP Attacks (All Attacks) Change and Percent Change from Baseline
(Excluding Outliers) by Baseline RCS. ITT Population - Part A.
Frequency of weekly RP attacks is defined as the total number of RP attacks divided by
the number of days with available diary data within the last 7 days of the dosing period
(excluding the last dosing day) then multiplied by 7. Baseline is calculated as total
number of RP attacks during the last 7 days of screening period (excluding the first
dosing day) divided by the number of days with available data then multiplied by 7.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 32.00 | 14.67 | | 9.00 | 19.00 | 17.83 |
| (SD) | | (7.542) | | | | (8.721) |
| Median | 32.00 | 14.67 | | 9.00 | 19.00 | 17.83 |
| Q1, Q3 | 32.00, | 9.33, 20.00 | | 9.00, 9.00 | 19.00, | 11.67, |
| | 32.00 | | | | 19.00 | 24.00 |
| Min, Max | 32.0, 32.0 | 9.3, 20.0 | | 9.0, 9.0 | 19.0, 19.0 | 11.7, 24.0 |
| Weekly RP | | | | | | |
| Attacks | | | | | | |
| n | 1 | 2 | | 1 | 1 | 2 |
| Mean | 23.00 | 8.00 | | 5.00 | 15.00 | 9.83 |
| (SD) | | (4.243) | | | | (7.307) |
| Median | 23.00 | 8.00 | | 5.00 | 15.00 | 9.83 |
| Q1, Q3 | 23.00, | 5.00, 11.00 | | 5.00, 5.00 | 15.00, | 4.67, 15.00 |
| | 23.00 | | | | 15.00 | |
| Min, Max | 23.0, 23.0 | 5.0, 11.0 | | 5.0, 5.0 | 15.0, 15.0 | 4.7, 15.0 |
| Change | | | | | | |
| from | | | | | | |
| Baseline | | | | | | |
| n | 1 | 2 | | 1 | 1 | 2 |
| Mean | −9.00 | −6.67 | | −4.00 | −4.00 | −8.00 |
| (SD) | | (3.300) | | | | (1.414) |
| Median | −9.00 | −6.67 | | −4.00 | −4.00 | −8.00 |
| Q1, Q3 | −9.00, −9.00 | −9.00, −4.33 | | −4.00, −4.00 | −4.00, −4.00 | −9.00, −7.00 |
| Min, Max | −9.0, −9.0 | −9.0, −4.3 | | −4.0, −4.0 | −4.0, −4.0 | −9.0, −7.0 |
| Percent | | | | | | |
| Change | | | | | | |
| from | | | | | | |
| Baseline | | | | | | |
| n | 1 | 2 | | 1 | 1 | 2 |
| Mean | −28.13 | −45.71 | | −44.44 | −21.05 | −48.75 |
| (SD) | | (1.010) | | | | (15.910) |
| Median | −28.13 | −45.71 | | −44.44 | −21.05 | −48.75 |
| Q1, Q3 | −28.13, | −46.43, | | −44.44, | −21.05, | −60.00, |
| | −28.13 | −45.00 | | −44.44 | −21.05 | −37.50 |
| Min, Max | −28.1, −28.1 | −46.4, −45.0 | | −44.4, −44.4 | −21.1, −21.1 | −60.0, −37.5 |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine + | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | 3 | 2 | 2 | 3 | 7 | |
| Mean | 20.44 | 14.00 | 20.50 | 16.11 | 17.86 | |
| (SD) | (11.340) | (7.071) | (16.263) | (5.891) | (8.496) | |
| Median | 20.00 | 14.00 | 20.50 | 19.00 | 19.00 | |
| Q1, Q3 | 9.33, 32.00 | 9.00, 19.00 | 9.00, 32.00 | 9.33, 20.00 | 9.33, 24.00 | |
| Min, Max | 9.3, 32.0 | 9.0, 19.0 | 9.0, 32.0 | 9.3, 20.0 | 9.0, 32.0 | |
| Weekly RP | | | | | | |
| Attacks | | | | | | |
| n | 3 | 2 | 2 | 3 | 7 | |
| Mean | 13.00 | 10.00 | 14.00 | 10.33 | 11.24 | |
| (SD) | (9.165) | (7.071) | (12.728) | (5.033) | (6.925) | |
| Median | 11.00 | 10.00 | 14.00 | 11.00 | 11.00 | |
| Q1, Q3 | 5.00, 23.00 | 5.00, 15.00 | 5.00, 23.00 | 5.00, 15.00 | 5.00, 15.00 | |
| Min, Max | 5.0, 23.0 | 5.0, 15.0 | 5.0, 23.0 | 5.0, 15.0 | 4.7, 23.0 | |
| Change | | | | | | |
| from | | | | | | |
| Baseline | | | | | | |
| n | 3 | 2 | 2 | 3 | 7 | |
| Mean | −7.44 | −4.00 | −6.50 | −5.78 | −6.62 | |
| (SD) | (2.694) | (0.000) | (3.536) | (2.795) | (2.453) | |
| Median | −9.00 | −4.00 | −6.50 | −4.33 | −7.00 | |
| Q1, Q3 | −9.00, −4.33 | −4.00, −4.00 | −9.00, −4.00 | −9.00, −4.00 | −9.00, −4.00 | |
| Min, Max | −9.0, −4.3 | −4.0, −4.0 | −9.0, −4.0 | −9.0, −4.0 | −9.0, −4.0 | |
| Percent | | | | | | |
| Change | | | | | | |
| from | | | | | | |
| Baseline | | | | | | |
| n | 3 | 2 | 2 | 3 | 7 | |
| Mean | −39.85 | −32.75 | −36.28 | −37.49 | −40.36 | |
| (SD) | (10.180) | (16.541) | (11.540) | (14.256) | (12.854) | |
| Median | −45.00 | −32.75 | −36.28 | −45.00 | −44.44 | |
| Q1, Q3 | −46.43, | −44.44, | −44.44, | −46.43, | −46.43, | |
| | −28.13 | −21.05 | −28.13 | −21.05 | −28.13 | |
| Min, Max | −46.4, −28.1 | −44.4, −21.1 | −44.4, −28.1 | −46.4, −21.1 | −60.0, −21.1 | |
| | | | | Cilnidipine | Cilnidipine | |
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 1 | 1 | 1 | |
| Mean | | | 13.00 | 11.00 | 12.83 | |
| (SD) | | | | | | |
| Median | | | 13.00 | 11.00 | 12.83 | |
| Q1, Q3 | | | 13.00, | 11.00, | 12.83, | |
| | | | 13.00 | 11.00 | 12.83 | |
| Min, Max | | | 13.0, 13.0 | 11.0, 11.0 | 12.8, 12.8 | |
| Weekly RP | | | | | | |
| Attacks | | | | | | |
| n | | | 1 | 1 | 1 | |
| Mean | | | 19.00 | 6.00 | 11.00 | |
| (SD) | | | | | | |
| Median | | | 19.00 | 6.00 | 11.00 | |
| Q1, Q3 | | | 19.00, | 6.00, 6.00 | 11.00, | |
| | | | 19.00 | | 11.00 | |
| Min, Max | | | 19.0, 19.0 | 6.0, 6.0 | 11.0, 11.0 | |
| Change | | | | | | |
| from | | | | | | |
| Baseline | | | | | | |
| n | | | 1 | 1 | 1 | |
| Mean | | | 6.00 | −5.00 | −1.83 | |
| (SD) | | | | | | |
| Median | | | 6.00 | −5.00 | −1.83 | |
| Q1, Q3 | | | 6.00, 6.00 | −5.00, −5.00 | −1.83, −1.83 | |
| Min, Max | | | 6.0, 6.0 | −5.0, −5.0 | −1.8, −1.8 | |
| Percent | | | | | | |
| Change | | | | | | |
| from | | | | | | |
| Baseline | | | | | | |
| n | | | 1 | 1 | 1 | |
| Mean | | | 46.15 | −45.45 | −14.29 | |
| (SD) | | | | | | |
| Median | | | 46.15 | −45.45 | −14.29 | |
| Q1, Q3 | | | 46.15, | −45.45, | −14.29, | |
| | | | 46.15 | −45.45 | −14.29 | |
| Min, Max | | | 46.2, 46.2 | −45.5, −45.5 | −14.3, −14.3 | |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine + | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | | 2 | 1 | 1 | 3 | |
| Mean | | 11.92 | 11.00 | 12.83 | 12.28 | |
| (SD) | | (1.296) | | | (1.110) | |
| Median | | 11.92 | 11.00 | 12.83 | 12.83 | |
| Q1, Q3 | | 11.00, | 11.00, | 12.83, | 11.00, | |
| | | 12.83 | 11.00 | 12.83 | 13.00 | |
| Min, Max | | 11.0, 12.8 | 11.0, 11.0 | 12.8, 12.8 | 11.0, 13.0 | |
| Weekly RP | | | | | | |
| Attacks | | | | | | |
| n | | 2 | 1 | 1 | 3 | |
| Mean | | 8.50 | 6.00 | 11.00 | 12.00 | |
| (SD) | | (3.536) | | | (6.557) | |
| Median | | 8.50 | 6.00 | 11.00 | 11.00 | |
| Q1, Q3 | | 6.00, 11.00 | 6.00, 6.00 | 11.00, | 6.00, 19.00 | |
| | | | | 11.00 | | |
| Min, Max | | 6.0, 11.0 | 6.0, 6.0 | 11.0, 11.0 | 6.0, 19.0 | |
| Change | | | | | | |
| from | | | | | | |
| Baseline | | | | | | |
| n | | 2 | 1 | 1 | 3 | |
| Mean | | −3.42 | −5.00 | −1.83 | −0.28 | |
| (SD) | | (2.239) | | | (5.663) | |
| Median | | −3.42 | −5.00 | −1.83 | −1.83 | |
| Q1, Q3 | | −5.00, −1.83 | −5.00, −5.00 | −1.83, −1.83 | −5.00, 6.00 | |
| Min, Max | | −5.0, −1.8 | −5.0, −5.0 | −1.8, −1.8 | −5.0, 6.0 | |
| Percent | | | | | | |
| Change | | | | | | |
| from | | | | | | |
| Baseline | | | | | | |
| n | | 2 | 1 | 1 | 3 | |
| Mean | | −29.87 | −45.45 | −14.29 | −4.53 | |
| (SD) | | (22.040) | | | (46.577) | |
| Median | | −29.87 | −45.45 | −14.29 | −14.29 | |
| Q1, Q3 | | −45.45, | −45.45, | −14.29, | −45.45, | |
| | | −14.29 | −45.45 | −14.29 | 46.15 | |
| Min, Max | | −45.5, −14.3 | −45.5, −45.5 | −14.3, −14.3 | −45.5, 46.2 | |

Tables B-1 to B-8 include data that relates to the duration of symptomatic Raynaud's attacks.

TABLE B-1
Average Duration of Weekly Symptomatic RP Attacks Change and Percent
Change from Baseline. ITT Population - Part A.
Average duration (minutes) of weekly symptomatic RP attacks is defined as the total
duration of symptomatic RP attacks divided by total number of symptomatic RP attacks
within the last 7 days of the dosing period (excluding the last dosing day). Baseline is
calculated as total duration of symptomatic RP attacks during the last 7 days of screening
period (excluding the first dosing day) divided by the total number of symptomatic RP
attacks. Only symptomatic RP attacks, defined as RP attacks with patient reported finger
color changes associated with at least one symptom (pain, numbness, tingling), were
included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 2 | 2 | 2 | 2 |
| Mean | 48.38 | 19.86 | 271.54 | 38.17 | 51.11 | 56.42 |
| (SD) | | (9.232) | (369.051) | (28.845) | (54.997) | (66.350) |
| Median | 48.38 | 19.86 | 271.54 | 38.17 | 51.11 | 56.42 |
| Q1, Q3 | 48.38, | 13.33, | 10.58, | 17.78, | 12.22, | 9.50, |
| | 48.38 | 26.39 | 532.50 | 58.57 | 90.00 | 103.33 |
| Min, Max | 48.4, 48.4 | 13.3, 26.4 | 10.6, 532.5 | 17.8, 58.6 | 12.2, 90.0 | 9.5, 103.3 |
| Average | | | | | | |
| Duration | | | | | | |
| n | 1 | 2 | 2 | 2 | 2 | 1 |
| Mean | 31.64 | 16.73 | 412.29 | 28.38 | 39.23 | 98.08 |
| (SD) | | (19.413) | (574.575) | (14.672) | (33.615) | |
| Median | 31.64 | 16.73 | 412.29 | 28.38 | 39.23 | 98.08 |
| Q1, Q3 | 31.64, | 3.00, 30.45 | 6.00, | 18.00, | 15.46, | 98.08, |
| | 31.64 | | 818.57 | 38.75 | 63.00 | 98.08 |
| Min, Max | 31.6, 31.6 | 3.0, 30.5 | 6.0, 818.6 | 18.0, 38.8 | 15.5, 63.0 | 98.1, 98.1 |
| Change | | | | | | |
| from | | | | | | |
| Baseline | | | | | | |
| n | 1 | 2 | 2 | 2 | 2 | 1 |
| Mean | −16.74 | −3.13 | 140.74 | −9.80 | −11.88 | −5.26 |
| (SD) | | (10.182) | (205.524) | (14.173) | (21.382) | |
| Median | −16.74 | −3.13 | 140.74 | −9.80 | −11.88 | −5.26 |
| Q1, Q3 | −16.74, | −10.33, | −4.58, | −19.82, | −27.00, | −5.26, −5.26 |
| | −16.74 | 4.07 | 286.07 | 0.22 | 3.24 | |
| Min, Max | −16.7, −16.7 | −10.3, 4.1 | −4.6, 286.1 | −19.8, 0.2 | −27.0, 3.2 | −5.3, −5.3 |
| Percent | | | | | | |
| Change | | | | | | |
| from | | | | | | |
| Baseline | | | | | | |
| n | 1 | 2 | 2 | 2 | 2 | 1 |
| Mean | −34.60 | −31.05 | 5.21 | −16.30 | −1.75 | −5.09 |
| (SD) | | (65.695) | (68.610) | (24.813) | (39.954) | |
| Median | −34.60 | −31.05 | 5.21 | −16.30 | −1.75 | −5.09 |
| Q1, Q3 | −34.60, | −77.50, | −43.31, | −33.84, | −30.00, | −5.09, −5.09 |
| | −34.60 | 15.41 | 53.72 | 1.25 | 26.50 | |
| Min, Max | −34.6, −34.6 | −77.5, 15.4 | −43.3, 53.7 | −33.8, 1.3 | −30.0, 26.5 | −5.1, −5.1 |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine + | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | 3 | 4 | 3 | 4 | 11 | |
| Mean | 29.37 | 44.64 | 41.58 | 35.49 | 83.87 | |
| (SD) | (17.713) | (36.625) | (21.231) | (36.907) | (152.403) | |
| Median | 26.39 | 38.17 | 48.38 | 19.86 | 26.39 | |
| Q1, Q3 | 13.33, | 15.00, | 17.78, | 12.78, | 12.22, | |
| | 48.38 | 74.29 | 58.57 | 58.19 | 90.00 | |
| Min, Max | 13.3, 48.4 | 12.2, 90.0 | 17.8, 58.6 | 12.2, 90.0 | 9.5, 532.5 | |
| Average | | | | | | |
| Duration | | | | | | |
| n | 3 | 4 | 3 | 4 | 10 | |
| Mean | 21.70 | 33.80 | 29.46 | 27.98 | 112.30 | |
| (SD) | (16.205) | (22.084) | (10.545) | (25.905) | (249.796) | |
| Median | 30.45 | 28.38 | 31.64 | 22.96 | 31.05 | |
| Q1, Q3 | 3.00, 31.64 | 16.73, | 18.00, | 9.23, 46.73 | 15.46, | |
| | | 50.88 | 38.75 | | 63.00 | |
| Min, Max | 3.0, 31.6 | 15.5, 63.0 | 18.0, 38.8 | 3.0, 63.0 | 3.0, 818.6 | |
| Change | | | | | | |
| from | | | | | | |
| Baseline | | | | | | |
| n | 3 | 4 | 3 | 4 | 10 | |
| Mean | −7.67 | −10.84 | −12.11 | −7.51 | 20.99 | |
| (SD) | (10.655) | (14.859) | (10.793) | (14.576) | (93.700) | |
| Median | −10.33 | −9.80 | −16.74 | −3.55 | −4.92 | |
| Q1, Q3 | −16.74, | −23.41, | −19.82, | −18.67, | −16.74, | |
| | 4.07 | 1.73 | 0.22 | 3.65 | 3.24 | |
| Min, Max | −16.7, 4.1 | −27.0, 3.2 | −19.8, 0.2 | −27.0, 4.1 | −27.0, | |
| | | | | | 286.1 | |
| Percent | | | | | | |
| Change | | | | | | |
| from | | | | | | |
| Baseline | | | | | | |
| n | 3 | 4 | 3 | 4 | 10 | |
| Mean | −32.23 | −9.02 | −22.40 | −16.40 | −12.74 | |
| (SD) | (46.499) | (28.423) | (20.481) | (47.506) | (38.518) | |
| Median | −34.60 | −14.38 | −33.84 | −7.30 | −17.54 | |
| Q1, Q3 | −77.50, | −31.92, | −34.60, | −53.75, | −34.60, | |
| | 15.41 | 13.88 | 1.25 | 20.96 | 15.41 | |
| Min, Max | −77.5, 15.4 | −33.8, 26.5 | −34.6, 1.3 | −77.5, 26.5 | −77.5, 53.7 | |

TABLE B-2
Average Duration of Weekly Symptomatic RP Attacks Change and Percent
Change from Baseline (Excluding Outliers). ITT Population - Part A.
Average duration (minutes) of weekly symptomatic RP attacks is defined as the total
duration of symptomatic RP attacks divided by total number of symptomatic RP attacks
within the last 7 days of the dosing period (excluding the last dosing day). Baseline is
calculated as total duration of symptomatic RP attacks during the last 7 days of screening
period (excluding the first dosing day) divided by the total number of symptomatic RP
attacks. Only symptomatic RP attacks, defined as RP attacks with patient reported finger
color changes associated with at least one symptom (pain, numbness, tingling), were
included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 1 | 2 | 2 | 2 |
| Mean | 48.38 | 19.86 | 10.58 | 38.17 | 51.11 | 56.42 |
| (SD) | | (9.232) | | (28.845) | (54.997) | (66.350) |
| Median | 48.38 | 19.86 | 10.58 | 38.17 | 51.11 | 56.42 |
| Q1, Q3 | 48.38, | 13.33, | 10.58, | 17.78, | 12.22, | 9.50, |
| | 48.38 | 26.39 | 10.58 | 58.57 | 90.00 | 103.33 |
| Min, Max | 48.4, 48.4 | 13.3, 26.4 | 10.6, 10.6 | 17.8, 58.6 | 12.2, 90.0 | 9.5, 103.3 |
| Average | | | | | | |
| Duration | | | | | | |
| n | 1 | 2 | 1 | 2 | 2 | 1 |
| Mean | 31.64 | 16.73 | 6.00 | 28.38 | 39.23 | 98.08 |
| (SD) | | (19.413) | | (14.672) | (33.615) | |
| Median | 31.64 | 16.73 | 6.00 | 28.38 | 39.23 | 98.08 |
| Q1, Q3 | 31.64, | 3.00, 30.45 | 6.00, 6.00 | 18.00, | 15.46, | 98.08, |
| | 31.64 | | | 38.75 | 63.00 | 98.08 |
| Min, Max | 31.6, 31.6 | 3.0, 30.5 | 6.0, 6.0 | 18.0, 38.8 | 15.5, 63.0 | 98.1, 98.1 |
| Change | | | | | | |
| from | | | | | | |
| Baseline | | | | | | |
| n | 1 | 2 | 1 | 2 | 2 | 1 |
| Mean | −16.74 | −3.13 | −4.58 | −9.80 | −11.88 | −5.26 |
| (SD) | | (10.182) | | (14.173) | (21.382) | |
| Median | −16.74 | −3.13 | −4.58 | −9.80 | −11.88 | −5.26 |
| Q1, Q3 | −16.74, | −10.33, | −4.58, −4.58 | −19.82, | −27.00, | −5.26, −5.26 |
| | −16.74 | 4.07 | | 0.22 | 3.24 | |
| Min, Max | −16.7, −16.7 | −10.3, 4.1 | −4.6, −4.6 | −19.8, 0.2 | −27.0, 3.2 | −5.3, −5.3 |
| Percent | | | | | | |
| Change | | | | | | |
| from | | | | | | |
| Baseline | | | | | | |
| n | 1 | 2 | 1 | 2 | 2 | 1 |
| Mean | −34.60 | −31.05 | −43.31 | −16.30 | −1.75 | −5.09 |
| (SD) | | (65.695) | | (24.813) | (39.954) | |
| Median | −34.60 | −31.05 | −43.31 | −16.30 | −1.75 | −5.09 |
| Q1, Q3 | −34.60, | −77.50, | −43.31, | −33.84, | −30.00, | −5.09, −5.09 |
| | −34.60 | 15.41 | −43.31 | 1.25 | 26.50 | |
| Min, Max | −34.6, −34.6 | −77.5, 15.4 | −43.3, −43.3 | −33.8, 1.3 | −30.0, 26.5 | −5.1, −5.1 |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine + | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | 3 | 4 | 3 | 4 | 10 | |
| Mean | 29.37 | 44.64 | 41.58 | 35.49 | 39.01 | |
| (SD) | (17.713) | (36.625) | (21.231) | (36.907) | (34.759) | |
| Median | 26.39 | 38.17 | 48.38 | 19.86 | 22.08 | |
| Q1, Q3 | 13.33, | 15.00, | 17.78, | 12.78, | 12.22, | |
| | 48.38 | 74.29 | 58.57 | 58.19 | 58.57 | |
| Min, Max | 13.3, 48.4 | 12.2, 90.0 | 17.8, 58.6 | 12.2, 90.0 | 9.5, 103.3 | |
| Average | | | | | | |
| Duration | | | | | | |
| n | 3 | 4 | 3 | 4 | 9 | |
| Mean | 21.70 | 33.80 | 29.46 | 27.98 | 33.82 | |
| (SD) | (16.205) | (22.084) | (10.545) | (25.905) | (30.274) | |
| Median | 30.45 | 28.38 | 31.64 | 22.96 | 30.45 | |
| Q1, Q3 | 3.00, 31.64 | 16.73, | 18.00, | 9.23, 46.73 | 15.46, | |
| | | 50.88 | 38.75 | | 38.75 | |
| Min, Max | 3.0, 31.6 | 15.5, 63.0 | 18.0, 38.8 | 3.0, 63.0 | 3.0, 98.1 | |
| Change | | | | | | |
| from | | | | | | |
| Baseline | | | | | | |
| n | 3 | 4 | 3 | 4 | 9 | |
| Mean | −7.67 | −10.84 | −12.11 | −7.51 | −8.47 | |
| (SD) | (10.655) | (14.859) | (10.793) | (14.576) | (10.836) | |
| Median | −10.33 | −9.80 | −16.74 | −3.55 | −5.26 | |
| Q1, Q3 | −16.74, | −23.41, | −19.82, | −18.67, | −16.74, | |
| | 4.07 | 1.73 | 0.22 | 3.65 | 0.22 | |
| Min, Max | −16.7, 4.1 | −27.0, 3.2 | −19.8, 0.2 | −27.0, 4.1 | −27.0, 4.1 | |
| Percent | | | | | | |
| Change | | | | | | |
| from | | | | | | |
| Baseline | | | | | | |
| n | 3 | 4 | 3 | 4 | 9 | |
| Mean | −32.23 | −9.02 | −22.40 | −16.40 | −20.13 | |
| (SD) | (46.499) | (28.423) | (20.481) | (47.506) | (32.489) | |
| Median | −34.60 | −14.38 | −33.84 | −7.30 | −30.00 | |
| Q1, Q3 | −77.50, | −31.92, | −34.60, | −53.75, | −34.60, | |
| | 15.41 | 13.88 | 1.25 | 20.96 | 1.25 | |
| Min, Max | −77.5, 15.4 | −33.8, 26.5 | −34.6, 1.3 | −77.5, 26.5 | −77.5, 26.5 | |

TABLE B-3
Average Duration of Weekly Symptomatic RP Attacks Change and Percent
Change from Baseline by Baseline RCS. ITT Population - Part A.
Average duration (minutes) of weekly symptomatic RP attacks is defined as the total
duration of symptomatic RP attacks divided by total number of symptomatic RP attacks
within the last 7 days of the dosing period (excluding the last dosing day). Baseline is
calculated as total duration of symptomatic RP attacks during the last 7 days of screening
period (excluding the first dosing day) divided by the total number of symptomatic RP
attacks. Only symptomatic RP attacks, defined as RP attacks with patient reported finger
color changes associated with at least one symptom (pain, numbness, tingling), were
included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 48.38 | 19.86 | | 17.78 | 12.22 | 56.42 |
| (SD) | | (9.232) | | | | (66.350) |
| Median | 48.38 | 19.86 | | 17.78 | 12.22 | 56.42 |
| Q1, Q3 | 48.38, | 13.33, | | 17.78, | 12.22, | 9.50, |
| | 48.38 | 26.39 | | 17.78 | 12.22 | 103.33 |
| Min, Max | 48.4, 48.4 | 13.3, 26.4 | | 17.8, 17.8 | 12.2, 12.2 | 9.5, 103.3 |
| Average | | | | | | |
| Duration | | | | | | |
| n | 1 | 2 | | 1 | 1 | 1 |
| Mean | 31.64 | 16.73 | | 18.00 | 15.46 | 98.08 |
| (SD) | | (19.413) | | | | |
| Median | 31.64 | 16.73 | | 18.00 | 15.46 | 98.08 |
| Q1, Q3 | 31.64, | 3.00, 30.45 | | 18.00, | 15.46, | 98.08, |
| | 31.64 | | | 18.00 | 15.46 | 98.08 |
| Min, Max | 31.6, 31.6 | 3.0, 30.5 | | 18.0, 18.0 | 15.5, 15.5 | 98.1, 98.1 |
| Change | | | | | | |
| from | | | | | | |
| Baseline | | | | | | |
| n | 1 | 2 | | 1 | 1 | 1 |
| Mean | −16.74 | −3.13 | | 0.22 | 3.24 | −5.26 |
| (SD) | | (10.182) | | | | |
| Median | −16.74 | −3.13 | | 0.22 | 3.24 | −5.26 |
| Q1, Q3 | −16.74, | −10.33, | | 0.22, 0.22 | 3.24, 3.24 | −5.26, −5.26 |
| | −16.74 | 4.07 | | | | |
| Min, Max | −16.7, −16.7 | −10.3, 4.1 | | 0.2, 0.2 | 3.2, 3.2 | −5.3, −5.3 |
| Percent | | | | | | |
| Change | | | | | | |
| from | | | | | | |
| Baseline | | | | | | |
| n | 1 | 2 | | 1 | 1 | 1 |
| Mean | −34.60 | −31.05 | | 1.25 | 26.50 | −5.09 |
| (SD) | | (65.695) | | | | |
| Median | −34.60 | −31.05 | | 1.25 | 26.50 | −5.09 |
| Q1, Q3 | −34.60, | −77.50, | | 1.25, 1.25 | 26.50, | −5.09, −5.09 |
| | −34.60 | 15.41 | | | 26.50 | |
| Min, Max | −34.6, −34.6 | −77.5, 15.4 | | 1.3, 1.3 | 26.5, 26.5 | −5.1, −5.1 |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine +30 | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | 3 | 2 | 2 | 3 | 7 | |
| Mean | 29.37 | 15.00 | 33.08 | 17.31 | 32.99 | |
| (SD) | (17.713) | (3.928) | (21.640) | (7.878) | (33.738) | |
| Median | 26.39 | 15.00 | 33.08 | 13.33 | 17.78 | |
| Q1, Q3 | 13.33, | 12.22, | 17.78, | 12.22, | 12.22, | |
| | 48.38 | 17.78 | 48.38 | 26.39 | 48.38 | |
| Min, Max | 13.3, 48.4 | 12.2, 17.8 | 17.8, 48.4 | 12.2, 26.4 | 9.5, 103.3 | |
| Average | | | | | | |
| Duration | | | | | | |
| n | 3 | 2 | 2 | 3 | 6 | |
| Mean | 21.70 | 16.73 | 24.82 | 16.31 | 32.77 | |
| (SD) | (16.205) | (1.795) | (9.647) | (13.747) | (33.692) | |
| Median | 30.45 | 16.73 | 24.82 | 15.46 | 24.23 | |
| Q1, Q3 | 3.00, 31.64 | 15.46, | 18.00, | 3.00, 30.45 | 15.46, | |
| | | 18.00 | 31.64 | | 31.64 | |
| Min, Max | 3.0, 31.6 | 15.5, 18.0 | 18.0, 31.6 | 3.0, 30.5 | 3.0, 98.1 | |
| Change | | | | | | |
| from | | | | | | |
| Baseline | | | | | | |
| n | 3 | 2 | 2 | 3 | 6 | |
| Mean | −7.67 | 1.73 | −8.26 | −1.01 | −4.13 | |
| (SD) | (10.655) | (2.133) | (11.993) | (8.085) | (8.236) | |
| Median | −10.33 | 1.73 | −8.26 | 3.24 | −2.52 | |
| Q1, Q3 | −16.74, | 0.22, 3.24 | −16.74, | −10.33, | −10.33, | |
| | 4.07 | | 0.22 | 4.07 | 3.24 | |
| Min, Max | −16.7, 4.1 | 0.2, 3.2 | −16.7, 0.2 | −10.3, 4.1 | −16.7, 4.1 | |
| Percent | | | | | | |
| Change | | | | | | |
| from | | | | | | |
| Baseline | | | | | | |
| n | 3 | 2 | 2 | 3 | 6 | |
| Mean | −32.23 | 13.88 | −16.67 | −11.86 | −12.34 | |
| (SD) | (46.499) | (17.857) | (25.347) | (57.113) | (38.098) | |
| Median | −34.60 | 13.88 | −16.67 | 15.41 | −1.92 | |
| Q1, Q3 | −77.50, | 1.25, 26.50 | −34.60, | −77.50, | −34.60, | |
| | 15.41 | | 1.25 | 26.50 | 15.41 | |
| Min, Max | −77.5, 15.4 | 1.3, 26.5 | −34.6, 1.3 | −77.5, 26.5 | −77.5, 26.5 | |
| | | | | Cilnidipine | Cilnidipine | |
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 2 | 1 | 1 | |
| Mean | | | 271.54 | 58.57 | 90.00 | |
| (SD) | | | (369.051) | | | |
| Median | | | 271.54 | 58.57 | 90.00 | |
| Q1, Q3 | | | 10.58, | 58.57, | 90.00, | |
| | | | 532.50 | 58.57 | 90.00 | |
| Min, Max | | | 10.6, 532.5 | 58.6, 58.6 | 90.0, 90.0 | |
| Average | | | | | | |
| Duration | | | | | | |
| n | | | 2 | 1 | 1 | |
| Mean | | | 412.29 | 38.75 | 63.00 | |
| (SD) | | | (574.575) | | | |
| Median | | | 412.29 | 38.75 | 63.00 | |
| Q1, Q3 | | | 6.00, | 38.75, | 63.00, | |
| | | | 818.57 | 38.75 | 63.00 | |
| Min, Max | | | 6.0, 818.6 | 38.8, 38.8 | 63.0, 63.0 | |
| Change | | | | | | |
| from | | | | | | |
| Baseline | | | | | | |
| n | | | 2 | 1 | 1 | |
| Mean | | | 140.74 | −19.82 | −27.00 | |
| (SD) | | | (205.524) | | | |
| Median | | | 140.74 | −19.82 | −27.00 | |
| Q1, Q3 | | | −4.58, | −19.82, | −27.00, | |
| | | | 286.07 | −19.82 | −27.00 | |
| Min, Max | | | −4.6, 286.1 | −19.8, −19.8 | −27.0, −27.0 | |
| Percent | | | | | | |
| Change | | | | | | |
| from | | | | | | |
| Baseline | | | | | | |
| n | | | 2 | 1 | 1 | |
| Mean | | | 5.21 | −33.84 | −30.00 | |
| (SD) | | | (68.610) | | | |
| Median | | | 5.21 | −33.84 | −30.00 | |
| Q1, Q3 | | | −43.31, | −33.84, | −30.00, | |
| | | | 53.72 | −33.84 | −30.00 | |
| Min, Max | | | −43.3, 53.7 | −33.8, −33.8 | −30.0, −30.0 | |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine + | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | | 2 | 1 | 1 | 4 | |
| Mean | | 74.29 | 58.57 | 90.00 | 172.91 | |
| (SD) | | (22.223) | | | (241.938) | |
| Median | | 74.29 | 58.57 | 90.00 | 74.29 | |
| Q1, Q3 | | 58.57, | 58.57, | 90.00, | 34.58, | |
| | | 90.00 | 58.57 | 90.00 | 311.25 | |
| Min, Max | | 58.6, 90.0 | 58.6, 58.6 | 90.0, 90.0 | 10.6, 532.5 | |
| Average | | | | | | |
| Duration | | | | | | |
| n | | 2 | 1 | 1 | 4 | |
| Mean | | 50.88 | 38.75 | 63.00 | 231.58 | |
| (SD) | | (17.147) | | | (392.024) | |
| Median | | 50.88 | 38.75 | 63.00 | 50.88 | |
| Q1, Q3 | | 38.75, | 38.75, | 63.00, | 22.38, | |
| | | 63.00 | 38.75 | 63.00 | 440.79 | |
| Min, Max | | 38.8, 63.0 | 38.8, 38.8 | 63.0, 63.0 | 6.0, 818.6 | |
| Change | | | | | | |
| from | | | | | | |
| Baseline | | | | | | |
| n | | 2 | 1 | 1 | 4 | |
| Mean | | −23.41 | −19.82 | −27.00 | 58.67 | |
| (SD) | | (5.076) | | | (151.891) | |
| Median | | −23.41 | −19.82 | −27.00 | −12.20 | |
| Q1, Q3 | | −27.00, | −19.82, | −27.00, | −23.41, | |
| | | −19.82 | −19.82 | −27.00 | 140.74 | |
| Min, Max | | −27.0, −19.8 | −19.8, −19.8 | −27.0, −27.0 | −27.0, | |
| | | | | | 286.1 | |
| Percent | | | | | | |
| Change | | | | | | |
| from | | | | | | |
| Baseline | | | | | | |
| n | | 2 | 1 | 1 | 4 | |
| Mean | | −31.92 | −33.84 | −30.00 | −13.36 | |
| (SD) | | (2.716) | | | (45.068) | |
| Median | | −31.92 | −33.84 | −30.00 | −31.92 | |
| Q1, Q3 | | −33.84, | −33.84, | −30.00, | −38.57, | |
| | | −30.00 | −33.84 | −30.00 | 11.86 | |
| Min, Max | | −33.8, −30.0 | −33.8, −33.8 | −30.0, −30.0 | −43.3, 53.7 | |

TABLE B-4
Average Duration of Weekly Symptomatic RP Attacks Change and Percent
Change from Baseline (Excluding Outliers) by Baseline RCS. ITT Population-Part A.
Average duration (minutes) of weekly symptomatic RP attacks is defined as the total
of symptomatic RP attacks divided by total number of symptomatic RP attacks
within the last 7 days of the dosing period (excluding the last dosing day). Baseline is
calculated as total duration of symptomatic RP attacks during the last 7 days of screening
period (excluding the first dosing day) divided by the total number of symptomatic RP
attacks. Only symptomatic RP attacks, defined as RP attacks with patient reported finger
color changes associated with at least one symptom (pain, numbness, tingling), were
included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 48.38 | 19.86 | | 17.78 | 12.22 | 56.42 |
| (SD) | | (9.232) | | | | (66.350) |
| Median | 48.38 | 19.86 | | 17.78 | 12.22 | 56.42 |
| Q1, Q3 | 48.38, 48.38 | 13.33, 26.39 | | 17.78, 17.78 | 12.22, 12.22 | 9.50, 103.33 |
| Min, Max | 48.4, 48.4 | 13.3, 26.4 | | 17.8, 17.8 | 12.2, 12.2 | 9.5, 103.3 |
| Average | | | | | | |
| Duration | | | | | | |
| n | 1 | 2 | | 1 | 1 | 1 |
| Mean | 31.64 | 16.73 | | 18.00 | 15.46 | 98.08 |
| (SD) | | (19.413) | | | | |
| Median | 31.64 | 16.73 | | 18.00 | 15.46 | 98.08 |
| Q1, Q3 | 31.64, 31.64 | 3.00, 30.45 | | 18.00, 18.00 | 15.46, 15.46 | 98.08, 98.08 |
| Min, Max | 31.6, 31.6 | 3.0, 30.5 | | 18.0, 18.0 | 15.5, 15.5 | 98.1, 98.1 |
| Change | | | | | | |
| from | | | | | | |
| Baseline | | | | | | |
| n | 1 | 2 | | 1 | 1 | 1 |
| Mean | −16.74 | −3.13 | | 0.22 | 3.24 | −5.26 |
| (SD) | | (10.182) | | | | |
| Median | −16.74 | −3.13 | | 0.22 | 3.24 | −5.26 |
| Q1, Q3 | −16.74, 16.74 | −10.33, 4.07 | | 0.22, 0.22 | 3.24, 3.24 | −5.26, −5.26 |
| Min, Max | −16.7, −16.7 | −10.3, 4.1 | | 0.2, 0.2 | 3.2, 3.2 | −5.3, −5.3 |
| Percent | | | | | | |
| Change | | | | | | |
| from | | | | | | |
| Baseline | | | | | | |
| n | 1 | 2 | | 1 | 1 | 1 |
| Mean | −34.60 | −31.05 | | 1.25 | 26.50 | −5.09 |
| (SD) | | (65.695) | | | | |
| Median | −34.60 | −31.05 | | 1.25 | 26.50 | −5.09 |
| Ql, Q3 | −34.60, −34.60 | −77.50, 15.41 | | 1.25, 1.25 | 26.50, 26.50 | −5.09, −5.09 |
| Min, Max | −34.6, −34.6 | −77.5, 15.4 | | 1.3, 1.3 | 26.5, 26.5 | −5.1, −5.1 |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine | Tadalafil | Tadalafil | All | |
| | Cilnidipine + | Tadalafil | 5 mg | 5 mg | Participants | |
| Statistics | (N = 3) | (N = 4) | (N = 3) | (N = 4) | (N = 11) | |
| Baseline | | | | | | |
| n | 3 | 2 | 2 | 3 | 7 | |
| Mean | 29.37 | 15.00 | 33.08 | 17.31 | 32.99 | |
| (SD) | (17.713) | (3.928) | (21.640) | (7.878) | (33.738) | |
| Median | 26.39 | 15.00 | 33.08 | 13.33 | 17.78 | |
| Q1, Q3 | 13.33, 48.38 | 12.22, 17.78 | 17.78, 48.38 | 12.22, 26.39 | 12.22, 48.38 | |
| Min, Max | 13.3, 48.4 | 12.2, 17.8 | 17.8, 48.4 | 12.2, 26.4 | 9.5, 103.3 | |
| Average | | | | | | |
| Duration | | | | | | |
| n | 3 | 2 | 2 | 3 | 6 | |
| Mean | 21.70 | 16.73 | 24.82 | 16.31 | 32.77 | |
| (SD) | (16.205) | (1.795) | (9.647) | (13.747) | (33.692) | |
| Median | 30.45 | 16.73 | 24.82 | 15.46 | 24.23 | |
| Q1, Q3 | 3.00, 31.64 | 15.46, 18.00 | 18.00, 31.64 | 3.00, 30.45 | 15.46, 31.64 | |
| Min, Max | 3.0, 31.6 | 15.5, 18.0 | 18.0, 31.6 | 3.0, 30.5 | 3.0, 98.1 | |
| Change | | | | | | |
| from | | | | | | |
| Baseline | | | | | | |
| n | 3 | 2 | 2 | 3 | 6 | |
| Mean | −7.67 | 1.73 | −8.26 | −1.01 | −4.13 | |
| (SD) | (10.655) | (2.133) | (11.993) | (8.085) | (8.236) | |
| Median | −10.33 | 1.73 | −8.26 | 3.24 | −2.52 | |
| Q1, Q3 | −16.74, 4.07 | 0.22, 3.24 | −16.74, 0.22 | −10.33, 4.07 | −10.33, 3.24 | |
| Min, Max | −16.7, 4.1 | 0.2, 3.2 | −16.7, 0.2 | −10.3, 4.1 | −16.7, 4.1 | |
| Percent | | | | | | |
| Change | | | | | | |
| from | | | | | | |
| Baseline | | | | | | |
| n | 3 | 2 | 2 | 3 | 6 | |
| Mean | −32.23 | 13.88 | −16.67 | −11.86 | −12.34 | |
| (SD) | (46.499) | (17.857) | (25.347) | (57.113) | (38.098) | |
| Median | −34.60 | 13.88 | −16.67 | 15.41 | −1.92 | |
| Q1, Q3 | −77.50, 15.41 | 1.25, 26.50 | −34.60, 1.25 | −77.50, 26.50 | −34.60, 15.41 | |
| Min, Max | −77.5, 15.4 | 1.3, 26.5 | −34.6, 1.3 | −77.5, 26.5 | −77.5, 26.5 | |
| | | | | Cilnidipine | Cilnidipine | |
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 1 | 1 | 1 | |
| Mean | | | 10.58 | 58.57 | 90.00 | |
| (SD) | | | | | | |
| Median | | | 10.58 | 58.57 | 90.00 | |
| Q1, Q3 | | | 10.58, 10.58 | 58.57, 58.57 | 90.00, 90.00 | |
| Min, Max | | | 10.6, 10.6 | 58.6, 58.6 | 90.0, 90.0 | |
| Average | | | | | | |
| Duration | | | | | | |
| n | | | 1 | 1 | 1 | |
| Mean | | | 6.00 | 38.75 | 63.00 | |
| (SD) | | | | | | |
| Median | | | 6.00 | 38.75 | 63.00 | |
| Q1, Q3 | | | 6.00, 6.00 | 38.75, 38.75 | 63.00, 63.00 | |
| Min, Max | | | 6.0, 6.0 | 38.8, 38.8 | 63.0, 63.0 | |
| Change | | | | | | |
| from | | | | | | |
| Baseline | | | | | | |
| n | | | 1 | 1 | 1 | |
| Mean | | | −4.58 | −19.82 | −27.00 | |
| (SD) | | | | | | |
| Median | | | −4.58 | −19.82 | −27.00 | |
| Q1, Q3 | | | −4.58, −4.58 | −19.82, −19.82 | −27.00, −27.00 | |
| Min, Max | | | −4.6, −4.6 | −19.8, −19.8 | −27.0, −27.0 | |
| Percent | | | | | | |
| Change | | | | | | |
| from | | | | | | |
| Baseline | | | | | | |
| n | | | 1 | 1 | 1 | |
| Mean | | | −43.31 | −33.84 | −30.00 | |
| (SD) | | | | | | |
| Median | | | −43.31 | −33.84 | −30.00 | |
| Q1, Q3 | | | −43.31, −43.31 | −33.84, −33.84 | −30.00, −30.00 | |
| Min, Max | | | −43.3, −43.3 | −33.8, −33.8 | −30.0, −30.0 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg | 20 mg | |
| | Pooled | Cilnidipine + | Tadalafil + | Tadalafil | All |
| | Cilnidipine + | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 3 |
| Mean | | 74.29 | 58.57 | 90.00 | 53.05 |
| (SD) | | (22.223) | | | (39.995) |
| Median | | 74.29 | 58.57 | 90.00 | 58.57 |
| Q1, Q3 | | 58.57, 90.00 | 58.57, 58.57 | 90.00, 90.00 | 10.58, 90.00 |
| Min, Max | | 58.6, 90.0 | 58.6, 58.6 | 90.0, 90.0 | 10.6, 90.0 |
| Average | | | | | |
| Duration | | | | | |
| n | | 2 | 1 | 1 | 3 |
| Mean | | 50.88 | 38.75 | 63.00 | 35.92 |
| (SD) | | (17.147) | | | (28.605) |
| Median | | 50.88 | 38.75 | 63.00 | 38.75 |
| Q1, Q3 | | 38.75, 63.00 | 38.75, 38.75 | 63.00, 63.00 | 6.00, 63.00 |
| Min, Max | | 38.8, 63.0 | 38.8, 38.8 | 63.0, 63.0 | 6.0, 63.0 |
| Change | | | | | |
| from | | | | | |
| Baseline | | | | | |
| n | | 2 | 1 | 1 | 3 |
| Mean | | −23.41 | −19.82 | −27.00 | −17.13 |
| (SD) | | (5.076) | | | (11.447) |
| Median | | −23.41 | −19.82 | −27.00 | −19.82 |
| Q1, Q3 | | −27.00, −19.82 | −19.82, −19.82 | −27.00, −27.00 | −27.00, −4.58 |
| Min, Max | | −27.0, −19.8 | −19.8, −19.8 | −27.0, −27.0 | −27.0, −4.6 |
| | | | Pooled | Pooled | |
| | | | Cilnidipine | Cilnidipine | |
| | | | 10 mg | 20 mg | |
| | | | and | and | |
| | | | Cilnidipine | Cilnidipine | |
| | | Pooled | 10 mg | 20 mg | |
| | Pooled | Cilnidipine + | Tadalafil + | Tadalafil | All |
| | Cilnidipine + | Tadalafil | 5 mg | 5 mg | Participants |
| Statistics | (N = 3) | (N = 4) | (N = 3) | (N = 4) | (N = 11) |
| Percent | | | | | |
| Change | | | | | |
| from | | | | | |
| Baseline | | | | | |
| n | | 2 | 1 | 1 | 3 |
| Mean | | −31.92 | −33.84 | −30.00 | −35.72 |
| (SD) | | (2.716) | | | (6.849) |
| Median | | −31.92 | −33.84 | −30.00 | −33.84 |
| Q1, Q3 | | −33.84, −30.00 | −33.84, −33.84 | −30.00, −30.00 | −43.31, −30.00 |
| Min, Max | | −33.8, −30.0 | −33.8, −33.8 | −30.0, −30.0 | −43.3, −30.0 |

TABLE B-5
Average Duration of Weekly RP Attacks (All Attacks)
Change and Percent Change from Baseline. ITT Population-Part A.
Average duration (minutes) of weekly RP attacks is defined as the total duration of RP
attacks divided by total number of symptomatic RP attacks within the last 7 days of the
dosing period (excluding the last dosing day). Baseline is calculated as total duration of
RP attacks during the last 7 days of screening period (excluding the first dosing day)
divided by the total number of RP attacks.

| | | | | Cilnidipine | Cilnidipine | |
|---|---|---|---|---|---|---|
| | | | | 10 mg | 20 mg | |
| | Cilnidipine | Cilnidipine | Tadalafil + | Tadalafil + | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 2 | 2 | 2 | 2 |
| Mean | 40.00 | 17.81 | 320.46 | 36.16 | 46.96 | 56.42 |
| (SD) | | (10.165) | (437.753) | (25.999) | (49.294) | (66.350) |
| Median | 40.00 | 17.81 | 320.46 | 36.16 | 46.96 | 56.42 |
| Q1, Q3 | 40.00, 40.00 | 10.63, 25.00 | 10.92, 630.00 | 17.78, 54.55 | 12.11, 81.82 | 9.50, 103.33 |
| Min, Max | 40.0, 40.0 | 10.6, 25.0 | 10.9, 630.0 | 17.8, 54.5 | 12.1, 81.8 | 9.5, 103.3 |
| Average | | | | | | |
| Duration | | | | | | |
| n | 1 | 2 | 2 | 2 | 2 | 1 |
| Mean | 31.52 | 16.73 | 412.26 | 26.08 | 38.50 | 92.33 |
| (SD) | | (19.413) | (574.612) | (11.432) | (34.267) | |
| Median | 31.52 | 16.73 | 412.26 | 26.08 | 38.50 | 92.33 |
| Q1, Q3 | 31.52, 31.52 | 3.00, 30.45 | 5.95, 818.57 | 18.00, 34.17 | 14.27, 62.73 | 92.33, 92.33 |
| Min, Max | 31.5, 31.5 | 3.0, 30.5 | 5.9, 818.6 | 18.0, 34.2 | 14.3, 62.7 | 92.3, 92.3 |
| Change | | | | | | |
| from | | | | | | |
| Baseline | | | | | | |
| n | 1 | 2 | 2 | 2 | 2 | 1 |
| Mean | −8.48 | −1.09 | 91.80 | −10.08 | −8.46 | −11.00 |
| (SD) | | (9.249) | (136.858) | (14.567) | (15.028) | |
| Median | −8.48 | −1.09 | 91.80 | −10.08 | −8.46 | −11.00 |
| Q1, Q3 | −8.48, −8.48 | −7.63, 5.45 | −4.98, 188.57 | −20.38, 0.22 | −19.09, 2.16 | −11.00, −11.00 |
| Min, Max | −8.5, −8.5 | −7.6, 5.5 | −5.0, 188.6 | −20.4, 0.2 | −19.1, 2.2 | −11.0, −11.0 |
| Percent | | | | | | |
| Change | | | | | | |
| from | | | | | | |
| Baseline | | | | | | |
| n | 1 | 2 | 2 | 2 | 2 | 1 |
| Mean | −21.20 | −24.97 | −7.81 | −18.06 | −2.74 | −10.65 |
| (SD) | | (66.173) | (53.375) | (27.302) | (29.125) | |
| Median | −21.20 | −24.97 | −7.81 | −18.06 | −2.74 | −10.65 |
| Ql, Q3 | −21.20, −21.20 | −71.76, 21.82 | −45.55, 29.93 | −37.36, 1.25 | −23.33, 17.86 | −10.65, −10.65 |
| Min, Max | −21.2, −21.2 | −71.8, 21.8 | −45.6, 29.9 | −37.4, 1.3 | −23.3, 17.9 | −10.6, −10.6 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg | 20 mg | |
| | Pooled | Cilnidipine + | Tadalafil + | Tadalafil | All |
| | Cilnidipine + | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 11 |
| Mean | 25.21 | 41.56 | 37.44 | 32.39 | 90.51 |
| (SD) | (14.689) | (32.775) | (18.517) | (33.580) | (181.698) |
| Median | 25.00 | 36.16 | 40.00 | 18.55 | 25.00 |
| Q1, Q3 | 10.63, 40.00 | 14.94, 68.18 | 17.78, 54.55 | 11.37, 53.41 | 10.92, 81.82 |
| Min, Max | 10.6, 40.0 | 12.1, 81.8 | 17.8, 54.5 | 10.6, 81.8 | 9.5, 630.0 |
| Average | | | | | |
| Duration | | | | | |
| n | 3 | 4 | 3 | 4 | 10 |
| Mean | 21.66 | 32.29 | 27.90 | 27.61 | 111.10 |
| (SD) | (16.168) | (22.053) | (8.672) | (25.981) | (250.056) |
| Median | 30.45 | 26.08 | 31.52 | 22.36 | 30.99 |
| Q1, Q3 | 3.00, 31.52 | 16.13, 48.45 | 18.00, 34.17 | 8.63, 46.59 | 14.27, 62.73 |
| Min, Max | 3.0, 31.5 | 14.3, 62.7 | 18.0, 34.2 | 3.0, 62.7 | 3.0, 818.6 |
| Change | | | | | |
| from | | | | | |
| Baseline | | | | | |
| n | 3 | 4 | 3 | 4 | 10 |
| Mean | −3.55 | −9.27 | −9.54 | −4.77 | 12.49 |
| (SD) | (7.809) | (12.119) | (10.342) | (11.043) | (62.441) |
| Median | −7.63 | −9.43 | −8.48 | −2.73 | −6.30 |
| Q1, Q3 | −8.48, 5.45 | −19.73, 1.19 | −20.38, 0.22 | −13.36, 3.81 | −11.00, 2.16 |
| Min, Max | −8.5, 5.5 | −20.4, 2.2 | −20.4, 0.2 | −19.1, 5.5 | −20.4, 188.6 |
| Percent | | | | | |
| Change | | | | | |
| from | | | | | |
| Baseline | | | | | |
| n | 3 | 4 | 3 | 4 | 10 |
| Mean | −23.71 | −10.40 | −19.10 | −13.86 | −13.90 |
| (SD) | (46.842) | (24.686) | (19.390) | (43.671) | (32.469) |
| Median | −21.20 | −11.04 | −21.20 | −2.74 | −15.92 |
| Q1, Q3 | −71.76, 21.82 | −30.35, 9.55 | −7.36, 1.25 | −47.55, 19.84 | −37.36, 17.86 |
| Min, Max | −71.8, 21.8 | −37.4, 17.9 | −37.4, 1.3 | −71.8, 21.8 | −71.8, 29.9 |

TABLE B-6
Average Duration of Weekly RP Attacks (All Attacks) Change and Percent
Change from Baseline (Excluding Outliers). ITT Population-Part A.
Average duration (minutes) of weekly RP attacks is defined as the total duration of RP
attacks divided by total number of symptomatic RP attacks within the last 7 days of the
dosing period (excluding the last dosing day). Baseline is calculated as total duration of
RP attacks during the last 7 days of screening period (excluding the first dosing day)
divided by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 1 | 2 | 2 | 2 |
| Mean | 40.00 | 17.81 | 10.92 | 36.16 | 46.96 | 56.42 |
| (SD) | | (10.165) | | (25.999) | (49.294) | (66.350) |
| Median | 40.00 | 17.81 | 10.92 | 36.16 | 46.96 | 56.42 |
| Q1, Q3 | 40.00, 40.00 | 10.63, 25.00 | 10.92, 10.92 | 17.78, 54.55 | 12.11, 81.82 | 9.50, 103.33 |
| Min, Max | 40.0, 40.0 | 10.6, 25.0 | 10.9, 10.9 | 17.8, 54.5 | 12.1, 81.8 | 9.5, 103.3 |
| Average | | | | | | |
| Duration | | | | | | |
| n | 1 | 2 | 1 | 2 | 2 | 1 |
| Mean | 31.52 | 16.73 | 5.95 | 26.08 | 38.50 | 92.33 |
| (SD) | | (19.413) | | (11.432) | (34.267) | |
| Median | 31.52 | 16.73 | 5.95 | 26.08 | 38.50 | 92.33 |
| Q1, Q3 | 31.52, 31.52 | 3.00, 30.45 | 5.95, 5.95 | 18.00, 34.17 | 14.27, 62.73 | 92.33, 92.33 |
| Min, Max | 31.5, 31.5 | 3.0, 30.5 | 5.9, 5.9 | 18.0, 34.2 | 14.3, 62.7 | 92.3, 92.3 |
| Change | | | | | | |
| from | | | | | | |
| Baseline | | | | | | |
| n | 1 | 2 | 1 | 2 | 2 | 1 |
| Mean | −8.48 | −1.09 | −4.98 | −10.08 | −8.46 | −11.00 |
| (SD) | | (9.249) | | (14.567) | (15.028) | |
| Median | −8.48 | −1.09 | −4.98 | −10.08 | −8.46 | −11.00 |
| Q1, Q3 | −8.48, −8.48 | −7.63, 5.45 | −4.98, −4.98 | −20.38, 0.22 | −19.09, 2.16 | −11.00, −11.00 |
| Min, Max | −8.5, −85 | −7.6, 5.5 | −5.0, 5.0 | −20.4, 0.2 | −19.1, 2.2 | −11.0, −11.0 |
| Percent | | | | | | |
| Change | | | | | | |
| from | | | | | | |
| Baseline | | | | | | |
| n | 1 | 2 | 1 | 2 | 2 | 1 |
| Mean | −21.20 | −24.97 | −45.55 | −18.06 | −2.74 | −10.65 |
| (SD) | | (66.173) | | (27.302) | (29.125) | |
| Median | −21.20 | −24.97 | −45.55 | −18.06 | −2.74 | −10.65 |
| Q1, Q3 | −21.20, −21.20 | −71.76, 21.82 | −45.55, −45.55 | −37.36, 1.25 | −23.33, 17.86 | −10.65, −10.65 |
| Min, Max | −21.2, −21.2 | −71.8, 21.8 | −45.6, −45.6 | −37.4, 1.3 | −23.3, 17.9 | −10.6, −10.6 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 10 |
| Mean | 25.21 | 41.56 | 37.44 | 32.39 | 36.56 |
| (SD) | (14.689) | (32.775) | (18.517) | (33.580) | (33.315) |
| Median | 25.00 | 36.16 | 40.00 | 18.55 | 21.39 |
| Q1, Q3 | 10.63, 40.00 | 14.94, 68.18 | 17.78, 54.55 | 11.37, 53.41 | 10.92, 54.55 |
| Min, Max | 10.6, 40.0 | 12.1, 81.8 | 17.8, 54.5 | 10.6, 81.8 | 9.5, 103.3 |
| Average | | | | | |
| Duration | | | | | |
| n | 3 | 4 | 3 | 4 | 9 |
| Mean | 21.66 | 32.29 | 27.90 | 27.61 | 32.49 |
| (SD) | (16.168) | (22.053) | (8.672) | (25.981) | (28.765) |
| Median | 30.45 | 26.08 | 31.52 | 22.36 | 30.45 |
| Q1, Q3 | 3.00, 31.52 | 16.13, 48.45 | 18.00, 34.17 | 8.63, 46.59 | 14.27, 34.17 |
| Min, Max | 3.0, 31.5 | 14.3, 62.7 | 18.0, 34.2 | 3.0, 62.7 | 3.0, 92.3 |
| Change | | | | | |
| from | | | | | |
| Baseline | | | | | |
| n | 3 | 4 | 3 | 4 | 9 |
| Mean | −3.55 | −9.27 | −9.54 | −4.77 | −7.08 |
| (SD) | (7.809) | (12.119) | (10.342) | (11.043) | (8.937) |
| Median | −7.63 | −9.43 | −8.48 | −2.73 | −7.63 |
| Q1, Q3 | −8.48, 5.45 | −19.73, 1.19 | −20.38, 0.22 | −13.36, 3.81 | −11.00, 0.22 |
| Min, Max | −8.5, 5.5 | −20.4, 2.2 | −20.4, 0.2 | −19.1, 5.5 | −20.4, 5.5 |
| Percent | | | | | |
| Change | | | | | |
| from | | | | | |
| Baseline | | | | | |
| n | 3 | 4 | 3 | 4 | 9 |
| Mean | −23.71 | −10.40 | −9.10 | −13.86 | −18.77 |
| (SD) | (46.842) | (24.686) | (19.390) | (43.671) | (30.318) |
| Median | −21.20 | −11.04 | −21.20 | −2.74 | −21.20 |
| Q1, Q3 | −71.76, 21.82 | −30.35, 9.55 | −7.36, 1.25 | −47.55, 19.84 | −37.36, 1.25 |
| Min, Max | −71.8, 21.8 | −37.4, 17.9 | −37.4, 1.3 | −71.8, 21.8 | −71.8, 21.8 |

TABEL B-7
Average Duration of Weekly RP Attacks (All Attacks) Change and Percent
Change from Baseline by Baseline RCS. ITT Population-Part A.
Average duration (minutes) of weekly RP attacks is defined as the total duration of RP
attacks divided by total number of symptomatic RP attacks within the last 7 days of the
dosing period (excluding the last dosing day). Baseline is calculated as total duration of
RP attacks during the last 7 days of screening period (excluding the first dosing day)
divided by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 40.00 | 17.81 | | 17.78 | 12.11 | 56.42 |
| (SD) | | (10.165) | | | | (66.350) |
| Median | 40.00 | 17.81 | | 17.78 | 12.11 | 56.42 |
| Q1, Q3 | 40.00, 40.00 | 10.63, 25.00 | | 17.78, 17.78 | 12.11, 12.11 | 9.50, 103.33 |
| Min, Max | 40.0, 40.0 | 10.6, 25.0 | | 17.8, 17.8 | 12.1, 12.1 | 9.5, 103.3 |
| Average | | | | | | |
| Duration | | | | | | |
| n | 1 | 2 | | 1 | 1 | 1 |
| Mean | 31.52 | 16.73 | | 18.00 | 14.27 | 92.33 |
| (SD) | | (19.413) | | | | |
| Median | 31.52 | 16.73 | | 18.00 | 14.27 | 92.33 |
| Q1, Q3 | 31.52, 31.52 | 3.00, 30.45 | | 18.00, 18.00 | 14.27, 14.27 | 92.33, 92.33 |
| Min, Max | 31.5, 31.5 | 3.0, 30.5 | | 18.0, 18.0 | 14.3, 14.3 | 92.3, 92.3 |
| Change | | | | | | |
| from | | | | | | |
| Baseline | | | | | | |
| n | 1 | 2 | | 1 | 1 | 1 |
| Mean | −8.48 | −1.09 | | 0.22 | 2.16 | −11.00 |
| (SD) | | (9.249) | | | | |
| Median | −8.48 | −1.09 | | 0.22 | 2.16 | −11.00 |
| Q1, Q3 | −8.48, −8.48 | −7.63, 5.45 | | 0.22, 0.22 | 2.16, 2.16 | −11.00, −11.00 |
| Min, Max | −8.5, −8.5 | −7.6, 5.5 | | 0.2, 0.2 | 2.2, 2.2 | −11.0, −11.0 |
| Percent | | | | | | |
| Change | | | | | | |
| from | | | | | | |
| Baseline | | | | | | |
| n | 1 | 2 | | 1 | 1 | 1 |
| Mean | −21.20 | −24.97 | | 1.25 | 17.86 | −10.65 |
| (SD) | | (66.173) | | | | |
| Median | −21.20 | −24.97 | | 1.25 | 17.86 | −10.65 |
| Q1, Q3 | −21.20, −21.20 | −71.76, 21.82 | | 1.25, 1.25 | 17.86, 17.86 | −10.65, −10.65 |
| Min, Max | −21.2, −21.2 | −71.8, 21.8 | | 1.3, 1.3 | 17.9, 17.9 | −10.6, −10.6 |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine + | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | 3 | 2 | 2 | 3 | 7 | |
| Mean | 25.21 | 14.94 | 28.89 | 15.91 | 31.19 | |
| (SD) | (14.689) | (4.011) | (15.713) | (7.907) | (33.561) | |
| Median | 25.00 | 14.94 | 28.89 | 12.11 | 17.78 | |
| Q1, Q3 | 10.63, 40.00 | 12.11, 17.78 | 17.78, 40.00 | 10.63, 25.00 | 10.63, 40.00 | |
| Min, Max | 10.6, 40.0 | 12.1, 17.8 | 17.8, 40.0 | 10.6, 25.0 | 9.5, 103.3 | |
| Average | | | | | | |
| Duration | | | | | | |
| n | 3 | 2 | 2 | 3 | 6 | |
| Mean | 21.66 | 16.13 | 24.76 | 15.91 | 31.60 | |
| (SD) | (16.168) | (2.640) | (9.561) | (13.801) | (31.601) | |
| Median | 30.45 | 16.13 | 24.76 | 14.27 | 24.23 | |
| Q1, Q3 | 3.00, 31.52 | 14.27, 18.00 | 18.00, 31.52 | 3.00, 30.45 | 14.27, 31.52 | |
| Min, Max | 3.0, 31.5 | 14.3, 18.0 | 18.0, 31.5 | 3.0, 30.5 | 3.0, 92.3 | |
| Change | | | | | | |
| from | | | | | | |
| Baseline | | | | | | |
| n | 3 | 2 | 2 | 3 | 6 | |
| Mean | −3.55 | 1.19 | −4.13 | 0.00 | −3.21 | |
| (SD) | (7.809) | (1.371) | (6.152) | (6.803) | (6.688) | |
| Median | −7.63 | 1.19 | −4.13 | 2.16 | −3.70 | |
| Q1, Q3 | −8.48, 5.45 | 0.22, 2.16 | −8.48, 0.22 | −7.63, 5.45 | −8.48, 2.16 | |
| Min, Max | −8.5, 5.5 | 0.2, 2.2 | −8.5, 0.2 | −7.6, 5.5 | −11.0, 5.5 | |
| Percent | | | | | | |
| Change | | | | | | |
| from | | | | | | |
| Baseline | | | | | | |
| n | 3 | 2 | 2 | 3 | 6 | |
| Mean | −23.71 | 9.55 | −9.97 | −10.70 | −10.45 | |
| (SD) | (46.842) | (11.742) | (15.871) | (52.923) | (34.218) | |
| Median | −21.20 | 9.55 | −9.97 | 17.86 | −4.70 | |
| Q1, Q3 | −71.76, 21.82 | 1.25, 17.86 | −21.20, 1.25 | −71.76, 21.82 | −21.20, 17.86 | |
| Min, Max | −71.8, 21.8 | 1.3, 17.9 | −21.2, 1.3 | −71.8, 21.8 | −71.8, 21.8 | |
| | | | | Cilnidipine | Cilnidipine | |
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 2 | 1 | 1 | |
| Mean | | | 320.46 | 54.55 | 81.82 | |
| (SD) | | | (437.753) | | | |
| Median | | | 320.46 | 54.55 | 81.82 | |
| Q1, Q3 | | | 10.92, 630.00 | 54.55, 54.55 | 81.82, 81.82 | |
| Min, Max | | | 10.9, 630.0 | 54.5, 54.5 | 81.8, 81.8 | |
| Average | | | | | | |
| Duration | | | | | | |
| n | | | 2 | 1 | 1 | |
| Mean | | | 412.26 | 34.17 | 62.73 | |
| (SD) | | | (574.612) | | | |
| Median | | | 412.26 | 34.17 | 62.73 | |
| Q1, Q3 | | | 5.95, 818.57 | 34.17, 34.17 | 62.73, 62.73 | |
| Min, Max | | | 5.9, 818.6 | 34.2, 34.2 | 62.7, 62.7 | |
| Change | | | | | | |
| from | | | | | | |
| Baseline | | | | | | |
| n | | | 2 | 1 | 1 | |
| Mean | | | 91.80 | −20.38 | −19.09 | |
| (SD) | | | (136.858) | | | |
| Median | | | 91.80 | −20.38 | −19.09 | |
| Q1, Q3 | | | −4.98, 188.57 | −20.38, −20.38 | −19.09, −19.09 | |
| Min, Max | | | −5.0, 188.6 | −20.4, −20.4 | −19.1, −19.1 | |
| Percent | | | | | | |
| Change | | | | | | |
| from | | | | | | |
| Baseline | | | | | | |
| n | | | 2 | 1 | 1 | |
| Mean | | | −7.81 | −37.36 | −23.33 | |
| (SD) | | | (53.375) | | | |
| Median | | | −7.81 | −37.36 | −23.33 | |
| Q1, Q3 | | | −45.55, 29.93 | −37.36, −37.36 | −23.33, −23.33 | |
| Min, Max | | | −45.6, 29.9 | −37.4, −37.4 | −23.3, −23.3 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 4 |
| Mean | | 68.18 | 54.55 | 81.82 | 194.32 |
| (SD) | | (19.285) | | | (291.916) |
| Median | | 68.18 | 54.55 | 81.82 | 68.18 |
| Q1, Q3 | | 54.55, 81.82 | 54.55, 54.55 | 81.82, 81.82 | 32.73, 355.91 |
| Min, Max | | 54.5, 81.8 | 54.5, 54.5 | 81.8, 81.8 | 10.9, 630.0 |
| Average | | | | | |
| Duration | | | | | |
| n | | 2 | 1 | 1 | 4 |
| Mean | | 48.45 | 34.17 | 62.73 | 230.35 |
| (SD) | | (20.195) | | | (392.830) |
| Median | | 48.45 | 34.17 | 62.73 | 48.45 |
| Q1, Q3 | | 34.17, 62.73 | 34.17, 34.17 | 62.73, 62.73 | 20.06, 440.65 |
| Min, Max | | 34.2, 62.7 | 34.2, 34.2 | 62.7, 62.7 | 5.9, 818.6 |
| Change | | | | | |
| from | | | | | |
| Baseline | | | | | |
| n | | 2 | 1 | 1 | 4 |
| Mean | | −19.73 | −20.38 | −19.09 | 36.03 |
| (SD) | | (0.911) | | | (101.932) |
| Median | | −19.73 | −20.38 | −19.09 | −12.03 |
| Q1, Q3 | | −20.38, −19.09 | −20.38, −20.38 | −19.09, −19.09 | −19.73, 91.80 |
| Min, Max | | −20.4, −19.1 | −20.4, −20.4 | −19.1, −19.1 | −20.4, 188.6 |
| Percent | | | | | |
| Change | | | | | |
| from | | | | | |
| Baseline | | | | | |
| n | | 2 | 1 | 1 | 4 |
| Mean | | −30.35 | −37.36 | −23.33 | −19.08 |
| (SD) | | (9.919) | | | (33.937) |
| Median | | −30.35 | −37.36 | −23.33 | −30.35 |
| Q1, Q3 | | −37.36, −23.33 | −37.36, −37.36 | −23.33, −23.33 | −41.46, 3.30 |
| Min, Max | | −37.4, −23.3 | −37.4, −37.4 | −23.3, −23.3 | −45.6, 29.9 |

TABLE B-8
Average Duration of Weekly RP Attacks (All Attacks) Change and Percent
Change from Baseline (Excluding Outliers) by Baseline RCS. ITT Population-Part A.
Average duration (minutes) of weekly RP attacks is defined as the total duration of RP
attacks divided by total number of symptomatic RP attacks within the last 7 days of the
dosing period (excluding the last dosing day). Baseline is calculated as total duration of
RP attacks during the last 7 days of screening period (excluding the first dosing day)
divided by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 40.00 | 17.81 | | 17.78 | 12.11 | 56.42 |
| (SD) | | (10.165) | | | | (66.350) |
| Median | 40.00 | 17.81 | | 17.78 | 12.11 | 56.42 |
| Q1, Q3 | 40.00, 40.00 | 10.63, 25.00 | | 17.78, 17.78 | 12.11, 12.11 | 9.50, 103.33 |
| Min, Max | 40.0, 40.0 | 10.6, 25.0 | | 17.8, 17.8 | 12.1, 12.1 | 9.5, 103.3 |
| Average | | | | | | |
| Duration | | | | | | |
| n | 1 | 2 | | 1 | 1 | 1 |
| Mean | 31.52 | 16.73 | | 18.00 | 14.27 | 92.33 |
| (SD) | | (19.413) | | | | |
| Median | 31.52 | 16.73 | | 18.00 | 14.27 | 92.33 |
| Q1, Q3 | 31.52, 31.52 | 3.00, 30.45 | | 18.00, 18.00 | 14.27, 14.27 | 92.33, 92.33 |
| Min, Max | 31.5, 31.5 | 3.0, 30.5 | | 18.0, 18.0 | 14.3, 14.3 | 92.3, 92.3 |
| Change | | | | | | |
| from | | | | | | |
| Baseline | | | | | | |
| n | 1 | 2 | | 1 | 1 | 1 |
| Mean | −8.48 | −1.09 | | 0.22 | 2.16 | −11.00 |
| (SD) | | (9.249) | | | | |
| Median | −8.48 | −1.09 | | 0.22 | 2.16 | −11.00 |
| Q1, Q3 | −8.48, −8.48 | −7.63, 5.45 | | 0.22, 0.22 | 2.16, 2.16 | −11.00, −11.00 |
| Min, Max | −8.5, −8.5 | −7.6, 5.5 | | 0.2, 0.2 | 2.2, 2.2 | −11.0, −11.0 |
| Percent | | | | | | |
| Change | | | | | | |
| from | | | | | | |
| Baseline | | | | | | |
| n | 1 | 2 | | 1 | 1 | 1 |
| Mean | −21.20 | −24.97 | | 1.25 | 17.86 | −10.65 |
| (SD) | | (66.173) | | | | |
| Median | −21.20 | −24.97 | | 1.25 | 17.86 | −10.65 |
| Q1, Q3 | −21.20, −21.20 | −71.76, 21.82 | | 1.25, 1.25 | 17.86, 17.86 | −10.65, −10.65 |
| Min, Max | −21.2, −21.2 | −71.8, 21.8 | | 1.3, 1.3 | 17.9, 17.9 | −10.6, −10.6 |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine + | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | 3 | 2 | 2 | 3 | 7 | |
| Mean | 25.21 | 14.94 | 28.89 | 15.91 | 31.19 | |
| (SD) | (14.689) | (4.011) | (15.713) | (7.907) | (33.561) | |
| Median | 25.00 | 14.94 | 28.89 | 12.11 | 17.78 | |
| Q1, Q3 | 10.63, 40.00 | 12.11, 17.78 | 17.78, 40.00 | 10.63, 25.00 | 10.63, 40.00 | |
| Min, Max | 10.6, 40.0 | 12.1, 17.8 | 17.8, 40.0 | 10.6, 25.0 | 9.5, 103.3 | |
| Average | | | | | | |
| Duration | | | | | | |
| n | 3 | 2 | 2 | 3 | 6 | |
| Mean | 21.66 | 16.13 | 24.76 | 15.91 | 31.60 | |
| (SD) | (16.168) | (2.640) | (9.561) | (13.801) | (31.601) | |
| Median | 30.45 | 16.13 | 24.76 | 14.27 | 24.23 | |
| Q1, Q3 | 3.00, 31.52 | 14.27, 18.00 | 18.00, 31.52 | 3.00, 30.45 | 14.27, 31.52 | |
| Min, Max | 3.0, 31.5 | 14.3, 18.0 | 18.0, 31.5 | 3.0, 30.5 | 3.0, 92.3 | |
| Change | | | | | | |
| from | | | | | | |
| Baseline | | | | | | |
| n | 3 | 2 | 2 | 3 | 6 | |
| Mean | −3.55 | 1.19 | −4.13 | 0.00 | −3.21 | |
| (SD) | (7.809) | (1.371) | (6.152) | (6.803) | (6.688) | |
| Median | −7.63 | 1.19 | −4.13 | 2.16 | −3.70 | |
| Q1, Q3 | −8.48, 5.45 | 0.22, 2.16 | −8.48, 0.22 | −7.63, 5.45 | −8.48, 2.16 | |
| Min, Max | −8.5, 5.5 | 0.2, 2.2 | −8.5, 0.2 | −7.6, 5.5 | −11.0, 5.5 | |
| Percent | | | | | | |
| Change | | | | | | |
| from | | | | | | |
| Baseline | | | | | | |
| n | 3 | 2 | 2 | 3 | 6 | |
| Mean | −23.71 | 9.55 | −9.97 | −10.70 | −10.45 | |
| (SD) | (46.842) | (11.742) | (15.871) | (52.923) | (34.218) | |
| Median | −21.20 | 9.55 | −9.97 | 17.86 | −4.70 | |
| Q1, Q3 | −71.76, 21.82 | 1.25, 17.86 | −21.20, 1.25 | −71.76, 21.82 | −21.20, 17.86 | |
| Min, Max | −71.8, 21.8 | 1.3, 17.9 | −21.2, 1.3 | −71.8, 21.8 | −71.8, 21.8 | |
| | | | | Cilnidipine | Cilnidipine | |
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 1 | 1 | 1 | |
| Mean | | | 10.92 | 54.55 | 81.82 | |
| (SD) | | | | | | |
| Median | | | 10.92 | 54.55 | 81.82 | |
| Q1, Q3 | | | 10.92, 10.92 | 54.55, 54.55 | 81.82, 81.82 | |
| Min, Max | | | 10.9, 10.9 | 54.5, 54.5 | 81.8, 81.8 | |
| Average | | | | | | |
| Duration | | | | | | |
| n | | | 1 | 1 | 1 | |
| Mean | | | 5.95 | 34.17 | 62.73 | |
| (SD) | | | | | | |
| Median | | | 5.95 | 34.17 | 62.73 | |
| Q1, Q3 | | | 5.95, 5.95 | 34.17, 34.17 | 62.73, 62.73 | |
| Min, Max | | | 5.9, 5.9 | 34.2, 34.2 | 62.7, 62.7 | |
| Change | | | | | | |
| from | | | | | | |
| Baseline | | | | | | |
| n | | | 1 | 1 | 1 | |
| Mean | | | −4.98 | −20.38 | −19.09 | |
| (SD) | | | | | | |
| Median | | | −4.98 | −20.38 | −19.09 | |
| Q1, Q3 | | | −4.98, −4.98 | −20.38, −20.38 | −19.09, −19.09 | |
| Min, Max | | | −5.0, −5.0 | −20.4, −20.4 | −19.1, −19.1 | |
| Percent | | | | | | |
| Change | | | | | | |
| from | | | | | | |
| Baseline | | | | | | |
| n | | | 1 | 1 | 1 | |
| Mean | | | −45.55 | −37.36 | −23.33 | |
| (SD) | | | | | | |
| Median | | | −45.55 | −37.36 | −23.33 | |
| Q1, Q3 | | | −45.55, −45.55 | −37.36, −37.36 | −23.33, −23.33 | |
| Min, Max | | | −45.6, −45.6 | −37.4, −37.4 | −23.3, −23.3 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 3 |
| Mean | | 68.18 | 54.55 | 81.82 | 49.10 |
| (SD) | | (19.285) | | | (35.760) |
| Median | | 68.18 | 54.55 | 81.82 | 54.55 |
| Q1, Q3 | | 54.55, 81.82 | 54.55, 54.55 | 81.82, 81.82 | 10.92, 81.82 |
| Min, Max | | 54.5, 81.8 | 54.5, 54.5 | 81.8, 81.8 | 10.9, 81.8 |
| Average | | | | | |
| Duration | | | | | |
| n | | 2 | 1 | 1 | 3 |
| Mean | | 48.45 | 34.17 | 62.73 | 34.28 |
| (SD) | | (20.195) | | | (28.390) |
| Median | | 48.45 | 34.17 | 62.73 | 34.17 |
| Q1, Q3 | | 34.17, 62.73 | 34.17, 34.17 | 62.73, 62.73 | 5.95, 62.73 |
| Min, Max | | 34.2, 62.7 | 34.2, 34.2 | 62.7, 62.7 | 5.9, 62.7 |
| Change | | | | | |
| from | | | | | |
| Baseline | | | | | |
| n | | 2 | 1 | 1 | 3 |
| Mean | | −19.73 | −20.38 | −19.09 | −14.82 |
| (SD) | | (0.911) | | | (8.545) |
| Median | | −19.73 | −20.38 | −19.09 | −19.09 |
| Q1, Q3 | | −20.38, −19.09 | −20.38, −20.38 | −19.09, −19.09 | −20.38, −4.98 |
| Min, Max | | −20.4, −19.1 | −20.4, −20.4 | −19.1, −19.1 | −20.4, −5.0 |
| Percent | | | | | |
| Change | | | | | |
| from | | | | | |
| Baseline | | | | | |
| n | | 2 | 1 | 1 | 3 |
| Mean | | −30.35 | −37.36 | −23.33 | −35.42 |
| (SD) | | (9.919) | | | (11.237) |
| Median | | −30.35 | −37.36 | −23.33 | −37.36 |
| Q1, Q3 | | −37.36, −23.33 | −37.36, −37.36 | −23.33, −23.33 | −45.55, −23.33 |
| Min, Max | | −37.4, −23.3 | −37.4, −37.4 | −23.3, −23.3 | −45.6, −23.3 |

Tables C-1 to C-8 include data that relates to the severity of symptomatic Raynaud's attacks.

TABLE C-1
Average Severity of Weekly Symptomatic RP Attacks Change and Percent Change from Baseline.
ITT Population - Part A.
Average severity (0-10) of weekly symptomatic RP attacks is defined as the total severity scores
of symptomatic RP attacks divided by total number of symptomatic RP attacks within the last 7
days of the dosing period (excluding the last dosing day). Baseline is calculated as total severity
scores of symptomatic RP attacks during the last 7 days of screening period (excluding the first
dosing day) divided by the total number of symptomatic RP attacks. Only symptomatic RP attacks,
defined as RP attacks with patient reported finger color changes associated with at least one
symptom (pain, numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 2 | 2 | 2 | 2 |
| Mean | 3.49 | 3.74 | 7.68 | 5.99 | 5.56 | 3.82 |
| (SD) | | (1.430) | (2.398) | (0.520) | (1.823) | (0.055) |
| Median | 3.49 | 3.74 | 7.68 | 5.99 | 5.56 | 3.82 |
| Q1, Q3 | 3.49, 3.49 | 2.73, 4.75 | 5.98, 9.38 | 5.62, 6.36 | 4.27, 6.84 | 3.78, 3.86 |
| Min, Max | 3.5, 3.5 | 2.7, 4.8 | 6.0, 9.4 | 5.6, 6.4 | 4.3, 6.8 | 3.8, 3.9 |
| Average | | | | | | |
| Severity | | | | | | |
| n | 1 | 2 | 2 | 2 | 2 | 2 |
| Mean | 3.77 | 2.30 | 7.08 | 4.06 | 4.51 | 3.43 |
| (SD) | | (0.141) | (3.406) | (1.644) | (0.774) | (0.601) |
| Median | 3.77 | 2.30 | 7.08 | 4.06 | 4.51 | 3.43 |
| Q1, Q3 | 3.77, 3.77 | 2.20, 2.40 | 4.67, 9.48 | 2.90, 5.23 | 3.96, 5.06 | 3.00, 3.85 |
| Min, Max | 3.8, 3.8 | 2.2, 2.4 | 4.7, 9.5 | 2.9, 5.2 | 4.0, 5.1 | 3.0, 3.9 |
| Change from | | | | | | |
| Baseline | | | | | | |
| n | 1 | 2 | 2 | 2 | 2 | 2 |
| Mean | 0.28 | −1.44 | −0.60 | −1.93 | −1.05 | −0.39 |
| (SD) | | (1.571) | (1.008) | (1.124) | (1.049) | (0.656) |
| Median | 0.28 | −1.44 | −0.60 | −1.93 | −1.05 | −0.39 |
| Q1, Q3 | 0.28, 0.28 | −2.55, −0.33 | −1.32, 0.11 | −2.72, −1.13 | −1.79, −0.31 | −0.86, 0.07 |
| Min, Max | 0.3, 0.3 | −2.6, −0.3 | −1.3, 0.1 | −2.7, −1.1 | −1.8, −0.3 | −0.9, 0.1 |
| Percent Change | | | | | | |
| from Baseline | | | | | | |
| n | 1 | 2 | 2 | 2 | 2 | 2 |
| Mean | 8.05 | −32.85 | −10.43 | −33.11 | −16.64 | −10.20 |
| (SD) | | (29.464) | (16.377) | (21.645) | (13.424) | (17.040) |
| Median | 8.05 | −32.85 | −10.43 | −33.11 | −16.64 | −10.20 |
| Q1, Q3 | 8.05, 8.05 | −53.68, −12.02 | −22.01, 1.16 | −48.42, −17.81 | −26.14, −7.15 | −22.25, 1.85 |
| Min, Max | 8.0, 8.0 | −53.7, −12.0 | −22.0, 1.2 | −48.4, −17.8 | −26.1, −7.2 | −22.2, 1.9 |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 11 |
| Mean | 3.66 | 5.77 | 5.16 | 4.65 | 5.19 |
| (SD) | (1.021) | (1.123) | (1.489) | (1.700) | (1.902) |
| Median | 3.49 | 5.99 | 5.62 | 4.51 | 4.75 |
| Q1, Q3 | 2.73, 4.75 | 4.94, 6.60 | 3.49, 6.36 | 3.50, 5.80 | 3.78, 6.36 |
| Min, Max | 2.7, 4.8 | 4.3, 6.8 | 3.5, 6.4 | 2.7, 6.8 | 2.7, 9.4 |
| Average | | | | | |
| Severity | | | | | |
| n | 3 | 4 | 3 | 4 | 11 |
| Mean | 2.79 | 4.29 | 3.97 | 3.40 | 4.23 |
| (SD) | (0.855) | (1.080) | (1.175) | (1.354) | (2.016) |
| Median | 2.40 | 4.51 | 3.77 | 3.18 | 3.85 |
| Q1, Q3 | 2.20, 3.77 | 3.43, 5.14 | 2.90, 5.23 | 2.30, 4.51 | 2.90, 5.06 |
| Min, Max | 2.2, 3.8 | 2.9, 5.2 | 2.9, 5.2 | 2.2, 5.1 | 2.2, 9.5 |
| Change from | | | | | |
| Baseline | | | | | |
| n | 3 | 4 | 3 | 4 | 11 |
| Mean | −0.87 | −1.49 | −1.19 | −1.24 | −0.96 |
| (SD) | (1.490) | (1.023) | (1.502) | (1.114) | (1.052) |
| Median | −0.33 | −1.46 | −1.13 | −1.06 | −0.86 |
| Q1, Q3 | −2.55, 0.28 | −2.26, −0.72 | −2.72, 0.28 | −2.17, −0.32 | −1.79, 0.07 |
| Min, Max | −2.6, 0.3 | −2.7, −0.3 | −2.7, 0.3 | −2.6, −0.3 | −2.7, 0.3 |
| Percent Change | | | | | |
| from Baseline | | | | | |
| n | 3 | 4 | 3 | 4 | 11 |
| Mean | −19.22 | −24.88 | −19.39 | −24.75 | −18.04 |
| (SD) | (31.490) | (17.512) | (28.267) | (20.904) | (19.728) |
| Median | −12.02 | −21.97 | −17.81 | −19.08 | −17.81 |
| Q1, Q3 | −53.68, 8.05 | −37.28, −12.48 | −48.42, 8.05 | −39.91, −9.58 | −26.14, 1.16 |
| Min, Max | −53.7, 8.0 | −48.4, −7.2 | −48.4, 8.0 | −53.7, −7.2 | −53.7, 8.0 |

TABLE C-2
Average Severity of Weekly Symptomatic RP Attacks Change and Percent Change from Baseline
(Excluding Outliers). ITT Population - Part A.
Average severity (0-10) of weekly symptomatic RP attacks is defined as the total severity scores of
symptomatic RP attacks divided by total number of symptomatic RP attacks within the last 7 days of the
dosing period (excluding the last dosing day). Baseline is calculated as total severity scores of symptomatic
RP attacks during the last 7 days of screening period (excluding the first dosing day) divided by the total
number of symptomatic RP attacks. Only symptomatic RP attacks, defined as RP attacks with patient
reported finger color changes associated with at least one symptom (pain, numbness, tingling), were
included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 1 | 2 | 2 | 2 |
| Mean | 3.49 | 3.74 | 5.98 | 5.99 | 5.56 | 3.82 |
| (SD) | | (1.430) | | (0.520) | (1.823) | (0.055) |
| Median | 3.49 | 3.74 | 5.98 | 5.99 | 5.56 | 3.82 |
| Q1, Q3 | 3.49, 3.49 | 2.73, 4.75 | 5.98, 5.98 | 5.62, 6.36 | 4.27, 6.84 | 3.78, 3.86 |
| Min, Max | 3.5, 3.5 | 2.7, 4.8 | 6.0, 6.0 | 5.6, 6.4 | 4.3, 6.8 | 3.8, 3.9 |
| Average | | | | | | |
| Severity | | | | | | |
| n | 1 | 2 | 1 | 2 | 2 | 2 |
| Mean | 3.77 | 2.30 | 4.67 | 4.06 | 4.51 | 3.43 |
| (SD) | | (0.141) | | (1.644) | (0.774) | (0.601) |
| Median | 3.77 | 2.30 | 4.67 | 4.06 | 4.51 | 3.43 |
| Q1, Q3 | 3.77, 3.77 | 2.20, 2.40 | 4.67, 4.67 | 2.90, 5.23 | 3.96, 5.06 | 3.00, 3.85 |
| Min, Max | 3.8, 3.8 | 2.2, 2.4 | 4.7, 4.7 | 2.9, 5.2 | 4.0, 5.1 | 3.0, 3.9 |
| Change from | | | | | | |
| Baseline | | | | | | |
| n | 1 | 2 | 1 | 2 | 2 | 2 |
| Mean | 0.28 | −1.44 | −1.32 | −1.93 | −1.05 | −0.39 |
| (SD) | | (1.571) | | (1.124) | (1.049) | (0.656) |
| Median | 0.28 | −1.44 | −1.32 | −1.93 | −1.05 | −0.39 |
| Q1, Q3 | 0.28, 0.28 | −2.55, −0.33 | −1.32, −1.32 | −2.72, −1.13 | −1.79, −0.31 | −0.86, 0.07 |
| Min, Max | 0.3, 0.3 | −2.6, −0.3 | −1.3, −1.3 | −2.7, −1.1 | −1.8, −0.3 | −0.9, 0.1 |
| Percent Change | | | | | | |
| from Baseline | | | | | | |
| n | 1 | 2 | 1 | 2 | 2 | 2 |
| Mean | 8.05 | −32.85 | −22.01 | −33.11 | −16.64 | −10.20 |
| (SD) | | (29.464) | | (21.645) | (13.424) | (17.040) |
| Median | 8.05 | −32.85 | −22.01 | −33.11 | −16.64 | −10.20 |
| Q1, Q3 | 8.05, 8.05 | −53.68, −12.02 | −22.01, −22.01 | −48.42, −17.81 | −26.14, −7.15 | −22.25, 1.85 |
| Min, Max | 8.0, 8.0 | −53.7, −12.0 | −22.0, −22.0 | −48.4, −17.8 | −26.1, −7.2 | −22.2, 1.9 |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 10 |
| Mean | 3.66 | 5.77 | 5.16 | 4.65 | 4.77 |
| (SD) | (1.021) | (1.123) | (1.489) | (1.700) | (1.370) |
| Median | 3.49 | 5.99 | 5.62 | 4.51 | 4.51 |
| Q1, Q3 | 2.73, 4.75 | 4.94, 6.60 | 3.49, 6.36 | 3.50, 5.80 | 3.78, 5.98 |
| Min, Max | 2.7, 4.8 | 4.3, 6.8 | 3.5, 6.4 | 2.7, 6.8 | 2.7, 6.8 |
| Average | | | | | |
| Severity | | | | | |
| n | 3 | 4 | 3 | 4 | 10 |
| Mean | 2.79 | 4.29 | 3.97 | 3.40 | 3.70 |
| (SD) | (0.855) | (1.080) | (1.175) | (1.354) | (1.068) |
| Median | 2.40 | 4.51 | 3.77 | 3.18 | 3.81 |
| Q1, Q3 | 2.20, 3.77 | 3.43, 5.14 | 2.90, 5.23 | 2.30, 4.51 | 2.90, 4.67 |
| Min, Max | 2.2, 3.8 | 2.9, 5.2 | 2.9, 5.2 | 2.2, 5.1 | 2.2, 5.2 |
| Change from | | | | | |
| Baseline | | | | | |
| n | 3 | 4 | 3 | 4 | 10 |
| Mean | −0.87 | −1.49 | −1.19 | −1.24 | −1.07 |
| (SD) | (1.490) | (1.023) | (1.502) | (1.114) | (1.044) |
| Median | −0.33 | −1.46 | −1.13 | −1.06 | −1.00 |
| Q1, Q3 | −2.55, 0.28 | −2.26, −0.72 | −2.72, 0.28 | −2.17, −0.32 | −1.79, −0.31 |
| Min, Max | −2.6, 0.3 | −2.7, −0.3 | −2.7, 0.3 | −2.6, −0.3 | −2.7, 0.3 |
| Percent Change | | | | | |
| from Baseline | | | | | |
| n | 3 | 4 | 3 | 4 | 10 |
| Mean | −19.22 | −24.88 | −19.39 | −24.75 | −19.96 |
| (SD) | (31.490) | (17.512) | (28.267) | (20.904) | (19.683) |
| Median | −12.02 | −21.97 | −17.81 | −19.08 | −19.91 |
| Q1, Q3 | −53.68, 8.05 | −37.28, −12.48 | −48.42, 8.05 | −39.91, −9.58 | −26.14, −7.15 |
| Min, Max | −53.7, 8.0 | −48.4, −7.2 | −48.4, 8.0 | −53.7, −7.2 | −53.7, 8.0 |

TABLE C-3
Average Severity of Weekly Symptomatic RP Attacks Change and Percent Change from Baseline by Baseline
RCS. ITT Population - Part A.
Average severity (0-10) of weekly symptomatic RP attacks is defined as the total severity scores of symptomatic
RP attacks divided by total number of symptomatic RP attacks
within the last 7 days of the dosing period (excluding the last dosing day). Baseline is calculated as total severity
scores of symptomatic RP attacks during the last 7 days of screening period (excluding the first dosing day)
divided by the total number of symptomatic RP attacks. Only symptomatic RP attacks, defined as RP attacks
with patient reported finger color changes associated with at least one symptom (pain, numbness, tingling),
were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 3.49 | 3.74 | | 5.62 | 4.27 | 3.82 |
| (SD) | | (1.430) | | | | (0.055) |
| Median | 3.49 | 3.74 | | 5.62 | 4.27 | 3.82 |
| Q1, Q3 | 3.49, 3.49 | 2.73, 4.75 | | 5.62, 5.62 | 4.27, 4.27 | 3.78, 3.86 |
| Min, Max | 3.5, 3.5 | 2.7, 4.8 | | 5.6, 5.6 | 4.3, 4.3 | 3.8, 3.9 |
| Average | | | | | | |
| Severity | | | | | | |
| n | 1 | 2 | | 1 | 1 | 2 |
| Mean | 3.77 | 2.30 | | 2.90 | 3.96 | 3.43 |
| (SD) | | (0.141) | | | | (0.601) |
| Median | 3.77 | 2.30 | | 2.90 | 3.96 | 3.43 |
| Q1, Q3 | 3.77, 3.77 | 2.20, 2.40 | | 2.90, 2.90 | 3.96, 3.96 | 3.00, 3.85 |
| Min, Max | 3.8, 3.8 | 2.2, 2.4 | | 2.9, 2.9 | 4.0, 4.0 | 3.0, 3.9 |
| Change from | | | | | | |
| Baseline | | | | | | |
| n | 1 | 2 | | 1 | 1 | 2 |
| Mean | 0.28 | −1.44 | | −2.72 | −0.31 | −0.39 |
| (SD) | | (1.571) | | | | (0.656) |
| Median | 0.28 | −1.44 | | −2.72 | −0.31 | −0.39 |
| Q1, Q3 | 0.28, 0.28 | −2.55, −0.33 | | −2.72, −2.72 | −0.31, −0.31 | −0.86, 0.07 |
| Min, Max | 0.3, 0.3 | −2.6, −0.3 | | −2.7, −2.7 | −0.3, −0.3 | −0.9, 0.1 |
| Percent Change | | | | | | |
| from Baseline | | | | | | |
| n | 1 | 2 | | 1 | 1 | 2 |
| Mean | 8.05 | −32.85 | | −48.42 | −7.15 | −10.20 |
| (SD) | | (29.464) | | | | (17.040) |
| Median | 8.05 | −32.85 | | −48.42 | −7.15 | −10.20 |
| Q1, Q3 | 8.05, 8.05 | −53.68, −12.02 | | −48.42, −48.42 | −7.15, −7.15 | −22.25, 1.85 |
| Min, Max | 8.0, 8.0 | −53.7, −12.0 | | −48.4, −48.4 | −7.2, −7.2 | −22.2, 1.9 |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean | 3.66 | 4.94 | 4.56 | 3.91 | 4.07 |
| (SD) | (1.021) | (0.959) | (1.507) | (1.056) | (0.929) |
| Median | 3.49 | 4.94 | 4.56 | 4.27 | 3.86 |
| Q1, Q3 | 2.73, 4.75 | 4.27, 5.62 | 3.49, 5.62 | 2.73, 4.75 | 3.49, 4.75 |
| Min, Max | 2.7, 4.8 | 4.3, 5.6 | 3.5, 5.6 | 2.7, 4.8 | 2.7, 5.6 |
| Average | | | | | |
| Severity | | | | | |
| n | 3 | 2 | 2 | 3 | 7 |
| Mean | 2.79 | 3.43 | 3.34 | 2.85 | 3.15 |
| (SD) | (0.855) | (0.751) | (0.616) | (0.964) | (0.717) |
| Median | 2.40 | 3.43 | 3.34 | 2.40 | 3.00 |
| Q1, Q3 | 2.20, 3.77 | 2.90, 3.96 | 2.90, 3.77 | 2.20, 3.96 | 2.40, 3.85 |
| Min, Max | 2.2, 3.8 | 2.9, 4.0 | 2.9, 3.8 | 2.2, 4.0 | 2.2, 4.0 |
| Change from | | | | | |
| Baseline | | | | | |
| n | 3 | 2 | 2 | 3 | 7 |
| Mean | −0.87 | −1.51 | −1.22 | −1.06 | −0.92 |
| (SD) | (1.490) | (1.709) | (2.124) | (1.290) | (1.229) |
| Median | −0.33 | −1.51 | −1.22 | −0.33 | −0.33 |
| Q1, Q3 | −2.55, 0.28 | −2.72, −0.31 | −2.72, 0.28 | −2.55, −0.31 | −2.55, 0.07 |
| Min, Max | −2.6, 0.3 | −2.7, −0.3 | −2.7, 0.3 | −2.6, −0.3 −2.7, 0.3 | |
| Percent Change | | | | | |
| from Baseline | | | | | |
| n | 3 | 2 | 2 | 3 | 7 |
| Mean | −19.22 | −27.79 | −20.18 | −24.28 | −19.09 |
| (SD) | (31.490) | (29.181) | (39.929) | (25.577) | (23.925) |
| Median | −12.02 | −27.79 | −20.18 | −12.02 | −12.02 |
| Q1, Q3 | −53.68, 8.05 | −48.42, −7.15 | −48.42, 8.05 | −53.68, −7.15 | −48.42, 1.85 |
| Min, Max | −53.7, 8.0 | −48.4, −7.2 | −48.4, 8.0 | −53.7, −7.2 | −53.7, 8.0 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 2 | 1 | 1 | |
| Mean | | | 7.68 | 6.36 | 6.84 | |
| (SD) | | | (2.398) | | | |
| Median | | | 7.68 | 6.36 | 6.84 | |
| Q1, Q3 | | | 5.98, 9.38 | 6.36, 6.36 | 6.84, 6.84 | |
| Min, Max | | | 6.0, 9.4 | 6.4, 6.4 | 6.8, 6.8 | |
| Average | | | | | | |
| Severity | | | | | | |
| n | | | 2 | 1 | 1 | |
| Mean | | | 7.08 | 5.23 | 5.06 | |
| (SD) | | | (3.406) | | | |
| Median | | | 7.08 | 5.23 | 5.06 | |
| Q1, Q3 | | | 4.67, 9.48 | 5.23, 5.23 | 5.06, 5.06 | |
| Min, Max | | | 4.7, 9.5 | 5.2, 5.2 | 5.1, 5.1 | |
| Change from | | | | | | |
| Baseline | | | | | | |
| n | | | 2 | 1 | 1 | |
| Mean | | | −0.60 | −1.13 | −1.79 | |
| (SD) | | | (1.008) | | | |
| Median | | | −0.60 | −1.13 | −1.79 | |
| Q1, Q3 | | | −1.32, 0.11 | −1.13, −1.13 | −1.79, −1.79 | |
| Min, Max | | | −1.3, 0.1 | −1.1, −1.1 | −1.8, −1.8 | |
| Percent Change | | | | | | |
| from Baseline | | | | | | |
| n | | | 2 | 1 | 1 | |
| Mean | | | −10.43 | −17.81 | −26.14 | |
| (SD) | | | (16.377) | | | |
| Median | | | −10.43 | −17.81 | −26.14 | |
| Q1, Q3 | | | −22.01, 1.16 | −17.81, −17.81 | −26.14, −26.14 | |
| Min, Max | | | −22.0, 1.2 | −17.8, −17.8 | −26.1, −26.1 | |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 4 |
| Mean | | 6.60 | 6.36 | 6.84 | 7.14 |
| (SD) | | (0.345) | | | (1.531) |
| Median | | 6.60 | 6.36 | 6.84 | 6.60 |
| Q1, Q3 | | 6.36, 6.84 | 6.36, 6.36 | 6.84, 6.84 | 6.17, 8.11 |
| Min, Max | | 6.4, 6.8 | 6.4, 6.4 | 6.8, 6.8 | 6.0, 9.4 |
| Average | | | | | |
| Severity | | | | | |
| n | | 2 | 1 | 1 | 4 |
| Mean | | 5.14 | 5.23 | 5.06 | 6.11 |
| (SD) | | (0.120) | | | (2.263) |
| Median | | 5.14 | 5.23 | 5.06 | 5.14 |
| Q1, Q3 | | 5.06, 5.23 | 5.23, 5.23 | 5.06, 5.06 | 4.86, 7.35 |
| Min, Max | | 5.1, 5.2 | 5.2, 5.2 | 5.1, 5.1 | 4.7, 9.5 |
| Change from | | | | | |
| Baseline | | | | | |
| n | | 2 | 1 | 1 | 4 |
| Mean | | −1.46 | −1.13 | −1.79 | −1.03 |
| (SD) | | (0.464) | | | (0.809) |
| Median | | −1.46 | −1.13 | −1.79 | −1.22 |
| Q1, Q3 | | −1.79, −1.13 | −1.13, −1.13 | −1.79, −1.79 | −1.55, −0.51 |
| Min, Max | | −1.8, −1.1 | −1.1, −1.1 | −1.8, −1.8 | −1.8, 0.1 |
| Percent Change | | | | | |
| from Baseline | | | | | |
| n | | 2 | 1 | 1 | 4 |
| Mean | | −21.97 | −17.81 | −26.14 | −16.20 |
| (SD) | | (5.888) | | | (12.059) |
| Median | | −21.97 | −17.81 | −26.14 | −19.91 |
| Q1, Q3 | | −26.14, −17.81 | −17.81, −17.81 | −26.14, −26.14 | −24.07, −8.33 |
| Min, Max | | −26.1, −17.8 | −17.8, −17.8 | −26.1, −26.1 | −26.1, 1.2 |

TABLE C-4
Average Severity of Weekly Symptomatic RP Attacks Change and Percent Change from Baseline (Excluding
Outliers) by Baseline RCS. ITT Population - Part A.
Average severity (0-10) of weekly symptomatic RP attacks is defined as the total severity scores of symptomatic
RP attacks divided by total number of symptomatic RP attacks within the last 7 days of the dosing period
(excluding the last dosing day). Baseline is calculated as total severity scores of symptomatic RP attacks during
the last 7 days of screening period (excluding the first dosing day) divided by the total number of symptomatic
RP attacks. Only symptomatic RP attacks, defined as RP attacks with patient reported finger color changes
associated with at least one symptom (pain, numbness, tingling), were included in the derivations.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 3.49 | 3.74 | | 5.62 | 4.27 | 3.82 |
| (SD) | | (1.430) | | | | (0.055) |
| Median | 3.49 | 3.74 | | 5.62 | 4.27 | 3.82 |
| Q1, Q3 | 3.49, 3.49 | 2.73, 4.75 | | 5.62, 5.62 | 4.27, 4.27 | 3.78, 3.86 |
| Min, Max | 3.5, 3.5 | 2.7, 4.8 | | 5.6, 5.6 | 4.3, 4.3 | 3.8, 3.9 |
| Average | | | | | | |
| Severity | | | | | | |
| n | 1 | 2 | | 1 | 1 | 2 |
| Mean | 3.77 | 2.30 | | 2.90 | 3.96 | 3.43 |
| (SD) | | (0.141) | | | | (0.601) |
| Median | 3.77 | 2.30 | | 2.90 | 3.96 | 3.43 |
| Q1, Q3 | 3.77, 3.77 | 2.20, 2.40 | | 2.90, 2.90 | 3.96, 3.96 | 3.00, 3.85 |
| Min, Max | 3.8, 3.8 | 2.2, 2.4 | | 2.9, 2.9 | 4.0, 4.0 | 3.0, 3.9 |
| Change from | | | | | | |
| Baseline | | | | | | |
| n | 1 | 2 | | 1 | 1 | 2 |
| Mean | 0.28 | −1.44 | | −2.72 | −0.31 | −0.39 |
| (SD) | | (1.571) | | | | (0.656) |
| Median | 0.28 | −1.44 | | −2.72 | −0.31 | −0.39 |
| Q1, Q3 | 0.28, 0.28 | −2.55, −0.33 | | −2.72, −2.72 | −0.31, −0.31 | −0.86, 0.07 |
| Min, Max | 0.3, 0.3 | −2.6, −0.3 | | −2.7, −2.7 | −0.3, −0.3 | −0.9, 0.1 |
| Percent Change | | | | | | |
| from Baseline | | | | | | |
| n | 1 | 2 | | 1 | 1 | 2 |
| Mean | 8.05 | −32.85 | | −48.42 | −7.15 | −10.20 |
| (SD) | | (29.464) | | | | (17.040) |
| Median | 8.05 | −32.85 | | −48.42 | −7.15 | −10.20 |
| Q1, Q3 | 8.05, 8.05 | −53.68, −12.02 | | −48.42, −48.42 | −7.15, −7.15 | −22.25, 1.85 |
| Min, Max | 8.0, 8.0 | −53.7, −12.0 | | −48.4, −48.4 | −7.2, −7.2 | −22.2, 1.9 |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 2 | 2 | 3 | 7 |
| Mean | 3.66 | 4.94 | 4.56 | 3.91 | 4.07 |
| (SD) | (1.021) | (0.959) | (1.507) | (1.056) | (0.929) |
| Median | 3.49 | 4.94 | 4.56 | 4.27 | 3.86 |
| Q1, Q3 | 2.73, 4.75 | 4.27, 5.62 | 3.49, 5.62 | 2.73, 4.75 | 3.49, 4.75 |
| Min, Max | 2.7, 4.8 | 4.3, 5.6 | 3.5, 5.6 | 2.7, 4.8 | 2.7, 5.6 |
| Average | | | | | |
| Severity | | | | | |
| n | 3 | 2 | 2 | 3 | 7 |
| Mean | 2.79 | 3.43 | 3.34 | 2.85 | 3.15 |
| (SD) | (0.855) | (0.751) | (0.616) | (0.964) | (0.717) |
| Median | 2.40 | 3.43 | 3.34 | 2.40 | 3.00 |
| Q1, Q3 | 2.20,3.77 | 2.90, 3.96 | 2.90,3.77 | 2.20, 3.96 | 2.40, 3.85 |
| Min, Max | 2.2, 3.8 | 2.9, 4.0 | 2.9, 3.8 | 2.2, 4.0 | 2.2, 4.0 |
| Change from | | | | | |
| Baseline | | | | | |
| n | 3 | 2 | 2 | 3 | 7 |
| Mean | −0.87 | −1.51 | −1.22 | −1.06 | −0.92 |
| (SD) | (1.490) | (1.709) | (2.124) | (1.290) | (1.229) |
| Median | −0.33 | −1.51 | −1.22 | −0.33 | −0.33 |
| Q1, Q3 | −2.55, 0.28 | −2.72, −0.31 | −2.72, 0.28 | −2.55, −0.31 | −2.55, 0.07 |
| Min, Max | −2.6, 0.3 | −2.7, −0.3 | −2.7, 0.3 | −2.6, −0.3 | −2.7, 0.3 |
| Percent Change | | | | | |
| from Baseline | | | | | |
| n | 3 | 2 | 2 | 3 | 7 |
| Mean | −19.22 | −27.79 | −20.18 | −24.28 | −19.09 |
| (SD) | (31.490) | (29.181) | (39.929) | (25.577) | (23.925) |
| Median | −12.02 | −27.79 | −20.18 | −12.02 | −12.02 |
| Q1, Q3 | −53.68, 8.05 | −48.42, −7.15 | −48.42, 8.05 | −53.68, −7.15 | −48.42, 1.85 |
| Min, Max | −53.7, 8.0 | −48.4, −7.2 | −48.4, 8.0 | −53.7, −7.2 | −53.7, 8.0 |

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 1 | 1 | 1 | |
| Mean | | | 5.98 | 6.36 | 6.84 | |
| (SD) | | | | | | |
| Median | | | 5.98 | 6.36 | 6.84 | |
| Q1, Q3 | | | 5.98, 5.98 | 6.36, 6.36 | 6.84, 6.84 | |
| Min, Max | | | 6.0, 6.0 | 6.4, 6.4 | 6.8, 6.8 | |
| Average | | | | | | |
| Severity | | | | | | |
| n | | | 1 | 1 | 1 | |
| Mean | | | 4.67 | 5.23 | 5.06 | |
| (SD) | | | | | | |
| Median | | | 4.67 | 5.23 | 5.06 | |
| Q1, Q3 | | | 4.67, 4.67 | 5.23, 5.23 | 5.06, 5.06 | |
| Min, Max | | | 4.7, 4.7 | 5.2, 5.2 | 5.1, 5.1 | |
| Change from | | | | | | |
| Baseline | | | | | | |
| n | | | 1 | 1 | 1 | |
| Mean | | | −1.32 | −1.13 | −1.79 | |
| (SD) | | | | | | |
| Median | | | −1.32 | −1.13 | −1.79 | |
| Q1, Q3 | | | −1.32, −1.32 | −1.13, −1.13 | −1.79, −1.79 | |
| Min, Max | | | −1.3, −1.3 | −1.1, −1.1 | −1.8, −1.8 | |
| Percent Change | | | | | | |
| from Baseline | | | | | | |
| n | | | 1 | 1 | 1 | |
| Mean | | | −22.01 | −17.81 | −26.14 | |
| (SD) | | | | | | |
| Median | | | −22.01 | −17.81 | −26.14 | |
| Q1, Q3 | | | −22.01, −22.01 | −17.81, −17.81 | −26.14, −26.14 | |
| Min, Max | | | −22.0, −22.0 | −17.8, −17.8 | −26.1, −26.1 | |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 3 |
| Mean | | 6.60 | 6.36 | 6.84 | 6.39 |
| (SD) | | (0.345) | | | (0.432) |
| Median | | 6.60 | 6.36 | 6.84 | 6.36 |
| Q1, Q3 | | 6.36, 6.84 | 6.36, 6.36 | 6.84, 6.84 | 5.98, 6.84 |
| Min, Max | | 6.4, 6.8 | 6.4, 6.4 | 6.8, 6.8 | 6.0, 6.8 |
| Average | | | | | |
| Severity | | | | | |
| n | | 2 | 1 | 1 | 3 |
| Mean | | 5.14 | 5.23 | 5.06 | 4.98 |
| (SD) | | (0.120) | | | (0.286) |
| Median | | 5.14 | 5.23 | 5.06 | 5.06 |
| Q1, Q3 | | 5.06, 5.23 | 5.23, 5.23 | 5.06, 5.06 | 4.67, 5.23 |
| Min, Max | | 5.1, 5.2 | 5.2, 5.2 | 5.1, 5.1 | 4.7, 5.2 |
| Change from | | | | | |
| Baseline | | | | | |
| n | | 2 | 1 | 1 | 3 |
| Mean | | −1.46 | −1.13 | −1.79 | −1.41 |
| (SD) | | (0.464) | | | (0.339) |
| Median | | −1.46 | −1.13 | −1.79 | −1.32 |
| Q1, Q3 | | −1.79, −1.13 | −1.13, −1.13 | −1.79, −1.79 | −1.79, −1.13 |
| Min, Max | | −1.8, −1.1 | −1.1, −1.1 | −1.8, −1.8 | −1.8, −1.1 |
| Percent Change | | | | | |
| from Baseline | | | | | |
| n | | 2 | 1 | 1 | 3 |
| Mean | | −21.97 | −17.81 | −26.14 | −21.98 |
| (SD) | | (5.888) | | | (4.164) |
| Median | | −21.97 | −17.81 | −26.14 | −22.01 |
| Q1, Q3 | | −26.14, −17.81 | −17.81, −17.81 | −26.14, −26.14 | −26.14, −17.81 |
| Min, Max | | −26.1, −17.8 | −17.8, −17.8 | −26.1, −26.1 | −26.1, −17.8 |

TABLE C-5
Average Severity of Weekly RP Attacks (All Attacks) Change and Percent Change from Baseline.
ITT Population - Part A.
Average severity (0-10) of weekly RP attacks is defined as the total severity scores of RP attacks divided
by total number of RP attacks within the last 7 days of the dosing period (excluding the last dosing day).
Baseline is calculated as total severity scores of RP attacks during the last 7 days of screening period
(excluding the first dosing day) divided by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 2 | 2 | 2 | 2 |
| Mean | 3.03 | 3.37 | 7.49 | 5.92 | 5.36 | 3.82 |
| (SD) | | (1.087) | (2.457) | (0.421) | (1.746) | (0.055) |
| Median | 3.03 | 3.37 | 7.49 | 5.92 | 5.36 | 3.82 |
| Q1, Q3 | 3.03, 3.03 | 2.60, 4.14 | 5.75, 9.23 | 5.62, 6.22 | 4.12, 6.59 | 3.78, 3.86 |
| Min, Max | 3.0, 3.0 | 2.6, 4.1 | 5.8, 9.2 | 5.6, 6.2 | 4.1, 6.6 | 3.8, 3.9 |
| Average | | | | | | |
| Severity | | | | | | |
| n | 1 | 2 | 2 | 2 | 2 | 2 |
| Mean | 3.05 | 2.30 | 7.02 | 3.90 | 4.39 | 3.37 |
| (SD) | | (0.141) | (3.479) | (1.414) | (0.943) | (0.676) |
| Median | 3.05 | 2.30 | 7.02 | 3.90 | 4.39 | 3.37 |
| Q1, Q3 | 3.05, 3.05 | 2.20, 2.40 8 | 4.56, 9.4 | 2.90, 4.90 | 3.73, 5.06 | 2.89, 3.85 |
| Min, Max | 3.0, 3.0 | 2.2, 2.4 | 4.6, 9.5 | 2.9, 4.9 | 3.7, 5.1 | 2.9, 3.9 |
| Change from | | | | | | |
| Baseline | | | | | | |
| n | 1 | 2 | 2 | 2 | 2 | 2 |
| Mean | 0.02 | −1.07 | −0.47 | −2.02 | −0.96 | −0.45 |
| (SD) | | (1.229) | (1.022) | (0.993) | (0.804) | (0.732) |
| Median | 0.02 | −1.07 | −0.47 | −2.02 | −0.96 | −0.45 |
| Q1, Q3 | 0.02, 0.02 | −1.94, −0.20 | −1.19, 0.25 | −2.72, −1.32 | −1.53, −0.39 | −0.97, 0.07 |
| Min, Max | 0.0, 0.0 | −1.9, −0.2 | −1.2, 0.3 | −2.7, −1.3 | −1.5, −0.4 | −1.0, 0.1 |
| Percent Change | | | | | | |
| from Baseline | | | | | | |
| n | 1 | 2 | 2 | 2 | 2 | 2 |
| Mean | 0.55 | −27.26 | −8.97 | −34.81 | −16.40 | −11.58 |
| (SD) | | (27.673) | (16.585) | (19.248) | (9.657) | (18.995) |
| Median | 0.55 | −27.26 | −8.97 | −34.81 | −16.40 | −11.58 |
| Q1, Q3 | 0.55, 0.55 | −46.83, −7.69 | −20.69, 2.76 | −48.42, −21.20 | −23.23, −9.57 | −25.01, 1.85 |
| Min, Max | 0.5, 0.5 | −46.8, −7.7 | −20.7, 2.8 | −48.4, −21.2 | −23.2, −9.6 | −25.0, 1.9 |

| | | | Pooled | Pooled | |
|---|---|---|---|---|---|
| | | | Cilnidipine | Cilnidipine | |
| | | | 10 mg and | 20 mg and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 11 |
| Mean | 3.26 | 5.64 | 4.96 | 4.36 | 4.99 |
| (SD) | (0.793) | (1.087) | (1.694) | (1.651) | (1.918) |
| Median | 3.03 | 5.92 | 5.62 | 4.13 | 4.14 |
| Q1, Q3 | 2.60, 4.14 | 4.87, 6.40 | 3.03, 6.22 | 3.36, 5.36 | 3.78, 6.22 |
| Min, Max | 2.6, 4.1 | 4.1, 6.6 | 3.0, 6.2 | 2.6, 6.6 | 2.6, 9.2 |
| Average | | | | | |
| Severity | | | | | |
| n | 3 | 4 | 3 | 4 | 11 |
| Mean | 2.55 | 4.15 | 3.62 | 3.35 | 4.09 |
| (SD) | (0.443) | (1.022) | (1.114) | (1.328) | (2.036) |
| Median | 2.40 | 4.31 | 3.05 | 3.06 | 3.73 |
| Q1, Q3 | 2.20, 3.05 | 3.31, 4.98 | 2.90, 4.90 | 2.30, 4.39 | 2.89, 4.90 |
| Min, Max | 2.2, 3.0 | 2.9, 5.1 | 2.9, 4.9 | 2.2, 5.1 | 2.2, 9.5 |
| Change from | | | | | |
| Baseline | | | | | |
| n | 3 | 4 | 3 | 4 | 11 |
| Mean | −0.71 | −1.49 | −1.34 | −1.02 | −0.90 |
| (SD) | (1.071) | (0.957) | (1.370) | (0.850) | (0.944) |
| Median | −0.20 | −1.42 | −1.32 | −0.96 | −0.97 |
| Q1, Q3 | −1.94, 0.02 | −2.13, −0.86 | −2.72, 0.02 | −1.73, −0.30 | −1.53, 0.02 |
| Min, Max | −1.9, 0.0 | −2.7, −0.4 | −2.7, 0.0 | −1.9, −0.2 | −2.7, 0.3 |
| Percent Change | | | | | |
| from Baseline | | | | | |
| n | 3 | 4 | 3 | 4 | 11 |
| Mean | −17.99 | −25.60 | −23.02 | −21.83 | −17.95 |
| (SD) | (25.311) | (16.357) | (24.534) | (18.046) | (17.879) |
| Median | −7.69 | −22.21 | −21.20 | −16.40 | −20.69 |
| Q1, Q3 | −46.83, 0.55 | −35.82, −15.38 | −48.42, 0.55 | −35.03, −8.63 | −25.01, 0.55 |
| Min, Max | −46.8, 0.5 | −48.4, −9.6 | −48.4, 0.5 | −46.8, −7.7 | −48.4, 2.8 |

TABLE C-6
Average Severity of Weekly RP Attacks (All Attacks) Change and Percent
Change from Baseline (Excluding Outliers). ITT Population-Part A.
Average severity (0-10) of weekly RP attacks is defined as the total severity scores of RP
attacks divided by total number of RP attacks within the last 7 days of the dosing period
(excluding the last dosing day). Baseline is calculated as total severity scores of RP
attacks during the last 7 days of screening period (excluding the first dosing day) divided
by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 1 | 2 | 2 | 2 |
| Mean | 3.03 | 3.37 | 5.75 | 5.92 | 5.36 | 3.82 |
| (SD) | | (1.087) | | (0.421) | (1.746) | (0.055) |
| Median | 3.03 | 3.37 | 5.75 | 5.92 | 5.36 | 3.82 |
| Q1, Q3 | 3.03, 3.03 | 2.60, 4.14 | 5.75, 5.75 | 5.62, 6.22 | 4.12, 6.59 | 3.78, 3.86 |
| Min, Max | 3.0, 3.0 | 2.6, 4.1 | 5.8, 5.8 | 5.6, 6.2 | 4.1, 6.6 | 3.8, 3.9 |
| Average | | | | | | |
| Severity | | | | | | |
| n | 1 | 2 | 1 | 2 | 2 | 2 |
| Mean | 3.05 | 2.30 | 4.56 | 3.90 | 4.39 | 3.37 |
| (SD) | | (0.141) | | (1.414) | (0.943) | (0.676) |
| Median | 3.05 | 2.30 | 4.56 | 3.90 | 4.39 | 3.37 |
| Q1, Q3 | 3.05, 3.05 | 2.20, 2.40 | 4.56, 4.56 | 2.90, 4.90 | 3.73, 5.06 | 2.89, 3.85 |
| Min, Max | 3.0, 3.0 | 2.2, 2.4 | 4.6, 4.6 | 2.9, 4.9 | 3.7, 5.1 | 2.9, 3.9 |
| Change | | | | | | |
| from | | | | | | |
| Baseline | | | | | | |
| n | 1 | 2 | 1 | 2 | 2 | 2 |
| Mean | 0.02 | −1.07 | −1.19 | −2.02 | −0.96 | −0.45 |
| (SD) | | (1.229) | | (0.993) | (0.804) | (0.732) |
| Median | 0.02 | −1.07 | −1.19 | −2.02 | −0.96 | −0.45 |
| Q1, Q3 | 0.02, 0.02 | −1.94, −0.20 | −1.19, −1.19 | −2.72, −1.32 | −1.53, −0.39 | −0.97, 0.07 |
| Min, Max | 0.0, 0.0 | −1.9, −0.2 | −1.2, −1.2 | −2.7, −1.3 | −1.5, −0.4 | −1.0, 0.1 |
| Percent | | | | | | |
| Change | | | | | | |
| from | | | | | | |
| Baseline | | | | | | |
| n | 1 | 2 | 1 | 2 | 2 | 2 |
| Mean | 0.55 | −27.26 | −20.69 | −34.81 | −16.40 | −11.58 |
| (SD) | | (27.673) | | (19.248) | (9.657) | (18.995) |
| Median | 0.55 | −27.26 | −20.69 | −34.81 | −16.40 | −11.58 |
| Q1, Q3 | 0.55, 0.55 | −46.83, −7.69 | −20.69, −20.69 | −48.42, −21.20 | −23.23, −9.57 | −25.01, 1.85 |
| Min, Max | 0.5, 0.5 | −46.8, −7.7 | −20.7, −20.7 | −48.4, −21.2 | −23.2, −9.6 | −25.0, 1.9 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine | Tadalafil | Tadalafil | All |
| | Cilnidipine + | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 10 |
| Mean | 3.26 | 5.64 | 4.96 | 4.36 | 4.57 |
| (SD) | (0.793) | (1.087) | (1.694) | (1.651) | (1.377) |
| Median | 3.03 | 5.92 | 5.62 | 4.13 | 4.13 |
| Q1, Q3 | 2.60, 4.14 | 4.87, 6.40 | 3.03, 6.22 | 3.36, 5.36 | 3.78, 5.75 |
| Min, Max | 2.6, 4.1 | 4.1, 6.6 | 3.0, 6.2 | 2.6, 6.6 | 2.6, 6.6 |
| Average | | | | | |
| Severity | | | | | |
| n | 3 | 4 | 3 | 4 | 10 |
| Mean | 2.55 | 4.15 | 3.62 | 3.35 | 3.55 |
| (SD) | (0.443) | (1.022) | (1.114) | (1.328) | (1.027) |
| Median | 2.40 | 4.31 | 3.05 | 3.06 | 3.39 |
| Q1, Q3 | 2.20, 3.05 | 3.31, 4.98 | 2.90, 4.90 | 2.30, 4.39 | 2.89, 4.56 |
| Min, Max | 2.2, 3.0 | 2.9, 5.1 | 2.9, 4.9 | 2.2, 5.1 | 2.2, 5.1 |
| Change | | | | | |
| from | | | | | |
| Baseline | | | | | |
| n | 3 | 4 | 3 | 4 | 10 |
| Mean | −0.71 | −1.49 | −1.34 | −1.02 | −1.02 |
| (SD) | (1.071) | (0.957) | (1.370) | (0.850) | (0.910) |
| Median | −0.20 | −1.42 | −1.32 | −0.96 | −1.08 |
| Q1, Q3 | −1.94, 0.02 | −2.13, −0.86 | −2.72, 0.02 | −1.73, −0.30 | −1.53, −0.20 |
| Min, Max | −1.9, 0.0 | −2.7, −0.4 | −2.7, 0.0 | −1.9, −0.2 | −2.7, 0.1 |
| Percent | | | | | |
| Change | | | | | |
| from | | | | | |
| Baseline | | | | | |
| n | 3 | 4 | 3 | 4 | 10 |
| Mean | −17.99 | −25.60 | −23.02 | −21.83 | −20.02 |
| (SD) | (25.311) | (16.357) | (24.534) | (18.046) | (17.400) |
| Median | −7.69 | −22.21 | −21.20 | −16.40 | −20.95 |
| Q1, Q3 | −46.83, 0.55 | −35.82, −15.38 | −48.42, 0.55 | −35.03, −8.63 | −25.01, −7.69 |
| Min, Max | −46.8, 0.5 | −48.4, −9.6 | −48.4, 0.5 | −46.8, −7.7 | −48.4, 1.9 |

TABLE C-7
Average Severity of Weekly RP Attacks (All Attacks) Change and Percent
Change from Baseline by Baseline RCS. ITT Population-Part A.
Average severity (0-10) of weekly RP attacks is defined as the total severity scores of RP
attacks divided by total number of RP attacks within the last 7 days of the dosing period
(excluding the last dosing day). Baseline is calculated as total severity scores of RP
attacks during the last 7 days of screening period (excluding the first dosing day) divided
by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 3.03 | 3.37 | | 5.62 | 4.12 | 3.82 |
| (SD) | | (1.087) | | | | (0.055) |
| Median | 3.03 | 3.37 | | 5.62 | 4.12 | 3.82 |
| Q1, Q3 | 3.03, 3.03 | 2.60, 4.14 | | 5.62, 5.62 | 4.12, 4.12 | 3.78, 3.86 |
| Min, Max | 3.0, 3.0 | 2.6, 4.1 | | 5.6, 5.6 | 4.1, 4.1 | 3.8, 3.9 |
| Average | | | | | | |
| Severity | | | | | | |
| n | 1 | 2 | | 1 | 1 | 2 |
| Mean | 3.05 | 2.30 | | 2.90 | 3.73 | 3.37 |
| (SD) | | (0.141) | | | | (0.676) |
| Median | 3.05 | 2.30 | | 2.90 | 3.73 | 3.37 |
| Q1, Q3 | 3.05, 3.05 | 2.20, 2.40 | | 2.90, 2.90 | 3.73, 3.73 | 2.89, 3.85 |
| Min, Max | 3.0, 3.0 | 2.2, 2.4 | | 2.9, 2.9 | 3.7, 3.7 | 2.9, 3.9 |
| Change | | | | | | |
| from | | | | | | |
| Baseline | | | | | | |
| n | 1 | 2 | | 1 | 1 | 2 |
| Mean | 0.02 | −1.07 | | −2.72 | −0.39 | −0.45 |
| (SD) | | (1.229) | | | | (0.732) |
| Median | 0.02 | −1.07 | | −2.72 | −0.39 | −0.45 |
| Q1, Q3 | 0.02, 0.02 | −1.94, −0.20 | | −2.72, −2.72 | −0.39, −0.39 | −0.97, 0.07 |
| Min, Max | 0.0, 0.0 | −1.9, −0.2 | | −2.7, −2.7 | −0.4, −0.4 | −1.0, 0.1 |
| Percent | | | | | | |
| Change | | | | | | |
| from | | | | | | |
| Baseline | | | | | | |
| n | 1 | 2 | | 1 | 1 | 2 |
| Mean | 0.55 | −27.26 | | −48.42 | −9.57 | −11.58 |
| (SD) | | (27.673) | | | | (18.995) |
| Median | 0.55 | −27.26 | | −48.42 | −9.57 | −11.58 |
| Q1, Q3 | 0.55, 0.55 | −46.83, −7.69 | | −48.42, −48.42 | −9.57, −9.57 | −25.01, 1.85 |
| Min, Max | 0.5, 0.5 | −46.8, −7.7 | | −48.4, −48.4 | −9.6, −9.6 | −25.0, 1.9 |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine + | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | 3 | 2 | 2 | 3 | 7 | |
| Mean | 3.26 | 4.87 | 4.33 | 3.62 | 3.88 | |
| (SD) | (0.793) | (1.061) | (1.832) | (0.883) | (0.960) | |
| Median | 3.03 | 4.87 | 4.33 | 4.12 | 3.86 | |
| Q1, Q3 | 2.60, 4.14 | 4.12, 5.62 | 3.03, 5.62 | 2.60, 4.14 | 3.03, 4.14 | |
| Min, Max | 2.6, 4.1 | 4.1, 5.6 | 3.0, 5.6 | 2.6, 4.1 | 2.6, 5.6 | |
| Average | | | | | | |
| Severity | | | | | | |
| n | 3 | 2 | 2 | 3 | 7 | |
| Mean | 2.55 | 3.31 | 2.97 | 2.78 | 3.00 | |
| (SD) | (0.443) | (0.585) | (0.105) | (0.830) | (0.616) | |
| Median | 2.40 | 3.31 | 2.97 | 2.40 | 2.90 | |
| Q1, Q3 | 2.20, 3.05 | 2.90, 3.73 | 2.90, 3.05 | 2.20, 3.73 | 2.40, 3.73 | |
| Min, Max | 2.2, 3.0 | 2.9, 3.7 | 2.9, 3.0 | 2.2, 3.7 | 2.2, 3.9 | |
| Change | | | | | | |
| from | | | | | | |
| Baseline | | | | | | |
| n | 3 | 2 | 2 | 3 | 7 | |
| Mean | −0.71 | −1.56 | −1.35 | −0.84 | −0.88 | |
| (SD) | (1.071) | (1.646) | (1.937) | (0.952) | (1.074) | |
| Median | −0.20 | −1.56 | −1.35 | −0.39 | −0.39 | |
| Q1, Q3 | −1.94, 0.02 | −2.72, −0.39 | −2.72, 0.02 | −1.94, −0.20 | −1.94, 0.02 | |
| Min, Max | −1.9, 0.0 | −2.7, −0.4 | −2.7, 0.0 | −1.9, −0.2 | −2.7, 0.1 | |
| Percent | | | | | | |
| Change | | | | | | |
| from | | | | | | |
| Baseline | | | | | | |
| n | 3 | 2 | 2 | 3 | 7 | |
| Mean | −17.99 | −28.99 | −23.94 | −21.36 | −19.30 | |
| (SD) | (25.311) | (27.470) | (34.624) | (22.073) | (21.249) | |
| Median | −7.69 | −28.99 | −23.94 | −9.57 | −9.57 | |
| Q1, Q3 | −46.83, 0.55 | −48.42, −9.57 | −48.42, 0.55 | −46.83, −7.69 | −46.83, 0.55 | |
| Min, Max | −46.8, 0.5 | −48.4, −9.6 | −48.4, 0.5 | −46.8, −7.7 | −48.4, 1.9 | |
| | | | | Cilnidipine | Cilnidipine | |
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 2 | 1 | 1 | |
| Mean | | | 7.49 | 6.22 | 6.59 | |
| (SD) | | | (2.457) | | | |
| Median | | | 7.49 | 6.22 | 6.59 | |
| Q1, Q3 | | | 5.75, 9.23 | 6.22, 6.22 | 6.59, 6.59 | |
| Min, Max | | | 5.8, 9.2 | 6.2, 6.2 | 6.6, 6.6 | |
| Average | | | | | | |
| Severity | | | | | | |
| n | | | 2 | 1 | 1 | |
| Mean | | | 7.02 | 4.90 | 5.06 | |
| (SD) | | | (3.479) | | | |
| Median | | | 7.02 | 4.90 | 5.06 | |
| Q1, Q3 | | | 4.56, 9.48 | 4.90, 4.90 | 5.06, 5.06 | |
| Min, Max | | | 4.6, 9.5 | 4.9, 4.9 | 5.1, 5.1 | |
| Change | | | | | | |
| from | | | | | | |
| Baseline | | | | | | |
| n | | | 2 | 1 | 1 | |
| Mean | | | −0.47 | −1.32 | −1.53 | |
| (SD) | | | (1.022) | | | |
| Median | | | −0.47 | −1.32 | −1.53 | |
| Q1, Q3 | | | −1.19, 0.25 | −1.32, −1.32 | −1.53, −1.53 | |
| Min, Max | | | −1.2, 0.3 | −1.3, −1.3 | −1.5, −1.5 | |
| Percent | | | | | | |
| Change | | | | | | |
| from | | | | | | |
| Baseline | | | | | | |
| n | | | 2 | 1 | 1 | |
| Mean | | | −8.97 | −21.20 | −23.23 | |
| (SD) | | | (16.585) | | | |
| Median | | | −8.97 | −21.20 | −23.23 | |
| Q1, Q3 | | | −20.69, 2.76 | −21.20, −21.20 | −23.23, −23.23 | |
| Min, Max | | | −20.7, 2.8 | −21.2, −21.2 | −23.2, −23.2 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 4 |
| Mean | | 6.40 | 6.22 | 6.59 | 6.95 |
| (SD) | | (0.264) | | | (1.559) |
| Median | | 6.40 | 6.22 | 6.59 | 6.40 |
| Q1, Q3 | | 6.22, 6.59 | 6.22, 6.22 | 6.59, 6.59 | 5.99, 7.91 |
| Min, Max | | 6.2, 6.6 | 6.2, 6.2 | 6.6, 6.6 | 5.8, 9.2 |
| Average | | | | | |
| Severity | | | | | |
| n | | 2 | 1 | 1 | 4 |
| Mean | | 4.98 | 4.90 | 5.06 | 6.00 |
| (SD) | | (0.113) | | | (2.330) |
| Median | | 4.98 | 4.90 | 5.06 | 4.98 |
| Q1, Q3 | | 4.90, 5.06 | 4.90, 4.90 | 5.06, 5.06 | 4.73, 7.27 |
| Min, Max | | 4.9, 5.1 | 4.9, 4.9 | 5.1, 5.1 | 4.6, 9.5 |
| Change | | | | | |
| from | | | | | |
| Baseline | | | | | |
| n | | 2 | 1 | 1 | 4 |
| Mean | | −1.42 | −1.32 | −1.53 | −0.95 |
| (SD) | | (0.150) | | | (0.813) |
| Median | | −1.42 | −1.32 | −1.53 | −1.25 |
| Q1, Q3 | | −1.53, −1.32 | −1.32, −1.32 | −1.53, −1.53 | −1.42, −0.47 |
| Min, Max | | −1.5, −1.3 | −1.3, −1.3 | −1.5, −1.5 | −1.5, 0.3 |
| Percent | | | | | |
| Change | | | | | |
| from | | | | | |
| Baseline | | | | | |
| n | | 2 | 1 | 1 | 4 |
| Mean | | −22.21 | −21.20 | −23.23 | −15.59 |
| (SD) | | (1.435) | | | (12.283) |
| Median | | −22.21 | −21.20 | −23.23 | −20.95 |
| Q1, Q3 | | −23.23, −21.20 | −21.20, −21.20 | −23.23, −23.23 | −22.21, −8.97 |
| Min, Max | | −23.2, −21.2 | −21.2, −21.2 | −23.2, −23.2 | −23.2, 2.8 |

TABLE C-8
Average Severity of Weekly RP Attacks (All Attacks) Change and Percent
Change from Baseline (Excluding Outliers) by Baseline RCS. ITT Population-Part A.
Average severity (0-10) of weekly RP attacks is defined as the total severity scores of RP
attacks divided by total number of RP attacks within the last 7 days of the dosing period
(excluding the last dosing day). Baseline is calculated as total severity scores of RP
attacks during the last 7 days of screening period (excluding the first dosing day) divided
by the total number of RP attacks.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | | 1 | 1 | 2 |
| Mean | 3.03 | 3.37 | | 5.62 | 4.12 | 3.82 |
| (SD) | | (1.087) | | | | (0.055) |
| Median | 3.03 | 3.37 | | 5.62 | 4.12 | 3.82 |
| Q1, Q3 | 3.03, 3.03 | 2.60, 4.14 | | 5.62, 5.62 | 4.12, 4.12 | 3.78, 3.86 |
| Min, Max | 3.0, 3.0 | 2.6, 4.1 | | 5.6, 5.6 | 4.1, 4.1 | 3.8, 3.9 |
| Average | | | | | | |
| Severity | | | | | | |
| n | 1 | 2 | | 1 | 1 | 2 |
| Mean | 3.05 | 2.30 | | 2.90 | 3.73 | 3.37 |
| (SD) | | (0.141) | | | | (0.676) |
| Median | 3.05 | 2.30 | | 2.90 | 3.73 | 3.37 |
| Q1, Q3 | 3.05, 3.05 | 2.20, 2.40 | | 2.90, 2.90 | 3.73, 3.73 | 2.89, 3.85 |
| Min, Max | 3.0, 3.0 | 2.2, 2.4 | | 2.9, 2.9 | 3.7, 3.7 | 2.9, 3.9 |
| Change | | | | | | |
| from | | | | | | |
| Baseline | | | | | | |
| n | 1 | 2 | | 1 | 1 | 2 |
| Mean | 0.02 | −1.07 | | −2.72 | −0.39 | −0.45 |
| (SD) | | (1.229) | | | | (0.732) |
| Median | 0.02 | −1.07 | | −2.72 | −0.39 | −0.45 |
| Q1, Q3 | 0.02, 0.02 | −1.94, −0.20 | | −2.72, −2.72 | −0.39, −0.39 | −0.97, 0.07 |
| Min, Max | 0.0, 0.0 | −1.9, −0.2 | | −2.7, −2.7 | −0.4, −0.4 | −1.0, 0.1 |
| Percent | | | | | | |
| Change | | | | | | |
| from | | | | | | |
| Baseline | | | | | | |
| n | 1 | 2 | | 1 | 1 | 2 |
| Mean | 0.55 | −27.26 | | −48.42 | −9.57 | −11.58 |
| (SD) | | (27.673) | | | | (18.995) |
| Median | 0.55 | −27.26 | | −48.42 | −9.57 | −11.58 |
| Q1, Q3 | 0.55, 0.55 | −46.83, −7.69 | | −48.42, −48.42 | −9.57, −9.57 | −25.01, 1.85 |
| Min, Max | 0.5, 0.5 | −46.8, −7.7 | | −48.4, −48.4 | −9.6, −9.6 | −25.0, 1.9 |
| | | | Pooled | Pooled | | |
| | | | Cilnidipine | Cilnidipine | | |
| | | | 10 mg | 20 mg | | |
| | | | and | and | | |
| | | | Cilnidipine | Cilnidipine | | |
| | | Pooled | 10 mg + | 20 mg + | | |
| | Pooled | Cilnidipine + | Tadalafil | Tadalafil | All | |
| | Cilnidipine | Tadalafil | 5 mg | 5 mg | Participants | |
| Statistics | (N = 3) | (N = 4) | (N = 3) | (N = 4) | (N = 11) | |
| Baseline | | | | | | |
| n | 3 | 2 | 2 | 3 | 7 | |
| Mean | 3.26 | 4.87 | 4.33 | 3.62 | 3.88 | |
| (SD) | (0.793) | (1.061) | (1.832) | (0.883) | (0.960) | |
| Median | 3.03 | 4.87 | 4.33 | 4.12 | 3.86 | |
| Q1, Q3 | 2.60, 4.14 | 4.12, 5.62 | 3.03, 5.62 | 2.60, 4.14 | 3.03, 4.14 | |
| Min, Max | 2.6, 4.1 | 4.1, 5.6 | 3.0, 5.6 | 2.6, 4.1 | 2.6, 5.6 | |
| Average | | | | | | |
| Severity | | | | | | |
| n | 3 | 2 | 2 | 3 | 7 | |
| Mean | 2.55 | 3.31 | 2.97 | 2.78 | 3.00 | |
| (SD) | (0.443) | (0.585) | (0.105) | (0.830) | (0.616) | |
| Median | 2.40 | 3.31 | 2.97 | 2.40 | 2.90 | |
| Q1, Q3 | 2.20, 3.05 | 2.90, 3.73 | 2.90, 3.05 | 2.20, 3.73 | 2.40, 3.73 | |
| Min, Max | 2.2, 3.0 | 2.9, 3.7 | 2.9, 3.0 | 2.2, 3.7 | 2.2, 3.9 | |
| Change | | | | | | |
| from | | | | | | |
| Baseline | | | | | | |
| n | 3 | 2 | 2 | 3 | 7 | |
| Mean | −0.71 | −1.56 | −1.35 | −0.84 | −0.88 | |
| (SD) | (1.071) | (1.646) | (1.937) | (0.952) | (1.074) | |
| Median | −0.20 | −1.56 | −1.35 | −0.39 | −0.39 | |
| Q1, Q3 | −1.94, 0.02 | −2.72, −0.39 | −2.72, 0.02 | −1.94, −0.20 | −1.94, 0.02 | |
| Min, Max | −1.9, 0.0 | −2.7, −0.4 | −2.7, 0.0 | −1.9, −0.2 | −2.7, 0.1 | |
| Percent | | | | | | |
| Change | | | | | | |
| from | | | | | | |
| Baseline | | | | | | |
| n | 3 | 2 | 2 | 3 | 7 | |
| Mean | −17.99 | −28.99 | −23.94 | −21.36 | −19.30 | |
| (SD) | (25.311) | (27.470) | (34.624) | (22.073) | (21.249) | |
| Median | −7.69 | −28.99 | −23.94 | −9.57 | −9.57 | |
| Q1, Q3 | −46.83, 0.55 | −48.42, −9.57 | −48.42, 0.55 | −46.83, −7.69 | −46.83, 0.55 | |
| Min, Max | −46.8, 0.5 | −48.4, −9.6 | −48.4, 0.5 | −46.8, −7.7 | −48.4, 1.9 | |
| | | | | Cilnidipine | Cilnidipine | |
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | | | 1 | 1 | 1 | |
| Mean | | | 5.75 | 6.22 | 6.59 | |
| (SD) | | | | | | |
| Median | | | 5.75 | 6.22 | 6.59 | |
| Ql, Q3 | | | 5.75, 5.75 | 6.22, 6.22 | 6.59, 6.59 | |
| Min, Max | | | 5.8, 5.8 | 6.2, 6.2 | 6.6, 6.6 | |
| Average | | | | | | |
| Severity | | | | | | |
| n | | | 1 | 1 | 1 | |
| Mean | | | 4.56 | 4.90 | 5.06 | |
| (SD) | | | | | | |
| Median | | | 4.56 | 4.90 | 5.06 | |
| Q1, Q3 | | | 4.56, 4.56 | 4.90, 4.90 | 5.06, 5.06 | |
| Min, Max | | | 4.6, 4.6 | 4.9, 4.9 | 5.1, 5.1 | |
| Change | | | | | | |
| from | | | | | | |
| Baseline | | | | | | |
| n | | | 1 | 1 | 1 | |
| Mean | | | −1.19 | −1.32 | −1.53 | |
| (SD) | | | | | | |
| Median | | | −1.19 | −1.32 | −1.53 | |
| Q1, Q3 | | | −1.19, −1.19 | −1.32, −1.32 | −1.53, −1.53 | |
| Min, Max | | | −1.2, −1.2 | −1.3, −1.3 | −1.5, −1.5 | |
| Percent | | | | | | |
| Change | | | | | | |
| from | | | | | | |
| Baseline | | | | | | |
| n | | | 1 | 1 | 1 | |
| Mean | | | −20.69 | −21.20 | −23.23 | |
| (SD) | | | | | | |
| Median | | | −20.69 | −21.20 | −23.23 | |
| Q1, Q3 | | | −20.69, −20.69 | −21.20, −21.20 | −23.23, −23.23 | |
| Min, Max | | | −20.7, −20.7 | −21.2, −21.2 | −23.2, −23.2 | |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | | 2 | 1 | 1 | 3 |
| Mean | | 6.40 | 6.22 | 6.59 | 6.19 |
| (SD) | | (0.264) | | | (0.419) |
| Median | | 6.40 | 6.22 | 6.59 | 6.22 |
| Q1, Q3 | | 6.22, 6.59 | 6.22, 6.22 | 6.59, 6.59 | 5.75, 6.59 |
| Min, Max | | 6.2, 6.6 | 6.2, 6.2 | 6.6, 6.6 | 5.8, 6.6 |
| Average | | | | | |
| Severity | | | | | |
| n | | 2 | 1 | 1 | 3 |
| Mean | | 4.98 | 4.90 | 5.06 | 4.84 |
| (SD) | | (0.113) | | | (0.254) |
| Median | | 4.98 | 4.90 | 5.06 | 4.90 |
| Q1, Q3 | | 4.90, 5.06 | 4.90, 4.90 | 5.06, 5.06 | 4.56, 5.06 |
| Min, Max | | 4.9, 5.1 | 4.9, 4.9 | 5.1, 5.1 | 4.6, 5.1 |
| Change | | | | | |
| from | | | | | |
| Baseline | | | | | |
| n | | 2 | 1 | 1 | 3 |
| Mean | | −1.42 | −1.32 | −1.53 | −1.35 |
| (SD) | | (0.150) | | | (0.172) |
| Median | | −1.42 | −1.32 | −1.53 | −1.32 |
| Q1, Q3 | | −1.53, −1.32 | −1.32, −1.32 | −1.53, −1.53 | −1.53, −1.19 |
| Min, Max | | −1.5, −1.3 | −1.3, −1.3 | −1.5, −1.5 | −1.5, −1.2 |
| Percent | | | | | |
| Change | | | | | |
| from | | | | | |
| Baseline | | | | | |
| n | | 2 | 1 | 1 | 3 |
| Mean | | −22.21 | −21.20 | −23.23 | −21.71 |
| (SD) | | (1.435) | | | (1.341) |
| Median | | −22.21 | −21.20 | −23.23 | −21.20 |
| Q1, Q3 | | −23.23, −21.20 | −21.20, −21.20 | −23.23, −23.23 | −23.23, −20.69 |
| Min, Max | | −23.2, −21.2 | −21.2, −21.2 | −23.2, −23.2 | −23.2, −20.7 |

Tables D-1 and D-2 include Raynaud's Condition Score (RCS) data.

TABLE D-1
Average Daily RCS Change and Percent Change from Baseline. ITT Population-Part A.
Average daily RCS is defined as the total RCS divided by the number of days with
available diary data during the dosing period. Baseline is calculated as the total RCS
divided by the number of days with available diary data during the screening period. If
there were multiple daily RCS scores, the latest daily RCS was used.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 2 | 2 | 2 | 2 |
| Mean | 2.74 | 3.35 | 7.72 | 4.73 | 6.01 | 3.18 |
| (SD) | | (0.778) | (2.378) | (2.660) | (3.445) | (0.089) |
| Median | 2.74 | 3.35 | 7.72 | 4.73 | 6.01 | 3.18 |
| Q1, Q3 | 2.74, 2.74 | 2.80, 3.90 | 6.04, 9.40 | 2.85, 6.61 | 3.58, 8.45 | 3.12, 3.24 |
| Min, Max | 2.7, 2.7 | 2.8, 3.9 | 6.0, 9.4 | 2.9, 6.6 | 3.6, 8.5 | 3.1, 3.2 |
| Average | | | | | | |
| Daily RCS | | | | | | |
| n | 1 | 2 | 2 | 2 | 2 | 2 |
| Mean | 1.76 | 2.90 | 7.05 | 2.77 | 4.30 | 2.71 |
| (SD) | | (1.730) | (3.672) | (2.025) | (1.112) | (0.507) |
| Median | 1.76 | 2.90 | 7.05 | 2.77 | 4.30 | 2.71 |
| Q1, Q3 | 1.76, 1.76 | 1.68, 4.13 | 4.46, 9.65 | 1.34, 4.20 | 3.52, 5.09 | 2.35, 3.07 |
| Min, Max | 1.8, 1.8 | 1.7, 4.1 | 4.5, 9.7 | 1.3, 4.2 | 3.5, 5.1 | 2.4, 3.1 |
| Change | | | | | | |
| from | | | | | | |
| Baseline | | | | | | |
| n | 1 | 2 | 2 | 2 | 2 | 2 |
| Mean | −0.98 | −0.45 | −0.67 | −1.96 | −1.71 | −0.47 |
| (SD) | | (0.953) | (1.294) | (0.635) | (2.333) | (0.418) |
| Median | −0.98 | −0.45 | −0.67 | −1.96 | −1.71 | −0.47 |
| Q1, Q3 | −0.98, −0.98 | −1.12, 0.22 | −1.58, 0.25 | −2.41, −1.51 | −3.36, −0.06 | −0.77, −0.18 |
| Min, Max | −1.0, −1.0 | −1.1, 0.2 | −1.6, 0.3 | −2.4, −1.5 | −3.4, −0.1 | −0.8, −0.2 |
| Percent | | | | | | |
| Change | | | | | | |
| from | | | | | | |
| Baseline | | | | | | |
| n | 1 | 2 | 2 | 2 | 2 | 2 |
| Mean | −35.74 | −17.16 | −11.76 | −44.79 | −20.71 | −15.03 |
| (SD) | | (32.420) | (20.390) | (11.766) | (26.931) | (13.569) |
| Median | −35.74 | −17.16 | −11.76 | −44.79 | −20.71 | −15.03 |
| Q1, Q3 | −35.74, −35.74 | −40.08, 5.77 | −26.18, 2.66 | −53.11, −36.47 | −39.75, −1.67 | −24.62, −5.43 |
| Min, Max | −35.7, −35.7 | −40.1, 5.8 | −26.2, 2.7 | −53.1, −36.5 | −39.8, −1.7 | −24.6, −5.4 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | Pooled | 10 mg | 20 mg + | All + | |
| | Pooled | Cilnidipine + | Tadalafil | Tadalafil | |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 11 |
| Mean | 3.15 | 5.37 | 4.07 | 4.68 | 4.79 |
| (SD) | (0.652) | (2.620) | (2.203) | (2.554) | (2.427) |
| Median | 2.80 | 5.09 | 2.85 | 3.74 | 3.58 |
| Q1, Q3 | 2.74, 3.90 | 3.21, 7.53 | 2.74, 6.61 | 3.19, 6.18 | 2.85, 6.61 |
| Min, Max | 2.7, 3.9 | 2.9, 8.5 | 2.7, 6.6 | 2.8, 8.5 | 2.7, 9.4 |
| Average | | | | | |
| Daily RCS | | | | | |
| n | 3 | 4 | 3 | 4 | 11 |
| Mean | 2.52 | 3.54 | 2.43 | 3.60 | 3.75 |
| (SD) | (1.389) | (1.602) | (1.545) | (1.438) | (2.324) |
| Median | 1.76 | 3.86 | 1.76 | 3.82 | 3.52 |
| Q1, Q3 | 1.68, 4.13 | 2.43, 4.65 | 1.34, 4.20 | 2.60, 4.61 | 1.76, 4.46 |
| Min, Max | 1.7, 4.1 | 1.3, 5.1 | 1.3, 4.2 | 1.7, 5.1 | 1.3, 9.7 |
| Change | | | | | |
| from | | | | | |
| Baseline | | | | | |
| n | 3 | 4 | 3 | 4 | 11 |
| Mean | −0.63 | −1.84 | −1.64 | −1.08 | −1.05 |
| (SD) | (0.740) | (1.404) | (0.723) | (1.627) | (1.133) |
| Median | −0.98 | −1.96 | −1.51 | −0.59 | −0.98 |
| Q1, Q3 | −1.12, 0.22 | −2.89, −0.79 | −2.41, −0.98 | −2.24, 0.08 | −1.58, −0.06 |
| Min, Max | −1.1, 0.2 | −3.4, −0.1 | −2.4, −1.0 | −3.4, 0.2 | −3.4, 0.3 |
| Percent | | | | | |
| Change | | | | | |
| from | | | | | |
| Baseline | | | | | |
| n | 3 | 4 | 3 | 4 | 11 |
| Mean | −23.35 | −32.75 | −41.77 | −18.93 | −23.15 |
| (SD) | (25.311) | (21.937) | (9.825) | (24.420) | (20.211) |
| Median | −35.74 | −38.11 | −36.47 | −20.71 | −26.18 |
| Q1, Q3 | −40.08, 5.77 | −46.43, −19.07 | −53.11, −35.74 | −39.92, 2.05 | −39.75, −1.67 |
| Min, Max | −40.1, 5.8 | −53.1, −1.7 | −53.1, −35.7 | −40.1, 5.8 | −53.1, 5.8 |

TABLE D-2
Average Daily RCS Change and Percent Change from Baseline (Excluding
Outliers). ITT Population-Part A.
Average daily RCS is defined as the total RCS divided by the number of days with
available diary data during the dosing period. Baseline is calculated as the total RCS
divided by the number of days with available diary data during the screening period. If
there were multiple daily RCS scores, the latest daily RCS was used.

| | | | | Cilnidipine | Cilnidipine | |
|---|---|---|---|---|---|---|
| | | | | 10 mg + | 20 mg + | |
| | Cilnidipine | Cilnidipine | Tadalafil | Tadalafil | Tadalafil | |
| | 10 mg | 20 mg | 5 mg | 5 mg | 5 mg | Placebo |
| Statistics | (N = 1) | (N = 2) | (N = 2) | (N = 2) | (N = 2) | (N = 2) |
| Baseline | | | | | | |
| n | 1 | 2 | 1 | 2 | 2 | 2 |
| Mean | 2.74 | 3.35 | 6.04 | 4.73 | 6.01 | 3.18 |
| (SD) | | (0.778) | | (2.660) | (3.445) | (0.089) |
| Median | 2.74 | 3.35 | 6.04 | 4.73 | 6.01 | 3.18 |
| Q1, Q3 | 2.74, 2.74 | 2.80, 3.90 | 6.04, 6.04 | 2.85, 6.61 | 3.58, 8.45 | 3.12, 3.24 |
| Min, Max | 2.7, 2.7 | 2.8, 3.9 | 6.0, 6.0 | 2.9, 6.6 | 3.6, 8.5 | 3.1, 3.2 |
| Average | | | | | | |
| Daily RCS | | | | | | |
| n | 1 | 2 | 1 | 2 | 2 | 2 |
| Mean | 1.76 | 2.90 | 4.46 | 2.77 | 4.30 | 2.71 |
| (SD) | | (1.730) | | (2.025) | (1.112) | (0.507) |
| Median | 1.76 | 2.90 | 4.46 | 2.77 | 4.30 | 2.71 |
| Q1, Q3 | 1.76, 1.76 | 1.68, 4.13 | 4.46, 4.46 | 1.34, 4.20 | 3.52, 5.09 | 2.35, 3.07 |
| Min, Max | 1.8, 1.8 | 1.7, 4.1 | 4.5, 4.5 | 1.3, 4.2 | 3.5, 5.1 | 2.4, 3.1 |
| Change | | | | | | |
| from | | | | | | |
| Baseline | | | | | | |
| n | 1 | 2 | 1 | 2 | 2 | 2 |
| Mean | −0.98 | −0.45 | −1.58 | −1.96 | −1.71 | −0.47 |
| (SD) | | (0.953) | | (0.635) | (2.333) | (0.418) |
| Median | −0.98 | −0.45 | −1.58 | −1.96 | −1.71 | −0.47 |
| Q1, Q3 | −0.98, −0.98 | −1.12, 0.22 | −1.58, −1.58 | −2.41, −1.51 | −3.36, −0.06 | −0.77, −0.18 |
| Min, Max | −1.0, −1.0 | −1.1, 0.2 | −1.6, −1.6 | −2.4, −1.5 | −3.4, −0.1 | −0.8, −0.2 |
| Percent | | | | | | |
| Change | | | | | | |
| from | | | | | | |
| Baseline | | | | | | |
| n | 1 | 2 | 1 | 2 | 2 | 2 |
| Mean | −35.74 | −17.16 | −26.18 | −44.79 | −20.71 | −15.03 |
| (SD) | | (32.420) | | (11.766) | (26.931) | (13.569) |
| Median | −35.74 | −17.16 | −26.18 | −44.79 | −20.71 | −15.03 |
| Q1, Q3 | −35.74, −35.74 | −40.08, 5.77 | −26.18, −26.18 | −53.11, −36.47 | −39.75, −1.67 | −24.62, −5.43 |
| Min, Max | −35.7, −35.7 | −40.1, 5.8 | −26.2, −26.2 | −53.1, −36.5 | −39.8, −1.7 | −24.6, −5.4 |

| | | | Pooled | Pooled | |
|---|---|---|---|---|---|
| | | | Cilnidipine | Cilnidipine | |
| | | | 10 mg | 20 mg | |
| | | | and | and | |
| | | | Cilnidipine | Cilnidipine | |
| | | Pooled | 10 mg + | 20 mg + | |
| | Pooled | Cilnidipine + | Tadalafil | Tadalafil | All |
| | Cilnidipine | Tadalafil | 5 mg | 5 mg | Participants |
| Statistics | (N = 3) | (N = 4) | (N = 3) | (N = 4) | (N = 11) |
| Baseline | | | | | |
| n | 3 | 4 | 3 | 4 | 10 |
| Mean | 3.15 | 5.37 | 4.07 | 4.68 | 4.33 |
| (SD) | (0.652) | (2.620) | (2.203) | (2.554) | (1.987) |
| Median | 2.80 | 5.09 | 2.85 | 3.74 | 3.41 |
| Q1, Q3 | 2.74, 3.90 | 3.21, 7.53 | 2.74, 6.61 | 3.19, 6.18 | 2.85, 6.04 |
| Min, Max | 2.7, 3.9 | 2.9, 8.5 | 2.7, 6.6 | 2.8, 8.5 | 2.7, 8.5 |
| Average | | | | | |
| Daily RCS | | | | | |
| n | 3 | 4 | 3 | 4 | 10 |
| Mean | 2.52 | 3.54 | 2.43 | 3.60 | 3.16 |
| (SD) | (1.389) | (1.602) | (1.545) | (1.438) | (1.320) |
| Median | 1.76 | 3.86 | 1.76 | 3.82 | 3.29 |
| Q1, Q3 | 1.68, 4.13 | 2.43, 4.65 | 1.34, 4.20 | 2.60, 4.61 | 1.76, 4.20 |
| Min, Max | 1.7, 4.1 | 1.3, 5.1 | 1.3, 4.2 | 1.7, 5.1 | 1.3, 5.1 |
| Change | | | | | |
| from | | | | | |
| Baseline | | | | | |
| n | 3 | 4 | 3 | 4 | 10 |
| Mean | −0.63 | −1.84 | −1.64 | −1.08 | −1.17 |
| (SD) | (0.740) | (1.404) | (0.723) | (1.627) | (1.105) |
| Median | −0.98 | −1.96 | −1.51 | −0.59 | −1.05 |
| Q1, Q3 | −1.12, 0.22 | −2.89, −0.79 | −2.41, −0.98 | −2.24, 0.08 | −1.58, −0.18 |
| Min, Max | −1.1, 0.2 | −3.4, −0.1 | −2.4, −1.0 | −3.4, 0.2 | −3.4, 0.2 |
| Percent | | | | | |
| Change | | | | | |
| from | | | | | |
| Baseline | | | | | |
| n | 3 | 4 | 3 | 4 | 10 |
| Mean | −23.35 | −32.75 | −41.77 | −18.93 | −25.73 |
| (SD) | (25.311) | (21.937) | (9.825) | (24.420) | (19.300) |
| Median | −35.74 | −38.11 | −36.47 | −20.71 | −30.96 |
| Q1, Q3 | −40.08, 5.77 | −46.43, −19.07 | −53.11, −35.74 | −39.92, 2.05 | −39.75, −5.43 |
| Min, Max | −40.1, 5.8 | −53.1, −1.7 | −53.1, −35.7 | −40.1, 5.8 | −53.1, 5.8 |

### Results and Discussion

The study treatment is well-tolerated and no adverse events or serious adverse events have been reported in any treated patients.

All cilnidipine and cilnidipine plus tadalafil treated patients (n=7) showed a decrease in the weekly frequency of Raynaud attacks in a population of both milder and more severe disease based on Raynaud Condition Scores at baseline. In 6 of 7 of these patients the average frequency of Raynaud's attacks decreased more than 25%, a clinically meaningful improvement. This improvement was not seen in patients treated with tadalafil alone, who demonstrated an increase in attack frequency.

When considering attacks reported by patients as their typical attacks, all pooled cilnidipine monotherapy or cilnidipine plus tadalafil treated patients (n=7) achieved the primary endpoint of the study producing a 25% or greater reduction in weekly attack frequency (−31 to −40%).

In patients with less severe disease at baseline, as delineated by a baseline Raynaud Condition Score of <5.0, all cilnidipine and cilnidipine plus tadalafil treated patients (n=5) demonstrated a reduction in the weekly frequency of attacks by at least 25% (range 28%−44% reduction).

In patients with more severe disease at baseline, as delineated by a Raynaud Condition Score of >5.0, (n=2) two of two cilnidipine plus tadalafil treated patients demonstrated a reduction in the weekly frequency of attacks but in only one of these patients was the reduction greater than the 25% threshold (43% reduction). Tadalafil monotherapy also increased the frequency of attacks in the one treated patient who had a baseline RCS>5.0.

When considering attacks reported by the patient as their typical Raynaud attacks, a dose response was seen (n=3) with the reduction in weekly frequency of attacks increasing to 46% from 28%, with an increase in dose from 10 mg to 20 mg of cilnidipine.

When considering attacks reported by the patient as their typical Raynaud attacks, in patients with more severe disease at baseline as determined by a baseline RCS>5.0, on average, cilnidipine plus tadalafil treated patients (cilnidipine at either 10 mg or 20 mg) had a significant reduction in the weekly frequency of attacks (−30%)

While tadalafil monotherapy at a 5 mg dose in this small sample of patients (n=2) seemed ineffective (frequency of attacks increased) adding this same dose of tadalafil to cilnidipine seemed to increase benefit with reductions in frequency, severity of attacks, duration of attacks and on RCS scores.

When considering attacks reported by the patient as their typical Raynaud attacks, pooled datasets of all cilnidipine monotherapy patients (n=3) (at either 10 or 20 mg daily) or all pooled cilnidipine plus tadalafil treated patients (n=4), or pooled cilnidipine 10 mg with or without tadalafil (n=3) or pooled cilnidipine 20 mg with or without tadalafil, all met the study primary endpoint of a reduction of at least 25% or greater in the weekly frequency of attacks (−33 to −40%)

Assessing the effect of treatment on the duration of Raynaud attacks, all cilnidipine or cilnidipine plus tadalafil treated patients (n=7) demonstrated a decrease greater than that seen in placebo treated patients. Tadalafil monotherapy patients (n=2) saw on average an increase in the duration of their reported attacks during treatment. Of the 7 cilnidipine or cilnidipine plus tadalafil treated patients, this reduction exceeded a 25% threshold in 4 of 7 patients treated. In the single placebo patient who complied with the study protocol, duration decreased only 5%. Pooled cilnidipine plus tadalafil treated patients (n=4) had a 24.3% average reduction in duration of attacks during treatment. The ability of cilnidipine to decrease duration of attacks occurred regardless of whether disease was mild or severe at baseline as determined by RCS score at baseline.

When considering the severity of attacks as reported by patients, a dose response appears to be present with 20 mg reducing severity more than 10 mg of cilnidipine as monotherapy (n=3). Cilnidipine plus tadalafil (n=4) decreased severity more than tadalafil monotherapy (n=2). Cilnidipine plus tadalafil treated patients (n=4) had a 26% reduction in severity as reported with attacks. The reduction in severity appeared to be slightly greater in patients with milder disease (RCS<5.0, (n=2), −31%) than patients with more severe disease (RCS>5.0, n=2, −22%). In patients with more severe disease at baseline (RCS>5.0, adding cilnidipine to tadalafil produced a greater reduction in severity than treating with tadalafil alone (n=2 for cilnidipine plus tadalafil, 22.2% reduction in severity, n=2 for tadalafil monotherapy, 6.3% reduction in severity.

The benefit of adding tadalafil to cilnidipine in combination appears to be seen in its effect on RCS during the study. At both 10 mg and 20 mg doses of cilnidipine, the reduction in RCS was greater in combination with tadalafil 5 mg. (n=3 cilnidipine monotherapy versus n=4 cilnidipine plus tadalafil dual therapy). 3 of 4 cilnidipine plus tadalafil treated patients achieved a clinically meaningful >25% reduction in their baseline RCS while on treatment. A result this positive has not been seen in previous drug trials in this population and the FDA considers improvements in RCS scores to be an approvable endpoint for a treatment for these patients.

The disclosures of all publications cited herein are expressly incorporated herein by reference, each in its entirety, to the same extent as if each were incorporated by reference individually.

### OTHER EMBODIMENTS

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

1. A method of treating Raynaud's syndrome in a human subject in need thereof, comprising administering to the human subject 20 mg cilnidipine or a pharmaceutically acceptable salt thereof and 5 mg tadalafil daily.

2. The method of claim 1, wherein the human subject has scleroderma and the Raynaud's syndrome is secondary Raynaud's syndrome.

3.-9. (canceled)

10. The method of claim 1, wherein the human subject also has interstitial lung disease.

11.-12. (canceled)

13. The method of claim 1, wherein the human subject has digital ulcerations.

14. The method of claim 13, wherein after administration of cilnidipine or a pharmaceutically acceptable salt thereof and the tadalafil the number and/or severity of the digital ulcerations is reduced.

15.-19. (canceled)

20. The method of claim 1, wherein the treating comprises reducing the frequency of one or more symptoms associated with Raynaud's syndrome in the human subject.

21. The method of claim 1, wherein the treating comprises reducing the severity of one or more symptoms associated with Raynaud's syndrome in the human subject.

22. The method of claim 1, wherein the treating comprises reducing the duration of one or more symptoms associated with Raynaud's syndrome in the human subject.

23.-34. (canceled)

35. A method of reducing pain or discomfort caused by a reduction of body temperature in a human subject, comprising administering to the human subject 20 mg cilnidipine or a pharmaceutically acceptable salt thereof and 5 mg tadalafil daily, wherein the reduction of body temperature in the human subject is caused by an exposure of the subject to air having a temperature of less than 25° C.

36. The method of claim 35, wherein the reduction of body temperature in the human subject is caused by an exposure of the human subject to air having a temperature of less than 10° C.

37. (canceled)

38. The method of claim 35, wherein the reduction of body temperature in the subject comprises reduction in the temperature of a hand of the human subject.

39. (canceled)

40. A method of reducing susceptibility of a human subject to cold-induced pain or discomfort, comprising administering to the human subject 20 mg cilnidipine or a pharmaceutically acceptable salt thereof and 5 mg tadalafil daily.

41. The method of claim 40, wherein the human subject has scleroderma.

42.-43. (canceled)

44. The method of claim 1, wherein vasoconstriction in the subject is reduced after administering the cilnidipine or a pharmaceutically acceptable salt thereof and the tadalafil to the human subject.

45.-64. (canceled)

65. The method of claim 1, wherein the cilnidipine or a pharmaceutically acceptable salt thereof and the tadalafil are administered in the morning.

66.-104. (canceled)

105. The method of claim 1, wherein the Raynaud's syndrome is primary Raynaud's syndrome.

106. The method of claim 1, wherein the human subject has scleroderma.

107. The method of claim 1, wherein the frequency of one or more symptoms associated with Raynaud's syndrome is reduced by at least 25%.
